(19) United States
(12) Patent Application Publication
(10) Pub. No.: US 2023/0287370 A1
(43) Pub. Date: Sep. 14, 2023

(54) NOVEL CAS ENZYMES AND METHODS OF PROFILING SPECIFICITY AND ACTIVITY

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Jonathan Leo Schmid-Burgk, Cambridge, MA (US); Linyi Gao, Cambridge, MA (US); David Li, Cambridge, MA (US)

(21) Appl. No.: 17/910,497

(22) PCT Filed: Mar. 11, 2021
(86) PCT No.: PCT/US2021/021973

§ 371 Date: Sep. 9, 2022

Related U.S. Application Data
(60) Provisional application No. 62/988,037, filed on Mar. 11, 2020.

(51) Int. Cl. C12N 9/22; C12N 15/11; C12N 15/90

(52) U.S. Cl. CPC C12N 9/22; C12N 15/11; C12N 15/907; C12N 2310/20; C12N 2310/315; C12N 2800/80

(57) ABSTRACT

A method of identifying and characterizing novel Cas protein and guide RNAs with desired activity and specificity. The disclosure further comprises compositions and systems comprising engineered Cas protein and guide RNAs with desired activity and specificity.

### CROSS-REFERENCE TO RELATED APPLICATIONS

This application claims the benefit of U.S. Provisional Application 62/988,037 filed Mar. 11, 2020. The entire contents of the above-identified application is hereby fully incorporated herein by reference.

### STATEMENT REGARDING FEDERALLY SPONSORED RESEARCH

This invention was made with government support under Grant Nos. MH110049, HL141201, and M1HG006193 awarded by the National Institutes of Health. The government has certain rights in the invention.

### TECHNICAL FIELD

The subject matter disclosed herein is generally directed to methods of identifying and characterizing Cas proteins.

### Reference to an Electronic Sequence Listing

The contents of the electronic sequence listing (“FINAL_BROD-5110WP_ST25.txt”; Size 291,887 bytes, created on Mar. 11, 2021) is herein incorporated by reference in its entirety.

### BACKGROUND

CRISPR-Cas technology is widely used for genome editing and is currently being tested in clinical trials as a therapeutic. The specificity of Cas proteins is a critical factor for application of the CRISPR-Cas technology. Although a number of techniques have been developed that assess off-target cleavage of Cas proteins, these techniques are relatively low-throughput and/or have low efficiency and accuracy. An efficient, rapid, scalable method to assess editing outcomes is needed.

### SUMMARY

In one aspect, the present disclosure provides a composition comprising an engineered Cas protein that comprises a RuvC domain and a HNH domain, wherein the engineered Cas protein has a nuclease activity substantially the same as a wildtype counterpart Cas protein and a specificity at least 30% higher than the wildtype counterpart Cas protein.

In some embodiments, the engineered Cas protein further comprises a first linker domain and a second linker domain that connects the RuvC domain and the HNH domain, and the engineered Cas protein comprises mutations in the RuvC domain, the first linker domain, and the second linker domain compared to the wildtype counterpart Cas protein. In some embodiments, the engineered Cas protein is an engineered class 2, Type II Cas protein. In some embodiments, the engineered class 2, Type II Cas protein is an engineered Cas9 protein. In some embodiments, the engineered Cas9 protein comprises one or more mutations of amino acids corresponding to the following amino acids of Streptococcus pyogenes Cas9 (SpCas9): N690, T769, G915, and N980 based on the amino acids at the sequence positions of wildtype SpCas9. In some embodiments, the engineered Cas9 protein comprises one or more mutations: N690C, T769I, G915M, N980K based on the amino acids at the sequence positions of wildtype SpCas9. In some embodiments, the engineered Cas protein is capable of generating a staggered 1 nucleotide overhang on a target polynucleotide. In some embodiments, the 1 nucleotide overhang is a 5′ overhang. In some embodiments, the engineered Cas protein has a +1 insertion frequency different from the wildtype counterpart Cas protein. In some embodiments, the +1 insertion frequency when a guanine is present in the -2 position with respect to PAM, is higher than the +1 insertion frequency when a thymidine, a cytidine, or a adenine is present in the -2 position with respect to the PAM. In some embodiments, the composition further comprises i) one or more guide sequences capable of complexing with the engineered Cas protein and directing binding of the guide-Cas protein complex to one or more target polynucleotides and ii) a donor polynucleotide.

In some embodiments, the donor polynucleotide: a. introduces one or more mutations to the target polynucleotide; b. corrects a premature stop codon in the target polynucleotide; c. disrupts a splicing site; d. restores a splicing site; e. corrects a naturally occurring 1-bp deletion; f. compensates for a naturally occurring frameshift mutation; or g. a combination thereof. In some embodiments, the one or more mutations introduced by the donor polynucleotide comprises substitutions, deletions, insertions, or a combination thereof. In some embodiments, the one or more mutations causes a shift in an open reading frame in the target polynucleotide.

In another aspect, the present disclosure provides an engineered cell comprising the composition herein.

In another aspect, the present disclosure provides a method of modifying a target polynucleotide sequence in a cell, comprising introducing the composition herein to the cell. In some embodiments, the cell is a prokaryotic cell, a eukaryotic cell, a mammalian cell, a plant cell, a cell of a non-human primate, or a human cell.

In another aspect, the present disclosure provides a method comprising: a. introducing into one or more cells: i) a Cas protein or a coding sequence thereof; ii) a plurality of guide RNAs or coding sequences thereof; and iii) a donor sequence; wherein the guide RNAs are capable of directing the Cas protein to cleave target polynucleotides in the one or more cells and the donor sequence is inserted to the cleaved target polynucleotides, thereby generating a plurality of donor-integrated target polynucleotides; b. tagmenting the donor-integrated target polynucleotides with a transposase or a transposon complex; c. sequencing the tagmented donor-integrated target polynucleotides; and d. analyzing specificity and activity of the Cas protein based on the sequences of the tagmented donor-integrated target polynucleotides.

In some embodiments, the method comprises introducing one or more polynucleotides into one or more cells, the one or more polynucleotides comprising: a coding sequence of a Cas protein; a plurality of guide RNAs or coding sequences thereof; and a donor sequence. In some embodiments, the donor sequence is a double-stranded DNA sequence. In some embodiments, the donor sequence comprises one or more modifications. In some embodiments, the one or more modifications comprises 5′ phosphorylation, phosphorothioate stabilization, or a combination thereof. In some embodiments, the tagmenting is performed using a Tn5 transposase or transposon complex.

In some embodiments, the Tn5 transposase is a hyperactive variant. In some embodiments, the method further comprises, prior to (b), lysing the one or more cells. In some embodiments, the sequencing comprises performing nested PCR. In some embodiments, (i), (ii), and (iii) are introduced using a viral vector.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1C – Method according to exemplary embodiment allows multiplexed assessment of nuclease off-targets. (1A) Schematic of exemplary Tagmentation-based Tag Integration Site Sequencing (TTISS) off-target detection method. (1B) Results from exemplary method for 59 guides from the GeCKO library tested across eight SpCas9 specificity variants and WT SpCas9. (1C) Specificity and activity scores for all tested SpCas9 variants. See also FIGS. 4A-4F, 5A-5E and Tables 3– 5.

FIGS. 2A-2E – High-throughput profiling of SpCas9 mutant fitness in human cells. (2A) Crystal structure of SpCas9 (PDB ID: 5F9R) showing the positions of 157 residues (dark gray) selected for mutagenesis. (2B) Sequences of target sites used for screening. (2C) Approach for pooled lentiviral screening of SpCas9 variants in HEK 293FT cells. (2D) Scatter plots of on-target vs. off-target activity scores for 2,420 SpCas9 single amino acid variants. The dashed box in each subplot contains all variants with ≥80% of the median wild-type on-target activity and ≤50% of the median wild-type off-target activity; activities were calculated after subtracting the median background activity of stop codon variants. The percentage within each box represents the percentage of all variants that lie within the box. (2E) On-target and off-target activity of 254 exemplary SpCas9 single amino acid variants, quantified by targeted deep sequencing of individually transfected constructs. See also FIGS. 4A-4F.

FIGS. 3A-3D – Multiplexed assessment of +1 indel frequencies using exemplary Tagmentation-based Tag Integration Site Sequencing approach (3A) Editing outcomes of nuclease-induced blunt or staggered cuts in the human genome. As a simplified exemplary model, blunt or staggered cuts can either be resected prior to re-ligation, creating random deletions (3A, top panel) or re-ligated without resection (3A, middle panel). Staggered 5′-overhangs can be filled in before re-ligation, causing duplication of base -4 respective to the PAM motif (3A, bottom panel). (3B) Schematic for convolution operation used to predict indel distributions by exemplary method. (3C) Representative examples of TTISS-predicted +1 insertion frequencies compared between specificity variants versus WT SpCas9 for 58 gRNAs. (3D) Differential +1 indel frequencies between LZ3 Cas9 and WT SpCas9 +1 insertion frequencies from targeted indel sequencing, grouped by the nucleotide identity at the -2 position relative to the PAM. Results from two-tailed t-test for significant divergence from zero are indicated by ** (p < 0.01), *** (p < 0.001), n.s. (not significant). See also FIGS. 6A-6E.

FIGS. 4A-4F – Extended validation and application of example method TTISS, related to FIGS. 1A-1C. (4A) TTISS results for multiplexing of 1, 3, 10, 30, and 60 gRNAs. The number of reads for each detected genomic locus is plotted. On-target sites are indicated as black dots (4B) Quantitative TTISS results from three cell lines using 59 guides. (4C) Detection of donor integration sites using prime editing targeting three genomic loci in HEK 293T cells. Spacer and extension sequences are provided in Table 6. (4D) Distribution of off-target sites per gRNA across 59 gRNAs detected by TTISS using WT SpCas9. (4E) Comparison of GuideScan-predicted specificity scores to TTISS measured on-target fractions for 59 guides. (4F) Comparison of Elevation specificity scores to TTISS example method embodiment measured on-target fractions for 47 guides which could be scored by the CRISPR ML online interface.

FIGS. 5A-5E – On-target and off-target activity of selected SpCas9 exemplary variants, related to FIGS. 1A-1C and 2A-2E. All indel frequencies were quantified by targeted deep sequencing. (5A) Normalized indel frequencies for 59 target sites for WT, LZ3 Cas9, and seven previously reported SpCas9 specificity-enhancing variants. Each dot represents a different guide (mean of n = 2 replicates). The horizontal gray bars/lines show the median activity for each Cas9 variant. Target sites were selected from the GeCKO library (Shalem et al. Science 2014), each targeting a different gene, without prior knowledge of activity. (5B) Activity of SpCas9 variants at additional on-target and off-target sites. Guides g5-g11 were selected based on prior knowledge of low activity for eSpCas9(1.1) and SpCas9-HF1. Shading in legend corresponds to reading the bars from left to right in all three panels. (5C) Crystal structure of SpCas9 (PDB ID: 5F9R) showing the position of the four mutations in LZ3. (5D) Activity of double mutants of selected specificity-enhancing single mutants. (5E) Epistasis plots of the variants shown in FIG. 5D for guides g1 and g2, where epistasis was calculated as fAB/(fA x fB), where fAB is the normalized indel frequency of the double mutant, and fA and fB are the normalized indel frequencies of the corresponding single mutants.

FIGS. 6A-6E – Extended assessment of +1 indel frequencies using TTISS, related to FIGS. 3A-3D. (6A) +1 insertion frequencies measured by TTISS or predicted by FORECasT, inDelphi, or Lindel are correlated to +1 frequencies measured by targeted indel sequencing for WT SpCas9 across 58 gRNAs. (6B) Predicted +1 frequencies according to example method for SpCas9 variants calculated for 58 gRNAs plotted against TTISS-predicted +1 frequencies for WT SpCas9. (6C) +1 indel frequencies measured by targeted sequencing for WT SpCas9 and LZ3 Cas9 across 59 guides, grouped by the nucleotide identity at the -4 position relative to the PAM. (6D) Plot of +1 frequencies for LZ3 against +1 frequencies for WT SpCas9 as measured by targeted sequencing for 59 gRNAs. (6E) Insertion and deletion length distributions of Cas9 variants across 59 guides from targeted sequencing. Indel length frequencies relative to total indels are shown on logarithmic scale.

FIG. 7 shows a map of the plasmid for expressing LZ3 Cas9.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F.M. Ausubel et al. eds.); the series Methods in Enzymology (Academic Press, Inc.): PCR 2: A Practical Approach (1995) (M.J. MacPherson, B.D. Hames, and G.R. Taylor eds.): Antibodies, A Laboratory Manual (1988) (Harlow and Lane, eds.): Antibodies A Laboratory Manual, 2^{nd} edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011) .

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/-10% or less, +/-5% or less, +/-1% or less, and +/-0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humor, vitreous humor, bile, blood serum, breast milk, cerebrospinal fluid, cerumen (earwax), Chile, chime, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, marines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides for methods of characterizing nuclease activity and specificity of Cas proteins and guide molecules, and methods for identifying novel CRISPR-Cas systems and Cas proteins with desired specificity and activity. The methods are high-throughput, efficient, rapid, scalable for assessing gene-editing outcomes.

In one aspect, the present disclosure provides methods for screening and characterizing nuclease specificity and activity of Cas proteins and/or guide molecules. In some cases, such methods may be used for identifying novel Cas protein or variants thereof with desired nuclease specificity and/or activity. In some embodiments, the methods comprise introducing a Cas protein (or a coding sequence thereof), a plurality of guide RNAs (or coding sequences thereof), and one or more donor sequences in one or more cells, where the Cas protein and the guide RNAs facilitate insertion of the donor sequence(s) to target polynucleotides in the cell(s); tagmenting the donor-integrated target polynucleotides; sequencing the tagmented donor-integrated target polynucleotides and analyzing the nuclease specificity and/or activity of the Cas protein based on the sequences of the tagmented donor-integrated target polynucleotides and guide RNAs.

In another aspect, the present disclosure provides engineered Cas proteins with desired nuclease specificity and activity. In some embodiments, the present disclosure provides a composition comprising an engineered Cas protein that comprises a RuvC domain and a HNH domain, wherein the engineered Cas protein has an nuclease activity is substantially the same as a wildtype counterpart Cas protein and a specificity at least 30% higher than the wildtype counterpart Cas protein. In some examples, the engineered Cas protein is a SpCas9 comprising N690C, T769I, G915M, and N980K mutations. In certain examples, the engineered Cas protein is capable of inserting a donor polynucleotide at a +1 insertion position with a frequency different from the wildtype counterpart Cas protein.

### Methods of Identifying and Characterizing Nuclease Specificity and Activity of Cas Proteins

The present disclosure provides methods for characterizing nuclease specificity and activity of Cas proteins and methods for identifying and characterizing Cas proteins with desired nuclease specificity and activity. In general, the methods comprise introducing a Cas protein, a plurality of gRNAs, and one or more donor sequences to one or more cells. In the cell(s), the Cas protein, directed by the gRNAs, may cleave one or more target polynucleotides. The donor sequences may then be integrated into the cleaved sites of the one or more target polynucleotides. The cells may be lysed and the donor sequences integrated target polynucleotides may be tagmented (e.g., by Tn5 transposase or a Tn5 transposon complex). The tagmented polynucleotides may be sequenced. The sequences may be used to determine the nuclease activity and specificity of the Cas protein. For example, the sequences may be compared to the sequences of gRNAs to determine off-target effects. The methodologies employed herein are applicable to Cas cleavage activity generating blunt or overhanging ends to improve on-target/reduce off-target specificity.

### Introducing Cas Protein, Guide RNAs, and Donor Sequences in Cells

The methods comprise introducing Cas protein(s), guide RNA(s), and donor sequences into one or more cells. In some cases, polynucleotides (e.g., on vectors) comprising the coding sequences of the Cas protein(s) and guide RNA(s) may be introduced into the cells. Introducing the proteins and nucleic acids may be performed using any methods in the delivery section described herein. In some embodiments, vectors comprising the coding sequences of Cas proteins, coding sequences of gRNAs, and donor sequences may be introduced into the cells.

Multiple Cas proteins and their nuclease specificity and activity on multiple target polynucleotides (directed by multiple guide RNAs) may be characterized. In some embodiments, a plurality of guide RNAs may be introduced at the same time. For example, at least 5, at least 10, at least 15, at least 20, at least 30, at least 40, at least 50, at least 60, at least 70, at least 80, at least 90, at least 100 guide RNAs may be introduced to the cells. A single Cas protein or multiple Cas proteins (e.g., Cas protein variants, homologs, and/or orthologs) may be introduced at the same time. In some examples, at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, at least 100, at least 200, at least 400, at least 600, at least 800, at least 1000, at least 1500, or at least 2000 Cas proteins may be introduced to the cells (e.g., at the same time). In one aspect, a multiplexed approach can enable the creation of large datasets that could aid in identification of high-specificity guides suitable for clinical applications and therapeutic/diagnostic approaches. Additionally, use of the methodologies across multiple Cas9 variant candidates facilitates identification of variants with desired activity and specificity profiles.

### Donor Polynucleotides

In certain embodiments, a donor polynucleotide or donor sequence is a polynucleotide that can be integrated into a target polynucleotide (e.g., a host cell genome). In some examples, the donor sequences may be double-stranded DNA. In certain cases, the donor sequences may comprise markers, barcodes, or other identifiers useful for further analysis of the integration.

In certain embodiments, the donor construct is a plasmid, vector, PCR product, viral genome, or synthesized polynucleotide sequence. The donor construct may be a plasmid and the plasmid may be cut to form the linear donor construct. The donor may be linearized with a restriction enzyme or a CRISPR system. The donor construct may be linearized in vitro. The donor construct plasmid may be introduced into a cell according to any method described herein (e.g., transfection) and linearized inside the cell to be tagged (e.g., CRISPR). The donor construct may be introduced by a vector. The donor construct may also be a PCR product amplified from a template DNA molecule. The donor construct may also be a synthesized polynucleotide sequence. The synthesized polynucleotide sequence can be amplified by PCR to generate the donor construct.

In certain embodiments, the donor construct may comprise a barcode sequence. The barcode sequence may be a unique molecular identifier (UMI). Nucleic acid barcode, barcode, unique molecular identifier, or UMI refer to a short sequence of nucleotides (for example, DNA or RNA) that is used as an identifier for an associated molecule, such as a target molecule and/or target nucleic acid. A nucleic acid barcode or UMI can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form.

Each donor construct may include a different UMI. The UMI can allow counting of every tagging event as each donor construct will have a different UMI. In certain embodiments, if a population of cells is tagged at a number of endogenous genes with donor constructs including a UMI it is possible to count how many times each of the genes is tagged. In certain embodiments, this information can be used to obtain more reliable protein expression data, ensuring independent tagging events in order to avoid clonal bias. In certain embodiments, the donor construct is obtained by PCR amplification of a template DNA molecule using 5′ forward primers each comprising a codon neutral UMI. Each primer can include a different codon neutral UMI, while the rest of the primer sequence is the same. In certain embodiments, the UMI of the present invention is codon-neutral. A codon neutral UMI allows for each donor construct to have a unique barcode nucleotide sequence, but express the same amino acid sequence for the integrated donor sequence. The UMI may include 3, 4, 5, 6, 7, 8, 9, 10 or more random nucleotide bases. In certain embodiments, the random bases are included in the third base of each codon (i.e., wobble base pair). An example of codon neutral UMI is incorporation of 9 codon-neutral random bases into the forward primer of the donor. Example forward primer for a neon donor (H, N and Y stand for random bases): /5phos/G*G*C GGH TCN GGN GGN AGY GGN GGN GGN TCN GTG AGC AAG GGC GAG GAG GAT AAC (SEQ ID NO: 1). In certain embodiments, software can be used that counts tagging events, while ignoring sequencing errors or uneven cellular expansion events that look like individual tagging events.

The insertion of the donor polynucleotide to a target polynucleotide may introduce one or more modifications into the target polynucleotide. For example, the donor polynucleotide may introduce one or more mutations to the target polynucleotide, corrects a premature stop codon in the target polynucleotide, disrupts a splicing site, restores a splicing site correcting a naturally occurring 1-bp deletion, compensating a naturally occurring frameshift mutation, or a combination thereof.

The donor polynucleotide may be a DNA, e.g., double-stranded DNA molecule. The donor polynucleotide may comprise one or more modifications, e.g., phosphorylation (e.g., 5′ phosphorylation or 3′ phosphorylation), methylation, phosphorothioate stabilization, or a combination thereof.

### Cells

The cells used in the methods may be prokaryotic cells or eukaryotic cells (animal cells or plant cells). In certain embodiments, the population of cells is derived from cells taken from a subject, such as a cell line. Examples of cell types and cell lines include, but are not limited to, HT115, RPE1, C8161, SCARFACE, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/ 3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T½, C6/36, Cal-27, CHO, CHO-7, CHO-IR, CHO-K1, CHO-K2, CHO-T, CHO Dhfr -/-, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN / OPCT cell lines, Peer, PNT-1A / PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassus, Va.)).

### Tagmentation

The donor-integrated target polynucleotides may be tagmented (i.e., fragmented and tagged with one or more oligonucleotides). In certain cases, the cells may be lysed and the tagmentation may be performed on nucleic acids in or from the lysed cells. In some examples, the fragmentation and tagging may be performed in the same reaction or by the same enzyme.

Tagmentation may include contacting the donor-integrated target polynucleotides with an insertional enzyme. The insertional enzyme may be any enzyme capable of inserting a nucleic acid sequence into a polynucleotide. In some examples, the DNA may be fragmented into a plurality of fragments during the insertion. In some cases, the insertional enzyme may insert the nucleic acid sequence into the polynucleotide in a substantially sequence-independent manner. The insertional enzyme may be prokaryotic or eukaryotic. Examples of insertional enzymes include transposases, HERMES, and HIV integrase.

In some cases, the insertional enzyme may be a transposase. The transposase may be an enzyme that binds to the end of a transposon and catalyzes its movement to another part of the genome by a cut and paste mechanism. The term “transposon”, as used herein, refers to a polynucleotide (or nucleic acid segment), which may be recognized by a transposase or an integrase enzyme and which is a component of a functional nucleic acid-protein complex (e.g., a transpososome, or transposon complex) capable of transposition. Transposons employ a variety of regulatory mechanisms to maintain transposition at a low frequency and sometimes coordinate transposition with various cell processes. Some prokaryotic transposons can also mobilize functions that benefit the host or otherwise help maintain the element. The term “transposase” as used herein refers to an enzyme, which is a component of a functional nucleic acid-protein complex capable of transposition and which mediates transposition. A transposon complex may comprise polynucleotide(s) of a transposon and transposase(s) for transposing the polynucleotide(s). The transposase may comprise a single protein or comprise multiple protein sub-units. A transposase may be an enzyme capable of forming a functional complex with a transposon end or transposon end sequences. The term “transposase” may also refer in certain embodiments to integrases. The expression “transposition reaction” used herein refers to a reaction wherein a transposase inserts a donor polynucleotide sequence in or adjacent to an insertion site on a target polynucleotide. The insertion site may contain a sequence or secondary structure recognized by the transposase and/or an insertion motif sequence where the transposase cuts or creates staggered breaks in the target polynucleotide into which the donor polynucleotide sequence may be inserted. Exemplary components in a transposition reaction include a transposon, comprising the donor polynucleotide sequence to be inserted, and a transposase or an integrase enzyme. The term “transposon end sequence” as used herein refers to the nucleotide sequences at the distal ends of a transposon. The transposon end sequences may be responsible for identifying the donor polynucleotide for transposition. The transposon end sequences may be the DNA sequences the transpose enzyme uses in order to form transpososome complex and to perform a transposition reaction.

Examples of transposases include a Tn transposase (e.g. Tn3, Tn5, Tn7, Tn10, Tn552, Tn903), a MuA transposase, a Vibhar transposase (e.g. from Vibrio harveyi), Ac-Ds, Ascot-1, Bs1, Cin4, Copia, En/Spm, F element, hobo, Hsmar1, Hsmar2, IN (HIV), IS1, IS2, IS3, IS4, IS5, IS6, IS10, IS21, IS30, IS50, IS51, IS150, IS256, IS407, IS427, IS630, IS903, IS911, IS982, IS1031, ISL2, L1, Mariner, P element, Tam3, Tc1, Tc3, Tel, THE-1, Tn/O, TnA, Tn3, Tn5, Tn7, Tn10, Tn552, Tn903, Tol1, Tol2, TnlO, Tyl, any prokaryotic transposase, or any transposase related to and/or derived from those listed above. In some cases, the Tn transposase may be a variant of a wildtype Tn transposase. For example, the Tn transposase may be a hyperactive variant. In certain cases, the transposase may be Tn5. In a particular example, the Tn transposase is a hyperactive Tn5 transposase. For example, the Tn5 may be the one described in Picelli, S. et al. Tn5 transposase and tagmentation procedures for massively scaled sequencing projects. Genome Res. 24, 2033-2040, doi:10.1101/gr.177881.114 (2014).

In some cases, tagmentation include contacting DNA with an insertional enzyme complex. The term “insertional enzyme complex,” as used herein, refers to a complex comprising an insertional enzyme and one or more (e.g., two) adaptor molecules (the “transposon tags”) that are combined with polynucleotides to fragment and add adaptors to the polynucleotides. Such a system is described in a variety of publications, including Caruccio (Methods Mol. Biol. 2011 733: 241-55) and US20100120098, which are incorporated by reference herein.

The tags attached to the DNA during tagmentation may be any barcode described herein. In some examples, the tags may comprise sequencing adaptors, locked nucleic acids (LNAs), zip nucleic acids (ZNAs), RNAs, affinity reactive molecules (e.g. biotin, dig), self-complementary molecules, phosphorothioate modifications, azide or alkyne groups. In some cases, the sequencing adaptors further comprise a barcode label. Further, the barcode labels may comprise a unique sequence. The unique sequences can be used to identify the individual insertion events. Any of the tags can further comprise fluorescence tags (e.g. fluorescein, rhodamine, Cy3, Cy5, thiazole orange, etc.).

The insertional enzyme may be assembled with one or more tags to be attached to the nucleic acids. One or more oligonucleotides may be assembled with the insertional enzyme. In some cases, the oligonucleotides comprise a first, a second and a third oligonucleotides. The second oligonucleotide may be phosphorylated, e.g., at the 5′ end. The phosphorylated oligonucleotide may be used for downstream ligation of cell barcodes. The third oligonucleotide may be a mosaic end compliment oligo (ME-comp). The ME-comp may be phosphorylated. Alternatively or additionally, the ME-comp may be modified to reduce extension of oligo by polymerase. For example, the ME-comp may comprise 3′ddC modification. One or more nucleotides in the ME-comp may be modified to prevent tagmentation of the oligo itself. For example, the one or more nucleotides in the ME-comp may have phosphorothioation. The first and the third, and the second and the third may be annealed before assembling with the insertional enzyme.

The insertional enzyme may further comprise an affinity tag. In some cases, the affinity tag is an antibody. The antibody may bind to, for example, a transcription factor, a modified nucleosome or a modified nucleic acid. Examples of modified nucleic acids include, but are not limited to, methylated or hydroxymethylated DNA. In other cases, the affinity tag may be a single-stranded nucleic acid (e.g. ssDNA, ssRNA). In some examples, the single-stranded nucleic acid may bind to a target nucleic acid. In further cases, the insertional enzyme may further comprise a nuclear localization signal. In some cases, the affinity tag may be one of the capture moieties or labels described herein. For example, the affinity tag may be biotin, FLAG tag, HaloTag, or V5 tag.

The insertional enzyme may be one used for Assay for Transposase Accessible Chromatin, e.g., as described in Buenrostro, J. D., Giresi, P. G., Zaba, L. C., Chang, H. Y., Greenleaf, W. J., Transposition of native chromatin for fast and sensitive epigenomic profiling of open chromatin, DNA-binding proteins and nucleosome position. Nature Methods 2013; 10 (12): 1213-1218). For example, the insertional enzyme may be a hyperactive Tn5 transposase loaded in vitro with adapters for high-throughput DNA sequencing, can simultaneously fragment and tag a genome with sequencing adapters. In one embodiment, the adapters are compatible with the methods described herein.

In some cases, the insertional enzyme may comprise two or more enzymatic moieties and the enzymatic moieties are linked together. An insert element can be bound to the insertional enzyme. The enzymatic moieties may be linked by using any suitable chemical synthesis or bioconjugation methods. For example, the enzymatic moieties may be linked via an ester/amide bond, a thiol addition into a maleimide, Native Chemical Ligation (NCL) techniques, Click Chemistry (i.e. an alkyne-azide pair), or a biotin-streptavidin pair. In some cases, each of the enzymatic moieties may insert a common sequence into the polynucleotide. The common sequence can comprise a common barcode. The enzymatic moieties may comprise transposases or derivatives thereof. In some embodiments, the polynucleotide may be fragmented into a plurality of fragments during the insertion. The fragments comprising the common barcode may be determined to be in proximity in the three-dimensional structure of the polynucleotide. The insertional enzyme may also be bound to the polynucleotide. In some cases, the polynucleotide may be further bound to a plurality of association molecules. The association molecules can be proteins (e.g. histones) or nucleic acids (e.g. aptamers).

### Tn5 Transposases

In certain embodiments, the transposase or transposon complex is a Tn5 transposase or Tn5 transposon complex. In some examples, the transposases may comprise TnpA. The transposase may be a Y1 transposase of the IS200/IS605 family, encoded by the insertion sequence (IS) IS608 from Helicobacter pylori, e.g., TnpAIS608. Examples of the transposases include those described in Barabas, O., Ronning, D.R., Guynet, C., Hickman, A.B., TonHoang, B., Chandler, M. and Dyda, F. (2008) Mechanism of IS200/ IS605 family DNA transposases: activation and transposon-directed target site selection. Cell, 132, 208-220. In certain example embodiments, the transposase is a single stranded DNA transposase. In certain example embodiments, the single stranded DNA transposase is TnpA or a functional fragment thereof.

In certain embodiments, the transposase is a single-stranded DNA transposase. The single stranded DNA transposase may be TnpA, a functional fragment thereof, or a variant thereof. In certain embodiments, the transposase is a Himar1 transposase, a fragment thereof, or a variant thereof. In certain examples, the transposase include one or more of Mu-transposase, TniQ, TniB, or functional domains thereof. In certain examples, the transposase include one or more of TniQ, a TniB, a TnpB, or functional domains thereof. In certain examples, the transposase include one or more of a rve integrase, TniQ, TniB, TnpB domain, or functional domains thereof.

In certain embodiments the system, more particularly the transposase, does not include an rve integrase, i.e., does not include an integrase of the family PFAM0065, which is part of the cl21549 superfamily; Lu, S. et al. (2020). “CDD/SPARCLE: The conserved domain database in 2020.” Nucleic Acids Research 48(D1): D265-D268. In certain embodiments the system, more particularly the transposase does not include one or more of Mu-transposase, TniQ, a TniB, a TnpB, a IstB domain or functional domains thereof. In certain embodiments, the system, more particularly the transposase does not include an rve integrase combined with one or more of a TniB, TniQ, TnpB or IstB domain.

In some embodiments, the method further comprises lysing the cell(s), e.g., before tagmentation. In some cases, the cell lysis may be performed using reagent(s) that are compatible with downstream tagmentation, e.g., without the need of purification before tagmentation. This can make the method scalable. In some examples, the cell lysis may be performed using Triton X-100 and Proteinase K.

### Sequencing

The methods herein may further comprise sequencing one or more nucleic acids processed by the steps herein. In some cases, the sequencing may be next generation sequencing. The terms “next-generation sequencing” or “high-throughput sequencing” refer to the so-called parallelized sequencing-by-synthesis or sequencing-by-ligation platforms currently employed by Illumina, Life Technologies, and Roche, etc. Next-generation sequencing methods may also include nanopore sequencing methods or electronic-detection based methods such as Ion Torrent technology commercialized by Life Technologies or single-molecule fluorescence-based method commercialized by Pacific Biosciences. Any method of sequencing known in the art can be used before and after isolation. In certain embodiments, a sequencing library is generated and sequenced.

At least a part of the processed nucleic acids and/or barcodes attached thereto may be sequenced to produce a plurality of sequence reads. The fragments may be sequenced using any convenient method. For example, the fragments may be sequenced using Illumina’s reversible terminator method, Roche’s pyrosequencing method (454), Life Technologies’ sequencing by ligation (the SOLiD platform) or Life Technologies’ Ion Torrent platform. Examples of such methods are described in the following references: Margulies et al (Nature 2005 437: 376-80); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol Biol. 2009; 553:79-108); Appleby et al (Methods Mol Biol. 2009; 513:19-39) and Morozova et al (Genomics. 2008 92:255-64), which are incorporated by reference for the general descriptions of the methods and the particular steps of the methods, including all starting products, methods for library preparation, reagents, and final products for each of the steps. As would be apparent, forward and reverse sequencing primer sites that are compatible with a selected next generation sequencing platform can be added to the ends of the fragments during the amplification step. In certain embodiments, the fragments may be amplified using PCR primers that hybridize to the tags that have been added to the fragments, where the primer used for PCR have 5′ tails that are compatible with a particular sequencing platform. In certain cases, the primers used may contain a molecular barcode (an “index”) so that different pools can be pooled together before sequencing, and the sequence reads can be traced to a particular sample using the barcode sequence.

In some cases, the sequencing may be performed at certain “depth.” The terms “depth” or “coverage” as used herein refers to the number of times a nucleotide is read during the sequencing process. In regards to single cell RNA sequencing, “depth” or “coverage” as used herein refers to the number of mapped reads per cell. Depth in regards to genome sequencing may be calculated from the length of the original genome (G), the number of reads(N), and the average read length(L) as N x L/G. For example, a hypothetical genome with 2,000 base pairs reconstructed from 8 reads with an average length of 500 nucleotides will have 2 x redundancy.

In some cases, the sequencing herein may be low-pass sequencing. The terms “low-pass sequencing” or “shallow sequencing” as used herein refers to a wide range of depths greater than or equal to 0.1 × up to 1 ×. Shallow sequencing may also refer to about 5000 reads per cell (e.g., 1,000 to 10,000 reads per cell).

In some cases, the sequencing herein may deep sequencing or ultra-deep sequencing. The term “deep sequencing” as used herein indicates that the total number of reads is many times larger than the length of the sequence under study. The term “deep” as used herein refers to a wide range of depths greater than 1 × up to 100 ×. Deep sequencing may also refer to 100 X coverage as compared to shallow sequencing (e.g., 100,000 to 1,000,000 reads per cell). The term “ultra-deep” as used herein refers to higher coverage (>100-fold), which allows for detection of sequence variants in mixed populations.

### Nested PCR

The sequencing may comprise amplifying the donor-integrated polynucleotides. The amplification may be performed by nested PCR, e.g., at least 2 rounds of nested PCR. The term “nested PCR” is understood below to mean a method in which an already duplicated DNA fragment is amplified a second time; this process is done with a second primer pair located within the primer pair used in the first reaction. Nested PCR may be polymerase chain reaction involving two or more sets of primers (three primers P1, P2 and P3 where P1+P2 is a first set and P1+P3 is a second set; or four primers P1, P2, P3 and P4 where P1+P2 is a first set and P3+P4 is a second set), used in two successive runs of or a single-pot of polymerase chain reaction, the second set being designed to amplify a secondary target within the first run product.

### Prime Editing

In some embodiments, methods may be used for characterizing donor integration in prime editing. In prime editing, the Cas protein may be associated with a reverse transcriptase. The reverse transcriptase may be fused to the C-terminus of a Cas protein. Alternatively or additionally, the reverse transcriptase may be fused to the N-terminus of a Cas protein. The fusion may be via a linker and/or an adaptor protein. In some examples, the reverse transcriptase may be an M-MLV reverse transcriptase or variant thereof. The M-MLV reverse transcriptase variant may comprise one or more mutations. For the examples, the M-MLV reverse transcriptase may comprise D200N, L603W, and T330P. In another example, the M-MLV reverse transcriptase may comprise D200N, L603W, T330P, T306K, and W313F. In a particular example, the fusion of Cas and reverse transcriptase is Cas (H840A) fused with M-MLV reverse transcriptase (D200N+L603W+T330P+T306K+W313F).

A reverse transcriptase domain may be a reverse transcriptase or a fragment thereof. A wide variety of reverse transcriptases (RT) may be used in alternative embodiments of the present invention, including prokaryotic and eukaryotic RT, provided that the RT functions within the host to generate a donor polynucleotide sequence from the RNA template. If desired, the nucleotide sequence of a native RT may be modified, for example, using known codon optimization techniques, so that expression within the desired host is optimized. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In certain embodiments, the RT domain of a reverse transcriptase is used in the present invention. The domain may include only the RNA-dependent DNA polymerase activity. In some examples, the RT domain is non-mutagenic, i.e., does not cause mutation in the donor polynucleotide (e.g., during the reverse transcriptase process). In some cases, in some examples, the RT domain may be non-retron RT, e.g., a viral RT or a human endogenous RTs. In some examples, the RT domain may be retron RT or DGRs RT. In some examples, the RT may be less mutagenic than a counterpart wildtype RT. In some embodiments, the RT herein is not mutagenic.

In some embodiments, the Cas protein may target DNA using a guide RNA containing a binding sequence that hybridizes to the target sequence on the DNA. The guide RNA may further comprise an editing sequence that contains new genetic information that replaces target DNA nucleotides.

A single-strand break (a nick) may be generated on the target DNA by the Cas protein at the target site to expose a 3′-hydroxyl group, thus priming the reverse transcription of an edit-encoding extension on the guide directly into the target site. These steps may result in a branched intermediate with two redundant single-stranded DNA flaps: a 5′ flap that contains the unedited DNA sequence, and a 3′ flap that contains the edited sequence copied from the guide RNA. The 5′ flaps may be removed by a structure-specific endonuclease, e.g., FEN122, which excises 5′ flaps generated during lagging-strand DNA synthesis and long-patch base excision repair. The non-edited DNA strand may be nicked to induce bias DNA repair to preferentially replace the non-edited strand. Examples of prime editing systems and methods include those described in Anzalone AV et al., Search-and-replace genome editing without double-strand breaks or donor DNA, Nature. 2019 Oct 21. doi: 10.1038/s41586-019-1711-4, which is incorporated by reference herein in its entirety.

### Analyzing Cas Nuclease Activity and Specificity

Analyzing Cas nuclease activity and specificity can be performed in exemplary embodiments according to methods detailed herein. The activity and specificity of a Cas protein can be consistent with those methods and approaches described in Hsu PD et al., DNA targeting specificity of RNA-guided Cas9 nucleases, Nat Biotechnol. 2013 Sep; 31(9): 827-832; and Slaymaker IM, et al., Rationally engineered Cas9 nucleases with improved specificity, Science. 2016 Jan 1; 351(6268): 84-88, which also describe examples of methods for detecting the activity and specificity of Cas proteins, and are incorporated herein by reference in their entireties.

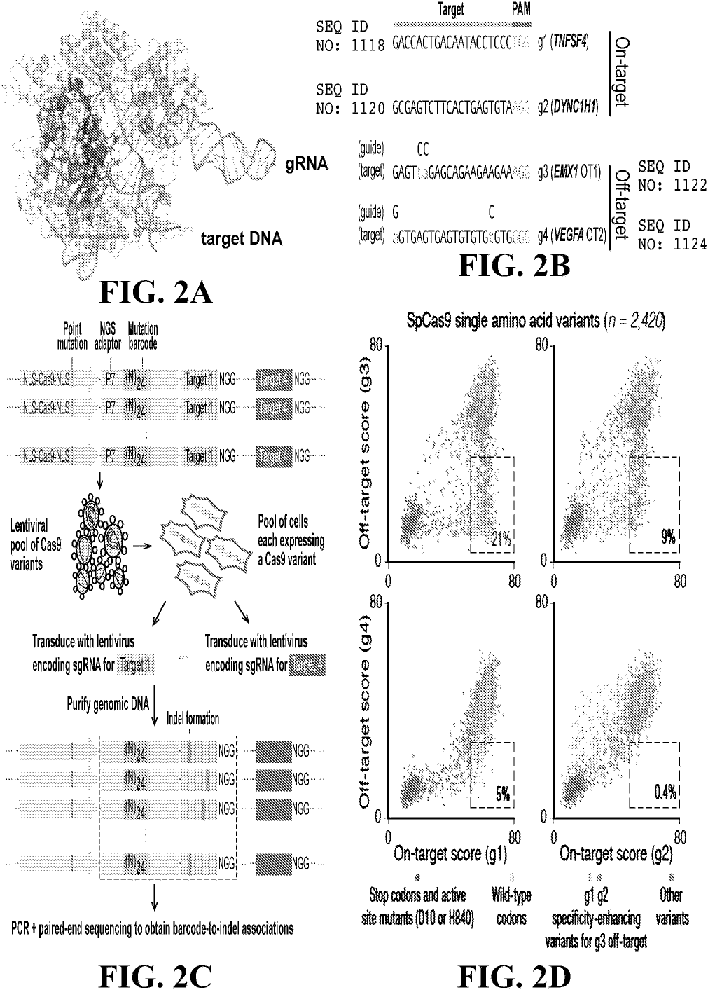

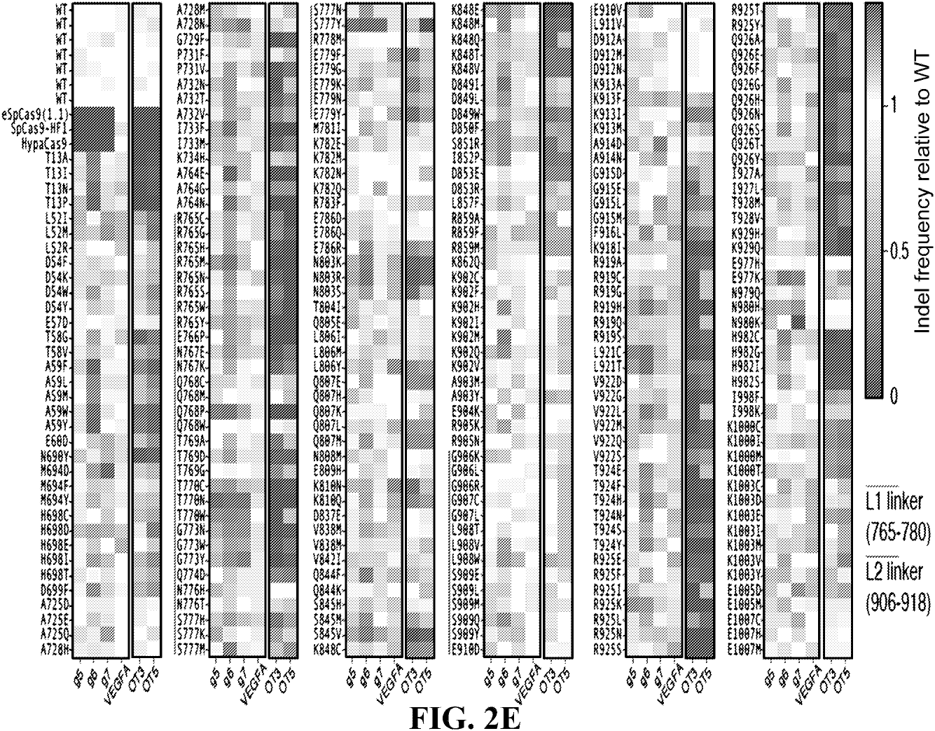

Exemplary methods for detecting Cas nuclease activity and measuring Cas target specificity can be employed for the methods detailed herein. For example, in vitro transcription and cleavage assays were employed to assess Cas9 nuclease activity and deep sequencing was used to assess Cas9 targeting specificity (Hsu et al., 2013; Slaymaker 2016). Further, as detailed herein, Applicants assessed the genome-wide editing specificity of SpCas9 using BLESS (direct in situ Breaks Labeling, Enrichment on Streptavidin and next-generation Sequencing), which quantifies DNA double-stranded breaks (DSBs) across the genome for one or more targets. In an example embodiment, assessment of specificity for at least two targets is performed for mutants, with results compared to wild-type Cas protein. In one embodiment, an established computational pipeline may be utilized for distinguishing Cas9 induced DSBs from background DSBs (see Ran FA, et al. (2015). “In vivo genome editing using Staphylococcus aureus Cas9.” Nature 520: 186-191. In an example embodiment, the exemplary method TTISS was successfully applied to detect off-targets using shCAST-mediated genome insertions for example, as described in International Patent Application No. P C T / U S 2 0 1 9 / 0 6 6 8 3 5. The methods for genome insertions described therein and the ShCAST system is hereby incorporated by reference. Briefly, the ShCAST system comprises comprising: a) one or more CRISPR-associated transposase proteins or functional fragments thereof, for example, a) TnsA, TnsB, TnsC, and TniQ, b) TnsA, TnsB, and TnsC, c) TnsB, TnsC, and TniQ, d) TnsA, TnsB, and TniQ, e) TnsE, f) TniA, TniB, and TniQ, g) TnsB, TnsC, and TnsD, h) TnsB and TnsC; i) TniA and TniB; or h) any combination thereof.; b) a Cas protein; and c) a guide molecule capable of complexing with the Cas protein and directing sequence specific binding of the guide-Cas protein complex to a target sequence of a target polynucleotide. In certain embodiments, the Cas proteins is a Type V-k protein. FIGS. 2A and 2B and Tables 26-29 of International Patent Application No. P C T / U S 2 0 1 9 / 0 6 6 8 3 5 are specifically inocorporated herein by reference for their teachings of components of the CAST system that can be used in the methods disclosed herein.

Further, it was proposed that off-target cutting occurs when the strength of Cas9 binding to the non-target DNA strand exceeds forces of DNA re-hybridization. Consistent with this model, mutations designed to weaken interactions between Cas9 and the non-complementary DNA strand led to a substantial improvement in specificity. The model also suggests that, conversely, specificity can be decreased by strengthening the interactions between Cas9 and the non-target strand, as detailed in the examples described herein.

In an example embodiment, and in accordance with working examples described herein, specificity scores were calculated by subtracting from 100 the percent of TTISS reads that corresponds to off-targets. Activity scores can be calculated as a mean indel percentage across a set of on-target sites, which may be normalized to the wild-type Cas protein utilized in the experiments. Accordingly, specificity, which may be considered to correspond to on-target activity, may be enhanced, and/or off-target activity reduced.

### Compositions and Systems

In another aspect, the present disclosure provides compositions comprising engineered Cas proteins and/or guide RNAs with desired nuclease specificity and/or activity. In some cases, the composition comprising an engineered Cas protein comprising a RuvC domain and a HNH domain, wherein the engineered Cas protein has an nuclease activity is substantially the same as a wildtype counterpart Cas protein and a specificity at least 30% higher than the wildtype counterpart Cas protein. Such engineered Cas protein may cause insertion of a donor sequence at +1 position from the cleavage site on a target polynucleotide with an insertion frequency different from a wildtype Cas protein counterpart. In some example, the Cas protein is an engineered Cas9, e.g., a mutated SpCas9. In a particular example, the engineered Cas protein is a mutated SpCas9 with N690C, T769I, G915M, and N980K.

### CRISPR-Cas System in General

The present disclosure provides a CRISPR-Cas system comprising engineered Cas proteins and/or guide RNAs with desired nuclease specificity and activity.

In general, a Cas protein (used interchangeably herein with CRISPR protein, CRISPR enzyme, CRISPR-Cas protein, CRISPR-Cas enzyme, Cas, CRISPR effector, or Cas effector protein) and/or a guide sequence is a component of a CRISPR-Cas system. ACRISPR-Cas system or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, e.g. CRISPR RNA and transactivating (tracr) RNA or a single guide RNA (aka sgRNA; chimeric RNA) or other sequences and transcripts from a CRISPR locus.

In general, a CRISPR system is characterized by elements that promote the formation of a CRISPR complex at the site of a target sequence (also referred to as a protospacer in the context of an endogenous CRISPR system). In an engineered system of the invention, the direct repeat may encompass naturally occurring sequences or non-naturally occurring sequences. The direct repeat of the invention is not limited to naturally occurring lengths and sequences. Furthermore, a direct repeat of the invention may include insertions of nucleotides such as an aptamer or sequences that bind to an adapter protein (for association with functional domains). In certain embodiments, one end of a direct repeat containing such an insertion is roughly the first half of a short DR and the end is roughly the second half of the short DR.

In the context of formation of a CRISPR complex, “target sequence” or “target polynucleotides” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell.

In general, a guide sequence (or spacer sequence) may be any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance a double mismatch is, the more cleavage efficiency is affected. Accordingly, by choosing mismatch position along the spacer, cleavage efficiency can be modulated. By means of example, if less than 100 % cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

A CRISPR-Cas system or components thereof may be used for introducing one or more mutations in a target locus or nucleic acid sequence. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s).

Typically, in the context of an endogenous CRISPR-Cas system, formation of a CRISPR complex (comprising a guide sequence hybridized to a target sequence and complexed with one or more Cas proteins) results in cleavage in or near (e.g. within 1, 2, 3, 4, 5, 6, 7, 8, 9, 10, 20, 50, or more base pairs from) the target sequence, but may depend on for instance secondary structure, in particular in the case of RNA targets. In some cases, in the context of an endogenous CRISPR system, formation of a CRISPR complex (comprising a guide sequence hybridized to a target sequence and complexed with one or more Cas proteins) results in cleavage of one or both strands (if applicable) in or near (e.g. within 1, 2, 3, 4, 5, 6, 7, 8, 9, 10, 20, 50, or more base pairs from) the target sequence.

In particularly preferred embodiments according to the invention, the guide RNA (capable of guiding Cas to a target locus) may comprise (1) a guide sequence capable of hybridizing to a target locus (a polynucleotide target locus, such as an RNA target locus) in the eukaryotic cell; (2) a direct repeat (DR) sequence) which reside in a single RNA, i.e. an sgRNA (arranged in a 5′ to 3′ orientation) or crRNA.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pats. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; U.S. Pat. Publications US 2014-0310830 (U.S. APP. Ser. No. 14/105,031), US 2014-0287938 A1 (U.S. App. Ser. No. 14/213,991), US 2014-0273234 A1 (U.S. App. Ser. No. 14/293,674), US2014-0273232 A1 (U.S. App. Ser. No. 14/290,575), US 2014-0273231 (U.S. App. Ser. No. 14/259,420), US 2014-0256046 A1 (U.S. App. Ser. No. 14/226,274), US 2014-0248702 A1 (U.S. App. Ser. No. 14/258,458), US 2014-0242700 A1 (U.S. App. Ser. No. 14/222,930), US 2014-0242699 A1 (U.S. App. Ser. No. 14/183,512), US 2014-0242664 A1 (U.S. App. Ser. No. 14/104,990), US 2014-0234972 A1 (U.S. App. Ser. No. 14/183,471), US 2014-0227787 A1 (U.S. App. Ser. No. 14/256,912), US 2014-0189896 A1 (U.S. App. Ser. No. 14/105,035), US 2014-0186958 (U.S. App. Ser. No. 14/105,017), US 2014-0186919 A1 (U.S. App. Ser. No. 14/104,977), US 2014-0186843 A1 (U.S. App. Ser. No. 14/104,900), US 2014-0179770 A1 (U.S. App. Ser. No. 14/104,837) and US 2014-0179006 A1 (U.S. App. Ser. No. 14/183,486), US 2014-0170753 (US App Ser No 14/183,429); European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809). Reference is also made to U.S. Provisional Pat. Applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. Provisional Pat. Application 61/836,123, filed on Jun. 17, 2013. Reference is additionally made to US provisional patent applications 61/835,931, 61/835,936, 61/836,127, 61/836,101, 61/836,080 and 61/835,973, each filed Jun. 17, 2013. Further reference is made to U.S. Provisional Pat. Applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent Applications Nos: PCT/US2014/041803, PCT/US2014/041800, PCT/US2014/041809, PCT/US2014/041804 and PCT/US2014/041806, each filed Jun. 10, 2014 6/10/14; PCT/US2014/041808 filed Jun. 11, 2014; and PCT/US2014/62558 filed Oct. 28, 2014, and U.S. Provisional Pat. Applications Serial Nos.: 61/915,150, 61/915,301, 61/915,267 and 61/915,260, each filed Dec. 12, 2013; 61/757,972and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. Provisional Pat. Applications Nos. 62/055,484, 62/055,460, and 62/055,487, filed Sep. 25, 2014; U.S. Provisional Pat. Application 61/980,012, filed Apr. 15, 2014; and U.S. Provisional Pat. Application 61/939,242 filed Feb. 12, 2014. Reference is made to PCT application designating, inter alia, the United States, application No. PCT/US14/41806, filed Jun. 10, 2014. Reference is made to U.S. Provisional Pat. Application 61/930,214 filed on Jan. 22, 2014. Reference is made to U.S. Provisional Pat. Applications 61/915,251; 61/915,260 and 61/915,267, each filed on Dec. 12, 2013. Reference is made to U.S. Provisional Pat. Application USSN 61/980,012 filed Apr. 15, 2014. Reference is made to PCT application designating, inter alia, the United States, Application No. PCT/US14/41806, filed Jun. 10, 2014. Reference is made to U.S. Provisional Pat. Application 61/930,214 filed on Jan. 22, 2014. Reference is made to U.S. Provisional Pat. Applications 61/915,251; 61/915,260 and 61/915,267, each filed on Dec. 12, 2013.

Mention is also made of U.S. Application 62/091,455, filed, 12-Dec-14 PROTECTED GUIDE RNAS (PGRNAS); U.S. Application 62/096,708, 24-Dec-14, PROTECTED GUIDE RNAS (PGRNAS); U.S. Application 62/091,462, 12-Dec-14, DEAD GUIDES FOR CRISPR TRANSCRIPTION FACTORS; U.S. Application 62/096,324, 23-Dec- 14, DEAD GUIDES FOR CRISPR TRANSCRIPTION FACTORS; U.S. Application 62/091,456, 12-Dec-14, ESCORTED AND FUNCTIONALIZED GUIDES FOR CRISPR- CAS SYSTEMS; U.S. Application 62/091,461, 12-Dec-14, DELIVERY, USE AND THERAPEUTIC APPLICATIONS OF THE CRISPR-CAS SYSTEMS AND COMPOSITIONS FOR GENOME EDITING AS TO HEMATOPOIETIC STEM CELLS (HSCs); U.S. Application 62/094,903, 19-Dec-14, UNBIASED IDENTIFICATION OF DOUBLE-STRAND BREAKS AND GENOMIC REARRANGEMENT BY GENOME- WISE INSERT CAPTURE SEQUENCING; U.S. Application 62/096,761, 24-Dec-14, ENGINEERING OF SYSTEMS, METHODS AND OPTIMIZED ENZYME AND GUIDE SCAFFOLDS FOR SEQUENCE MANIPULATION; U.S. Application 62/098,059, 30-Dec-14, RNA-TARGETING SYSTEM; US application 62/096,656, 24-Dec-14, CRISPR HAVING OR ASSOCIATED WITH DESTABILIZATION DOMAINS; U.S. Application 62/096,697, 24-Dec-14, CRISPR HAVING OR ASSOCIATED WITH AAV; U.S. Application 62/098,158, 30-Dec-14, ENGINEERED CRISPR COMPLEX INSERTIONAL TARGETING SYSTEMS; U.S. Application 62/151,052, 22-Apr-15, CELLULAR TARGETING FOR EXTRACELLULAR EXOSOMAL REPORTING; U.S. Application 62/054,490, 24-Sep-14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. Application 62/055,484, 25-Sep-14, SYSTEMS, METHODS AND COMPOSITIONS FOR SEQUENCE MANIPULATION WITH OPTIMIZED FUNCTIONAL CRISPR-CAS SYSTEMS; U.S. Application 62/087,537, 4-Dec-14, SYSTEMS, METHODS AND COMPOSITIONS FOR SEQUENCE MANIPULATION WITH OPTIMIZED FUNCTIONAL CRISPR-CAS SYSTEMS; U.S. Application 62/054,651, 24-Sep-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. Application 62/067,886, 23-Oct-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. Application 62/054,675, 24-Sep-14, DELIVERY, USE AND THERAPEUTIC APPLICATIONS OF THE CRISPR-CAS SYSTEMS AND COMPOSITIONS IN NEURONAL CELLS/TISSUES; U.S. Application 62/054,528, 24-Sep-14, DELIVERY, USE AND THERAPEUTIC APPLICATIONS OF THE CRISPR-CAS SYSTEMS AND COMPOSITIONS IN IMMUNE DISEASES OR DISORDERS; U.S. Application 62/055,454, 25-Sep-14, DELIVERY, USE AND THERAPEUTIC APPLICATIONS OF THE CRISPR- CAS SYSTEMS AND COMPOSITIONS FOR TARGETING DISORDERS AND DISEASES USING CELL PENETRATION PEPTIDES (CPP); U.S. Application 62/055,460, 25-Sep-14, MULTIFUNCTIONAL-CRISPR COMPLEXES AND/OR OPTIMIZED ENZYME LINKED FUNCTIONAL-CRISPR COMPLEXES; U.S. Application 62/087,475, 4- Dec-14, FUNCTIONAL SCREENING WITH OPTIMIZED FUNCTIONAL CRISPR-CAS SYSTEMS; US application 62/055,487, 25-Sep-14, FUNCTIONAL SCREENING WITH OPTIMIZED FUNCTIONAL CRISPR-CAS SYSTEMS; U.S. Application 62/087,546, 4-Dec- 14, MULTIFUNCTIONAL CRISPR COMPLEXES AND/OR OPTIMIZED ENZYME LINKED FUNCTIONAL-CRISPR COMPLEXES; and U.S. Application 62/098,285, 30-Dec- 14, CRISPR MEDIATED IN VIVO MODELING AND GENETIC SCREENING OF TUMOR GROWTH AND METASTASIS.

Also, with respect to general information on CRISPR-Cas Systems, mention is made of the following (also hereby incorporated herein by reference):
Multiplex genome engineering using CRISPR/Cas systems. Cong, L., Ran, F.A., Cox, D., Lin, S., Barretto, R., Habib, N., Hsu, P.D., Wu, X., Jiang, W., Marraffini, L.A., & Zhang, F. Science Feb 15;339(6121):819-23 (2013);
RNA-guided editing of bacterial genomes using CRISPR-Cas systems. Jiang W., Bikard D., Cox D., Zhang F, Marraffini LA. Nat Biotechnol Mar;31(3):233-9 (2013);
One-Step Generation of Mice Carrying Mutations in Multiple Genes by CRISPR/Cas-Mediated Genome Engineering. Wang H., Yang H., Shivalila CS., Dawlaty MM., Cheng AW., Zhang F., Jaenisch R. Cell May 9;153(4):910-8 (2013);
Optical control of mammalian endogenous transcription and epigenetic states. Konermann S, Brigham MD, Trevino AE, Hsu PD, Heidenreich M, Cong L, Platt RJ, Scott DA, Church GM, Zhang F. Nature. Aug 22;500(7463):472-6. doi: 10.1038/Nature12466. Epub 2013 Aug 23 (2013);
Double Nicking by RNA-Guided CRISPR Cas9 for Enhanced Genome Editing Specificity. Ran, FA., Hsu, PD., Lin, CY., Gootenberg, JS., Konermann, S., Trevino, AE., Scott, DA., Inoue, A., Matoba, S., Zhang, Y., & Zhang, F. Cell Aug 28. pii: S0092-8674(13)01015-5 (2013-A);
DNA targeting specificity of RNA-guided Cas9 nucleases. Hsu, P., Scott, D., Weinstein, J., Ran, FA., Konermann, S., Agarwala, V., Li, Y., Fine, E., Wu, X., Shalem, O., Cradick, TJ., Marraffini, LA., Bao, G., & Zhang, F. Nat Biotechnol doi:10.1038/nbt.2647 (2013);
Genome engineering using the CRISPR-Cas9 system. Ran, FA., Hsu, PD., Wright, J., Agarwala, V., Scott, DA., Zhang, F. Nature Protocols Nov;8(11):2281-308 (2013-B);
Genome-Scale CRISPR-Cas9 Knockout Screening in Human Cells. Shalem, O., Sanjana, NE., Hartenian, E., Shi, X., Scott, DA., Mikkelson, T., Heckl, D., Ebert, BL., Root, DE., Doench, JG., Zhang, F. Science Dec 12. (2013). [Epub ahead of print];
Crystal structure of cas9 in complex with guide RNA and target DNA. Nishimasu, H., Ran, FA., Hsu, PD., Konermann, S., Shehata, SI., Dohmae, N., Ishitani, R., Zhang, F., Nureki, O. Cell Feb 27, 156(5):935-49 (2014);
Genome-wide binding of the CRISPR endonuclease Cas9 in mammalian cells. Wu X., Scott DA., Kriz AJ., Chiu AC., Hsu PD., Dadon DB., Cheng AW., Trevino AE., Konermann S., Chen S., Jaenisch R., Zhang F., Sharp PA. Nat Biotechnol. Apr 20. doi: 10.1038/nbt.2889 (2014);
CRISPR-Cas9 Knockin Mice for Genome Editing and Cancer Modeling. Platt RJ, Chen S, Zhou Y, Yim MJ, Swiech L, Kempton HR, Dahlman JE, Parnas O, Eisenhaure TM, Jovanovic M, Graham DB, Jhunjhunwala S, Heidenreich M, Xavier RJ, Langer R, Anderson DG, Hacohen N, Regev A, Feng G, Sharp PA, Zhang F. Cell 159(2): 440-455 DOI: 10.1016/j.cell.2014.09.014(2014);
Development and Applications of CRISPR-Cas9 for Genome Engineering, Hsu PD, Lander ES, Zhang F., Cell. Jun 5;157(6):1262-78 (2014).
Genetic screens in human cells using the CRISPR/Cas9 system, Wang T, Wei JJ, Sabatini DM, Lander ES., Science. January 3; 343(6166): 80-84. doi:10.1126/science.1246981 (2014);
Rational design of highly active sgRNAs for CRISPR-Cas9-mediated gene inactivation, Doench JG, Hartenian E, Graham DB, Tothova Z, Hegde M, Smith I, Sullender M, Ebert BL, Xavier RJ, Root DE., (published online 3 Sep. 2014) Nat Biotechnol. Dec;32(12): 1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. Jan;33(1):102-6 (2015);
Genome-scale transcriptional activation by an engineered CRISPR-Cas9 complex, Konermann S, Brigham MD, Trevino AE, Joung J, Abudayyeh OO, Barcena C, Hsu PD, Habib N, Gootenberg JS, Nishimasu H, Nureki O, Zhang F., Nature. Jan 29;517(7536):583-8 (2015).
A split-Cas9 architecture for inducible genome editing and transcription modulation, Zetsche B, Volz SE, Zhang F., (published online 02 Feb. 2015) Nat Biotechnol. Feb;33(2):139-42 (2015);
Genome-wide CRISPR Screen in a Mouse Model of Tumor Growth and Metastasis, Chen S, Sanjana NE, Zheng K, Shalem O, Lee K, Shi X, Scott DA, Song J, Pan JQ, Weissleder R, Lee H, Zhang F, Sharp PA. Cell 160, 1246-1260, Mar. 12, 2015 (multiplex screen in mouse), and
In vivo genome editing using Staphylococcus aureus Cas9, Ran FA, Cong L, Yan WX, Scott DA, Gootenberg JS, Kriz AJ, Zetsche B, Shalem O, Wu X, Makarova KS, Koonin EV, Sharp PA, Zhang F., (published online 01 Apr. 2015), Nature. Apr 9;520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR/Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
Zetsche et al. (2015), “Cpf1 is a single RNA-guided endonuclease of a class 2 CRISPR- Cas system,” Cell 163, 759-771 (Oct. 22, 2015) doi: 10.1016/j.cell.2015.09.038. Epub Sep. 25, 2015
Shmakov et al. (2015), “Discovery and Functional Characterization of Diverse Class 2 CRISPR-Cas Systems,” Molecular Cell 60, 385-397 (Nov. 5, 2015) doi: 10.1016/j.molcel.2015.10.008. Epub Oct. 22, 2015
Dahlman et al., “Orthogonal gene control with a catalytically active Cas9 nuclease,” Nature Biotechnology 33, 1159-1161 (November, 2015)
Gao et al, “Engineered Cpf1 Enzymes with Altered PAM Specificities,” bioRxiv 091611; doi: dx.doi.org/10.1101/091611 Epub Dec. 4, 2016
Smargon et al. (2017), “Cas13b Is a Type VI-B CRISPR-Associated RNA-Guided RNase Differentially Regulated by Accessory Proteins Csx27 and Csx28,” Molecular Cell 65, 618-630 (Feb. 16, 2017) doi: 10.1016/j.molcel.2016.12.023. Epub Jan. 5, 2017 each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors mentioned that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells. In addition, mention is made of PCT application PCT/US14/70057, Attorney Reference 47627.99.2060 and BI-2013/107 entitled “DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS (claiming priority from one or more or all of U.S. Provisional patent applications: 62/054,490, filed Sep. 24, 2014; 62/010,441, filed Jun. 10, 2014; and 61/915,118, 61/915,215 and 61/915,148, each filed on Dec. 12, 2013) (“the Particle Delivery PCT”), incorporated herein by reference, with respect to a method of preparing an sgRNA-and-Cas9 protein containing particle comprising admixing a mixture comprising an sgRNA and Cas protein (and optionally HDR template) with a mixture comprising or consisting essentially of or consisting of surfactant, phospholipid, biodegradable polymer, lipoprotein and alcohol; and particles from such a process. For example, wherein Cas protein and sgRNA were mixed together at a suitable, e.g., 3:1 to 1:3 or 2:1 to 1:2 or 1:1 molar ratio, at a suitable temperature, e.g., 15-30C, e.g., 20-25C, e.g., room temperature, for a suitable time, e.g., 15-45, such as 30 minutes, advantageously in sterile, nuclease free buffer, e.g., 1X PBS. Separately, particle components such as or comprising: a surfactant, e.g., cationic lipid, e.g., 1,2-dioleoyl-3-trimethylammonium-propane (DOTAP); phospholipid, e.g., dimyristoylphosphatidylcholine (DMPC); biodegradable polymer, such as an ethylene-glycol polymer or PEG, and a lipoprotein, such as a low-density lipoprotein, e.g., cholesterol were dissolved in an alcohol, advantageously a C₁₋₆ alkyl alcohol, such as methanol, ethanol, isopropanol, e.g., 100% ethanol. The two solutions were mixed together to form particles containing the Cas-sgRNA complexes. Accordingly, sgRNA may be pre-complexed with the Cas protein, before formulating the entire complex in a particle. Formulations may be made with a different molar ratio of different components known to promote delivery of nucleic acids into cells (e.g. 1,2-dioleoyl-3-trimethylammonium-propane (DOTAP), 1,2-ditetradecanoyl-sn-glycero-3-phosphocholine (DMPC), polyethylene glycol (PEG), and cholesterol) For example DOTAP : DMPC : PEG: Cholesterol Molar Ratios may be DOTAP 100, DMPC 0, PEG 0, Cholesterol 0; or DOTAP 90, DMPC 0, PEG 10, Cholesterol 0; or DOTAP 90, DMPC 0, PEG 5, Cholesterol 5. DOTAP 100, DMPC 0, PEG 0, Cholesterol 0. That application accordingly comprehends admixing sgRNA, Cas protein and components that form a particle; as well as particles from such admixing. Aspects of the instant invention can involve particles; for example, particles using a process analogous to that of the Particle Delivery PCT, e.g., by admixing a mixture comprising crRNA and/or CRISPR-Cas as in the instant invention and components that form a particle, e.g., as in the Particle Delivery PCT, to form a particle and particles from such admixing (or, of course, other particles involving crRNA and/or CRISPR-Cas as in the instant invention).

### Cas Proteins

The Cas protein (e.g., engineered Cas protein) may have a nuclease activity that is substantially the same (e.g., between 80% and 100%, between 90% and 100%, between 95% and 100%, between 98% and 100%, between 99% and 100%, between 99.9% and 100%, or about 100%) as a wildtype counterpart Cas protein. In certain cases, the engineered Cas protein has a nuclease activity that is higher than (e.g., at least 5%, at least 10%, at least 20%, at least 30%, at least 40%, at least 50%, at least 60%, at least 70%, at least 80%, or at least 90% higher than) a wildtype counterpart Cas protein.

Alternatively or additionally, the Cas protein (e.g., engineered Cas protein) may have a specificity at least 5%, at least 10%, at least 20%, at least 30%, at least 40%, at least 50%, at least 60%, at least 70%, at least 80%, or at least 90% higher than the wildtype counterpart Cas protein. In a particular example, the Cas protein (e.g., engineered Cas protein) may have a specificity at least 30% higher than the wildtype counterpart Cas protein. As used herein, the term “specificity” of a Cas may correspond to the number or percentage of on-target polynucleotide cleavage events relative to the number or percentage of all polynucleotide cleavage events, including on-target and off-target events. The activity and specificity of a Cas protein are consistent with those described in Hsu PD et al., DNA targeting specificity of RNA-guided Cas9 nucleases, Nat Biotechnol. 2013 Sep; 31(9): 827-832; and Slaymaker IM, et al., Rationally engineered Cas9 nucleases with improved specificity, Science. 2016 Jan 1; 351(6268): 84-88, which also describe examples of methods for detecting the activity and specificity of Cas proteins, and are incorporated herein by reference in their entireties, and are detailed elsewhere herein.

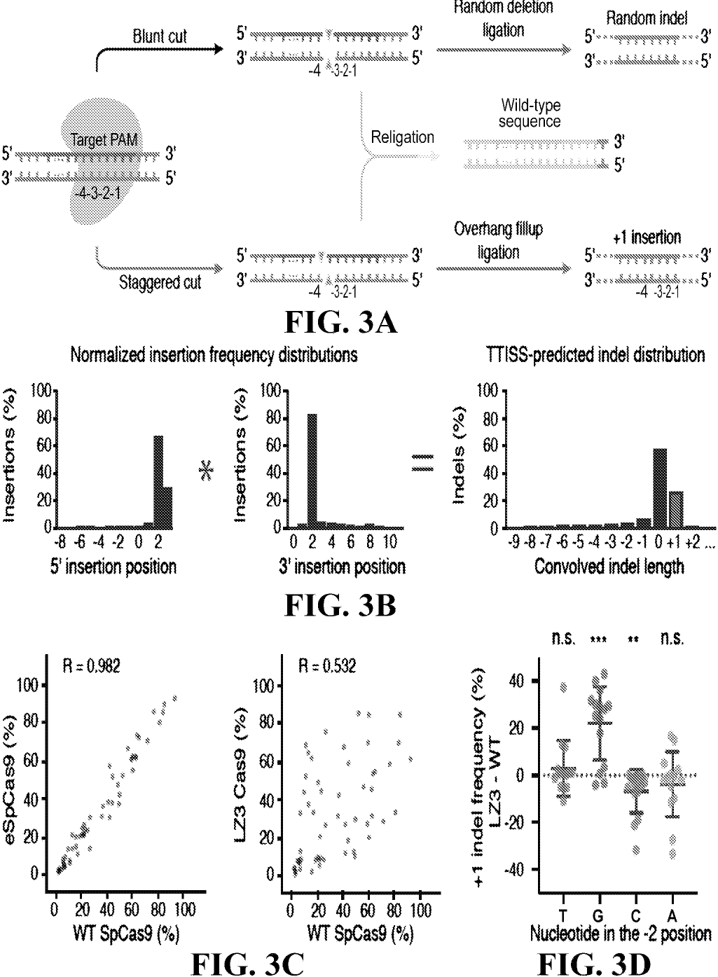

In some embodiments, the Cas protein (e.g., its RuvC domain) may slide one base upstream (with respective to the PAM), and produce a staggered cut, which may be filled and lead to duplication of a single base (i.e., +1 insertion). An example of a +1 insertion position is shown in FIG. 3A and described in Zuo, Z., and Liu, J. (2016). Cas9-catalyzed DNA Cleavage Generates Staggered Ends: Evidence from Molecular Dynamics Simulations. Scientific Reports 6, 37584. In some embodiments, the engineered Cas protein has a +1 insertion frequency different from the wildtype counterpart Cas protein. For example, the +1 insertion frequency when a guanine is present in the -2 position with respect a PAM is higher than the +1 insertion frequency when a thymidine, a cytidine, or a adenine is present in the -2 position with respect the PAM. In some cases, the +1 insertions depend on host machinery in human cells. In some examples, the Cas protein may generate a staggered cut. The staggered cut may be a 1-bp or 1- nucleotide 5′ overhang. The staggered cut may be a 1-bp or 1-nucleotide 3′ overhang.

The nucleic acid molecule encoding a Cas may be codon optimized. An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a Cas is codon optimized for expression in particular cells, such as eukaryotic cells. The eukaryotic cells may be those of or derived from a particular organism, such as a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In some embodiments, the Cas proteins may have nucleic acid cleavage activity. The Cas proteins may have RNA binding and DNA cleaving function. In some embodiments, Cas may direct cleavage of one or two nucleic acid strands at the location of or near a target sequence, such as within the target sequence and/or within the complement of the target sequence or at sequences associated with the target sequence, e.g., within about 1, 2, 3, 4, 5, 6, 7, 8, 9, 10, 15, 20, 25, 50, 100, 200, 500, or more base pairs from the first or last nucleotide of a target sequence. In some embodiments, the Cas protein may direct more than one cleavage (such as one, two three, four, five, or more cleavages) of one or two strands within the target sequence and/or within the complement of the target sequence or at sequences associated with the target sequence and/or within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt, i.e., generating blunt ends. In some embodiments, the cleavage may be staggered, i.e., generating sticky ends. Advantageously, the methods and systems detailed herein can be utilized with both staggered and blunt end cleavage applications. In some embodiments, a vector encodes a nucleic acid-targeting Cas protein that may be mutated with respect to a corresponding wild-type enzyme such that the mutated nucleic acid-targeting Cas protein lacks the ability to cleave one or two strands of a target polynucleotide containing a target sequence, e.g., alteration or mutation in a HNH domain to produce a mutated Cas substantially lacking all DNA cleavage activity, e.g., the DNA cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. By derived, Applicants mean that the derived enzyme is largely based, in the sense of having a high degree of sequence homology with, a wildtype enzyme, but that it has been mutated (modified) in some way as known in the art or as described herein.

Typically, in the context of an endogenous nucleic acid-targeting system, formation of a nucleic acid-targeting complex (comprising a guide RNA or crRNA hybridized to a target sequence and complexed with one or more nucleic acid-targeting effector proteins) results in cleavage of DNA strand(s) in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest).

It will be appreciated that the effector protein is based on or derived from an enzyme, so the term ‘effector protein’ certainly includes ‘enzyme’ in some embodiments. However, it will also be appreciated that the effector protein may, as required in some embodiments, have DNA or RNA binding, but not necessarily cutting or nicking, activity, including a dead-Cas protein function.

In some embodiments, a Cas protein may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the CRISPR effector protein may be a part of a Light Inducible Transcriptional Effector (LITE) to direct changes in transcriptional activity in a sequence-specific manner. The components of a light may include a CRISPR effector protein, a light-responsive cytochrome heterodimer (e.g. from Arabidopsis thaliana), and a transcriptional activation/repression domain. Further examples of inducible DNA binding proteins and methods for their use are provided in US 61/736465 and US 61/721,283, and WO 2014018423 A2 which is hereby incorporated by reference in its entirety.

In one aspect, the invention provides a mutated Cas as described herein elsewhere, having one or more mutations resulting in reduced off-target effects, e.g., improved CRISPR enzymes for use in effecting modifications to target loci but which reduce or eliminate activity towards off-targets, such as when complexed to guide RNAs, as well as improved CRISPR enzymes for increasing the activity of CRISPR enzymes, such as when complexed with guide RNAs. It is to be understood that mutated enzymes as described herein below may be used in any of the methods according to the invention as described herein elsewhere. Any of the methods, products, compositions and uses as described herein elsewhere are equally applicable with the mutated CRISPR enzymes as further detailed below.

The methods and mutations which can be employed in various combinations to increase or decrease activity and/or specificity of on-target vs. off-target activity, or increase or decrease binding and/or specificity of on-target vs. off-target binding, can be used to compensate or enhance mutations or modifications made to promote other effects. Such mutations or modifications made to promote other effects in include mutations or modification to the Cas and or mutation or modification made to a guide RNA. The methods and mutations of the invention are used to modulate Cas nuclease activity and/or binding with chemically modified guide RNAs.

In certain embodiments, the catalytic activity of the Cas protein of the invention is altered or modified. It is to be understood that mutated Cas has an altered or modified catalytic activity if the catalytic activity is different than the catalytic activity of the corresponding wild type Cas protein (e.g., unmutated Cas protein). Catalytic activity can be determined by means known in the art. By means of example, and without limitation, catalytic activity can be determined in vitro or in vivo by determination of indel percentage (for instance after a given time, or at a given dose). In certain embodiments, catalytic activity is increased. In certain embodiments, catalytic activity is increased by at least 5%, preferably at least 10%, more preferably at least 20%, such as at least 30%, at least 40%, at least 50%, at least 60%, at least 70%, at least 80%, at least 90%, or at least 100%. In certain embodiments, catalytic activity is decreased. In certain embodiments, catalytic activity is decreased by at least 5%, preferably at least 10%, more preferably at least 20%, such as at least 30%, at least 40%, at least 50%, at least 60%, at least 70%, at least 80%, at least 90%, or (substantially) 100%. The one or more mutations herein may inactivate the catalytic activity, which may substantially all catalytic activity, below detectable levels, or no measurable catalytic activity.

One or more characteristics of the engineered Cas protein may be different from a corresponding wiled type Cas protein. Examples of such characteristics include catalytic activity, gRNA binding, specificity of the Cas protein (e.g., specificity of editing a defined target), stability of the Cas protein, off-target binding, target binding, protease activity, nickase activity, PFS recognition. In some examples, a engineered Cas protein may comprise one or more mutations of the corresponding wild type Cas protein. In some embodiments, the catalytic activity of the engineered Cas protein is increased as compared to a corresponding wildtype Cas protein. In some embodiments, the catalytic activity of the engineered Cas protein is decreased as compared to a corresponding wildtype Cas protein. In some embodiments, the gRNA binding of the engineered Cas protein is increased as compared to a corresponding wildtype Cas protein. In some embodiments, the gRNA binding of the engineered Cas protein is decreased as compared to a corresponding wildtype Cas protein. In some embodiments, the specificity of the Cas protein is increased as compared to a corresponding wildtype Cas protein. In some embodiments, the specificity of the Cas protein is decreased as compared to a corresponding wildtype Cas protein. In some embodiments, the stability of the Cas protein is increased as compared to a corresponding wildtype Cas protein. In some embodiments, the stability of the Cas protein is decreased as compared to a corresponding wildtype Cas protein. In some embodiments, the engineered Cas protein further comprises one or more mutations which inactivate catalytic activity. In some embodiments, the off-target binding of the Cas protein is increased as compared to a corresponding wildtype Cas protein. In some embodiments, the off-target binding of the Cas protein is decreased as compared to a corresponding wildtype Cas protein. In some embodiments, the target binding of the Cas protein is increased as compared to a corresponding wildtype Cas protein. In some embodiments, the target binding of the Cas protein is decreased as compared to a corresponding wildtype Cas protein. In some embodiments, the engineered Cas protein has a higher protease activity or polynucleotide-binding capability compared with a corresponding wildtype Cas protein. In some embodiments, the PFS recognition is altered as compared to a corresponding wildtype Cas protein.

### Examples of Cas Proteins

Examples of Cas proteins include those of Class 1 (e.g., Type I, Type III, and Type IV) and Class 2 (e.g., Type II, Type V, and Type VI) Cas proteins, e.g., Cas9, Cas12 (e.g., Cas12a, Cas12b, Cas12c, Cas12d), Cas13 (e.g., Cas13a, Cas13b, Cas13c, Cas13d,), CasX, CasY, Cas14, variants thereof (e.g., mutated forms, truncated forms), homologs thereof, and orthologs thereof. The terms “ortholog” and “homolog” are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of. Orthologous proteins may but need not be structurally related, or are only partially structurally related.

### Class 2 Cas Proteins

In certain example embodiments, the Cas protein is a class 2 Cas protein, i.e., a Cas protein of a class 2 CRISPR-Cas system. A class 2 CRISPR-Cas system may be of a subtype, e.g., Type II-A, Type II-B, Type II-C, Type V-A, Type V-B, Type V-C, or Type V-U, [symbol] In certain example embodiments, the Cas protein is Cas9, Cas12a, Cas12b, Cas12c, or Cas12d. In some embodiments, Cas9 may be SpCas9, SaCas9, StCas9 and other Cas9 orthologs. Cas 12 may be Cas12a, Cas12b, and Cas12c, including FnCas12a, or homology or orthologs thereof. The definition and exemplary members of the CRISPR-Cas system include those described in Kira S. Makarova and Eugene V. Koonin, Annotation and Classification of CRISPR-Cas systems, Methods Mol Biol. 2015; 1311: 47-75; and Sergey Shmakov et al., Diversity and evolution of class 2 CRISPR-Cas systems, Nat Rev Microbiol. 2017 Mar; 15(3): 169-182.

### Cas Protein Linkers

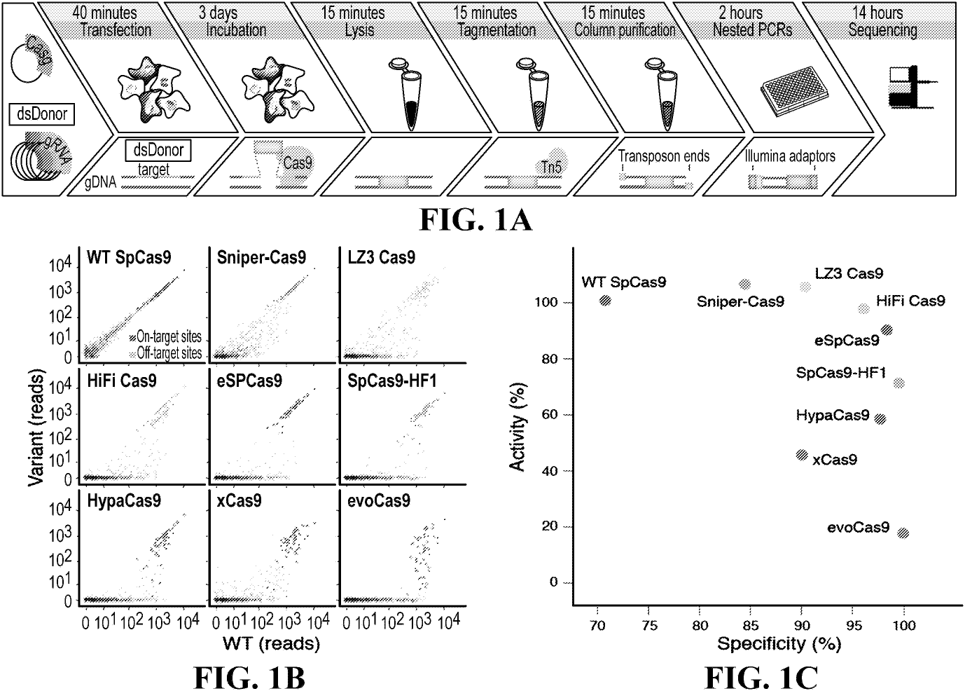

In some examples, the Cas protein comprises at least one RuvC domain and at least one HNH domain. The Cas protein may further comprise a first and a second linker domain connecting the RuvC domain and the HNH domain. The first linker (L1) and second linker (L2) connecting the HNH and RuvC domains in Cas9 are described in studies by Nishimasu, H. et al. “Crystal structure of Cas9 in complex with guide RNA and target RNA” Cell 156 (Feb. 27, 2014): 935-949 and Ribeiro, L. et al. (2018) “Protein engineering strategies to expand CRISPR-Cas9 applications” International Journal of Genomics Volume 2018, Article ID 1652567 (doi.org/10.1155/2018/1652567). FIG. 1 of Ribeiro shows the overall organization, structure and function of Cas9, incorporated specifically herein by reference. Specifically, FIG. 1A shows a schematic representation of the domain organization of SpCas9 indicating the genetic architecture of the HNH and RuvC domains including the linkers L1 (spanning amino acids 765-780) and L2 (spanning amino acids 906-918) as described herein.

Similarly, the domain organization of Staphylococcus aureus Cas9 (SaCas9) can be utilized when referencing the first and second linker domains. In an aspect, the Linker 1 domain region spans residues 481-519, and connects the RuvC-II domain to the HNH domain in SaCas9. In an aspect, Linker 2 region spans residues 629-649, and connects the RuvC-III domain and the HNH domain of SasCas9. Accordingly, the first and/or second linker domain may be mutated in a Cas9 ortholog, and reference may be made to amino acid residues corresponding to the amino acids of a wild-type SaCas9. See, Nishimasu, Cell. 2015 Aug 27; 162(5): 1113-1126; doi: 10.1016/j.cell.2015.08.007, incorporated by reference. In particular, FIG. 1, S1-S3 of Nishimasu detail domain organization of Cas9 proteins, and are incorporated specifically by reference herein for their teachings.

The first and second linker may comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 or more amino acids. The first and second linker may correspond to wild-type linkers. In an aspect, the first and second linkers may comprise one or more mutations in the first and/or second linker. In an aspect the first and/or second linker comprise one or more mutations that improve specificity of the Cas9 protein.

In some embodiments, the linkers, L1 and L2, connecting the HNH and RuvC domains of Cas9 contain the wild-type amino acid sequences. In some embodiments, the linkers connecting the HNH and RuvC domains contain mutations in one or more amino acids. In an example embodiment, the first linker (L1) contains the mutation corresponding to amino acid T769I of SpCas9 and/or the second linker (L2) contains the mutation corresponding to amino acid G915M of SpCas9. In an example embodiment, one or more linker mutations, e.g., T769I and G915M, confer improved specificity upon the Cas9 protein.

In one embodiment, one or mutations in the first and second linker may be combined with one or more mutations in other portions of the Cas9 protein for further improved specificity and/or retention of activity that is substantially equivalent to a wild-type Cas9 protein, as described herein. In one embodiment, mutations in the linker and/or additional mutations within the Cas protein can be identified utilizing the methods detailed herein that enhance/improve specificity and substantially retain wild-type activity to the wild-type Cas9. In one example embodiment, the crystal structure of the Cas protein of interest is identified, with mutations and identification of desired traits of specificity and activity screened according to exemplary embodiments detailed herein, (see, e.g FIGS. 2A-2E for exemplary initial screening), and as detailed in the examples provided herein. Such methods detailed allow for scalable assessment of desired specificity for Cas9 variants.

### Class 2, Type II Cas Proteins

In some embodiments, the Cas protein may be a Cas protein of a Class 2, Type II CRISPR-Cas system (a Type II Cas protein). In some embodiments, the Cas protein may be a class 2 Type II Cas protein, e.g., Cas9. By “Cas9 (CRISPR associated protein 9)” is meant a polypeptide or fragment thereof having at least about 85% amino acid identity to NCBI Accession No. NP_269215 and having RNA binding activity, DNA binding activity, and/or DNA cleavage activity (e.g., endonuclease or nickase activity). “Cas9 function” can be defined by any of a number of assays including, but not limited to, fluorescence polarization-based nucleic acid bind assays, fluorescence polarization-based strand invasion assays, transcription assays, EGFP disruption assays, DNA cleavage assays, and/or Surveyor assays, for example, as described herein. By “Cas9 nucleic acid molecule” is meant a polynucleotide encoding a Cas9 polypeptide or fragment thereof. An exemplary Cas9 nucleic acid molecule sequence is provided at NCBI Accession No. NC_002737. In some embodiments, disclosed herein are inhibitors of Cas9, e.g., naturally occurring Cas9 in S. pyogenes (SpCas9) or S. aureus (SaCas9), or variants thereof. Cas9 recognizes foreign DNA using Protospacer Adjacent Motif (PAM) sequence and the base pairing of the target DNA by the guide RNA (gRNA). The relative ease of inducing targeted strand breaks at any genomic loci by Cas9 has enabled efficient genome editing in multiple cell types and organisms. Cas9 derivatives can also be used as transcriptional activators/repressors.

### Cas9

In some cases, the CRISPR-Cas protein is Cas9 or a variant thereof. In some examples, Cas9 may be wildtype Cas9 including any naturally occurring bacterial Cas9. Cas9 orthologs typically share the general organization of 3-4 RuvC domains and a HNH domain. The 5′ most RuvC domain cleaves the non-complementary strand, and the HNH domain cleaves the complementary strand. All notations are in reference to the guide sequence. The catalytic residue in the 5′ RuvC domain is identified through homology comparison of the Cas9 of interest with other Cas9 orthologs (from S. pyogenes type II CRISPR locus, S. thermophilus CRISPR locus 1, S. thermophilus CRISPR locus 3, and Franciscilla novicida type II CRISPR locus), and the conserved Asp residue (D10) is mutated to alanine to convert Cas9 into a complementary-strand nicking enzyme. Accordingly, the Cas enzyme can be wildtype Cas9 including any naturally occurring bacterial Cas9. The CRISPR, Cas or Cas9 enzyme can be codon optimized, or a modified version, including any chimaeras, mutants, homologs or orthologs. In an additional aspect of the disclosure, a Cas9 enzyme may comprise one or more mutations and may be used as a generic DNA binding protein with or without fusion to a functional domain. The mutations may be artificially introduced mutations or gain- or loss-of-function mutations. In one aspect of the disclosure, the transcriptional activation domain may be VP64. In other aspects of the disclosure, the transcriptional repressor domain may be KRAB or SID4X. Other aspects of the disclosure relate to the mutated Cas 9 enzyme being fused to domains which include but are not limited to a nuclease, a transcriptional activator, repressor, a recombinase, a transposase, a histone remodeler, a demethylase, a DNA methyltransferase, a cryptochrome, a light inducible/controllable domain or a chemically inducible/controllable domain. The disclosure can involve sgRNAs or tracrRNAs or guide or chimeric guide sequences that allow for enhancing performance of these RNAs in cells. This type II CRISPR enzyme may be any Cas enzyme. In some cases, the Cas9 enzyme is from, or is derived from, SpCas9 or SaCas9. By derived, Applicants mean that the derived enzyme is largely based, in the sense of having a high degree of sequence homology with, a wildtype enzyme, but that it has been mutated (modified) in some way as described herein. In an example the mutation may comprise one or more mutations in a first linker domain, a second linker domain, and/or other portions of the protein. The high degree of sequence homology may comprise at least 80%, 85%, 86%, 87%, 88%, 89%, 90%, 91%, 92%, 93%, 94%, 95%, 96%, 97%, 98%, 99% or more relative to a wildtype enzyme.

A Cas enzyme may be identified Cas9 as this can refer to the general class of enzymes that share homology to the biggest nuclease with multiple nuclease domains from the type II CRISPR system. In some cases, the Cas9 enzyme is from, or is derived from, SpCas9 (S. pyogenes Cas9) or saCas9 (S. aureus Cas9). StCas9″ refers to wild type Cas9 from S. thermophilus, the protein sequence of which is given in the SwissProt database under accession number G3ECR1. Similarly, S pyogenes Cas9 or SpCas9 is included in SwissProt under accession number Q99ZW2. By derived, Applicants mean that the derived enzyme is largely based, in the sense of having a high degree of sequence homology with, a wildtype enzyme, but that it has been mutated (modified) in some way as described herein. It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes. However, it will be appreciated that this disclosure includes many more Cas9s from other species of microbes, such as SpCas9, SaCa9, St1Cas9 and so forth. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defence in bacteria and archaea, Mole Cell 2010, January 15; 37(1): 7. The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. A pre-crRNA array consisting of a single spacer flanked by two direct repeats (DRs) is also encompassed by the term “tracr-mate sequences”). In certain embodiments, Cas9 may be constitutively present or inducibly present or conditionally present or administered or delivered. Cas9 optimization may be used to enhance function or to develop new functions, one can generate chimeric Cas9 proteins. And Cas9 may be used as a generic DNA binding protein.

The structural information provided for Cas9 (e.g. S. pyogenes Cas9) as the CRISPR enzyme in the present invention may be used to further engineer and optimize the CRISPR-Cas system and this may be extrapolated to interrogate structure-function relationships in other CRISPR enzyme systems as well, particularly structure-function relationships in other Type II CRISPR enzymes or Cas9 orthologs. The crystal structure information (described in U.S. Provisional Applications 61/915,251 filed Dec. 12, 2013, 61/930,214 filed on Jan. 22, 2014, 61/980,012 filed Apr. 15, 2014; and Nishimasu et al, “Crystal Structure of Cas9 in Complex with Guide RNA and Target DNA,” Cell 156(5):935-949, DOI: http://dx.doi.org/10.1016/j.cell.2014.02.001 (2014), each and all of which are incorporated herein by reference) provides structural information to truncate and create modular or multi-part CRISPR enzymes which may be incorporated into inducible CRISPR-Cas systems. In particular, structural information is provided for S. pyogenes Cas9 (SpCas9) and this may be extrapolated to other Cas9 orthologs or other Type II CRISPR enzymes.

The Cas9 gene is found in several diverse bacterial genomes, typically in the same locus with cas1, cas2, and cas4 genes and a CRISPR cassette. Furthermore, the Cas9 protein contains a readily identifiable C-terminal region that is homologous to the transposon ORF-B and includes an active RuvC-like nuclease, an arginine-rich region.

In particular embodiments, the effector protein is a Cas9 effector protein from or originated from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacte, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus, Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, or Campylobacter.

In further particular embodiments, the Cas9 effector protein is from or originatedfrom an organism selected from S. mutans, S. agalactiae, S. equisimilis, S. sanguinis, S. pneumonia, C. jejuni, C. coli; N. salsuginis, N. tergarcus; S. auricularis, S. carnosus; N. meningitides, N. gonorrhoeae, L. monocytogenes, L. ivanovii; C. botulinum, C. difficile, C. tetani, or C. sordellii, Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2 44 17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. In particular embodiments, the effector protein is a Cas9 effector protein from an organism from or originated from Streptococcus pyogenes, Staphylococcus aureus, or Streptococcus thermophilus Cas9. In a more preferred embodiment, the Cas9 is derived from a bacterial species selected from Streptococcus pyogenes, Staphylococcus aureus, or Streptococcus thermophilus Cas9. In certain embodiments, the Cas9 is derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2 44 17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cas9p is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

### Cas Variants

The engineered Cas protein may comprise one or more mutations, e.g., in RuvC domain, HNH domain, one or more of the linker domains. In some examples, the engineered Cas9 protein comprises one or more mutations of amino acids corresponding to the following amino acids of SpCas9: N690, T769, G915, and N980 based on amino acid of sequence positions of wildtype SpCas9. For example, the engineered Cas9 protein comprises one or more mutations: N690C, T769I, G915M, N980K based on amino acid of sequence positions of wildtype SpCas9.

Additional examples of mutations on engineered Cas protein include those described in FIG. 2E. An example of the Cas protein is LZ3 Cas9 described herein. In one embodiment, the LZ3 Cas9 comprises SEQ ID NO: 1300 or is encoded by SEQ ID NO: 1299.

### Guide Molecule

The CRISPR-Cas systems herein may comprise one or more guide molecules (e.g., guide RNAs) or a nucleotide sequence encoding thereof. In some cases, the guide molecule comprises a guide sequence and a direct repeat sequence. The guide sequence and the direct repeat sequence may be linked. Examples and features of guide molecules include those described in paragraphs [0266]-[0467] of Zhang et al., WO2019126774, which is incorporated in reference herein in its entirety.

As used herein, the term “guide sequence” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequence may form a duplex with a target sequence. The duplex may be a DNA duplex, an RNA duplex, or a RNA/DNA duplex. The terms “guide molecule” and “guide RNA” are used interchangeably herein to refer to RNA-based molecules that are capable of forming a complex with a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA specifically encompasses RNA-based molecules having one or more chemically modifications (e.g., by chemical linking two ribonucleotides or by replacement of one or more ribonucleotides with one or more deoxyribonucleotides), as described herein.

The guide molecule or guide RNA of a CRISPR-Cas protein may comprise a tracr-mate sequence (encompassing a “direct repeat” in the context of an endogenous CRISPR system) and a guide sequence (also referred to as a “spacer” in the context of an endogenous CRISPR system). In some embodiments, the CRISPR-Cas system or complex as described herein does not comprise and/or does not rely on the presence of a tracr sequence. In certain embodiments, the guide molecule may comprise, consist essentially of, or consist of a direct repeat sequence fused or linked to a guide sequence or spacer sequence.

In general, a CRISPR-Cas system is characterized by elements that promote the formation of a CRISPR complex at the site of a target sequence. In the context of formation of a CRISPR complex, “target sequence” refers to a sequence to which a guide sequence is designed to have complementarity, where hybridization between a target DNA sequence and a guide sequence promotes the formation of a CRISPR complex.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A.R. Gruber et al., 2008, Cell 106(1): 23-24; and PA Carr and GM Church, 2009, Nature Biotechnology 27(12): 1151-62).

### Delivery Systems

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs [00117] to [00278] of Feng Zhang et al., (WO2016106236A1), and pages 1241-1251 and Table 1 of Lino CA et al., Delivering CRISPR: a review of the challenges and approaches, DRUG DELIVERY, 2018, VOL. 25, NO. 1, 1234-1257, which are incorporated by reference herein in their entireties.

### Cargos

The delivery systems may comprise one or more cargos. The cargos may comprise one or more components of the systems and compositions herein. A cargo may comprise one or more of the following: i) a plasmid encoding one or more Cas proteins; ii) a plasmid encoding one or more guide RNAs, iii) mRNA of one or more Cas proteins; iv) one or more guide RNAs; v) one or more Cas proteins; vi) any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNAs.

In some examples, a cargo may comprise one or more Cas proteins and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516.

### Physical Delivery

In some embodiments, the cargos may be introduced to cells by physical delivery methods. Examples of physical methods include microinjection, electroporation, and hydrodynamic delivery.

### Microinjection

Microinjection of the cargo directly to cells can achieve high efficiency, e.g., above 90% or about 100%. In some embodiments, microinjection may be performed using a microscope and a needle (e.g., with 0.5-5.0 µm in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down- regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi PS, Meyerson M. (2014). Nat Commun 5:3728; Wang J, Quake SR. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may a greatest dimension (e.g. diameter) of less than 100 microns (µm). In some embodiments, the delivery vehicles have a greatest dimension of less than 10 µm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

### Vectors

The systems, compositions, and/or delivery systems may comprise one or more vectors. The present disclosure also include vector systems. A vector system may comprise one or more vectors. In some embodiments, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) Cas encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

### Regulatory Elements

A vector may comprise one or more regulatory elements. The regulatory element(s) may be operably linked to coding sequences of Cas proteins, accessary proteins, guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In certain examples, a vector may comprise: a first regulatory element operably linked to a nucleotide sequence encoding a Cas protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In some embodiments, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno-Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA. In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82: 5887-5911 (2008)), and shown below in Table 1:

TABLE 1

Examples of AAV that can be used with the cell lines described herein

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

CRISPR-Cas AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those describe in US Patent Nos. 8,454,972 and 8,404,658.

Various strategies may be used for delivery the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In some embodiments, adenoviruses do not integrate into the genome of host cells, which may be used for limiting off-target effects of CRISPR-Cas systems in gene editing applications.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

Components in LNPs may comprise cationic lipids 1,2- dilineoyl-3-dimethylammonium-propane (DLinDAP), 1,2-dilinoleyloxy-3-N,N- dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3- o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3 -phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3- phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG- cDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl-[1,3]- dioxolane (DLin-KC2-DMA), DLin-KC2-DMA4, C12- 200 and colipids disteroylphosphatidyl choline, cholesterol, and PEG-DMG.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca²⁺ microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl). Examples of CPPs and related applications also include those described in U.S. Pat. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct 22;136(42):14722-5; and Sun W et al, Angew Chem Int Ed Engl. 2015 Oct 5;54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics’ Spherical Nucleic Acid (SNA™) constructs, and those described in Mout R, et al. (2017). ACS Nano 11:2452-8; Lee K, et al. (2017). Nat Biomed Eng 1:889-901.

### iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D′Astolfo DS, Pagliero RJ, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage SS et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection - Factbook 2018: technology, product overview, users’ data., doi:10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci U S A 98:3185-90; Teng KW, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee PN, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo GF, et al. (2014). Sci Rep 4:6064), and dense silica nanoparticles (SiNPs) (as described in Luo D and Saltzman WM. (2000). Nat Biotechnol 18:893-5).

### Methods of Use

The compositions and systems herein may be used for a variety of applications, including modifying non-animal organisms such as plants and fungi, and modifying animals, treating and diagnosing diseases in plants, animals, and humans. In general, the compositions and systems may be introduced to cells, tissues, organs, or organisms, where they modify the expression and/or activity of one or more genes. Examples of applications include those described in [0874] - [1064] of Zhang et al., WO2019126774, which is incorporated in reference herein in its entirety.

### Cells and Organisms

The present disclosure provides cells, tissues, organisms comprising the engineered Cas protein, the CRISPR-Cas systems, the polynucleotides encoding one or more components of the CRISPR-Cas systems, and/or vectors comprising the polynucleotides. The invention also provides for the nucleotide sequence encoding the effector protein being codon optimized for expression in a eukaryote or eukaryotic cell in any of the herein described methods or compositions. In an embodiment of the invention, the codon optimized effector protein is any Cas protein discussed herein and is codon optimized for operability in a eukaryotic cell or organism, e.g., such cell or organism as elsewhere herein mentioned, for instance, without limitation, a yeast cell, or a mammalian cell or organism, including a mouse cell, a rat cell, and a human cell or non-human eukaryote organism, e.g., plant.

In certain embodiments, the modification of the target locus of interest may result in: the eukaryotic cell comprising altered expression of at least one gene product; the eukaryotic cell comprising altered expression of at least one gene product, wherein the expression of the at least one gene product is increased; the eukaryotic cell comprising altered expression of at least one gene product, wherein the expression of the at least one gene product is decreased; or the eukaryotic cell comprising an edited genome.

In certain embodiments, the eukaryotic cell may be a mammalian cell or a human cell.

In further embodiments, the non-naturally occurring or engineered compositions, the vector systems, or the delivery systems as described in the present specification may be used for: site-specific gene knockout; site-specific genome editing; RNA sequence-specific interference; or multiplexed genome engineering.

Also provided is a gene product from the cell, the cell line, or the organism as described herein. In certain embodiments, the amount of gene product expressed may be greater than or less than the amount of gene product from a cell that does not have altered expression or edited genome. In certain embodiments, the gene product may be altered in comparison with the gene product from a cell that does not have altered expression or edited genome.

### Exemplary Therapies

The present invention also contemplates use of the CRISPR-Cas system and the base editor described herein, for treatment in a variety of diseases and disorders. In some embodiments, the invention described herein relates to a method for therapy in which cells are edited ex vivo by CRISPR or the base editor to modulate at least one gene, with subsequent administration of the edited cells to a patient in need thereof. In some embodiments, the editing involves knocking in, knocking out or knocking down expression of at least one target gene in a cell. In particular embodiments, the editing inserts an exogenous, gene, minigene or sequence, which may comprise one or more exons and introns or natural or synthetic introns into the locus of a target gene, a hot-spot locus, a safe harbor locus of the gene genomic locations where new genes or genetic elements can be introduced without disrupting the expression or regulation of adjacent genes, or correction by insertions or deletions one or more mutations in DNA sequences that encode regulatory elements of a target gene. In some embodiment, the editing comprise introducing one or more point mutations in a nucleic acid (e.g., a genomic DNA) in a target cell.

In embodiments, the treatment is for disease/disorder of an organ, including liver disease, eye disease, muscle disease, heart disease, blood disease, brain disease, kidney disease, or may comprise treatment for an autoimmune disease, central nervous system disease, cancer and other proliferative diseases, neurodegenerative disorders, inflammatory disease, metabolic disorder, musculoskeletal disorder and the like.

Particular diseases/disorders include chondroplasia, achromatopsia, acid maltase deficiency, adrenoleukodystrophy, aicardi syndrome, alpha- 1 antitrypsin deficiency, alpha-thalassemia, androgen insensitivity syndrome, apert syndrome, arrhythmogenic right ventricular, dysplasia, ataxia telangictasia, barth syndrome, beta-thalassemia, blue rubber bleb nevus syndrome, canavan disease, chronic granulomatous diseases (CGD), cri du chat syndrome, cystic fibrosis, dercum’s disease, ectodermal dysplasia, fanconi anemia, fibrodysplasia ossificans progressive, fragile X syndrome, galactosemis, Gaucher’s disease, generalized gangliosidoses (e.g., GM1), hemochromatosis, the hemoglobin C mutation in the 6th codon of beta-globin (HbC), hemophilia, Huntington’s disease, Hurler Syndrome, hypophosphatasia, Klinefleter syndrome, Krabbes Disease, Langer-Giedion Syndrome, leukodystrophy, long QT syndrome, Marfan syndrome, Moebius syndrome, mucopolysaccharidosis (MPS), nail patella syndrome, nephrogenic diabetes insipdius, neurofibromatosis, Neimann-Pick disease, osteogenesis imperfecta, porphyria, Prader- Willi syndrome, progeria, Proteus syndrome, retinoblastoma, Rett syndrome, Rubinstein-Taybi syndrome, Sanfilippo syndrome, severe combined immunodeficiency (SCID), Shwachman syndrome, sickle cell disease (sickle cell anemia), Smith-Magenis syndrome, Stickler syndrome, Tay-Sachs disease, Thrombocytopenia Absent Radius (TAR) syndrome, Treacher Collins syndrome, trisomy, tuberous sclerosis, Turner’s syndrome, urea cycle disorder, von Hippel- Landau disease, Waardenburg syndrome, Williams syndrome, Wilson’s disease, and Wiskott- Aldrich syndrome.

In embodiments, the disease is associated with expression of a tumor antigen, e.g., a proliferative disease, a precancerous condition, a cancer, or a non-cancer related indication associated with expression of the tumor antigen, which may in some embodiments comprise a target selected from B2M, CD247, CD3D, CD3E, CD3G, TRAC, TRBC1, TRBC2, HLA-A, HLA-B, HLA-C, DCK, CD52, FKBP1A, CIITA, NLRC5, RFXANK, RFX5, RFXAP, or NR3C1, HAVCR2, LAG3, PDCD1, PD-L2, CTLA4, CEACAM (CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, or PTPN11 DCK, CD52, NR3C1, LILRB1, CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); n kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1), CD19, BCMA, CD70, G6PC, Dystrophin, including modification of exon 51 by deletion or excision, DMPK, CFTR (cystic fibrosis transmembrane conductance regulator). In embodiments, the targets comprise CD70, or a Knock-in of CD33 and Knockout of B2M. In embodiments, the targets comprise a knockout of TRAC and B2M, or TRAC B2M and PD1, with or without additional target genes. In certain embodiments, the disease is cystic fibrosis with targeting of the SCNN1A gene, e.g., the non-coding or coding regions, e.g., a promoter region, or a transcribed sequence, e.g., intronic or exonic sequence, targeted knock-in at CFTR sequence within intron 2, into which, e.g., can be introduced CFTR sequence that codes for CFTR exons 3-27; and sequence within CFTR intron 10, into which sequence that codes for CFTR exons 11-27 can be introduced.

In embodiments, the disease is Metachromatic Leukodystrophy, and the target is Arylsulfatase A, the disease is Wiskott-Aldrich Syndrome and the target is Wiskott-Aldrich Syndrome protein, the disease is Adreno leukodystrophy and the target is ATP-binding cassette DI, the disease is Human Immunodeficiency Virus and the target is receptor type 5-C-C chemokine or CXCR4 gene, the disease is Beta-thalassemia and the target is Hemoglobin beta subunit, the disease is X-linked Severe Combined ID receptor subunit gamma and the target is interelukin-2 receptor subunit gamma, the disease is Multisystemic Lysosomal Storage Disorder cystinosis and the target is cystinosin, the disease is Diamon-Blackfan anemia and the target is Ribosomal protein S19, the disease is Fanconi Anemia and the target is Fanconi anemia complementation groups (e.g. FNACA, FNACB, FANCC, FANCD1, FANCD2, FANCE, FANCF, RAD51C), the disease is Shwachman-Bodian-Diamond Bodian-Diamond syndrome and the target is Shwachman syndrome gene, the disease is Gaucher’s disease and the target is Glucocerebrosidase, the disease is Hemophilia A and the target is Anti-hemophiliac factor OR Factor VIII, Christmas factor, Serine protease, Factor Hemophilia B IX, the disease is Adenosine deaminase deficiency (ADA-SCID) and the target is Adenosine deaminase, the disease is GM1 gangliosidoses and the target is beta-galactosidase, the disease is Glycogen storage disease type II, Pompe disease, the disease is acid maltase deficiency acid and the target is alpha-glucosidase, the disease is Niemann-Pick disease, SMPD1 -associated (Types Sphingomyelin phosphodiesterase 1 OR A and B) acid and the target is sphingomyelinase, the disease is Krabbe disease, globoid cell leukodystrophy and the target is Galactosylceramidase or galactosylceramide lipidosis and the target is galactercerebrosidease, Human leukocyte antigens DR-15, DQ-6, the disease is Multiple Sclerosis (MS) DRB1, the disease is Herpes Simplex Virus 1 or 2 and the target is knocking down of one, two or three of RS1, RL2 and/or LAT genes. In embodiments, the disease is an HPV associated cancer with treatment including edited cells comprising binding molecules, such as TCRs or antigen binding fragments thereof and antibodies and antigen-binding fragments thereof, such as those that recognize or bind human papilloma virus. The disease can be Hepatitis B with a target of one or more of PreC, C, X, PreS1, PreS2, S, P and/or SP gene(s).

In embodiments, the immune disease is severe combined immunodeficiency (SCID), Omenn syndrome, and in one aspect the target is Recombination Activating Gene 1 (RAG1) or an interleukin-7 receptor (IL7R). In particular embodiments, the disease is Transthyretin Amyloidosis (ATTR), Familial amyloid cardiomyopathy, and in one aspect, the target is the TTR gene, including one or more mutations in the TTR gene. In embodiments, the disease is Alpha-1 Antitrypsin Deficiency (AATD) or another disease in which Alpha-1 Antitrypsin is implicated, for example GvHD, Organ transplant rejection, diabetes, liver disease, COPD, Emphysema and Cystic Fibrosis, in particular embodiments, the target is SERPINA1.

In embodiments, the disease is primary hyperoxaluria, which, in certain embodiments, the target comprises one or more of Lactate dehydrogenase A (LDHA) and hydroxy Acid Oxidase 1 (HAO 1). In embodiments, the disease is primary hyperoxaluria type 1 (ph1) and other alanine-glyoxylate aminotransferase (agxt) gene related conditions or disorders, such as Adenocarcinoma, Chronic Alcoholic Intoxication, Alzheimer’s Disease, Cooley’s anemia, Aneurysm, Anxiety Disorders, Asthma, Malignant neoplasm of breast, Malignant neoplasm of skin, Renal Cell Carcinoma, Cardiovascular Diseases, Malignant tumor of cervix, Coronary Arteriosclerosis, Coronary heart disease, Diabetes, Diabetes Mellitus, Diabetes Mellitus Non- Insulin-Dependent, Diabetic Nephropathy, Eclampsia, Eczema, Subacute Bacterial Endocarditis, Glioblastoma, Glycogen storage disease type II, Sensorineural Hearing Loss (disorder), Hepatitis, Hepatitis A, Hepatitis B, Homocystinuria, Hereditary Sensory Autonomic Neuropathy Type 1, Hyperaldosteronism, Hypercholesterolemia, Hyperoxaluria, Primary Hyperoxaluria, Hypertensive disease, Inflammatory Bowel Diseases, Kidney Calculi, Kidney Diseases, Chronic Kidney Failure, leiomyosarcoma, Metabolic Diseases, Inborn Errors of Metabolism, Mitral Valve Prolapse Syndrome, Myocardial Infarction, Neoplasm Metastasis, Nephrotic Syndrome, Obesity, Ovarian Diseases, Periodontitis, Polycystic Ovary Syndrome, Kidney Failure, Adult Respiratory Distress Syndrome, Retinal Diseases, Cerebrovascular accident, Turner Syndrome, Viral hepatitis, Tooth Loss, Premature Ovarian Failure, Essential Hypertension, Left Ventricular Hypertrophy, Migraine Disorders, Cutaneous Melanoma, Hypertensive heart disease, Chronic glomerulonephritis, Migraine with Aura, Secondary hypertension, Acute myocardial infarction, Atherosclerosis of aorta, Allergic asthma, pineoblastoma, Malignant neoplasm of lung, Primary hyperoxaluria type I, Primary hyperoxaluria type 2, Inflammatory Breast Carcinoma, Cervix carcinoma, Restenosis, Bleeding ulcer, Generalized glycogen storage disease of infants, Nephrolithiasis, Chronic rejection of renal transplant, Urolithiasis, pricking of skin, Metabolic Syndrome X, Maternal hypertension, Carotid Atherosclerosis, Carcinogenesis, Breast Carcinoma, Carcinoma of lung, Nephronophthisis, Microalbuminuria, Familial Retinoblastoma, Systolic Heart Failure Ischemic stroke, Left ventricular systolic dysfunction, Cauda Equina Paraganglioma, Hepatocarcinogenesis, Chronic Kidney Diseases, Glioblastoma Multiforme, Non-Neoplastic Disorder, Calcium Oxalate Nephrolithiasis, Ablepharon-Macrostomia Syndrome, Coronary Artery Disease, Liver carcinoma, Chronic kidney disease stage 5, Allergic rhinitis (disorder), Crigler Najjar syndrome type 2, and Ischemic Cerebrovascular Accident. In certain embodiments, treatment is targeted to the liver. In embodiments, the gene is AGXT, with a cytogenetic location of 2q37.3 and the genomic coordinate are on Chromosome 2 on the forward strand at position 240,868,479-240,880,502.

Treatment can also target collagen type vii alpha 1 chain (col7a1) gene related conditions or disorders, such as Malignant neoplasm of skin, Squamous cell carcinoma, Colorectal Neoplasms, Crohn Disease, Epidermolysis Bullosa, Indirect Inguinal Hernia, Pruritus, Schizophrenia, Dermatologic disorders, Genetic Skin Diseases, Teratoma, Cockayne-Touraine Disease, Epidermolysis Bullosa Acquisita, Epidermolysis Bullosa Dystrophica, Junctional Epidermolysis Bullosa, Hallopeau- Siemens Disease, Bullous Skin Diseases, Agenesis of corpus callosum, Dystrophia unguium, Vesicular Stomatitis, Epidermolysis Bullosa With Congenital Localized Absence Of Skin And Deformity Of Nails, Juvenile Myoclonic Epilepsy, Squamous cell carcinoma of esophagus, Poikiloderma of Kindler, pretibial Epidermolysis bullosa, Dominant dystrophic epidermolysis bullosa albopapular type (disorder), Localized recessive dystrophic epidermolysis bullosa, Generalized dystrophic epidermolysis bullosa, Squamous cell carcinoma of skin, Epidermolysis Bullosa Pruriginosa, Mammary Neoplasms, Epidermolysis Bullosa Simplex Superficialis, Isolated Toenail Dystrophy, Transient bullous dermolysis of the newborn, Autosomal Recessive Epidermolysis Bullosa Dystrophica Localisata Variant, and Autosomal Recessive Epidermolysis Bullosa Dystrophica Inversa.

In embodiments, the disease is acute myeloid leukemia (AML), targeting Wilms Tumor I (WTI) and HLA expressing cells. In embodiments, the therapy is T cell therapy, as described elsewhere herein, comprising engineered T cells with WTI specific TCRs. In certain embodiments, the target is CD157 in AML.

In embodiments, the disease is a blood disease. In certain embodiments, the disease is hemophilia, in one aspect the target is Factor XI. In other embodiments, the disease is a hemoglobinopathy, such as sickle cell disease, sickle cell trait, hemoglobin C disease, hemoglobin C trait, hemoglobin S/C disease, hemoglobin D disease, hemoglobin E disease, a thalassemia, a condition associated with hemoglobin with increased oxygen affinity, a condition associated with hemoglobin with decreased oxygen affinity, unstable hemoglobin disease, methemoglobinemia. Hemostasis and Factor X and XII deficiencies can also be treated. In embodiments, the target is BCL11A gene (e.g., a human BCL11a gene), a BCL11a enhancer (e.g., a human BCL11a enhancer), or a HFPH region (e.g., a human HPFH region), beta globulin, fetal hemoglobin, γ-globin genes (e.g., HBG1, HBG2, or HBG1 and HBG2), the erythroid specific enhancer of the BCL11A gene (BCL11Ae), or a combination thereof.

In embodiments, the target locus can be one or more of RAC, TRBCl, TRBC2, CD3E, CD3G, CD3D, B2M, CIITA, CD247, HLA-A, HLA-B, HLA-C, DCK, CD52, FKBP1A, NLRC5, RFXANK, RFX5, RFXAP, NR3C1, CD274, HAVCR2, LAG3, PDCD1, PD-L2, HCF2, PAI, TFPI, PLAT, PLAU, PLG, RPOZ, F7, F8, F9, F2, F5, F7, F10, F11, F12, F13A1, F13B, STAT1, FOXP3, IL2RG, DCLRE1C, ICOS, MHC2TA, GALNS, HGSNAT, ARSB, RFXAP, CD20, CD81, TNFRSF13B, SEC23B, PKLR, IFNG, SPTB, SPTA, SLC4A1, EPO, EPB42, CSF2 CSF3, VFW, SERPINCA1, CTLA4, CEACAM (e.g., CEACAM-1, CEACAM-3 and/or CEACAM-5), VISTA, BTLA, TIGIT, LAIR1, CD160, 2B4, CD80, CD86, B7-H3 (CD113), B7-H4 (VTCN1), HVEM (TNFRSF14 or CD107), KIR, A2aR, MHC class I, MHC class II, GAL9, adenosine, and TGF beta, PTPN11, and combinations thereof. In embodiments, the target sequence within the genomic nucleic acid sequence at Chr1 1:5,250,094-5,250,237, - strand, hg38; Chr1 1:5,255,022-5,255,164, - strand, hg38; nondeletional HFPH region; Chr1 1:5,249,833 to Chr1 1:5,250,237, - strand, hg38; Chr1 1:5,254,738 to Chr1 1:5,255, 164, - strand, hg38; Chr1 1 : 5,249,833-5,249,927, - strand, hg3; Chr1 1 : 5,254,738-5,254,851, - strand, hg38; Chr1 1:5,250, 139-5,250,237, - strand, hg38.

In embodiments, the disease is associated with high cholesterol, and regulation of cholesterol is provided, in some embodiments, regulation is affected by modification in the target PCSK9. Other diseases in which PCSK9 can be implicated, and thus would be a target for the systems and methods described herein include Abetaiipoproteinemia, Adenoma, Arteriosclerosis, Atherosclerosis, Cardiovascular Diseases, Cholelithiasis, Coronary Arteriosclerosis, Coronary heart disease, Non-Insulin-Dependent Diabetes Meliitus, Hypercholesterolemia, Familial Hypercholesterolemia, Hyperinsuiinism, Hyperlipidemia, Familial Combined Hyperlipidemia, Hypobetalipoproteinemias, Chronic Kidney Failure, Liver diseases, Liver neoplasms, melanoma, Myocardial Infarction, Narcolepsy, Neoplasm Metastasis, Nephroblastoma, Obesity, Peritonitis, Pseudoxanthoma Elasticum, Cerebrovascular accident, Vascular Diseases, Xanthomatosis, Peripheral Vascular Diseases, Myocardial Ischemia, Dyslipidemias, Impaired glucose tolerance, Xanthoma, Polygenic hypercholesterolemia, Secondary malignant neoplasm of liver, Dementia, Overweight, Hepatitis C, Chronic, Carotid Atherosclerosis, Hyperlipoproteinemia Type Ha, Intracranial Atherosclerosis, Ischemic stroke, Acute Coronary Syndrome, Aortic calcification, Cardiovascular morbidity, Hyperlipoproteinemia Type lib, Peripheral Arterial Diseases, Familial Hyperaldosteronism Type II, Familial hypobetalipoproteinemia, Autosomal Recessive Hypercholesterolemia, Autosomal Dominant Hypercholesterolemia 3, Coronary Artery Disease, Liver carcinoma, Ischemic Cerebrovascular Accident, and Arteriosclerotic cardiovascular disease NOS. In embodiments, the treatment can be targeted to the liver, the primary location of activity of PCSK9.

In embodiments, the disease or disorder is Hyper IGM syndrome or a disorder characterized by defective CD40 signaling. In certain embodiments, the insertion of CD40L exons are used to restore proper CD40 signaling and B cell class switch recombination. In particular embodiments, the target is CD40 ligand (CD40L)-edited at one or more of exons 2-5 of the CD40L gene, in cells, e.g., T cells or hematopoietic stem cells (HSCs).

In embodiments, the disease is merosin-deficient congenital muscular dystrophy (mdcmd) and other laminin, alpha 2 (lama2) gene related conditions or disorders. The therapy can be targeted to the muscle, for example, skeletal muscle, smooth muscle, and/or cardiac muscle. In certain embodiments, the target is Laminin, Alpha 2 (LAMA2) which may also be referred to as Laminin- 12 Subunit Alpha, Laminin-2 Subunit Alpha, Laminin-4 Subunit Alpha 3, Merosin Heavy Chain, Laminin M Chain, LAMM, Congenital Muscular Dystrophy and Merosin. LAMA2 has a cytogenetic location of 6q22.33 and the genomic coordinate are on Chromosome 6 on the forward strand at position 128,883, 141-129,516,563. In embodiments, the disease treated can be Merosin-Deficient Congenital Muscular Dystrophy (MDCMD), Amyotrophic Lateral Sclerosis, Bladder Neoplasm, Charcot-Marie-Tooth Disease, Colorectal Carcinoma, Contracture, Cyst, Duchenne Muscular Dystrophy, Fatigue, Hyperopia, Renovascular Hypertension, melanoma, Mental Retardation, Myopathy, Muscular Dystrophy, Myopia, Myositis, Neuromuscular Diseases, Peripheral Neuropathy, Refractive Errors, Schizophrenia, Severe mental retardation (I.Q. 20-34), Thyroid Neoplasm, Tobacco Use Disorder, Severe Combined Immunodeficiency, Synovial Cyst, Adenocarcinoma of lung (disorder), Tumor Progression, Strawberry nevus of skin, Muscle degeneration, Microdontia (disorder), Walker-Warburg congenital muscular dystrophy, Chronic Periodontitis, Leukoencephalopathies, Impaired cognition, Fukuyama Type Congenital Muscular Dystrophy, Scleroatonic muscular dystrophy, Eichsfeld type congenital muscular dystrophy, Neuropathy, Muscle eye brain disease, Limb-Muscular Dystrophies, Girdle, Congenital muscular dystrophy (disorder), Muscle fibrosis, cancer recurrence, Drug Resistant Epilepsy, Respiratory Failure, Myxoid cyst, Abnormal breathing, Muscular dystrophy congenital merosin negative, Colorectal Cancer, Congenital Muscular Dystrophy due to Partial LAMA2 Deficiency, and Autosomal Dominant Craniometaphyseal Dysplasia.

In certain embodiments, the target is an AAVS1 (PPPIR12C), an ALB gene, an Angptl3 gene, an ApoC3 gene, an ASGR2 gene, a CCR5 gene, a FIX (F9) gene, a G6PC gene, a Gys2 gene, an HGD gene, a Lp(a) gene, a Pcsk9 gene, a Serpinal gene, a TF gene, and a TTR gene). Assessment of efficiency of HDR/NHEJ mediated knock-in of cDNA into the first exon can utilize cDNA knock-in into “safe harbor” sites such as: single-stranded or double-stranded DNA having homologous arms to one of the following regions, for example: ApoC3 (chr11:116829908-116833071), Angptl3 (chr1:62,597,487-62,606,305), Serpinal (chr14:94376747-94390692), Lp(a) (chr6:160531483-160664259), Pcsk9 (chr1:55,039,475-55,064,852), FIX (chrX:139,530,736-139,563,458), ALB (chr4:73,404,254-73,421,411), TTR (chr1 8:31,591,766-31,599,023), TF (chr3:133,661,997-133,779,005), G6PC (chr17:42,900,796-42,914,432), Gys2 (chr12:21,536,188-21,604,857), AAVS1 (PPP1R12C) (chr19:55,090,912-55,117,599), HGD (chr3:120,628,167-120,682,570), CCR5 (chr3:46,370,854-46,376,206), or ASGR2 (chr17:7,101,322-7,114,310).

In one aspect, the target is superoxide dismutase 1, soluble (SOD1), which can aid in treatment of a disease or disorder associated with the gene. In particular embodiments, the disease or disorder is associated with SOD1, and can be, for example, Adenocarcinoma, Albuminuria, Chronic Alcoholic Intoxication, Alzheimer’s Disease, Amnesia, Amyloidosis, Amyotrophic Lateral Sclerosis, Anemia, Autoimmune hemolytic anemia, Sickle Cell Anemia, Anoxia, Anxiety Disorders, Aortic Diseases, Arteriosclerosis, Rheumatoid Arthritis, Asphyxia Neonatorum, Asthma, Atherosclerosis, Autistic Disorder, Autoimmune Diseases, Barrett Esophagus, Behcet Syndrome, Malignant neoplasm of urinary bladder, Brain Neoplasms, Malignant neoplasm of breast, Oral candidiasis, Malignant tumor of colon, Bronchogenic Carcinoma, Non-Small Cell Lung Carcinoma, Squamous cell carcinoma, Transitional Cell Carcinoma, Cardiovascular Diseases, Carotid Artery Thrombosis, Neoplastic Cell Transformation, Cerebral Infarction, Brain Ischemia, Transient Ischemic Attack, Charcot-Marie-Tooth Disease, Cholera, Colitis, Colorectal Carcinoma, Coronary Arteriosclerosis, Coronary heart disease, Infection by Cryptococcus neoformans, Deafness, Cessation of life, Deglutition Disorders, Presenile dementia, Depressive disorder, Contact Dermatitis, Diabetes, Diabetes Mellitus, Experimental Diabetes Mellitus, Insulin-Dependent Diabetes Mellitus, Non-Insulin-Dependent Diabetes Mellitus, Diabetic Angiopathies, Diabetic Nephropathy, Diabetic Retinopathy, Down Syndrome, Dwarfism, Edema, Japanese Encephalitis, Toxic Epidermal Necrolysis, Temporal Lobe Epilepsy, Exanthema, Muscular fasciculation, Alcoholic Fatty Liver, Fetal Growth Retardation, Fibromyalgia, Fibrosarcoma, Fragile X Syndrome, Giardiasis, Glioblastoma, Glioma, Headache, Partial Hearing Loss, Cardiac Arrest, Heart failure, Atrial Septal Defects, Helminthiasis, Hemochromatosis, Hemolysis (disorder), Chronic Hepatitis, HIV Infections, Huntington Disease, Hypercholesterolemia, Hyperglycemia, Hyperplasia, Hypertensive disease, Hyperthyroidism, Hypopituitarism, Hypoproteinemia, Hypotension, natural Hypothermia, Hypothyroidism, Immunologic Deficiency Syndromes, Immune System Diseases, Inflammation, Inflammatory Bowel Diseases, Influenza, Intestinal Diseases, Ischemia, Kearns-Sayre syndrome, Keratoconus, Kidney Calculi, Kidney Diseases, Acute Kidney Failure, Chronic Kidney Failure, Polycystic Kidney Diseases, leukemia, Myeloid Leukemia, Acute Promyelocytic Leukemia, Liver Cirrhosis, Liver diseases, Liver neoplasms, Locked-In Syndrome, Chronic Obstructive Airway Disease, Lung Neoplasms, Systemic Lupus Erythematosus, Non-Hodgkin Lymphoma, Machado- Joseph Disease, Malaria, Malignant neoplasm of stomach, Animal Mammary Neoplasms, Marfan Syndrome, Meningomyelocele, Mental Retardation, Mitral Valve Stenosis, Acquired Dental Fluorosis, Movement Disorders, Multiple Sclerosis, Muscle Rigidity, Muscle Spasticity, Muscular Atrophy, Spinal Muscular Atrophy, Myopathy, Mycoses, Myocardial Infarction, Myocardial Reperfusion Injury, Necrosis, Nephrosis, Nephrotic Syndrome, Nerve Degeneration, nervous system disorder, Neuralgia, Neuroblastoma, Neuroma, Neuromuscular Diseases, Obesity, Occupational Diseases, Ocular Hypertension, Oligospermia, Degenerative polyarthritis, Osteoporosis, Ovarian Carcinoma, Pain, Pancreatitis, Papillon-Lefevre Disease, Paresis, Parkinson Disease, Phenylketonurias, Pituitary Diseases, Pre-Eclampsia, Prostatic Neoplasms, Protein Deficiency, Proteinuria, Psoriasis, Pulmonary Fibrosis, Renal Artery Obstruction, Reperfusion Injury, Retinal Degeneration, Retinal Diseases, Retinoblastoma, Schistosomiasis, Schistosomiasis mansoni, Schizophrenia, Scrapie, Seizures, Age-related cataract, Compression of spinal cord, Cerebrovascular accident, Subarachnoid Hemorrhage, Progressive supranuclear palsy, Tetanus, Trisomy, Turner Syndrome, Unipolar Depression, Urticaria, Vitiligo, Vocal Cord Paralysis, Intestinal Volvulus, Weight Gain, HMN (Hereditary Motor Neuropathy) Proximal Type I, Holoprosencephaly, Motor Neuron Disease, Neurofibrillary degeneration (morphologic abnormality), Burning sensation, Apathy, Mood swings, Synovial Cyst, Cataract, Migraine Disorders, Sciatic Neuropathy, Sensory neuropathy, Atrophic condition of skin, Muscle Weakness, Esophageal carcinoma, Lingual-Facial-Buccal Dyskinesia, Idiopathic pulmonary hypertension, Lateral Sclerosis, Migraine with Aura, Mixed Conductive-Sensorineural Hearing Loss, Iron deficiency anemia, Malnutrition, Prion Diseases, Mitochondrial Myopathies, MELAS Syndrome, Chronic progressive external ophthalmoplegia, General Paralysis, Premature aging syndrome, Fibrillation, Psychiatric symptom, Memory impairment, Muscle degeneration, Neurologic Symptoms, Gastric hemorrhage, Pancreatic carcinoma, Pick Disease of the Brain, Liver Fibrosis, Malignant neoplasm of lung, Age related macular degeneration, Parkinsonian Disorders, Disease Progression, Hypocupremia, Cytochrome-c Oxidase Deficiency, Essential Tremor, Familial Motor Neuron Disease, Lower Motor Neuron Disease, Degenerative myelopathy, Diabetic Polyneuropathies, Liver and Intrahepatic Biliary Tract Carcinoma, Persian Gulf Syndrome, Senile Plaques, Atrophic, Frontotemporal dementia, Semantic Dementia, Common Migraine, Impaired cognition, Malignant neoplasm of liver, Malignant neoplasm of pancreas, Malignant neoplasm of prostate, Pure Autonomic Failure, Motor symptoms, Spastic, Dementia, Neurodegenerative Disorders, Chronic Hepatitis C, Guam Form Amyotrophic Lateral Sclerosis, Stiff limbs, Multisystem disorder, Loss of scalp hair, Prostate carcinoma, Hepatopulmonary Syndrome, Hashimoto Disease, Progressive Neoplastic Disease, Breast Carcinoma, Terminal illness, Carcinoma of lung, Tardive Dyskinesia, Secondary malignant neoplasm of lymph node, Colon Carcinoma, Stomach Carcinoma, Central neuroblastoma, Dissecting aneurysm of the thoracic aorta, Diabetic macular edema, Microalbuminuria, Middle Cerebral Artery Occlusion, Middle Cerebral Artery Infarction, Upper motor neuron signs, Frontotemporal Lobar Degeneration, Memory Loss, Classical phenylketonuria, CADASIL Syndrome, Neurologic Gait Disorders, Spinocerebellar Ataxia Type 2, Spinal Cord Ischemia, Lewy Body Disease, Muscular Atrophy, Spinobulbar, Chromosome 21 monosomy, Thrombocytosis, Spots on skin, Drug-Induced Liver Injury, Hereditary Leber Optic Atrophy, Cerebral Ischemia, ovarian neoplasm, Tauopathies, Macroangiopathy, Persistent pulmonary hypertension, Malignant neoplasm of ovary, Myxoid cyst, Drusen, Sarcoma, Weight decreased, Major Depressive Disorder, Mild cognitive disorder, Degenerative disorder, Partial Trisomy, Cardiovascular morbidity, hearing impairment, Cognitive changes, Ureteral Calculi, Mammary Neoplasms, Colorectal Cancer, Chronic Kidney Diseases, Minimal Change Nephrotic Syndrome, Non-Neoplastic Disorder, X-Linked Bulbo- Spinal Atrophy, Mammographic Density, Normal Tension Glaucoma Susceptibility To Finding), Vitiligo-Associated Multiple Autoimmune Disease Susceptibility 1 (Finding), Amyotrophic Lateral Sclerosis And/Or Frontotemporal Dementia 1, Amyotrophic Lateral Sclerosis 1, Sporadic Amyotrophic Lateral Sclerosis, monomelic Amyotrophy, Coronary Artery Disease, Transformed migraine, Regurgitation, Urothelial Carcinoma, Motor disturbances, Liver carcinoma, Protein Misfolding Disorders, TDP-43 Proteinopathies, Promyelocytic leukemia, Weight Gain Adverse Event, Mitochondrial cytopathy, Idiopathic pulmonary arterial hypertension, Progressive cGVHD, Infection, GRN-related frontotemporal dementia, Mitochondrial pathology, and Hearing Loss.

In particular embodiments, the disease is associated with the gene ATXN1, ATXN2, or ATXN3, which may be targeted for treatment. In some embodiments, the CAG repeat region located in exon 8 of ATXN1, exon 1 of ATXN2, or exon 10 of the ATXN3 is targeted. In embodiments, the disease is spinocerebellar ataxia 3 (sca3), scal, or sca2 and other related disorders, such as Congenital Abnormality, Alzheimer’s Disease, Amyotrophic Lateral Sclerosis, Ataxia, Ataxia Telangiectasia, Cerebellar Ataxia, Cerebellar Diseases, Chorea, Cleft Palate, Cystic Fibrosis, Mental Depression, Depressive disorder, Dystonia, Esophageal Neoplasms, Exotropia, Cardiac Arrest, Huntington Disease, Machado- Joseph Disease, Movement Disorders, Muscular Dystrophy, Myotonic Dystrophy, Narcolepsy, Nerve Degeneration, Neuroblastoma, Parkinson Disease, Peripheral Neuropathy, Restless Legs Syndrome, Retinal Degeneration, Retinitis Pigmentosa, Schizophrenia, Shy-Drager Syndrome, Sleep disturbances, Hereditary Spastic Paraplegia, Thromboembolism, Stiff-Person Syndrome, Spinocerebellar Ataxia, Esophageal carcinoma, Polyneuropathy, Effects of heat, Muscle twitch, Extrapyramidal sign, Ataxic, Neurologic Symptoms, Cerebral atrophy, Parkinsonian Disorders, Protein S Deficiency, Cerebellar degeneration, Familial Amyloid Neuropathy Portuguese Type, Spastic syndrome, Vertical Nystagmus, Nystagmus End-Position, Antithrombin III Deficiency, Atrophic, Complicated hereditary spastic paraplegia, Multiple System Atrophy, Pallidoluysian degeneration, Dystonia Disorders, Pure Autonomic Failure, Thrombophilia, Protein C, Deficiency, Congenital Myotonic Dystrophy, Motor symptoms, Neuropathy, Neurodegenerative Disorders, Malignant neoplasm of esophagus, Visual disturbance, Activated Protein C Resistance, Terminal illness, Myokymia, Central neuroblastoma, Dyssomnias, Appendicular Ataxia, Narcolepsy-Cataplexy Syndrome, Machado- Joseph Disease Type I, Machado- Joseph Disease Type II, Machado- Joseph Disease Type III, Dentatorubral-Pallidoluysian Atrophy, Gait Ataxia, Spinocerebellar Ataxia Type 1, Spinocerebellar Ataxia Type 2, Spinocerebellar Ataxia Type 6 (disorder), Spinocerebellar Ataxia Type 7, Muscular Spinobulbar Atrophy, Genomic Instability, Episodic ataxia type 2 (disorder), Bulbo-Spinal Atrophy X-Linked, Fragile X Tremor/ Ataxia Syndrome, Thrombophilia Due to Activated Protein C Resistance (Disorder), Amyotrophic Lateral Sclerosis 1, Neuronal Intranuclear Inclusion Disease, Hereditary Antithrombin Iii Deficiency, and Late-Onset Parkinson Disease.

In embodiments, the disease is associated with expression of a tumor antigen-cancer or non-cancer related indication, for example acute lymphoid leukemia, diffuse large B cell lymphoma, follicular lymphoma, chronic lymphocytic leukemia, Hodgkin lymphoma, non-Hodgkin lymphoma. In embodiments, the target can be TET2 intron, a TET2 intron-exon junction, a sequence within a genomic region of chr4.

In embodiments, neurodegenerative diseases can be treated. In particular embodiments, the target is Synuclein, Alpha (SNCA). In certain embodiments, the disorder treated is a pain related disorder, including congenital pain insensitivity, Compressive Neuropathies, Paroxysmal Extreme Pain Disorder, High grade atrioventricular block, Small Fiber Neuropathy, and Familial Episodic Pain Syndrome 2. In certain embodiments, the target is Sodium Channel, Voltage Gated, Type X Alpha Subunit (SCNIOA).

In certain embodiments, hematopoietic stem cells and progenitor stem cells are edited, including knock-ins. In particular embodiments, the knock-in is for treatment of lysosomal storage diseases, glycogen storage diseases, mucopolysaccharoidoses, or any disease in which the secretion of a protein will ameliorate the disease. In one embodiment, the disease is sickle cell disease (SCD). In another embodiment, the disease is β-thalassemia.

In certain embodiments, the T cell or NK cell is used for cancer treatment and may include T cells comprising the recombinant receptor (e.g. CAR) and one or more phenotypic markers selected from CCR7+, 4-1BB+ (CD137+), TIM3+, CD27+, CD62L+, CD127+, CD45RA+, CD45RO-, t-betl′w, IL-7Ra+, CD95+, IL-2RP+, CXCR3+ or LFA-1+. In certain embodiments the editing of a T cell for caner immunotherapy comprises altering one or more T-cell expressed gene, e.g., one or more of FAS, BID, CTLA4, PDCD1, CBLB, PTPN6, B2M, TRAC and TRBC gene. In some embodiments, editing includes alterations introduced into, or proximate to, the CBLB target sites to reduce CBLB gene expression in T cells for treatment of proliferative diseases and may include larger insertions or deletions at one or more CBLB target sites. T cell editing of TGFBR2 target sequence can be, for example, located in exon 3, 4, or 5 of the TGFBR2 gene and utilized for cancers and lymphoma treatment.

Cells for transplantation can be edited and may include allele-specific modification of one or more immunogenicity genes (e.g., an HLA gene) of a cell, e.g., HLA-A, HLA-B, HLA-C, HLA-DRB1, HLA-DRB3/4/5, HLA-DQ, and HLA-DP MiHAs, and any other MHC Class I or Class II genes or loci, which may include delivery of one or more matched recipient HLA alleles into the original position(s) where the one or more mismatched donor HLA alleles are located, and may include inserting one or more matched recipient HLA alleles into a “safe harbor” locus. In an embodiment, the method further includes introducing a chemotherapy resistance gene for in vivo selection in a gene.

Methods and systems can target Dystrophia Myotonica-Protein Kinase (DMPK) for editing, in particular embodiments, the target is the CTG trinucleotide repeat in the 3′ untranslated region (UTR) of the DMPK gene. Disorders or diseases associated with DMPK include Atherosclerosis, Azoospermia, Hypertrophic Cardiomyopathy, Celiac Disease, Congenital chromosomal disease, Diabetes Mellitus, Focal glomerulosclerosis, Huntington Disease, Hypogonadism, Muscular Atrophy, Myopathy, Muscular Dystrophy, Myotonia, Myotonic Dystrophy, Neuromuscular Diseases, Optic Atrophy, Paresis, Schizophrenia, Cataract, Spinocerebellar Ataxia, Muscle Weakness, Adrenoleukodystrophy, Centronuclear myopathy, Interstitial fibrosis, myotonic muscular dystrophy, Abnormal mental state, X-linked Charcot- Marie-Tooth disease 1, Congenital Myotonic Dystrophy, Bilateral cataracts (disorder), Congenital Fiber Type Disproportion, Myotonic Disorders, Multisystem disorder, 3- Methylglutaconic aciduria type 3, cardiac event, Cardiogenic Syncope, Congenital Structural Myopathy, Mental handicap, Adrenomyeloneuropathy, Dystrophia myotonica 2, and Intellectual Disability.

In embodiments, the disease is an inborn error of metabolism. The disease may be selected from Disorders of Carbohydrate Metabolism (glycogen storage disease, G6PD deficiency), Disorders of Amino Acid Metabolism (phenylketonuria, maple syrup urine disease, glutaric acidemia type 1), Urea Cycle Disorder or Urea Cycle Defects (carbamoyl phosphate synthease I deficiency), Disorders of Organic Acid Metabolism (alkaptonuria, 2-hydroxyglutaric acidurias), Disorders of Fatty Acid Oxidation/Mitochondrial Metabolism (Medium-chain acyl-coenzyme A dehydrogenase deficiency), Disorders of Porphyrin metabolism (acute intermittent porphyria), Disorders of Purine/Pyrimidine Metabolism (Lesch-Nynan syndrome), Disorders of Steroid Metabolism (lipoid congenital adrenal hyperplasia, congenital adrenal hyperplasia), Disorders of Mitochondrial Function (Kearns-Sayre syndrome), Disorders of Peroxisomal function (Zellweger syndrome), or Lysosomal Storage Disorders (Gaucher’s disease, Niemann-Pick disease).

In embodiments, the target can comprise Recombination Activating Gene 1 (RAG1), BCL11 A, PCSK9, laminin, alpha 2 (lama2), ATXN3, alanine-glyoxylate aminotransferase (AGXT), collagen type vii alpha 1 chain (COL7a1), spinocerebellar ataxia type 1 protein (ATXN1), Angiopoietin-like 3 (ANGPTL3), Frataxin (FXN), Superoxidase Dismutase 1, soluble (SOD1), Synuclein, Alpha (SNCA), Sodium Channel, Voltage Gated, Type X Alpha Subunit (SCN10A), Spinocerebellar Ataxia Type 2 Protein (ATXN2), Dystrophia Myotonica-Protein Kinase (DMPK), beta globin locus on chromosome 11, acyl-coenzyme A dehydrogenase for medium chain fatty acids (ACADM), long- chain 3-hydroxyl-coenzyme A dehydrogenase for long chain fatty acids (HADHA), acyl-coenzyme A dehydrogenase for very long-chain fatty acids (ACADVL), Apolipoprotein C3 (APOCIII), Transthyretin (TTR), Angiopoietin-like 4 (ANGPTL4), Sodium Voltage-Gated Channel Alpha Subunit 9 (SCN9A), Interleukin-7 receptor (IL7R), glucose-6-phosphatase, catalytic (G6PC), haemochromatosis (HFE), SERPINA1, C9ORF72, β-globin, dystrophin, γ-globin.

In certain embodiments, the disease or disorder is associated with Apolipoprotein C3 (APOCIII), which can be targeted for editing. In embodiments, the disease or disorder may be Dyslipidemias, Hyperalphalipoproteinemia Type 2, Lupus Nephritis, Wilms Tumor 5, Morbid obesity and spermatogenic, Glaucoma, Diabetic Retinopathy, Arthrogryposis renal dysfunction cholestasis syndrome, Cognition Disorders, Altered response to myocardial infarction, Glucose Intolerance, Positive regulation of triglyceride biosynthetic process, Renal Insufficiency, Chronic, Hyperlipidemias, Chronic Kidney Failure, Apolipoprotein C-III Deficiency, Coronary Disease, Neonatal Diabetes Mellitus, Neonatal, with Congenital Hypothyroidism, Hypercholesterolemia Autosomal Dominant 3, Hyperlipoproteinemia Type III, Hyperthyroidism, Coronary Artery Disease, Renal Artery Obstruction, Metabolic Syndrome X, Hyperlipidemia, Familial Combined, Insulin Resistance, Transient infantile hypertriglyceridemia, Diabetic Nephropathies, Diabetes Mellitus (Type 1), Nephrotic Syndrome Type 5 with or without ocular abnormalities, and Hemorrhagic Fever with renal syndrome.

In certain embodiments, the target is Angiopoietin-like 4(ANGPTL4). Diseases or disorders associated with ANGPTL4 that can be treated include ANGPTL4 is associated with dyslipidemias, low plasma triglyceride levels, regulator of angiogenesis and modulate tumorigenesis, and severe diabetic retinopathy. both proliferative diabetic retinopathy and non-proliferative diabetic retinopathy.

In embodiments, editing can be used for the treatment of fatty acid disorders. In certain embodiments, the target is one or more of ACADM, HADHA, ACADVL. In embodiments, the targeted edit is the activity of a gene in a cell selected from the acyl-coenzyme A dehydrogenase for medium chain fatty acids (ACADM) gene, the long- chain 3-hydroxyl-coenzyme A dehydrogenase for long chain fatty acids (HADHA) gene, and the acyl-coenzyme A dehydrogenase for very long-chain fatty acids (ACADVL) gene. In one aspect, the disease is medium chain acyl-coenzyme A dehydrogenase deficiency (MCADD), long-chain 3-hydroxyl-coenzyme A dehydrogenase deficiency (LCHADD), and/or very long-chain acyl-coenzyme A dehydrogenase deficiency (VLCADD).

### Immune Orthogonal Orthologs

In some embodiments, when Cas proteins need to be expressed or administered in a subject, immunogenicity of Cas proteins may be reduced by sequentially expressing or administering immune orthogonal orthologs of the CRISPR enzymes to the subject. As used herein, the term “immune orthogonal orthologs” refer to orthologous proteins that have similar or substantially the same function or activity, but have no or low cross-reactivity with the immune response generated by one another. In some embodiments, sequential expression or administration of such orthologs elicits low or no secondary immune response. The immune orthogonal orthologs can avoid being neutralized by antibodies (e.g., existing antibodies in the host before the orthologs are expressed or administered). Cells expressing the orthologs can avoid being cleared by the host’s immune system (e.g., by activated CTLs). In some examples, CRISPR enzyme orthologs from different species may be immune orthogonal orthologs.

Immune orthogonal orthologs may be identified by analyzing the sequences, structures, and/or immunogenicity of a set of candidates orthologs. In an example method, a set of immune orthogonal orthologs may be identified by a) comparing the sequences of a set of candidate orthologs (e.g., orthologs from different species) to identify a subset of candidates that have low or no sequence similarity; b) assessing immune overlap among the members of the subset of candidates to identify candidates that have no or low immune overlap. In some cases, immune overlap among candidates may be assessed by determining the binding (e.g., affinity) between a candidate ortholog and MHC (e.g., MHC type I and/or MHC II) of the host. Alternatively or additionally, immune overlap among candidates may be assessed by determining B-cell epitopes for the candidate orthologs. In one example, immune orthogonal orthologs may be identified using the method described in Moreno AM et al., BioRxiv, published online Jan. 10, 2018, doi: doi.org/10.1101/245985.

### EXAMPLES

### Example 1 - Highly Parallel Profiling of Cas9 Variant Specificity

Determining the off-target cleavage profile of programmable nucleases is an important consideration for any genome editing experiment, and a number of Cas9 variants have been reported that improve specificity. Applicants described here Tagmentation-based Tag Integration Site Sequencing (TTISS), an efficient, scalable method for analyzing double-strand breaks that Applicants applied in parallel to eight Cas9 variants across 59 targets. Additionally, Applicants generated thousands of other Cas9 variants and screened for variants with enhanced specificity and activity, identifying LZ3 Cas9, a high-specificity variant with a unique +1 insertion profile. This comprehensive comparison revealed a general trade-off between Cas9 activity and specificity and provides information about the frequency of generation of +1 insertions, which has implications for correcting frameshift mutations.

CRISPR-Cas9 technology is widely used for genome editing and is currently being tested in clinical trials as a therapeutic. Many applications of this technology rely on Cas9 from Streptococcus pyogenes (SpCas9), and a number of engineered or evolved SpCas9 variants have been reported that impact Cas9 specificity. Although a number of techniques have been developed that assess off-target cleavage (Tsai and Joung, 2016), these techniques are relatively low-throughput-limited to one guide per barcoded sample. Applicants therefore developed Tagmentation-based Tag Integration Site Sequencing (TTISS), an efficient, rapid, scalable method to assess editing outcomes.

### Experimental Design

Applicants’ method made use of guide multiplexing and bulk tagmentation by Tn5, which can be performed directly in lysed cells, leading to an efficient, rapid protocol (FIG. 1A). Following tagmentation, DNA was quickly purified using a spin column. Integration sites were enriched using two nested PCRs, which provided sufficient specificity to allow direct sequencing of the final product without further enrichment. Assigning the sequenced integration sites to guides by sequence similarity generated a list of off-target sites for each guide in parallel.

### Results

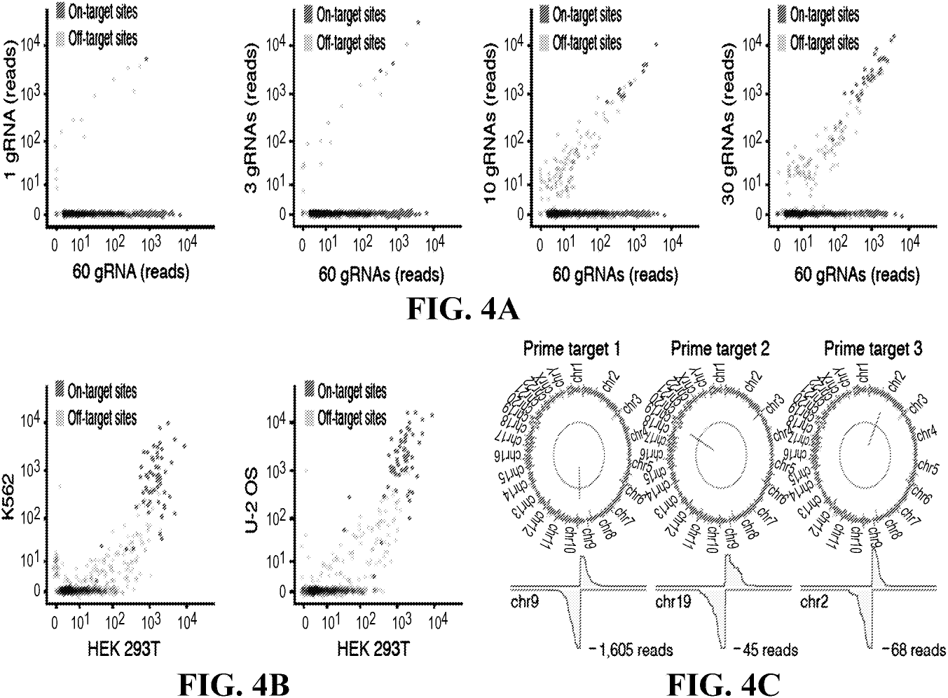

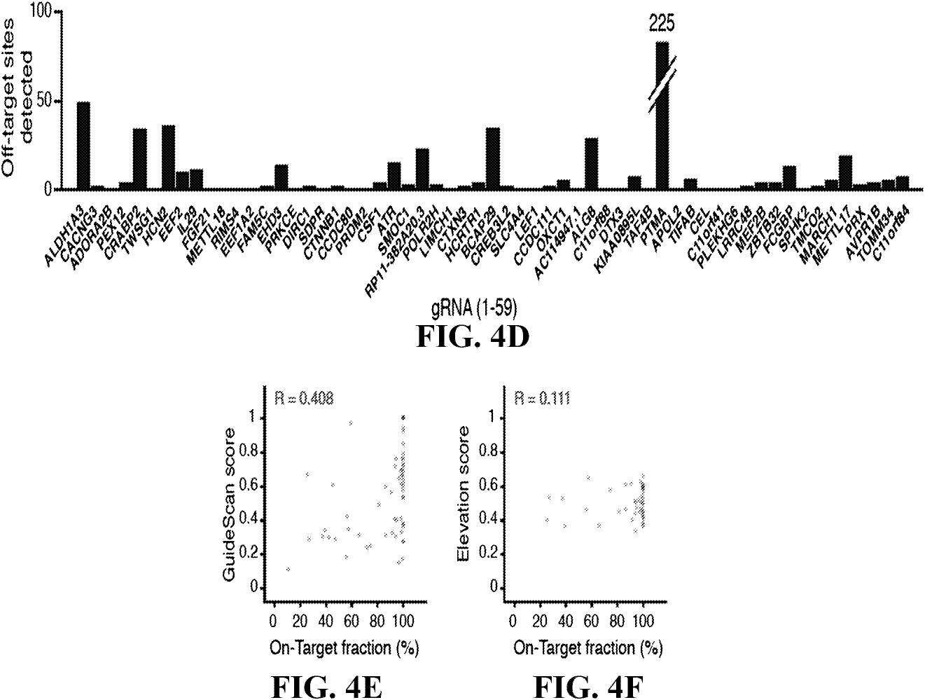

The sensitivity of TTISS was comparable to GUIDE-seq (Table 3, note GUIDE-seq data is from U-2 OS cells using matched single guides) and DISCOVER-Seq (Table 3, using matched single guides) (Wienert et al., 2019). TTISS was scalable to at least 60 guides per transfection in HEK 293T cells (FIG. 4A), while retaining 71.4% of off-target sites detected in a single guide experiment and was compatible with multiple cell types (FIG. 4B). Additionally, TTISS can be extended to profiling of prime editing-mediated donor integration (Anzalone et al., 2019), which showed no off-target integration events for three integration sites tested (FIG. 4C).

Applicants used TTISS to assess the specificity of WT SpCas9 and eight SpCas9 specificity variants - eSpCas9(1.1) (Slaymaker et al., 2015), SpCas9-HF1 (Kleinstiver et al., 2016), HypaCas9 (Chen et al., 2017), evoCas9 (Casini et al., 2018), xCas9(3.7) (Hu et al., 2018), Sniper-Cas9 (Lee et al., 2018), HiFi Cas9 (Vakulskas et al., 2018) - and one newly generated specificity variant, LZ3 Cas9 (see Methods, FIGS. 2A-2E) in parallel using 59 guides in two pools randomly selected from the GeCKO library (Shalem et al., 2014) that all start with a guanine to improve U6 transcription (FIG. 1B). For WT SpCas9, TTISS detected 607 total off-target sites across two technical replicates, with individual guides contributing 0-225 off-target sites (FIG. 4D, Table 5). Although each specificity variant showed improvement relative to WT SpCas9, a systematic comparison of these variants had not been reported. Using TTISS, Applicants found that, although each specificity variant eliminated at least half of the WT SpCas9 off-targets, there was a wide range of specificities among variants, with evoCas9 being most specific (4 detected off-targets) and SniperCas9 being least specific (287 detected off-targets) (FIG. 1B).

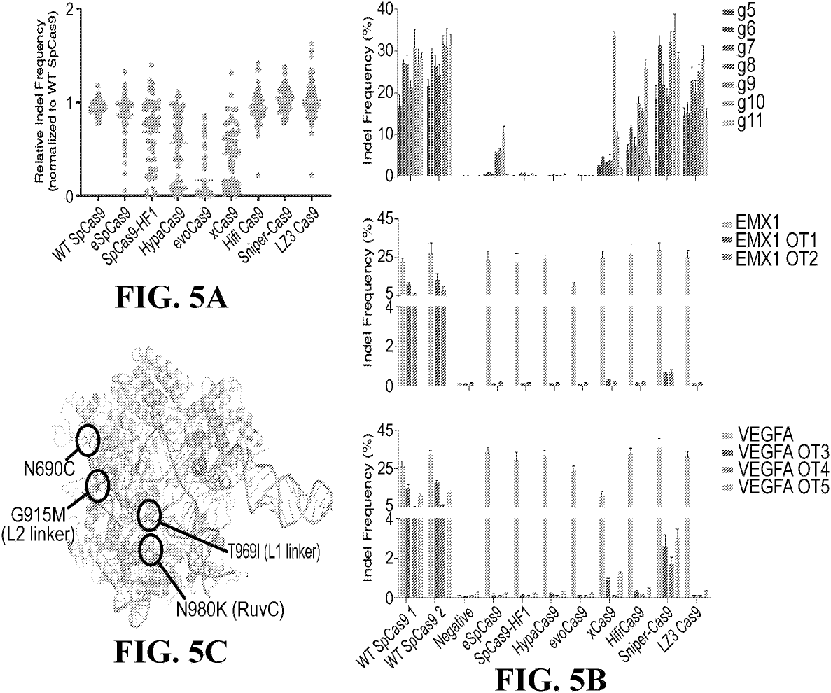

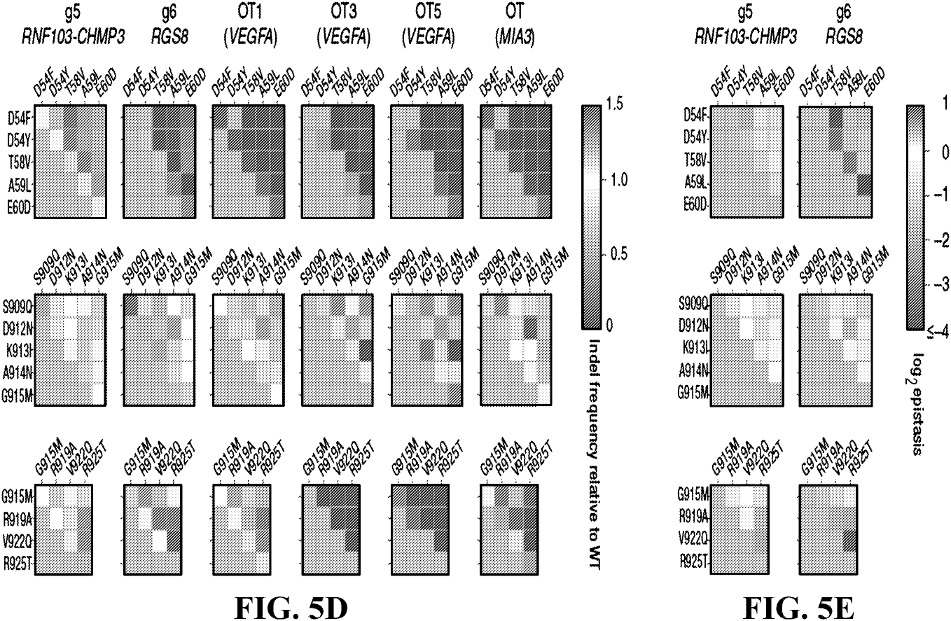

Measuring on-target indel frequencies by targeted sequencing revealed that evoCas9 and xCas9(3.7) had the lowest on-target activity, while LZ3 Cas9, HiFi Cas9 and Sniper-Cas9 had on-target activity comparable to WT SpCas9 (FIGS. 5A, 5B). To compare specificity variants more broadly, Applicants calculated an activity and a specificity score for each variant (FIG. 1C), revealing a general trade-off between activity and specificity among all variants.

To assess whether this observed trade-off between activity and specificity was a general feature of the SpCas9 mutation space, Applicants performed a high-throughput pooled lentiviral screen to comprehensively profile variant activity in human cells. Applicants selected 157 residues for mutagenesis (FIG. 2A), focusing on the HNH and RuvC nuclease domains, as well as the L1 and L2 linkers connecting them, as these regions played a key role in the conformational activation of Cas9 to license target cleavage (Palermo et al., 2016). Applicants selected four diverse target sites to assay the variants on: a putative ‘permissive’ guide (g1) known to be highly active for eSpCas9(1.1) and SpCas9-HF1; a ‘difficult’ guide (g2) with no activity for eSpCas9(1.1) and SpCas9-HF1; and two simulated off-targets (g3 and g4) bearing two mismatches each (FIG. 2B). Barcoded variants were cloned into a lentiviral vector and transduced into HEK 293FT cells (FIG. 2C), along with a guide RNA cassette and cognate target site. A total of 2,420 single amino acids variants exceeded the minimum read threshold for all four targets, representing 9.2% of all possible single amino acid variants of SpCas9. The activity of these variants was highly guide-dependent: over 20% of the variants improved specificity (≤50% activity at mismatched off-target; ≥80% activity on-target) when comparing g1 vs. g3, while <1% of variants met these criteria when comparing g2 vs. g4 (FIG. 2D). Applicants validated the performance of 254 variants on a broader range of targets (including three targets known to have low activity for eSpCas9(1.1) and SpCas9-HF1) by individual transfections and targeted deep sequencing (FIG. 2E). Overall, these results suggested that a simple guide-dependent trade-off describes the performance of a broad range of Cas9 variants.

A number of algorithms had been developed that aim to predict editing outcomes, including specificity and, more recently, indel distributions. Comparison of TTISS specificity data to two published computational tools that provide specificity scores for guides -GuideScan (guidescan.com) (Perez et al., 2017) and CRISPR ML (crispr.ml) (Listgarten et al., 2018) showed a weak correlation (GuideScan, n = 59, R = 0.408, CRISPR ML, n = 47, R = 0.111) between the predicted metric and empirical observation (FIGS. 4E, 4F).

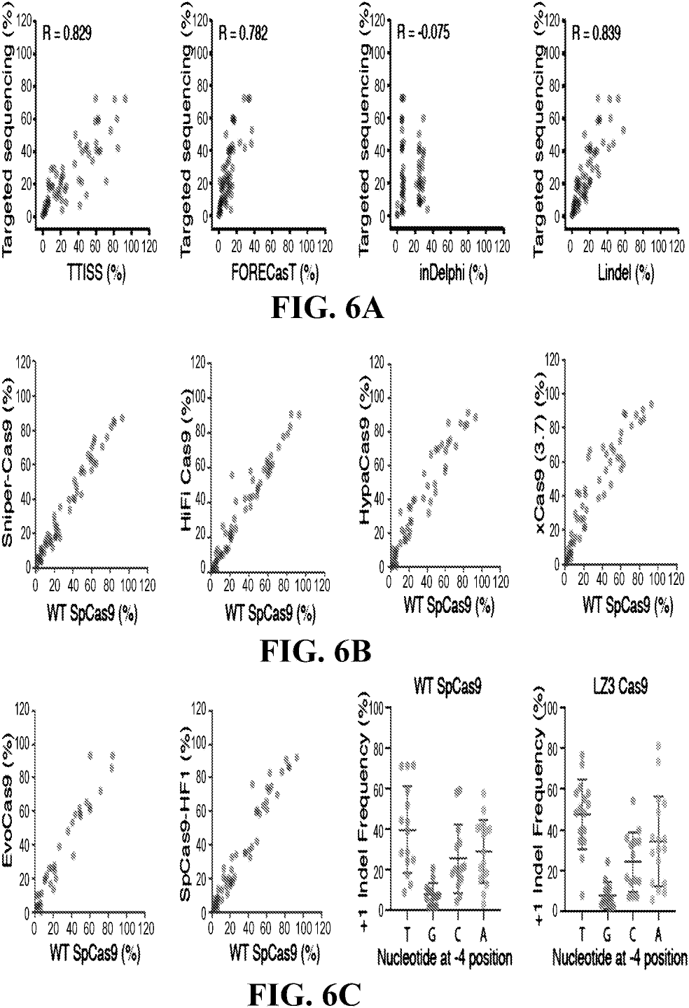

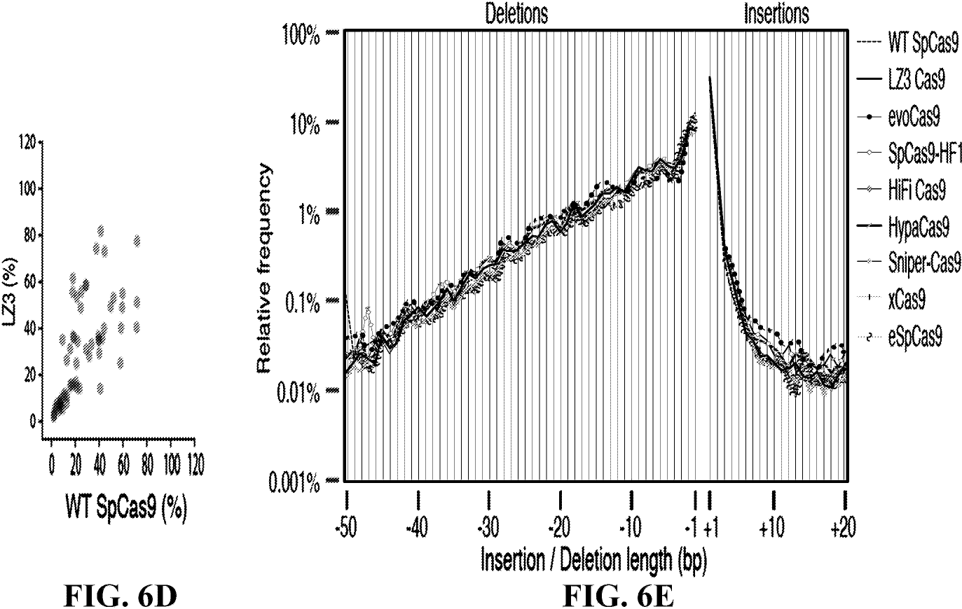

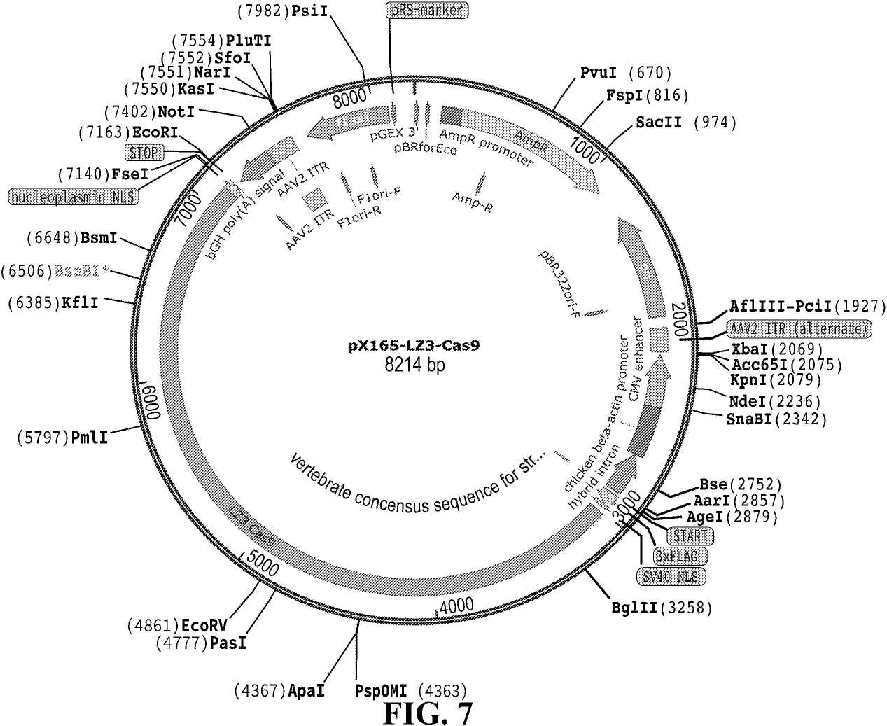

Although the predominant outcome of Cas9 cleavage was a blunt DSB created by the concerted effort of the two nuclease domains, HNH and RuvC, the RuvC domain was not as rigidly positioned and it can slide one base upstream (distal to the PAM), giving rise to a staggered cut that was filled in by the cellular repair machinery and led to duplication of a single base (+1 insertion) (FIG. 3A) (Zuo and Liu, 2016). This property was particularly useful in the genome engineering context because +1 insertions in protein-coding regions guarantee frameshifts, which had utility either for knocking out a gene or for the correction of a genetic variant. Applicants therefore examined whether Applicants could predict the relative frequencies of +1 insertions in the indel distribution for a given on-target site from multiplex TTISS data. Because TTISS relied on integration of a donor, Applicants developed an algorithm to predict +1 insertions based on the distribution of the position of the donor relative to the cut site. To obtain the distribution for each cut site, Applicants compiled the number of donor integrations at each nucleotide position relative to the cut site for both ends of the donor. Applicants then used a convolution operation to merge these two distributions to model the situation in which no donor is integrated, allowing to predict +1 frequencies (FIG. 3B). To validate the approach, Applicants compared the +1 frequencies obtained by TTISS for WT SpCas9 for 58 guides to those measured by targeted indel sequencing (FIG. 6A) and found a high correlation (r = 0.829), suggesting TTISS can be used to predict +1 frequency of a given guide. Prediction tools for Cas9-induced indel length distributions performed heterogeneously in predicting +1 frequencies compared to the empirical data (FORECasT (Allen et al., 2018), R = 0.782; inDelphi (Shen et al., 2018), R = -0.075; Lindel (Chen et al., 2019), R = 0.839)(FIG. 6A).

Given that many of the Cas9 variants contained mutations impacting DNA binding, which could potentially affect RuvC positioning, Applicants compared the indel patterns of Cas9 specificity variants across a set of 58 guides. While most variants closely mirrored +1 frequencies of WT SpCas9 across on-target sites by TTISS (FIG. 6B), the variant LZ3 Cas9 exhibited a markedly different +1 frequency profile relative to WT SpCas9 (FIG. 3C), which was confirmed by targeted sequencing data (FIG. 6D). Exploring sequence determinants for +1 frequencies of LZ3 Cas9 and WT SpCas9 revealed that for both enzymes, the presence of a thymidine or a guanine in the -4 position with respect to the PAM led to the highest and lowest rates of +1 insertion respectively (FIG. 6C). However, when comparing LZ3 Cas9 to WT SpCas9, LZ3 Cas9 showed elevated +1 frequency given a guanine at position -2 (FIG. 3D). Overall indel profiles were not found to be altered for any of the Cas9 variants tested (FIG. 6E).

Here Applicants show that TTISS was a scalable, accessible, and cost-effective method for examining off-targets and +1 insertion frequencies of programmable nucleases. Beyond these applications, TTISS was successfully applied to detect off-targets in other genome editing contexts, including editing by Cas enzymes creating overhanging, rather than blunt, ends, Cas enzymes delivered as ribonucleoprotein complexes, and ShCAST-mediated genome insertions. Multiplex TTISS enabled the creation of substantially larger sets of empirical data that could contribute to improved predictive algorithms or identify high-specificity guides suitable for clinical applications. Applying TTISS example embodiments across a panel of SpCas9 variants revealed a tradeoff between activity and specificity, which is also supported by the Cas9 mutational screening results. Applicants also showed that the newly evolved LZ3 Cas9 variant exhibits high activity, increased specificity, and a differential +1 insertion profile as compared to WT SpCas9.

### Experimental Model and Subject Details

### HEK 293T Cells

HEK 293T cells were maintained at 37C, 5% CO₂ in DMEM-GlutaMAX (Gibco) supplemented with 10% FBS (Seradigm) and 10 µg/ml Ciprofloxacin (Sigma-Aldrich). HEK 293T cells were originally derived from a female human embryo. Cells were obtained from the lab of Veit Hornung.

### U-2 OS Cells

U-2 OS cells were maintained at 37C, 5% CO₂ in DMEM-GlutaMAX (Gibco) supplemented with 10% FBS (Seradigm) and 10 µg/ml Ciprofloxacin (Sigma-Aldrich). U-2 OS were originally established from the osteosarcoma of female patient. Cells were obtained from ATCC. Cell line authentication was performed by the vendor.

### K562 Cells

K562 cells were maintained at 37C, 5% CO2 in RPMI-GlutaMAX (Gibco) supplemented with 10% FBS and 10 µg/ml Ciprofloxacin (Sigma-Aldrich). K562 cells were originally established from the chronic myelogenous leukemia of a female patient. Cells were obtained from Sigma-Aldrich. Cell line authentication was performed by the vendor.

### E. Coli Strains

STBL3 E. coli cells (ThermoFisher) were grown in LB media at 37C overnight. Chemo-competent cells were generated using the Mix&Go kit (Zymo).

### Method Details

### Tn5 Purification

Tn5 was purified as previously described (Picelli et al., 2014). E. coli cells (NEB C3013) harboring pTBX1-Tn5 were grown in terrific broth to an OD of 0.65 before addition of IPTG at 0.25 mM. Protein expression was induced at 23° C. overnight, and cells were harvested and stored at -80° C. until purification. 20 g of E. coli pellet was lysed in 200 mL HEGX buffer (20 mM HEPES-KOH pH 7.2, 800 mM NaCl, 1 mM EDTA, 0.2% Triton, 10% glycerol) with cOmplete protease inhibitor (Roche) and 10 uL of benzonase (Sigma-Aldrich). Cells were lysed using a LM20 microfluidizer device (Microfluidics) and cleared by centrifugation at max speed for 30 min. 5.25 mL of 10% PEI (pH 7) was added dropwise to a stirring solution to remove E. coli DNA and the resulting precipitation removed after centrifugation for 10 min. Cleared supernatant was added to 30 mL of equilibrated chitin resin (NEB), mixed end-over-end for 30 min, added to column, washed with 1 L HEGX buffer. 75 mL HEGX buffer with 100 mM DTT was added to column, 30 mL drawn through the resin before sealing the column and storing at 4° C. for 48 h to allow for intein cleavage and elution of free Tn5. Eluted Tn5 was dialyzed into 2xTn5 dialysis buffer (100 HEPES, 200 NaCl, 2 EDTA, 0.2 Triton, 20% glycerol), with two exchanges of 1 L of buffer. The final solution was concentrated to 50 mg/mL as determined by A280 absorbance (A280 = 1 = 0.616 mg/mL = 11.56 mM) and flash frozen in liquid nitrogen before storage at -80° C.

### Tn5 Loading With Single Handle

Oligonucleotides Transposon ME and Transposon read 2 were annealed at a concentration of 42 µM each in annealing buffer (1.5 mM Tris-HCl pH 8.0, 150 µM EDTA, 30 mM NaCl) by heating to 95° C. for 3 minutes, and subsequently ramping the temperature from 70C to 25° C. at a rate of 1° C. per minute. 1 ml of purified Tn5 (50 mg/ml) were incubated with 355 µl of annealed oligonucleotides for 1 hour at room temperature. Of note, loaded Tn5 can crash out as white precipitate, but retains activity. Loaded Tn5 is stored at -20° C. and ready to be thawed on ice for later use.

### Cas9 Variant Cloning

Cas9 variants were cloned by site-directed mutagenesis into pX165 (Addgene #48137), which encodes a CBh promoter-driven SpCas9 containing a 3xFLAG tag and SV40 NLS on the N terminus and a nucleoplasmin NLS on the C terminus.

### Cell Transfection

HEK 293T cells were seeded in poly-D-lysine coated 96-well plates (Corning) at a density of 25,000 cells in 100 µl medium per well. The next day, 250 µl OptiMEM (Thermo) were mixed with 1 µg of oligonucleotide donor (TTISS donor sense and TTISS donor antisense, annealed in 0.1x IDT Nuclease-Free Duplex Buffer by ramping the temperature from 95° C. to 25° C. at a rate of 1° C. per minute), 750 ng Cas9 expression plasmid, and a total of 250 ng of 1-60 different gRNA expression plasmids (sequences in Table 5). In parallel, 250 µl OptiMEM were mixed with 5 µl GeneJuice (Millipore) and incubated at room temperature for 5 minutes. After mixing all components and incubating them for 20 minutes, 50 µl were added drop-wise per 96-well of cells in a total of ten wells per condition. For prime editing, the same transfection protocol was used with 1.5 µg pCMV-PE2 plasmid and 500 ng pU6-pegRNA. For TTISS in K562 and U-2 OS cells, one million cells were nucleofected with pulse code FF-120 (K562) or CM-104 (U-2 OS) using a Lonza 4D-Nucleofector X unit in 100 µl buffer SF (K562) or SE (U-2 OS) with the same amounts of Cas9, gRNA, and donor as listed above.

### Cell Lysis and Genome Tagmentation

Three days after transfection, cells were washed with PBS, trypsinized, and washed again in a 1.5 ml tube. Pelleted cells were lysed by re-suspending one million cells in 100 µl lysis buffer (1 mM CaCl2, 3 mM MgCl2, 1 mM EDTA, 1% Triton X-100, 10 mM Tris pH 7.5, 8 units/ml Proteinase K (NEB)) and heating to 65° C. for 10 minutes. For tagmentation, 80 µl crude lysate were mixed with 25 µl 5x TAPS buffer (50 mM TAPS-NaOH pH 8.5 at room temperature, 25 mM MgCl2) and 20 µl hyperactive loaded Tn5 transposase and were heated to 55° C. for 10 minutes. Reactions were mixed with 625 µl PB buffer (Qiagen) and purified on a mini-prep silica spin column according to the protocol (Qiagen). DNA was eluted in 50 µl water (typical concentration: 200-300 ng/µl).

### PCR Amplification

Total eluates were denatured at 95° C. for 5 minutes, snap-cooled on ice, and amplified in 200 µl PCR reactions using KOD Hot Start polymerase (Millipore) according to the manufacturer’s protocol (12 cycles, Ta = 60° C., one minute elongation, primers: TTISS PCR fwd. 1, Transposon read 2). For each sample, a secondary 50 µl KOD PCR was templated with 3 µl of the first PCR reaction and a unique barcoding primer (20 cycles, Ta = 65° C., one minute elongation, primers: TTISS PCR fwd. 2, TTISS PCR rev BC1-24). For mapping prime-mediated insertions, primers TTISS PCR prime +24 fwd. a, b or TTISS PCR prime +38 fwd. a1, a2, b1, b2 were used instead.

### Deep Sequencing

PCRs were pooled, column-purified, and 250-1,000 bp fragments were enriched using a 2% agarose gel. After two consecutive column purifications, the library was quantified using a NanoDrop spectrometer (Thermo) and sequenced using an Illumina NextSeq 500 sequencer with a 75-cycle high-output v2 kit (cycle numbers: read 1 = 59, index 1 = 8, read 2 = 25, no index 2).

### Read Mapping

Reads were mapped to human genome version hg38 using BrowserGenome.org (Schmid-Burgk and Hornung, 2015) with mapping parameters: read filter = NNNNNNNNNNNNNNNNNNNNNNNAAC (SEQ ID NO: 2), forward mapping start = 26 bp, forward mapping length = 25 bp, reverse mapping length = 15 bp, max forward/reverse span = 1000 bp. For mapping prime-mediated insertions, read filters CTTATCGTCGTCATCCTTGTAATC (SEQ ID NO: 3) (+24 a, forward mapping start = 25), GATTACAAGGATGACGACGATAAG (SEQ ID NO: 4) (+24 b, forward mapping start = 25), GACGGCGGTCTCCGTCGTCAGGATCAT (SEQ ID NO: 5) (+38 a, forward mapping start = 28), or GACGGAGACCGCCGTCGTCGACAAGCC (SEQ ID NO: 6) (+38 b, forward mapping start = 28) were used instead. Mapped read pairs spanning fewer than 37 genome bases were discarded in order to omit signal from the pegRNA expression plasmid.

### Integration Site Detection

Common break sites, common mispriming sites and reads mapping to the human U6 promoter were filtered out. These were detected by TTISS in the absence of a nuclease, donor, and/or gRNA plasmid. Following removal of non-overlapping single-read noise, putative break sites were identified by the presence of two or more unique reads mapping to the reference sequence within a window of 20 nucleotides. For all sites passing filters, TTISS read counts mapping to a 60-nucleotide window were tabulated and stored for downstream analysis.

### gRNA Assignment

For each 60-nucleotide window, peaks were identified in both the sense and antisense reads, and each peak was grouped with all gRNA sequences used in the respective experiment whose spacers had an edit distance less than or equal to 6 mismatches for any 20-mer in a window of 25 nucleotides on either side of the detected peak site. If a given peak site had at least one such gRNA, then a cut site score was calculated for each putative gRNA match. The cut site score was defined as the distance between the expected cut site of the spacer and the peak. Each remaining peak site was then assigned to gRNA with the lowest cut site score and all peak sites with a cut site score of between -3 and 3 were retained and reported for each individual gRNA. This allows for the possibility of multiple cut sites within the same window, as well as for the removal of false hits where the apparent cut site does not line up with the expected cut site from the spacer sequence.

### Prediction of Indel Length Distributions

Genomic positions of TTISS-detected donor integration events were tabulated for each gRNA target site with more than 50 reads mapping in each orientation. Obtained distributions were normalized to their total number of reads in order to obtain two frequency distributions per target site. TTISS-predicted indel length distributions were calculated by numerically convolving the two directional distributions for each target site. From each indel length distribution, relative +1 frequencies were calculated as the ratio of +1 frequency to the sum of all non-+0 repair frequencies.

### Variant Scoring

Specificity scores were calculated by subtracting from 100 the percent of TTISS reads that corresponds to off-targets. Activity scores were calculated as the mean indel percentage across all 59 on-target sites, normalized to WT SpCas9.

### Cas9 Variant Library Construction

SpCas9 variants were screened using a pool of self-targeting lentiviral vectors in which each lentiviral insert contained a Cas9 variant and a constant target site, allowing indel formation at the target site to be coupled to its corresponding Cas9 variant. For the variant pool, >150 residue positions, concentrated in the HNH and RuvC nuclease domains, were selected for single amino acid saturation mutagenesis. For each residue, a mutagenic insert was synthesized as short complementary oligonucleotides, with the mutated codon replaced by a degenerate NNK mixture of bases, as previously described in (Gao et al., 2017). Furthermore, variants were barcoded with a random 24-nt sequence placed in close proximity to the target site in order to allow direct variant-to-indel association by short-read paired-end sequencing. Barcode-to-variant associations were determined by targeted deep sequencing prior to performing the screen.

### Lentiviral Cas9 Variant Library Screen

HEK 293FT cells were transduced with the variant library at MOI <0.1 and selected with puromycin at 1 µg/mL over several passages to eliminate non-transduced cells. Variant library-transduced cells were subsequently transduced with a second lentivirus containing an U6-sgRNA expression cassette at MOI >> 1 and >1000 cells/variant, in order to initiate indel formation at the target site. After approximately 4 days, genomic DNA from cells were isolated, and the target site and corresponding barcodes were PCR-amplified and paired-end sequenced with a 150-cycle NextSeq 500/550 High Output Kit v2 (Illumina). This procedure was repeated for four different sgRNAs: Two fully matched sgRNAs, to assess on-target efficiency of the variants; and two sgRNA bearing double base mismatches, to assess specificity (all guide sequences in Table 5). Highly abundant barcodes (above 50 reads; comprising 5%, 2%, 3% and 3% of all barcodes for g1, g2, g3 and g4, respectively) were discarded to reduce noise. For each guide, the score of a variant was calculated as 100 * (number of reads containing an indel) / (total number of reads pooled across all retained barcodes for that variant). Variants with fewer than 100 reads for any of the four target sites were discarded, resulting in a final set of 130 wild-type, 112 stop codons, and 2,420 single amino acid variants.

### Cas9 Variant Validation and Combinatorial Mutagenesis

Top hits from the pooled variant screen that exhibited both high on-target efficiency and high specificity were individually cloned into pX165 (Ran et al., 2013) and tested at additional target sites in HEK 293T cells, including sites that were previously observed to have substantially reduced activity with eSpCas9, SpCas9-HF1, and HypaCas9. Top-performing variants were combined to produce combination mutants, including LZ3 Cas9, which were re-tested as described and refined over 10 subsequent rounds of mutagenesis.

### Prime Editing Constructs

The following pegRNA sequences were cloned into pU6-pegRNA-GG-acceptor according to the protocol described in Anzalone et al., 2019 (Table 5).

### Targeted Indel Sequencing

Indel frequencies were quantified by targeted deep sequencing (Illumina) as previously described in (Gao et al., 2017). Indel distribution profiles were analyzed using OutKnocker.org (Schmid-Burgk et al., 2014).

### Indel Distribution and Specificity Predictors

Elevation scores (Listgarten et al., 2018) and GuideScan (Perez et al., 2017) scores were calculated by inputting the gene into the online interfaces (crispr.ml and guidescan.com) and storing the Elevation aggregate value and specificity value for the correct gRNA respectively. Predicted +1 insertion frequencies from FORECasT (Allen et al., 2018) and inDelphi (Shen et al., 2018) were evaluated by inputting the genomic locus (FORECasT) or 30 bp on either side of the cut site (inDelphi) into the correct online interface (partslab.sanger.ac.uk/FORECasT and the HEK 293 predictor on indelphi.giffordlab.mit.edu/single) and recording the total predicted % of 1-bp insertions Lindel-predicted values (Chen et al., 2019) were calculated similarly to inDelphi using the Python library (github.com/shendurelab/Lindel).

The sequencing data generated during this study are available at SRA (BioProject PRJNA602092). The code used for read post-processing used in this study is available at GitHub (schmidburgk/TTISS).

TABLE 2

Key resources used in this study

| REAGENT or RESOURCE | SOURCE | IDENTIFIER |
|---|---|---|
| Bacterial and Virus Strains | | |
| STBL3 | ThermoFisher | C737303 |
| T7 Express lysY/l^{q} Competent E. coli (High Efficiency) | NEB | C3013 |
| Chemicals, Peptides, and Recombinant Proteins | | |
| FBS, USA, Seradigm Premium | VWR | 97068-085 |
| KOD Hot Start DNA Polymerase | Millipore Sigma | 71086-3 |
| Proteinase K | NEB | P8107S |
| Tn5 | F. Zhang Lab | - |
| Qiaprep spin miniprep kit | Qiagen | 27106 |
| IPTG | Millipore Sigma | I6758 |
| cOmplete protease inhibitor | Millipore Sigma | 11697498001 |
| Benzonase | Millipore Sigma | E1014-25KU |
| Chitin resin | NEB | S6651L |
| OptiMEM | ThermoFisher | 31985070 |
| E-Gel ™ EX Agarose Gels, 2% | ThermoFisher | G402002 |
| GeneJuice | Millipore Sigma | 70967-3 |
| SF Cell Line 4D-Nucleofector® X Kit | Lonza | V4XC-2012 |
| SE Cell Line 4D-Nucleofector® X Kit | Lonza | V4XC-1012 |
| Puromycin | ThermoFisher | A1113802 |
| NextSeq 500/550 High Output Kit v2, 75 cycles | Illumina | FC-404-2005 |
| NextSeq 500/550 High Output Kit v2, 150 cycles | Illumina | FC-404-2002 |
| Nuclease-Free Duplex Buffer | IDT | 11-01-03-01 |
| Deposited Data | | |
| Deep Sequencing data | SRA | PRJNA602092 |
| Experimental Models: Cell Lines | | |
| HEK 293T | Gift from Veit Hornung | - |
| U-2 OS | ATCC | HTB-96 |
| K562 | Millipore Sigma | 89121407-1VL |
| Oligonucleotides | | |
| /5Phos/CTGTCTCTTATACA/3ddC/ (SEQ ID NO: 7) | IDT | Transposon ME |
| GTCTCGTGGGCTCGGAGATGTGTATAAGAGACAG (SEQ ID NO: 8) | IDT | Transposon read 2 |
| /5phos/G*T*TGTGAGCAAGGGCGAGGAGGATAACGCCTCTCTCCCAGCGACT*A*T (SEQ ID NO: 9) | IDT | TTISS donor sense |
| /5phos/A*T*AGTCGCTGGGAGAGAGGCGTTATCCTCCTCGCCCTTGCTCACA*A*C (SEQ ID NO: 10) | IDT | TTISS donor antisense |
| GTCGCTGGGAGAGAGGCGTTATC (SEQ ID NO: 11) | IDT | TTISS PCR fwd. 1 |
| AATGATACGGCGACCACCGAGATCTACACTATAGCCTACACTCTTTCCCTACACGACGCTCTTCCGATCTTTATCCTCCTCGCCCTTGCTCAC (SEQ ID NO: 12) | IDT | TTISS PCR fwd. 2 |
| CAAGCAGAAGACGGCATACGAGATCGAGTAATGTCTCGTGGGCTCGGAGATGTGT (SEQ ID NO: 13) | IDT | TTISS PCR rev BC1 |
| CAAGCAGAAGACGGCATACGAGATTCTCCGGAGTCTCGTGGGCTCGGAGATGTGT (SEQ ID NO: 14) | IDT | TTISS PCR rev BC2 |
| CAAGCAGAAGACGGCATACGAGATAATGAGCGGTCTCGTGGGCTCGGAGATGTGT (SEQ ID NO: 15) | IDT | TTISS PCR rev BC3 |
| CAAGCAGAAGACGGCATACGAGATGGAATCTCGTCTCGTGGGCTCGGAGATGTGT (SEQ ID NO: 16) | IDT | TTISS PCR rev BC4 |
| CAAGCAGAAGACGGCATACGAGATTTCTGAATGTCTCGTGGGCTCGGAGATGTGT (SEQ ID NO: 17) | IDT | TTISS PCR rev BC5 |
| CAAGCAGAAGACGGCATACGAGATACGAATTCGTCTCGTGGGCTCGGAGATGTGT (SEQ ID NO: 18) | IDT | TTISS PCR rev BC6 |
| CAAGCAGAAGACGGCATACGAGATAGCTTCAGGTCTCGTGGGCTCGGAGATGTGT (SEQ ID NO: 19) | IDT | TTISS PCR rev BC7 |
| CAAGCAGAAGACGGCATACGAGATGCGCATTAGTCTCGTGGGCTCGGAGATGTGT (SEQ ID NO: 20) | IDT | TTISS PCR rev BC8 |
| CAAGCAGAAGACGGCATACGAGATCATAGCCGGTCTCGTGGGCTCGGAGATGTGT (SEQ ID NO: 21) | IDT | TTISS PCR rev BC9 |
| CAAGCAGAAGACGGCATACGAGATTTCGCGGAGTCTCGTGGGCTCGGAGATGTGT (SEQ ID NO: 22) | IDT | TTISS PCR rev BC10 |
| CAAGCAGAAGACGGCATACGAGATGCGCGAGAGTCTCGTGGGCTCGGAGATGTGT (SEQ ID NO: 23) | IDT | TTISS PCR rev BC11 |
| CAAGCAGAAGACGGCATACGAGATCTATCGCTGTCTCGTGGGCTCGGAGATGTGT (SEQ ID NO: 24) | IDT | TTISS PCR rev BC12 |
| CAAGCAGAAGACGGCATACGAGATTGTAGTGCGTCTCGTGGGCTCGGAGATGTGT (SEQ ID NO: 25) | IDT | TTISS PCR rev BC13 |
| CAAGCAGAAGACGGCATACGAGATGCGTCGACGTCTCGTGGGCTCGGAGATGTGT (SEQ ID NO: 26) | IDT | TTISS PCR rev BC14 |
| CAAGCAGAAGACGGCATACGAGATGGTCTTCTGTCTCGTGGGCTCGGAGATGTGT (SEQ ID NO: 27) | IDT | TTISS PCR rev BC15 |
| CAAGCAGAAGACGGCATACGAGATAAATGTCCGTCTCGTGGGCTCGGAGATGTGT (SEQ ID NO: 28) | IDT | TTISS PCR rev BC16 |
| CAAGCAGAAGACGGCATACGAGATGTTGAAACGTCTCGTGGGCTCGGAGATGTGT (SEQ ID NO: 29) | IDT | TTISS PCR rev BC17 |
| CAAGCAGAAGACGGCATACGAGATTCTTTACGGTCT CGTGGGCTCGGAGATGTGT (SEQ ID NO: 30) | IDT | TTISS PCR rev BC18 |
| CAAGCAGAAGACGGCATACGAGATATGCCTGGGTCTCGTGGGCTCGGAGATGTGT (SEQ ID NO: 31) | IDT | TTISS PCR rev BC19 |
| CAAGCAGAAGACGGCATACGAGATCAATAAGGGTCTCGTGGGCTCGGAGATGTGT (SEQ ID NO: 32) | IDT | TTISS PCR rev BC20 |
| CAAGCAGAAGACGGCATACGAGATCGCCGTAAGTCTCGTGGGCTCGGAGATGTGT (SEQ ID NO: 33) | IDT | TTISS PCR rev BC21 |
| CAAGCAGAAGACGGCATACGAGATTAAGGCTTGTCTCGTGGGCTCGGAGATGTGT (SEQ ID NO: 34) | IDT | TTISS PCR rev BC22 |
| CAAGCAGAAGACGGCATACGAGATTTGCTGCCGTCTCGTGGGCTCGGAGATGTGT (SEQ ID NO: 35) | IDT | TTISS PCR rev BC23 |
| CAAGCAGAAGACGGCATACGAGATCTCAATGTGTCTCGTGGGCTCGGAGATGTGT (SEQ ID NO: 36) | IDT | TTISS PCR rev BC24 |
| AATGATACGGCGACCACCGAGATCTACACTATAGCCTACACTCTTTCCCTACACGACGctcttccgatctCTTATCGTCGTCATCCTTGT (SEQ ID NO: 37) | IDT | TTISS PCR prime +24 fwd. a |
| AATGATACGGCGACCACCGAGATCTACACTATAGCCTACACTCTTTCCCTACACGACGctcttccgatctGATTACAAGGATGACGACGA (SEQ ID NO: 38) | IDT | TTISS PCR prime +24 fwd. b |
| GGCTTGTCGACGACGGCGGTC (SEQ ID NO: 39) | IDT | TTISS PCR prime +38 fwd. a1 |
| AATGATACGGCGACCACCGAGATCTACACTATAGCCTACACTCTTTCCCTACACGACGctcttccgatctGACGGCGGTCTCCGTCGTCAG (SEQ ID NO: 40) | IDT | TTISS PCR prime +38 fwd. a2 |
| ATGATCCTGACGACGGAGACCG (SEQ ID NO: 41) | IDT | TTISS PCR prime +38 fwd. b1 |
| AATGATACGGCGACCACCGAGATCTACACTATAGCCTACACTCTTTCCCTACACGACGctcttccgatctGACGGAGACCGCCGTCGTCGA (SEQ ID NO: 42) | IDT | TTISS PCR prime +38 fwd. b2 |
| Recombinant DNA | | |
| pTBX1-Tn5 | Addgene | #60240 |
| pX165 | Addgene | #48137 |
| pCMV-PE2 | Addgene | #132775 |
| pU6-pegRNA-GG-acceptor | Addgene | #132777 |
| pX165-Sniper-Cas9 | This study | - |
| pX165-LZ3 Cas9 | This study | - |
| pX165-HiFi Cas9 | This study | - |
| pX165-eSpCas9 | This study | - |
| pX165-Cas9-HF1 | This study | - |
| pX165-HypaCas9 | This study | - |
| pX165-xCas9 | This study | - |
| pX165-evoCas9 | This study | - |
| Software and Algorithms | | |
| BrowserGenome | BrowserGenome.org | - |
| Elevation scoring | crispr.ml | - |
| GuideScan | guidescan.com | - |
| FORECasT | partslab.sanger.ac.uk/FORECasT | - |
| inDelphi | indelphi.giffordlab.mit.edu/single | - |
| Lindel | github.com/shendurelab/Lindel | - |

TABLE 3

Comparison of TTISS to GUIDE-Seq and DISCOVER-Seq. (related to FIGS. 1A-1C). List of target sites detected for the EMX1 and VEGFA 3 gRNAs from single-guide TTISS runs in HEK 293T cells. (Bolded nucleotides represent variant bases and unbolded nucleotides represent WT bases.)
EMX1

| Genome Position | GAGTCCGAGCAGAAGAAGAAGGG (SEQ ID NO: 43) | TTISS | GUIDE-seq |
|---|---|---|---|
| chr2:72933868 | GAGTCCGAGCAGAAGAAGAAGGG (SEQ ID NO: 44) | 1017 | 4521 |
| chr5:45358964 | GAGTTAGAGCAGAAGAAGAAAGG (SEQ ID NO: 45) | 1092 | 3123 |
| chr15:43817564 | GAGTCTAAGCAGAAGAAGAAGAG (SEQ ID NO: 46) | 862 | 1445 |
| chr2:218980348 | GAGGCCGAGCAGAAGAAAGACGG (SEQ ID NO: 47) | 411 | 700 |
| chr8:127789010 | GAGTCCTAGCAGGAGAAGAAGAG (SEQ ID NO: 48) | 584 | 390 |
| chr5:9227049 | AAGTCTGAGCACAAGAAGAATGG (SEQ ID NO: 49) | 180 | 258 |
| chrX:53440763 | GAGTCCGGGAAGGAGAAGAAAGG (SEQ ID NO: 50) | 239 | 216 |
| chr5:147453626 | GAGCCGGAGCAGAAGAAGGAGGG (SEQ ID NO: 51) | 31 | 143 |
| chr1:23394123 | AAGTCCGAGGAGAGGAAGAAAGG (SEQ ID NO: 52) | 58 | 102 |
| chr3:4989928 | GAATCCAAGCAGGAGAAGAAGGA (SEQ ID NO: 53) | 77 | 67 |
| chr6:9118565 | ACGTCTGAGCAGAAGAAGAATGG (SEQ ID NO: 54) | 20 | 38 |
| chr13:27195519 | GAGTAGCGAGCAGAGAAGAAGGA (SEQ ID NO: 55) | 12 | 7 |
| chr15:99752272 | AAGTCCCGGCAGAGGAAGAAGGG (SEQ ID NO: 56) | 8 | 6 |
| chr3:95971336 | TCATCCAAGCAGAAGAAGAAGAG (SEQ ID NO: 57) | 0 | 5 |
| chr10:57088967 | GAGCACGAGCAAGAGAAGAAGGG (SEQ ID NO: 58) | 10 | 2 |
| chr2:217513384 | GAGTCTAAGCAGGAGAATAAAGG (SEQ ID NO: 59) | 10 | 2 |
| chr17:76881488 | GAGGCCGGGCAGGAGAAGGAGGG (SEQ ID NO: 60) | 64 | 0 |
| chr6:110170207 | AAGTCAGAGCAGAAAGAAGGAGG (SEQ ID NO: 61) | 15 | 0 |
| chr11:43726397 | AAGCCCGAGCAAAGGAAGAAAGG (SEQ ID NO: 62) | 10 | 0 |
| chr4:21139710 | AAGCCCGAGCAGAAGAAGTTGAG (SEQ ID NO: 63) | 6 | 0 |

VEGFA 3

| | | | |
|---|---|---|---|
| Genome Position | GGTGAGTGAGTGTGTGCGTGTGG (SEQ ID NO: 64) | TTISS | GUIDE-seq |
| chr14:65102441 | AGTGAGTGAGTGTGTGTGTGGGG (SEQ ID NO: 65) | 933 | 3125 |
| chr5:90145150 | AGAGAGTGAGTGTGTGCATGAGG (SEQ ID NO: 66) | 1407 | 2559 |
| chr6:43769733 | GGTGAGTGAGTGTGTGCGTGTGG (SEQ ID NO: 67) | 417 | 2440 |
| chr5:116098978 | TGTGGGTGAGTGTGTGCGTGAGG (SEQ ID NO: 68) | 1819 | 2200 |
| chr22:37266781 | GCTGAGTGAGTGTATGCGTGTGG (SEQ ID NO: 69) | 2008 | 1997 |
| chr11:69083670 | GGTGAGTGAGTGCGTGCGGGTGG (SEQ ID NO: 70) | 805 | 1535 |
| chr10:97000829 | GTTGAGTGAATGTGTGCGTGAGG (SEQ ID NO: 71) | 446 | 1437 |
| chr3:194276094 | AGTGAATGAGTGTGTGTGTGTGG (SEQ ID NO: 72) | 340 | 1315 |
| chr14:61612055 | TGTGAGTAAGTGTGTGTGTGTGG (SEQ ID NO: 73) | 165 | 1170 |
| chr19:40055958 | ACTGTGTGAGTGTGTGCGTGAGG (SEQ ID NO: 74) | 139 | 796 |
| chr14:73886793 | AGCGAGTGGGTGTGTGCGTGGGG (SEQ ID NO: 75) | 436 | 790 |
| chr20:20197638 | AGTGTGTGAGTGTGTGCGTGTGG (SEQ ID NO: 76) | 536 | 686 |
| chr9:23824555 | TGTGGGTGAGTGTGTGCGTGAGA (SEQ ID NO: 77) | 298 | 643 |
| chr3:71583657 | CGCGAGTGAGTGTGTGCGCGGGG (SEQ ID NO: 78) | 25 | 215 |
| chr14:105562693 | GGTGAGTGAGTGTGTGTGTGAGG (SEQ ID NO: 79) | 272 | 199 |
| chr19:47229236 | CTGGAGTGAGTGTGTGTGTGTGG (SEQ ID NO: 80) | 30 | 193 |
| chr9:18733631 | AGCGAGTGAGTGTGTGTGTGGGG (SEQ ID NO: 81) | 0 | 149 |
| chr2:73089923 | GGTGAGTCAGTGTGTGAGTGAGG (SEQ ID NO: 82) | 20 | 122 |
| chr22:49344074 | GGTGTGTGAGTGTGTGTGTGTGG (SEQ ID NO: 83) | 25 | 115 |
| chr8:23074984 | TGTGAGTGAGTGTGTGTGTGTGA (SEQ ID NO: 84) | 0 | 111 |
| chr5:29367266 | TGTGAGTGAGTGTGTGCATGGGG (SEQ ID NO: 85) | 0 | 103 |
| chr4:57460425 | AGTGAGTGAGTGAGTGAGTGAGG (SEQ ID NO: 86) | 0 | 97 |
| chr13:114117523 | TGTGGGTGAGCATGTGCGTGAGG (SEQ ID NO: 87) | 6 | 83 |
| chr8:48085244 | GTAGAGTGAGTGTGTGTGTGTGG (SEQ ID NO: 88) | 61 | 82 |
| chr12:6827889 | GGTGGATGAGTGTGTGTGTGGGG (SEQ ID NO: 89) | 185 | 61 |
| chr16:79982434 | TGTGAGTGAGTGTGTGCGTGTGA (SEQ ID NO: 90) | 188 | 50 |
| chr19:1716790 | CATGAGTGAGTGTGTGGGTGGGG (SEQ ID NO: 91) | 38 | 45 |
| chr10:5707687 | AGTGAGTATGTGTGTGTGTGGGG (SEQ ID NO: 92) | 0 | 41 |
| chr6:156757193 | GATGAGTGAGTGAGTGAGTGGGG (SEQ ID NO: 93) | 197 | 37 |
| chr14:57651723 | TGTGAGTGAGTGTGTGTGTGTGA (SEQ ID NO: 94) | 38 | 37 |
| chr5:131521907 | GGAGAGTGAGTGTGTGTGTGAGA (SEQ ID NO: 95) | 19 | 35 |
| chr18:76391217 | GGTGAGTAAGTGTGAGCGTAAGG (SEQ ID NO: 96) | 334 | 33 |
| chr2:176598697 | GGTGAGTGTGTGTGTGCATGTGG (SEQ ID NO: 97) | 283 | 33 |
| chr11:79467476 | AGTGAGTGAGTGAGTGAGTGGGG (SEQ ID NO: 98) | 74 | 32 |
| chr4:61201901 | GATGAGTGTGTGTGTGTGTGAGG (SEQ ID NO: 99) | 50 | 29 |
| ch16:83999040 | GGTGAATGAGTGTGTGCTCTGGG (SEQ ID NO: 100) | 74 | 26 |
| chr10:128430090 | AGGGAGTGACTGTGTGCGTGTGG (SEQ ID NO: 101) | 241 | 24 |
| chr3:5063255 | AGTGAGTGAGTGTGTGTGTGAGA (SEQ ID NO: 102) | 84 | 22 |
| chr2:229641524 | GGTGAGCAAGTGTGTGTGTGTGG (SEQ ID NO: 103) | 93 | 20 |
| chr20:52107864 | CGTGAGTGAGTGTGTACCTGGGG (SEQ ID NO: 104) | 253 | 19 |
| chr11:75436718 | GGTGGATGACTGTGTGTGTGGGG (SEQ ID NO: 105) | 0 | 18 |
| chr1:47839367 | TGTGGGTGAGTGTGTGTGTGTGG (SEQ ID NO: 106) | 45 | 17 |
| chr8:142809408 | GGTGTATGAGTGTGTGTGTGAGG (SEQ ID NO: 107) | 19 | 17 |
| chr17:34996248 | TGTGAGTGAGTATGTACATGTGG (SEQ ID NO: 108) | 12 | 17 |
| chr7:51226565 | AGTGAGTAAGTGAGTGAGTGAGG (SEQ ID NO: 109) | 0 | 17 |
| chr19:17483422 | TGTGAGTGGGTGTGTGTGTGGGG (SEQ ID NO: 110) | 13 | 16 |
| chr16:73552025 | AATGAGTGAGTGTGTGTGTGTGA (SEQ ID NO: 111) | 45 | 13 |
| chr16:74864221 | GGTGAGAGAGTGTGTGCGTAGGA (SEQ ID NO: 112) | 397 | 11 |
| chr17:80980639 | TGTGAGTGAGTGTGTGTGTGTGA (SEQ ID NO: 113) | 35 | 11 |
| chr2:18514959 | AGTGAGAAAGTGTGTGCATGCGG (SEQ ID NO: 114) | 28 | 9 |
| chr16:12170754 | AGTGAGTGAGTGTGTGTGTGTGA (SEQ ID NO: 115) | 70 | 6 |
| chr19:6109019 | TGTGAGTGAGTGTGTGTGTGTGA (SEQ ID NO: 116) | 63 | 6 |
| chr8:66667192 | AGTGAGTGAGTGTGAGTGCGGGG (SEQ ID NO: 117) | 25 | 6 |
| chr1:181588066 | GGAGAGTGAGTGTGTGCATGTGC (SEQ ID NO: 118) | 135 | 5 |
| chr18:14871045 | GGTGTGTGGGTGGGGGTGTGTGG (SEQ ID NO: 119) | 0 | 5 |
| chr6:144137152 | AGGGAGTGAGTGTGAGAGTGCGG (SEQ ID NO: 120) | 79 | 4 |
| chr22:43543415 | GGTGAGAGAGTGTGTGCACGGGG (SEQ ID NO: 121) | 60 | 4 |
| chr9:136328986 | TGTGAGAGAGTGTGTGTGTGGAG (SEQ ID NO: 122) | 0 | 4 |
| chr1:47225214 | TGTGAGAGAGAGTGTGCGTGTGG (SEQ ID NO: 123) | 6 | 3 |
| chr1:32273146 | GGGGGGTGAGTGTGTGTGTGGGG (SEQ ID NO: 124) | 0 | 3 |
| chr1:212466434 | GGGGAATGAGTGTGTGCATGGAG (SEQ ID NO: 125) | 244 | 0 |
| chr19:16458676 | TGTGAGTGAGTGTGTGTGTGGAG (SEQ ID NO: 126) | 181 | 0 |
| chrX:106371183 | AGTGAATGAGTGTGTGCATGTGA (SEQ ID NO: 127) | 115 | 0 |
| chr4:57460440 | GGTGAGTGAGTGAGTGAGTGAGT (SEQ ID NO: 128) | 107 | 0 |
| chr5:150122131 | GATGAGTGAGTGTGTGAGTGAGA (SEQ ID NO: 129) | 107 | 0 |
| chr7:39301525 | GGTGTGTGAGTGTGTGTGTGTGA (SEQ ID NO: 130) | 105 | 0 |
| chr7:152974293 | AGTGAGTGAGTGAGTGAGTGAGG (SEQ ID NO: 131) | 72 | 0 |
| chr5:29367271 | GGTGTGTGAGTGAGTGTGTGTAT (SEQ ID NO: 132) | 65 | 0 |
| chr7:98769618 | AGTGAGTGAGTGAGTGAGTGAGG (SEQ ID NO: 133) | 65 | 0 |
| chr11:7604564 | GGTGAGTAGGTGTGTGTGTGGGG (SEQ ID NO: 134) | 61 | 0 |
| chr16:67249216 | GGTGAGTGCGTGTGTGCGTGCGC (SEQ ID NO: 135) | 58 | 0 |
| chr17:19238254 | GGTGGGTGAATGGGTGCGTGGGG (SEQ ID NO: 136) | 49 | 0 |
| chr5:150845157 | GGTGAGTGAGAGTGTGTGTGTGG (SEQ ID NO: 137) | 49 | 0 |
| chr10:107618309 | GGTGAGTGAGTGAGTGAGTGAGG (SEQ ID NO: 138) | 48 | 0 |
| chr1:32273161 | GGTGAGTGTGTGTGTGGGGGGGC (SEQ ID NO: 139) | 46 | 0 |
| chr4:182960564 | TGTGTGTGAGTGTGTGAGTGTGA (SEQ ID NO: 140) | 46 | 0 |
| chr12:130712119 | GGTGGGTGAGTGAGTGAGTGAGG (SEQ ID NO: 141) | 43 | 0 |
| chr10:106107619 | AGAGAGTGAGTGTGTGTGTTGGG (SEQ ID NO: 142) | 40 | 0 |
| chr6:39060862 | GGTGTGTGAGTGTGTGCATTGGG (SEQ ID NO: 143) | 35 | 0 |
| chr3:194352921 | ACTGAGTGAGTGTGAGTGTGAGG (SEQ ID NO: 144 | 34 | 0 |
| chr12:114315130 | TGTGAGTGAGTGTGTGCATGTGA (SEQ ID NO: 145) | 32 | 0 |
| chrX:42571581 | AGTGAGTGAGTGTGAGCGTGAAG (SEQ ID NO: 146) | 30 | 0 |
| chr1:236052776 | TGTGAGTGAGTGTGGGTGTGTGG (SEQ ID NO: 147) | 28 | 0 |
| chr17:36650349 | AGAGAGTGAGTGTGTGTGTGAGA (SEQ ID NO: 148) | 28 | 0 |
| chr8:140027829 | AGTGAGTGAGTGTGTGTGTGAAG (SEQ ID NO: 149) | 25 | 0 |
| chr11:69704135 | TGTGAGTGGGTGTGTGCGGGGGG (SEQ ID NO: 150) | 22 | 0 |
| chr5:179319537 | TGTGAGTGAGTGCATGTGTGTGG (SEQ ID NO: 151) | 22 | 0 |
| chr1:244885164 | AGAGAGTGAGTGTGTGTGTGAGA (SEQ ID NO: 152) | 21 | 0 |
| chrX:41866964 | GGTGAGTGAGTGAGTGAGTGAGG (SEQ ID NO: 153) | 21 | 0 |
| chr10:5707695 | GGAGAGTGAGTATGTGTGTGTGT (SEQ ID NO: 154) | 20 | 0 |
| chr22:48754271 | GGAGAGCGAGTGTGTGCGTGTGA (SEQ ID NO: 155) | 20 | 0 |
| chrX:150212100 | AATGAGTGAGTGTGTGAGTGGAG (SEQ ID NO: 156) | 19 | 0 |
| chr11:69272225 | GGTGGATGAGTGAATGCGTGAGG (SEQ ID NO: 157) | 16 | 0 |
| chr11:63598868 | ATTGAGTGAGTATGTGTGTGAGG (SEQ ID NO: 158) | 15 | 0 |
| chr7:23237113 | TTTGAGTGAGTGTGTGTGTGTGT (SEQ ID NO: 159) | 15 | 0 |
| chr15:92320981 | TGTGAGTGAGTGTGTGTGTGTGA (SEQ ID NO: 160) | 14 | 0 |
| chr16:79982326 | TGTGAGTGAGAGTGTGCATTGGG (SEQ ID NO: 161) | 14 | 0 |
| chrX:86148551 | AGTGAGGGAGTGAGTGCGAGGGG (SEQ ID NO: 162) | 14 | 0 |
| chr12:57218632 | CTTGAGTGAGAGTGAGCGTGAGG (SEQ ID NO: 163) | 13 | 0 |
| chr17:1275504 | AGTGTGTGAGTGTGTGTGTGAGG (SEQ ID NO: 164) | 13 | 0 |
| chr8:11456535 | GGTGTGTGAGTGTGAGTGTGGGG (SEQ ID NO: 165) | 13 | 0 |
| chrX:39746896 | GGAGAGTCAGTGTGTGCGTATGG (SEQ ID NO: 166) | 13 | 0 |
| chr1:115943020 | AATGAGTGAGTGTGTGAGTGAAG (SEQ ID NO: 167) | 12 | 0 |
| chr12:11106290 | AGTGAGTGAGTATGTGTGTATGG (SEQ ID NO: 168) | 11 | 0 |
| chr12:99263738 | AGAGAGTGAGTGTGTGTGTAGGA (SEQ ID NO: 169) | 11 | 0 |
| chr21:42759866 | TGTGAGTGGGTGTGTGCATGTGG (SEQ ID NO: 170) | 11 | 0 |
| chr3:179710986 | GGTGAGTCAGTGAGTGAGTGGGG (SEQ ID NO: 171) | 11 | 0 |
| chr3:40328393 | GGGGAATGAGTGTGTGTGTGGGG (SEQ ID NO: 172) | 11 | 0 |
| chr19:38649361 | GGTGAGTGGGTGTGTGTGGGGGG (SEQ ID NO: 173) | 9 | 0 |
| chr19:49016344 | GGGGAATGAGCATGTGCCTGAGG (SEQ ID NO: 174) | 9 | 0 |
| chr13:67829070 | GGTGAGTCAGTGAGTGAGTGGGG (SEQ ID NO: 175) | 8 | 0 |
| chr14:100167889 | GGTGAGTGTGTGTGTGTGTTGGG (SEQ ID NO: 176) | 8 | 0 |
| chr20:63837633 | AGTGAGTGAGTGAGTGAATGAGG (SEQ ID NO: 177) | 8 | 0 |
| chr21:44637351 | TGTGAGTGAGTGTGTGTGTGAGC (SEQ ID NO: 178) | 8 | 0 |
| chr12:124671956 | GATGAGTGTGTGTGTGTGCGGGT (SEQ ID NO: 179) | 7 | 0 |
| chr6:10696478 | AGTGAGTGAGTGTGTGTGTGTGT (SEQ ID NO: 180) | 7 | 0 |
| chr6:144631221 | AGAGAGTGAGTGTGTGTGTGTGA (SEQ ID NO: 181) | 6 | 0 |
| chr14:97976195 | GGTGAGTGTGTGTGTGAGTGTGG (SEQ ID NO: 182) | 5 | 0 |
| chr17:78994319 | AGTGACTGAGTCTGTGCCTGGGG (SEQ ID NO: 183) | 5 | 0 |
| chr19:49152088 | GGGGAGAGAGAGTGAGCGTGGGG (SEQ ID NO: 184) | 5 | 0 |
| chr6:19675343 | GGTGAGTGAATGTGTGTGTGTGA (SEQ ID NO: 185) | 5 | 0 |
| chr8:141901925 | GGTGAGTGAGTGTGTGTGGGGTG (SEQ ID NO: 186) | 5 | 0 |
| chr10:1642777 | TGTGAGTGGGTGTGTGAGTGAGG (SEQ ID NO: 187) | 4 | 0 |
| chr13:26254780 | GGTGAGTGTGTGTGTCTGGGCCG (SEQ ID NO: 188) | 4 | 0 |
| chr13:29706701 | GATAAGTGAGTATGTGTGTGTGG (SEQ ID NO: 189) | 4 | 0 |
| chr13:60108887 | GGTGAGTGGGTGTGTGTGTTGGG (SEQ ID NO: 190) | 4 | 0 |
| chr13:66816459 | GGTGAGTGTGAGTGTGTGTGGGG (SEQ ID NO: 191) | 4 | 0 |
| chr14:104735501 | TGTGAGTGAGTATGTGCTTGCGA (SEQ ID NO: 192) | 4 | 0 |
| chr16:82720515 | TATGAGTGAGTGTGAGCGTGGGT (SEQ ID NO: 193) | 4 | 0 |
| chr19:6109096 | TGCGAGTGCGTGTGTGTGTTTGT (SEQ ID NO: 194) | 4 | 0 |
| chr19:7197354 | AGCGAGTGAGTGAGTGAGTGGGG (SEQ ID NO: 195) | 4 | 0 |
| chr5:6007116 | AGTGAGTGAGTGAGTGAGTGAGG (SEQ ID NO: 196) | 4 | 0 |
| chr10:97546894 | AGAGAGAGAGTGTGTGTGTGAGG (SEQ ID NO: 197) | 3 | 0 |
| chr15:83282870 | GGAGAGAGAGAGTGTGTGTGTGA (SEQ ID NO: 198) | 3 | 0 |
| chr2:216752547 | AGGGAGTGAGTGTGTAAGTGTGG (SEQ ID NO: 199) | 3 | 0 |
| chr4:182960502 | TGTGAGAGAGTGTGTGCGTGTGA (SEQ ID NO: 200) | 3 | 0 |
| chr5:180595164 | AGTGAGTGGGTGTGAGCTTGTGG (SEQ ID NO: 201) | 3 | 0 |
| chr6:150585785 | GGTGAGTGAGTGACTGAGTGAGT (SEQ ID NO: 202) | 3 | 0 |

TTISS reads and published GUIDE-seq read counts from an experiment using the same gRNAs in U2OS cells are listed in Table 4. List of target sites detected for the RNF2 and VEGFA gRNAs from single-guide TTISS runs in K562 cells. TTISS reads and published DISCOVER-seq read counts from an experiment using the same gRNAs in K562 cells are listed.

TABLE 4

GUIDE-seq read counts from an experiment using the same gRNAs in U2OS cells. (Bolded nucleotides represent variant bases and unbolded nucleotides represent WT bases)
RNF2

| Genome Position | GTCATCTTAGTCATTACCTGAGG (SEQ ID NO: 203) | TTISS | DISCOVER-seq |
|---|---|---|---|
| chr1:185087639 | GTCATCTTAGTCATTACCTGAGG (SEQ ID NO: 204) | 1914 | 100 |
| VEGFA | | | |
| Genome Position | GACCCCCTCCACCCCGCCTCCGG (SEQ ID NO: 205) | TTISS | DISCOVER-seq |
| chr6:43770824 | GACCCCCTCCACCCCGCCTCCGG (SEQ ID NO: 206) | 807 | 1046 |
| chr5:6715005 | CTACCCCTCCACCCCGCCTCCGG (SEQ ID NO: 207) | 2230 | 486 |
| chr2:241275191 | ATTCCCCCCCACCCCGCCTCAGG (SEQ ID NO: 208) | 566 | 347 |
| chr11:31795933 | GGGCCCCTCCACCCCGCCTCTGG (SEQ ID NO: 209) | 187 | 242 |
| chr4:38536006 | CTCCCCACCCACCCCGCCTCAGG (SEQ ID NO: 210) | 750 | 233 |
| chr1:151059409 | CCTCCCCCACACCCCGCATCCGG (SEQ ID NO: 211) | 87 | 214 |
| chr5:139648671 | CTCCCCCCCCTCCCCGCCTCGGG (SEQ ID NO: 212) | 106 | 212 |
| chr10:133336442 | CGCCCTCCCCACCCCGCCTCCGG (SEQ ID NO: 213) | 166 | 208 |
| chr18:23779593 | GCCCCCACCCACCCCGCCTCTGG (SEQ ID NO: 214) | 443 | 172 |
| chr17:41888502 | TGCCCCTCCCACCCCGCCTCTGG (SEQ ID NO: 215) | 294 | 122 |
| chr9:100837365 | ACACCCCCCCACCCCGCCTCAGG (SEQ ID NO: 216) | 212 | 108 |
| chr2:12604649 | GACACACCCCACCCCACCTCAGG (SEQ ID NO: 217) | 144 | 93 |
| chr11:374664 | AGGCCCCCCCGCCCCGCCTCAGG (SEQ ID NO: 218) | 136 | 71 |
| chr22:50446375 | CCCCCCCCCCCCCCCGCCTCCGG (SEQ ID NO: 219) | 159 | 63 |
| chr16:56929515 | TGCCCCCCCCACCCCACCTCTGG (SEQ ID NO: 220) | 287 | 58 |
| chr11:72237759 | GCTTCCCTCCACCCCGCATCCGG (SEQ ID NO: 221) | 81 | 51 |
| chr9:136546388 | CGCCCTCCCCATTCCGCCCCGGG (SEQ ID NO: 222) | 0 | 47 |
| chr11:76784742 | CACCCCCCCCCCCCCACCTCCGG (SEQ ID NO: 223) | 53 | 46 |
| chr17:4455455 | TACCCCCCACACCCCGCCTCTGG (SEQ ID NO: 224 | 80 | 41 |
| chr10:70778461 | CAGTCCCCCCACCCCACCTCTGG (SEQ ID NO: 225) | 28 | 40 |
| chr9:123375900 | CACTCCCCCCACCCCGCCCCAGG (SEQ ID NO: 226) | 107 | 36 |
| chr13:99894731 | CCCCCCCCCCCCCCCGCCTCAGG (SEQ ID NO: 227) | 41 | 33 |
| chr12:25872159 | CATTCCCCCCACCCCACCTCAGG (SEQ ID NO: 228) | 33 | 24 |
| chr16:69132801 | AGTAGCCCCCACCCCGCCTCGGG (SEQ ID NO: 229) | 0 | 24 |
| chr19:42302642 | TTCTCCCTCCTCCCCGCCTCGGG (SEQ ID NO: 230) | 0 | 24 |
| chr1:939957 | GACCCTGTCCACCCCACCTCAGG (SEQ ID NO: 231) | 30 | 21 |
| chrX:129906663 | TGCCCCCCCCACCCCGCCCCCGG (SEQ ID NO: 232) | 48 | 19 |
| chr9:27338876 | GACCCCTCCCACCCCGACTCCGG (SEQ ID NO: 233) | 41 | 18 |
| chr3:140679958 | CAACCCCCCCACCCCGCTTCAGG (SEQ ID NO: 234) | 38 | 17 |
| chr15:32993905 | GACCCCCCCCACCCCGCCCCCGG (SEQ ID NO: 235) | 41 | 14 |
| chr19:14032161 | GAGCTCCCCCACCCCGCCCCGGG (SEQ ID NO: 236) | 37 | 14 |
| chr17:57663166 | CCGCCCCTCCACCCCGCCACTGG (SEQ ID NO: 237) | 22 | 12 |
| chr19:18522671 | AGTCCCATCCACCCCGCCTAAGG (SEQ ID NO: 238) | 8 | 12 |
| chr9:137368989 | AAGCCCCCCCACCCCGCCCCGGG (SEQ ID NO: 239) | 12 | 10 |
| chr13:26052087 | TCCCCCCCACCCCCGACCTCAGG (SEQ ID NO: 240) | 0 | 10 |
| chr1:50976519 | GACCCCTCCCTCCCCACCTCAGG (SEQ ID NO: 241) | 34 | 9 |
| chr11:2665017 | CTCACCCCCCACCCCACCTCTGG (SEQ ID NO: 242) | 37 | 8 |
| chr4:1494530 | AGGCCCCCACACCCCGCCTCAGG (SEQ ID NO: 243) | 16 | 8 |
| chr9:128944301 | AGCCAACCCCACCCCGCCTCTGG (SEQ ID NO: 244) | 3 | 8 |
| chr7:123534791 | CGGCCCCACCTCCCCGCCTCTGG (SEQ ID NO: 245) | 0 | 8 |
| chr7:105293508 | TCCACCCCCCACCCCGCCCCGGG (SEQ ID NO: 246) | 74 | 7 |
| chr5:133524683 | TGCACCCCCCACCCCGCCCCTGG (SEQ ID NO: 247) | 4 | 7 |
| chrX:150764054 | CTGCCCCCCCACCCCGCCACTGG (SEQ ID NO: 248) | 138 | 6 |
| chr10:132143139 | AGCCCCCCCCACCCCGACTCAGG (SEQ ID NO: 249) | 28 | 5 |
| chr10:114534495 | CCCCACCCCCACCCCGCCTCAGG (SEQ ID NO: 250) | 16 | 5 |
| chr4:8840190 | CATACCCCCCACCCCGCCCCGGG (SEQ ID NO: 251) | 16 | 5 |
| chr11:63623616 | GACACCTTCCACCCCGTCTCTGG (SEQ ID NO: 252) | 71 | 4 |
| chr1:11654487 | GACCCGCCCCGCCCCGCCTCTGG (SEQ ID NO: 253) | 4 | 4 |
| chr3:48078006 | CCCTTCATTCACCCAGCCTCTGG (SEQ ID NO: 254) | 0 | 4 |
| chr4:77066020 | AACCCCTGCCTCCCGGGCTCAAG (SEQ ID NO: 255) | 0 | 4 |
| chr6:44624466 | GCTCCACACCACCCCCACTCTGG (SEQ ID NO: 256) | 0 | 4 |
| chr7:139353712 | AACCTCCACCTCCCGGATTCAAG (SEQ ID NO: 257) | 0 | 4 |
| chr19:13011374 | GCCCCCCACCACCCCACCTCGGG (SEQ ID NO: 258) | 125 | 3 |
| chr8:143740792 | GTACCCCACCACCCCGCCCCAGG (SEQ ID NO: 259) | 73 | 3 |
| chr2:169716840 | CCACCCCCCCACCCCGCCCCAGG (SEQ ID NO: 260) | 33 | 3 |
| chr11:83722550 | GTCACTCCCCACCCCGCCTCTGG (SEQ ID NO: 261) | 0 | 3 |
| chr6:160131527 | TCAGACCTCCACCCCGCCTCAGG (SEQ ID NO: 262) | 0 | 3 |
| chr17:17051536 | CTCCCCCGCCACCCCGCCCCAGG (SEQ ID NO: 263 | 27 | 0 |
| chr7:102479107 | GCCACCCCGCACCCCGCCCCCCG (SEQ ID NO: 264) | 25 | 0 |
| chr19:1028249 | ACCCCACCCCACCCCGTCTCCGG (SEQ ID NO: 265) | 23 | 0 |
| chr6:26570645 | GACCCCCCCACCCCACCCTCCGG (SEQ ID NO: 266) | 21 | 0 |
| chr11:12287387 | ATCCCCCTCCACCCCACCCCTGG (SEQ ID NO: 267) | 19 | 0 |
| chr7:95690362 | GACCCCTCACACCCCGCCCCTGG (SEQ ID NO: 268) | 19 | 0 |
| chr11:13926823 | TACCCCCCCCACCCCGCCACAGG (SEQ ID NO: 269) | 18 | 0 |
| chr2:128486626 | CCCCCCCCCCACCCCGCCCCCGG (SEQ ID NO: 270) | 16 | 0 |
| chr2:11559837 | CTCCCTCCCCACCCCACCTCTGG (SEQ ID NO: 271) | 12 | 0 |
| chr2:24634727 | ACCCCCCCCCCCCCCGCCCCCGG (SEQ ID NO: 272) | 12 | 0 |
| chr8:18184036 | CCCCCCCACCACCCCGCCCCGGG (SEQ ID NO: 273) | 12 | 0 |
| chr6:26470395 | GACCCCCCCCACCCCACCCCAGG (SEQ ID NO: 274) | 11 | 0 |
| chr15:78565380 | TCCCCACCCCGCCCCGCCTCTGG (SEQ ID NO: 275) | 10 | 0 |
| chr17:64089693 | ACTCCCCTCCACCCCGGCTCGGG (SEQ ID NO: 276) | 10 | 0 |
| chr22:43288489 | AGCCCCCACCTCCCCGCCTCGGG (SEQ ID NO: 277) | 10 | 0 |
| chr1:23435756 | ACTCCCCTCCACCCCACCTCTGA (SEQ ID NO: 278) | 9 | 0 |
| chr11:46120302 | CATCCCCCCCACCCCACCCCGGG (SEQ ID NO: 279) | 9 | 0 |
| chr7:50697831 | AACCACCCCCACCCCACCCCAGG (SEQ ID NO: 280) | 9 | 0 |
| chr8:39981565 | CACACCCACCACCCCGCCTCAGA (SEQ ID NO: 281) | 9 | 0 |
| chr9:37465368 | CCCCCCTCCCACCCCGCCTCTAG (SEQ ID NO: 282) | 9 | 0 |
| chr16:82700974 | CCCCCCCCCCCCCCCGCCCCGGG (SEQ ID NO: 283) | 8 | 0 |
| chr17:48026480 | AACCTCCCCCACCCCACCCCAGG (SEQ ID NO: 284) | 7 | 0 |
| chr3:195762349 | CACCACCCCCACCCCGCCCCTGG (SEQ ID NO: 285) | 7 | 0 |
| chr3:31417164 | CTTCCCCCACACCCCGCCCCAGG (SEQ ID NO: 286) | 7 | 0 |
| chr5:171451065 | CCGCCCCCCCACCCCGCCGCCGG (SEQ ID NO: 287) | 7 | 0 |
| chr7:131106816 | GGCCCCACCCACCCCGCCTTCTG (SEQ ID NO: 288) | 7 | 0 |
| chr9:133572196 | CCCACCCCCCACCCCGCCCCAGG (SEQ ID NO: 289) | 7 | 0 |
| chr1:178769590 | GGCCCTCTCCACTCCACCTCAGG (SEQ ID NO: 290) | 6 | 0 |
| chr13:99894755 | CCCCCCCCCCCCCCCGCCTCAGG (SEQ ID NO: 291) | 6 | 0 |
| chr17:30648222 | TACCCCCTCCACCCCGCTCCAGG (SEQ ID NO: 292) | 6 | 0 |
| chr17:60327509 | CGCCCACCCCACCCCACCTCAGG (SEQ ID NO: 293) | 6 | 0 |
| chr19:45448795 | AAGACCCCCCACCCCGCCCCAGG (SEQ ID NO: 294) | 6 | 0 |
| chr3:13145801 | GGACCCCCCCCCCCCGCCCCCGG (SEQ ID NO: 295) | 6 | 0 |
| chr11:65712299 | GGCTCCCTCCGCCCCGCCCCGGG (SEQ ID NO: 296) | 5 | 0 |
| chr20:10933316 | CCACCCCCCCACCCCGCCCCTGG (SEQ ID NO: 297) | 5 | 0 |
| chr6:31495048 | CTCCCCCTCCACCCCACCTCCAG (SEQ ID NO: 298) | 5 | 0 |
| chr10:100969500 | CCCCCCCCCCGCCCCGCCTCCAG (SEQ ID NO: 299) | 4 | 0 |
| chr10:101061759 | CTACCCCCACTCCCCGCCTCCGG (SEQ ID NO: 300) | 4 | 0 |
| chr11:61553965 | CACCCCCTCCCCTCCGCCTCAGG (SEQ ID NO: 301) | 4 | 0 |
| chr16:85304598 | ATGCCCCACCCCCCCGCCCCCGG (SEQ ID NO: 302) | 4 | 0 |
| chr19:51412260 | AACACCCCCCACCCCACCCCGGG (SEQ ID NO: 303) | 4 | 0 |
| chr20:37362728 | AGACCCCCCCACCCCACCCCAGG (SEQ ID NO: 304) | 4 | 0 |
| chr5:180161300 | GACTCCCTCCGCCCCGCTTCCAG (SEQ ID NO: 305) | 4 | 0 |
| chr19:44821323 | CCCCCCCCTCACCCCGCCCCTGG (SEQ ID NO: 306) | 3 | 0 |
| chr5:156894131 | GACCCCACCTACCCCACCTCAGG (SEQ ID NO: 307) | 2 | 0 |
| chrX:153571670 | GTCCCCCTCCTCCCCACCTCCGG (SEQ ID NO: 308) | 2 | 0 |
| chrX:119731518 | GTCCTCCACCACCCCGCCTCTGG (SEQ ID NO: 309) | 1 | 0 |

TABLE 5

TTISS-detected target sites across 59 guides and Cas9 variants used in this study (related to FIGS. 1A-1C; (Bolded nucleotides represent variant bases and unbolded nucleotides represent WT bases)
On- and off-target sites detected for at least one variant of SpCas9 (including WT) from 59gRNA pool with read counts

| Genome Position | Site Sequence | MMs | Cut Site Score | gRNA Original Target Gene |
|---|---|---|---|---|
| chr15:100887703 | GGAGAGGGACCGCGCCACCTTGG (SEQ ID NO: 310) | 0 | -1 | ALDH1A3 |
| chr9:88260748 | GGTGAGGCACCGTGCCACCTGGG (SEQ ID NO: 311) | 3 | -1 | ALDH1A3 |
| chr20:62909596 | GGAGAGGCACCGCCCCACATGGG (SEQ ID NO: 312) | 3 | -1 | ALDH1A3 |
| chr16:70756728 | GGGGAGGCACCGGGCCACCTTGG (SEQ ID NO: 313) | 3 | -1 | ALDH1A3 |
| chr2:122079778 | GGTGAGGGACCGAGTCACCTAGG (SEQ ID NO: 314) | 3 | -1 | ALDH1A3 |
| chr11:71080469 | CAAGAGGAACGGCGCCACCTGGG (SEQ ID NO: 315) | 4 | -1 | ALDH1A3 |
| chr2:127027939 | AGAAAGTGACAGCGCCACCTAGG (SEQ ID NO: 316) | 4 | -1 | ALDH1A3 |
| chr22:50299901 | GGGGAGGGGCTGTGCCACCTGGG (SEQ ID NO: 317) | 4 | -1 | ALDH1A3 |
| chr5:181217678 | GGAGGAGGACTGCGCCACTTCGG (SEQ ID NO: 318) | 4 | -1 | ALDH1A3 |
| chr14:76119243 | GGAAAGGGACCCCACCACCCAGG (SEQ ID NO: 319) | 4 | -1 | ALDH1A3 |
| chr8:10730582 | AGGGAGGGGCCGCGCCGCCTTGG (SEQ ID NO: 320) | 4 | -1 | ALDH1A3 |
| chr7:73573965 | GGAGCTGGACCACGCCACCCTGG (SEQ ID NO: 321) | 4 | -1 | ALDH1A3 |
| chr1:180199900 | CAAGAGGGGCAGCGCCACCTTGG (SEQ ID NO: 322) | 4 | -1 | ALDH1A3 |
| chr10:127739369 | GGAAAGGGCCCCCACCACCTGGG (SEQ ID NO: 323) | 4 | -1 | ALDH1A3 |
| chr13:99318774 | GGAGAGCAATGGCGCCACCTCGG (SEQ ID NO: 324) | 4 | -1 | ALDH1A3 |
| chr7:150942359 | GGGGAGGGACTGCACCACCACGG (SEQ ID NO: 325) | 4 | -1 | ALDH1A3 |
| chr22:24418547 | TGGGAGTGACCGCCCCACCTGGG (SEQ ID NO: 326) | 4 | -1 | ALDH1A3 |
| chr22:50148344 | GCAGAGGGGCCACCCCACCTGGG (SEQ ID NO: 327) | 4 | -1 | ALDH1A3 |
| chr1:154852904 | GGTGAGGGATCCAGCCACCTGGG (SEQ ID NO: 328) | 4 | -1 | ALDH1A3 |
| chr2:64907510 | CTTGAGGGACTGCGCCACCTGGA (SEQ ID NO: 329) | 4 | -1 | ALDH1A3 |
| chr1:1374359 | GGAGAGAGGCCGCCCTACCTGGG (SEQ ID NO: 330) | 4 | -1 | ALDH1A3 |
| chr7:776786 | GGACAGGGCCCCCGCCACCCAGG (SEQ ID NO: 331) | 4 | -1 | ALDH1A3 |
| chrX:81940428 | GGTGAGGCATCGCCCCACCTGGG (SEQ ID NO: 332) | 4 | -1 | ALDH1A3 |
| chr1:21845933 | GGACAGGAACCACTCCACCTGAG (SEQ ID NO: 333) | 4 | -1 | ALDH1A3 |
| chr19:29639960 | GGAGAGCAAAGGCGCCACCTCGG (SEQ ID NO: 334) | 4 | -1 | ALDH1A3 |
| chr2:66472709 | GCAGAGGGACAGCACTACCTTGG (SEQ ID NO: 335) | 4 | -1 | ALDH1A3 |
| chr6:138292022 | GGAGAGGGTGAGCACCACCTTGG (SEQ ID NO: 336) | 4 | -1 | ALDH1A3 |
| chr1:27563573 | GCAGAGGGACGGCACCACCCAGG (SEQ ID NO: 337) | 4 | -1 | ALDH1A3 |
| chr2:230250898 | GGTGATGGACAGCCCCACCTAGG (SEQ ID NO: 338) | 4 | 0 | ALDH1A3 |
| chr12:49540928 | GGGGAAGAGCCCCGCCACCTGGG (SEQ ID NO: 339) | 5 | -1 | ALDH1A3 |
| chr9:88145188 | GGAGGAAGACCACGCCACCCTGG (SEQ ID NO: 340) | 5 | -1 | ALDH1A3 |
| chr1:151805904 | ACTGAGGGACTGCTCCACCTGGG (SEQ ID NO: 341) | 5 | 0 | ALDH1A3 |
| chr7:16912739 | CCTGAGGGACCTCGCCACCCTGG (SEQ ID NO: 342) | 5 | -1 | ALDH1A3 |
| chr1:51315173 | AAAGAGGGACAGCCCCACCCGGG (SEQ ID NO: 343) | 5 | -1 | ALDH1A3 |
| chr10:76013221 | GATTAAGGACAGCGCCACCTGGG (SEQ ID NO: 344) | 5 | -1 | ALDH1A3 |
| chr17:47281556 | TGAAGGGGACCACGCCACCCTGG (SEQ ID NO: 345) | 5 | -1 | ALDH1A3 |
| chr2:42361225 | AGAGAAGGACCCCGCCTCCCCGG (SEQ ID NO: 346) | 5 | 0 | ALDH1A3 |
| chr1:101370101 | GCAGAAGGACCATGCCACCCGGG (SEQ ID NO: 347) | 5 | -1 | ALDH1A3 |
| chr19:44903312 | AAGGAGGGACCCCGCCACCCCAG (SEQ ID NO: 348) | 5 | 1 | ALDH1A3 |
| chrX:154344396 | AGAGAGAGGCTGCCCCACCTGGG (SEQ ID NO: 349) | 5 | -1 | ALDH1A3 |
| chr3:194761975 | AGAGGGGTACAGTGCCACCTTGG (SEQ ID NO: 350) | 5 | -1 | ALDH1A3 |
| chr16:66697171 | AGAGACGGGCTGCGCCACCCGGG (SEQ ID NO: 351) | 5 | -1 | ALDH1A3 |
| chr19:33801411 | GGGGAGAGACCCCACCCCCTAGG (SEQ ID NO: 352) | 5 | -1 | ALDH1A3 |
| chr19:4932665 | CGGGAGGGGCCGTCCCACCTCGG (SEQ ID NO: 353) | 5 | -1 | ALDH1A3 |
| chr3:34200454 | GGAGAAAGGCCAAGCCACCTAGG (SEQ ID NO: 354) | 5 | -1 | ALDH1A3 |
| chr4:56842835 | GGAGAGGAGTCCCCCCACCTAGG (SEQ ID NO: 355) | 5 | -1 | ALDH1A3 |
| chr11:69005013 | AAGGAGGGGCCCCACCACCTGGG (SEQ ID NO: 356) | 6 | -1 | ALDH1A3 |
| chr19:3543730 | CCAGGGGGACAAGGCCACCTAGG (SEQ ID NO: 357) | 6 | -1 | ALDH1A3 |
| chr14:69952349 | GGAGAGGTTCCTGGGCACCCCAG (SEQ ID NO: 358) | 6 | -2 | ALDH1A3 |
| chr20:62318929 | CCAGAGCAGCCGCTCCACCTCGG (SEQ ID NO: 359) | 6 | -1 | ALDH1A3 |
| chr4:41650466 | GGAGTGGGCAGGTGCCACCGTGG (SEQ ID NO: 360) | 6 | -2 | ALDH1A3 |
| chr16:24346808 | GAACTTACGCAGGAGATATTCGG (SEQ ID NO: 361) | 0 | -1 | CACNG3 |
| chr8:42916049 | GCATTTAGGCAGGAGATATTTGG (SEQ ID NO: 362) | 3 | -2 | CACNG3 |
| chr3:72489097 | CCCCTTACGCAGGGGATATTTGG (SEQ ID NO: 363) | 4 | -1 | CACNG3 |
| chr17:15975208 | GTTCCGGTAAGCATAGACAATGG (SEQ ID NO: 364) | 0 | -1 | ADORA2B |
| chrX:111330681 | ATTACAGCAAGCATAGACAATGG (SEQ ID NO: 365) | 4 | -1 | ADORA2B |
| chr17:35577906 | GAGACCCGCTCTTCAGCATGTGG (SEQ ID NO: 366) | 0 | -1 | PEX12 |
| chr17:76400901 | GAGCCCCGCTCCTCAGCATCTGG (SEQ ID NO: 367) | 3 | -1 | PEX12 |
| chr14:105006302 | GGGACCCGATCTTCAGCTTGTGG (SEQ ID NO: 368) | 3 | -1 | PEX12 |
| chr17:32794027 | GAGACCCATTGTTCAGCATGCGG (SEQ ID NO: 369) | 3 | -1 | PEX12 |
| chr2:232227298 | GAGACTCGCCCCTCAGCATCGGG (SEQ ID NO: 370) | 4 | -1 | PEX12 |
| chr9:91502545 | AAAACCCGCTCCTAAGCATGTGG (SEQ ID NO: 371) | 4 | -1 | PEX12 |
| chr2:42043074 | GGCTCCCGCTCTCCAGCATGCGG (SEQ ID NO: 372) | 4 | -1 | PEX12 |
| chr1:156700582 | GAGAGGGCCCCAAGACCTCGTGG (SEQ ID NO: 373) | 0 | -1 | CRABP2 |
| chr19:1354470 | GGGAGGGTCCCAAGACCCCGGGG (SEQ ID NO: 374) | 3 | -1 | CRABP2 |
| chr12:115433379 | AATAGGGCCCCAAGGCCTCGGGG (SEQ ID NO: 375) | 3 | 0 | CRABP2 |
| chr7:156217669 | GAGAGGGACCCAAGGCCTCCGGG (SEQ ID NO: 376) | 3 | -1 | CRABP2 |
| chr1:88498406 | AAGAGGGCCCCAAGACCGCAGAG (SEQ ID NO: 377) | 3 | -1 | CRABP2 |
| chr20:39269227 | GAGGGGGCCCCAAGACCCCAAGC (SEQ ID NO: 378) | 3 | -1 | CRABP2 |
| chr11:409426 | CAGAGGGCCCCAAGACCCCCAAG (SEQ ID NO: 379) | 3 | -1 | CRABP2 |
| chr19:10567098 | GAGAGGGGCTCAGGACCTCGTGG (SEQ ID NO: 380) | 3 | -1 | CRABP2 |
| chr16:71442596 | GAGAGGGCCCCCAGGCCTCCGGG (SEQ ID NO: 381) | 3 | -1 | CRABP2 |
| chr11:2301205 | GAGGGGGCCCCAAGACCTGCAGG (SEQ ID NO: 382) | 3 | -1 | CRABP2 |
| chr1:26698013 | AAGAGGGCCCCTAGAGCTCGAGG (SEQ ID NO: 383) | 3 | 0 | CRABP2 |
| chr21:44367598 | GAGGGGGCCCCAAGTCCTCAAGG (SEQ ID NO: 384) | 3 | -1 | CRABP2 |
| chr17:82619638 | AAGAGGTGCCCAAGACCTCAGGG (SEQ ID NO: 385) | 4 | 0 | CRABP2 |
| chr17:77483305 | GAGAGGACACCAAGACCCCAGGG (SEQ ID NO: 386) | 4 | -1 | CRABP2 |
| chr8:140656645 | GAGGGAGCCCCAGGACCTCTGGG (SEQ ID NO: 387) | 4 | 0 | CRABP2 |
| chr20:49407849 | GGGAAGGCCCCAGGACCCCGTGG (SEQ ID NO: 388) | 4 | -1 | CRABP2 |
| chr19:47676174 | CCCAGGGCCCCAAGGCCTCGGGG (SEQ ID NO: 389) | 4 | -1 | CRABP2 |
| chr12:132805178 | CAGAGGACCCCAAGACCCCCAGG (SEQ ID NO: 390) | 4 | -1 | CRABP2 |
| chr1:231728533 | GATAGAGCTCCAAGACCTCTGAG (SEQ ID NO: 391) | 4 | -1 | CRABP2 |
| chr12:108427354 | TAGAGGGTCCCAGGACCTTGTGG (SEQ ID NO: 392) | 4 | 0 | CRABP2 |
| chrX:108568789 | GATGGGGCCCCAGGACCTCAAGG (SEQ ID NO: 393) | 4 | 0 | CRABP2 |
| chr5:72673878 | AAGAGGGCTCCAAGATCTCATGG (SEQ ID NO: 394) | 4 | -1 | CRABP2 |
| chr7:76067772 | ATGAGAGGCCCAAGACCTCGGGG (SEQ ID NO: 395) | 4 | -1 | CRABP2 |
| chr17:73508691 | GAGGGGACACCAAGGCCTCGAGG (SEQ ID NO: 396) | 4 | -1 | CRABP2 |
| chr9:137476980 | GAGGTGGCCCCAGGGCCTCGAGG (SEQ ID NO: 397) | 4 | -1 | CRABP2 |
| chr7:157779083 | TTGAGGGTCCCAAGACCCCAGGG (SEQ ID NO: 398) | 5 | -1 | CRABP2 |
| chr5:125076149 | AAGAAGACTCCAAGACCTCACGG (SEQ ID NO: 399) | 5 | 0 | CRABP2 |
| chrX:153875482 | GGAGGAGGCCCAAGACCTCGGGG (SEQ ID NO: 400) | 5 | 0 | CRABP2 |
| chr6:151734546 | GAGAGGGACTCACCACCTGGGTG (SEQ ID NO: 401) | 5 | 2 | CRABP2 |
| chr22:37062762 | AGGTGGGCCCCAGGACCTCTGGG (SEQ ID NO: 402) | 5 | -1 | CRABP2 |
| chr8:58128329 | AAGAAGGCCCTAAGACCCCTAGG (SEQ ID NO: 403) | 5 | -1 | CRABP2 |
| chr18:77603659 | GAGAGGGCCCTGCCACCTGGGCC (SEQ ID NO: 404) | 5 | 1 | CRABP2 |
| chr19:51108434 | AAGAAAGCCCCAAGACCTTATGG (SEQ ID NO: 405) | 5 | -1 | CRABP2 |
| chr19:4472896 | CCCAGGGCCCCCAGACCCCGGGG (SEQ ID NO: 406) | 5 | -1 | CRABP2 |
| chr21:8253330 | GGCCGGGCCCCGGGCCCTCGACC (SEQ ID NO: 407) | 6 | -1 | CRABP2 |
| chr18:9396540 | GCGCCTTATTCCAGTGACAAAGG (SEQ ID NO: 408) | 0 | -1 | TWSG1 |
| chr19:605090 | GCAGATCCTCATCACCGCGCTGG (SEQ ID NO: 409) | 0 | -1 | HCN2 |
| chr15:32314698 | GCAGAACCGCATCACCGCGCTGG (SEQ ID NO: 410) | 2 | -1 | HCN2 |
| chr15:30223990 | GCAGAACCGCATCACCGCGCTGG (SEQ ID NO: 411) | 2 | -1 | HCN2 |
| chr9:63160274 | GCAGACTCTCATCACCGCTCAGG (SEQ ID NO: 412) | 3 | -1 | HCN2 |
| chr2:94618897 | GCAGACTCTCATCACCGCTCAGG (SEQ ID NO: 413) | 3 | -1 | HCN2 |
| chr9:63300227 | GCAGACTCTCATCACCGCTCAGG (SEQ ID NO: 414) | 3 | -1 | HCN2 |
| chr9:65911627 | GCAGACTCTCATCACCGCTCAGG (SEQ ID NO: 415) | 3 | -1 | HCN2 |
| chr9:40464689 | GCAGACTCTCATCACCGCTCAGG (SEQ ID NO: 416) | 3 | 1 | HCN2 |
| chr19:12991491 | AAAGATCCTCATCACCGCCCTAG (SEQ ID NO: 417) | 3 | -1 | HCN2 |
| chr14:27849168 | GCAGACTATCATCACCGCTCAGG (SEQ ID NO: 418) | 4 | -1 | HCN2 |
| chr19:21070517 | GCAGATGCCCACCACCACGCTGG (SEQ ID NO: 419) | 4 | -1 | HCN2 |
| chrX:94505843 | CCAGATCCACATCACCAAGCTGG (SEQ ID NO: 420) | 4 | -1 | HCN2 |
| chr11:117458879 | GCAGAACATCACCACCACGCGGG (SEQ ID NO: 421) | 4 | -1 | HCN2 |
| chr10:130911421 | ACAGATGCTCACCACCACGCCGG (SEQ ID NO: 422) | 4 | -1 | HCN2 |
| chr19:52433522 | ACAGACCCCCACCACCGCGCCTG (SEQ ID NO: 423) | 4 | -1 | HCN2 |
| chr3:140933802 | GCAGAGCCCCACCACAGCGCTGG (SEQ ID NO: 424) | 4 | -1 | HCN2 |
| chr13:18242232 | ACAGATACTCACCACCACGCAGG (SEQ ID NO: 425) | 4 | 0 | HCN2 |
| chr5:69097271 | ACAGACGCCCACCACCGCGCCGG (SEQ ID NO: 426) | 5 | -1 | HCN2 |
| chr7:99560239 | ACAGACCCGCACCACCACGCTGG (SEQ ID NO: 427) | 5 | -1 | HCN2 |
| chr22:20692917 | ACAGGTACTCACCACCACGCAGG (SEQ ID NO: 428) | 5 | -1 | HCN2 |
| chr15:28877472 | GCAGATGCCCACCACCAAGCCCG (SEQ ID NO: 429) | 5 | -1 | HCN2 |
| chr17:81881334 | ACAGACACCCACCACCGCGCCTG (SEQ ID NO: 430) | 5 | -1 | HCN2 |
| chr19:49093540 | ACAGGTACACATCACCACGCCGG (SEQ ID NO: 431) | 5 | -1 | HCN2 |
| chr9:43093041 | GCAGACTCTCATCGCCACTCAGG (SEQ ID NO: 432) | 5 | 0 | HCN2 |
| chr10:112228898 | ACAGATGCTCACCACCACGGACA (SEQ ID NO: 433) | 5 | -1 | HCN2 |
| chr12:38167952 | ACAGGTCCTCACCACCATGCCGG (SEQ ID NO: 434) | 5 | -1 | HCN2 |
| chr15:23345235 | ACAGATGTTCACCACCACGCCGG (SEQ ID NO: 435) | 5 | -1 | HCN2 |
| chr17:47159881 | GTAGATTCCCATCACCAAGCTGG (SEQ ID NO: 436) | 5 | -1 | HCN2 |
| chr5:55887911 | ACAGGTCCGCACCACCACGCCGG (SEQ ID NO: 437) | 5 | -1 | HCN2 |
| chr20:33285579 | ACAGACACCCACCACCGCGCCAG (SEQ ID NO: 438) | 5 | -1 | HCN2 |
| chr5:154856276 | ACAGACCTGAACCACCGCGCCGG (SEQ ID NO: 439) | 6 | -1 | HCN2 |
| chr5:90055256 | ACAGACGCCCACCACCGTGCCCA (SEQ ID NO: 440) | 6 | -1 | HCN2 |
| chr11:112277687 | ACAGACGCCCACCACCGTGCCCG (SEQ ID NO: 441) | 6 | -1 | HCN2 |
| chr9:133240280 | ACAGACACCCACCACCACGCGGG (SEQ ID NO: 442) | 6 | -1 | HCN2 |
| chr4:153003433 | ACAGACCCACACCACCACACTGG (SEQ ID NO: 443) | 6 | -1 | HCN2 |
| chr12:101422512 | ACAGACACACACCACCACGCCGG (SEQ ID NO: 444) | 6 | -1 | HCN2 |
| chr10:29439456 | ACAAATCCACACCACCATGCAGG (SEQ ID NO: 445) | 6 | -1 | HCN2 |
| chr13:40788915 | ACAGACACGCACCACCACGCTGG (SEQ ID NO: 446) | 6 | -1 | HCN2 |
| chr13:25429231 | ACAGATACCCACCACCACACCGG (SEQ ID NO: 447) | 6 | -1 | HCN2 |
| chr19:3983171 | GCATGTCGACTTCTCCTCGGAGG (SEQ ID NO: 448) | 0 | -1 | EEF2 |
| chr12:112318875 | TTATGTCTACTTCTCCTAGGAGG (SEQ ID NO: 449) | 4 | -1 | EEF2 |
| chr6:28225261 | AGATGCCGACCTCTCCTCGAAGG (SEQ ID NO: 450) | 5 | -1 | EEF2 |
| chr17:49326601 | ACATGTGAACTACTCCTCAGGGG (SEQ ID NO: 451) | 5 | -1 | EEF2 |
| chr6:27251978 | CTCTGCGGACTTCTCCTCGGGGG (SEQ ID NO: 452) | 5 | 1 | EEF2 |
| chr8:143977089 | GCACCCCGACGCCTCCTCGGAAG (SEQ ID NO: 453) | 5 | -1 | EEF2 |
| chr2:241767549 | ACGTGCCGACCCCTCCTCTGGGG (SEQ ID NO: 454) | 6 | -1 | EEF2 |
| chr19:43533502 | GCAGGACGGCCCCTCCCCGGGGG (SEQ ID NO: 455) | 6 | -1 | EEF2 |
| chr4:190203697 | GCACGCCGGCGCCTCCCCGGAGG (SEQ ID NO: 456) | 6 | -1 | EEF2 |
| chr22:50807161 | GCACGCCGGCACCTCCCCGGAGG (SEQ ID NO: 457) | 6 | -1 | EEF2 |
| chr17:75061968 | ACAGGCCCATTTCTCCCCGGGGG (SEQ ID NO: 458) | 6 | 0 | EEF2 |
| chr19:39298045 | GCTGGTCTAGGACGTCCTCCAGG (SEQ ID NO: 459) | 0 | -1 | IL29 |
| chr13:77472463 | CCTGGTCTATGACGTCCTCCTGC (SEQ ID NO: 460) | 2 | -1 | IL29 |
| chr19:39236866 | GCTGGTCCAGGACATCCCCCAGG (SEQ ID NO: 461) | 3 | -1 | IL29 |
| chr19:39269576 | GCTGGTCCAAGACGTCCACCAGG (SEQ ID NO: 462) | 3 | -1 | IL29 |
| chr12:51527538 | GCTGGGCTAGGGCCTCCTCCAGG (SEQ ID NO: 463) | 3 | -1 | IL29 |
| chr2:232649161 | GCTGGTCTCCGGCGTCCTCCCGG (SEQ ID NO: 464) | 3 | -1 | IL29 |
| chr10:124559698 | ACTGGCCGAGGAAGTCCTCCAGG (SEQ ID NO: (465) | 4 | -1 | IL29 |
| chr17:77931434 | GCTGGGGAAGGACGTCCCCCGGG (SEQ ID NO: 466) | 4 | -1 | IL29 |
| chr19:39244071 | GCTGGTCCAAGACATCCCCCAGG (SEQ ID NO: 467) | 4 | -1 | IL29 |
| chr1:14763373 | GCTGGGTTAGAATGTCCTCCAGG (SEQ ID NO: 468) | 4 | 0 | IL29 |
| chr13:81317427 | ACTGGTTTATAACGTCCTCCTGG (SEQ ID NO: 469) | 4 | -1 | IL29 |
| chr11:112769315 | GCTAGTCCAGAACGGCCTCCAGG (SEQ ID NO: 470) | 4 | -1 | IL29 |
| chr9:75409486 | ACTGGTCTAGGACATTCCCCCGG (SEQ ID NO: 471) | 4 | -1 | IL29 |
| chr14:106399152 | GCAGGCCCAGAGCGTCCTCCTGG (SEQ ID NO: 472) | 5 | -1 | IL29 |
| chr19:48757022 | GGAAACTCACCGATCCATACAGG (SEQ ID NO: 473) | 0 | -1 | FGF21 |
| chr1:169792715 | GCCAGCAAAGCACATTATTTTGG (SEQ ID NO: 474) | 0 | -1 | METTL18 |
| chr20:44771378 | GGCCCGTCTCCGTGCTCCTCTGG (SEQ ID NO: 475) | 0 | -1 | RIMS4 |
| chr1:25544959 | GGCCCGCCTCCCTCCTCCTCTGG (SEQ ID NO: 476) | 3 | -1 | RIMS4 |
| chr21:8440015 | GGGGTGCCTCCGGGCTCCTCGGG (SEQ ID NO: 477) | 5 | -3 | RIMS4 |
| chr20:63494913 | GCGCTACGACGAGATCGTCAAGG (SEQ ID NO: 478) | 0 | -1 | EEF1A2 |
| chr1:190234376 | GAGAATAAGATTCAGTTGCAAGG (SEQ ID NO: 479) | 0 | -1 | FAM5C |
| chr22:43956592 | GAGAAAGAGTTTCAGTTGCAGGG (SEQ ID NO: 480) | 3 | 0 | FAM5C |
| chr5:91688081 | AAGAATAAGAGTCAGTTGTAGGG (SEQ ID NO: 481) | 3 | -1 | FAM5C |
| chr2:31244390 | GTTTCTTGGGATCCACCACCAGG (SEQ ID NO: 482) | 0 | -1 | EHD3 |
| chr7:148568380 | GTTTATTAGGATCCACCACCTGA (SEQ ID NO: 483) | 2 | -1 | EHD3 |
| chr12:119154770 | GCTGCTCGGGATCCACCACCAGG (SEQ ID NO: 484) | 3 | -1 | EHD3 |
| chr11:134028043 | GCTTCTTGGGAGTCACCACCAGG (SEQ ID NO: 485) | 3 | -1 | EHD3 |
| chr15:84154968 | GCTCCTTGGGATCCACCGCCTGG (SEQ ID NO: 486) | 3 | 0 | EHD3 |
| chr9:106941860 | GTTTCTAGGAATCCACCATCCGG (SEQ ID NO: 487) | 3 | -1 | EHD3 |
| chr12:1846328 | TGTTCTAGGGACCCACCACCAGG (SEQ ID NO: 488) | 4 | 0 | EHD3 |
| chr19:56098961 | CTTCCTGGGGACCCACCACCTGG (SEQ ID NO: 489) | 4 | -1 | EHD3 |
| chr11:67201411 | GCCTCAAGGGATCCACCACCTGG (SEQ ID NO: 490) | 4 | -1 | EHD3 |
| chr1:53537504 | TGTGCTGGGGATCCACCACCGGG (SEQ ID NO: 491) | 4 | 0 | EHD3 |
| chr14:100281903 | GCTTCCTGGCATCCACCCCCAGG (SEQ ID NO: 492) | 4 | -1 | EHD3 |
| chr8:127124187 | ACTACCTGGGATCCACCACCAGA (SEQ ID NO: 493) | 4 | -1 | EHD3 |
| chr20:46782557 | AGACCTTGGGATCCACCACCTGT (SEQ ID NO: 494) | 4 | -1 | EHD3 |
| chr16:2686162 | CCAGCTTGGGACCCACCACCCGC (SEQ ID NO: 495) | 5 | -1 | EHD3 |
| chr19:10203524 | GATTCCAGGCACCCACCACCTGG (SEQ ID NO: 496) | 5 | -1 | EHD3 |
| chr14:95895923 | CCATCATGGCATCCACCACCAGG (SEQ ID NO: 497) | 5 | -1 | EHD3 |
| chr2:45976545 | GTAGGTGGGCTGCCGAAGATAGG (SEQ ID NO: 498) | 0 | -1 | PRKCE |
| chr2:188734617 | GTAATTAGGTAAGGCTTAGTTGG (SEQ ID NO: 499) | 0 | -1 | DIRC1 |
| chrX:42678955 | CCATTTAGGTAAAGCTTAGTGGG (SEQ ID NO: 500) | 4 | -1 | DIRC1 |
| chr9:2824054 | GTGATAGGGTTAGGGTTAGGGTT (SEQ ID NO: 501) | 6 | -2 | DIRC1 |
| chr2:191846550 | GCTCTTTGACCGCGCGCGTGTGG (SEQ ID NO: 502) | 0 | 0 | SDPR |
| chr2:123804334 | GATCTTGGACTGCTCCCCTGGCA (SEQ ID NO: 503) | 6 | 0 | SDPR |
| chr3:41225478 | GAAACAGCTCGTTGTACCGCTGG (SEQ ID NO: 504) | 0 | -1 | CTNNB1 |
| chr6:95084930 | GAAGCAGCTTGTTGTACCTCTGG (SEQ ID NO: 505) | 3 | -1 | CTNNB1 |
| chr9:128999980 | GAAGCAGCCCATTGTACTGCAGG (SEQ ID NO: 506) | 4 | -1 | CTNNB1 |
| chr6:28834918 | GAAACACCTCCTTGTGGGGAACT (SEQ ID NO: 507) | 6 | -1 | CTNNB1 |
| chr3:112630214 | GCAACAACGTGATGAATATCTGG (SEQ ID NO: 508) | 0 | -1 | CCDC80 |
| chr1:13780118 | GTCGCTGTGACTTTCTAATTTGG (SEQ ID NO: 509) | 0 | -1 | PRDM2 |
| chr1:109917360 | GGTGTTATCTCTGAAGCGCATGG (SEQ ID NO: 510) | 0 | -1 | CSF1 |
| chr3:68183902 | GTGGTTATCTCTGAAGCACATGG (SEQ ID NO: 511) | 3 | -1 | CSF1 |
| chr16:31042502 | AGTGTTGTCTCTGAAGAGCATGG (SEQ ID NO: 512) | 3 | 0 | CSF1 |
| chr7:43989251 | AGTCCTATCTCTGAAGCCCAGGG (SEQ ID NO: 513) | 4 | -1 | CSF1 |
| chr7:102542665 | AGTCCTATCTCTGAAGCCCAGGG (SEQ ID NO: 514) | 4 | -1 | CSF1 |
| chr3:142578684 | GGATCATGGAAGCCAGCTCCAGG (SEQ ID NO: 515) | 0 | -1 | ATR |
| chr2:233171850 | GGATCAGGGAAGCCAGCCCCTGG (SEQ ID NO: 516) | 2 | -1 | ATR |
| chr14:50951971 | TGATCAAGGAAGCCAGCTCCAGG (SEQ ID NO: 517) | 2 | -1 | ATR |
| chr20:39151104 | GGAGCATGGAGGCCAGCTCTGGG (SEQ ID NO: 518) | 3 | -1 | ATR |
| chr17:81142981 | GGAACAGGGAGGCCAGCTCCAGG (SEQ ID NO: 519) | 3 | -1 | ATR |
| chr13:109235830 | AGAACAAGGAAGCCAGCTCCAGG (SEQ ID NO: 520) | 3 | -1 | ATR |
| chr18:50338139 | GGATAATAGAAGCCAGCTGCTGG (SEQ ID NO: 521) | 3 | -1 | ATR |
| chr8:4522880 | GGATTATGGAAGTAAGCTCCTGG (SEQ ID NO: 522) | 3 | -1 | ATR |
| chr3 :44419764 | GTAGCATGGAAGTCAGCCCCAGG (SEQ ID NO: 523) | 4 | -1 | ATR |
| chr22:38026445 | GGATCATGAAGACCAGCCCCTGG (SEQ ID NO: 524) | 4 | -1 | ATR |
| chr8:142873256 | AGATCACAGCAGCCAGCTCCTGG (SEQ ID NO: 525) | 4 | -1 | ATR |
| chr19:13883875 | GAATCAGGGAAGCCACCACCAGG (SEQ ID NO: 526) | 4 | -1 | ATR |
| chr7:70956569 | GGAAGACGGAAGCCAGATCCAGG (SEQ ID NO: 527) | 4 | -1 | ATR |
| chr19:30854246 | GGATCAAGTAAGTCAGCACCAGG (SEQ ID NO: 528) | 4 | -1 | ATR |
| chr17:19715202 | AGATCATAAAAGTCAGCACCTGG (SEQ ID NO: 529) | 5 | -1 | ATR |
| chr8:37451030 | CAGCAATGGAAGCCAGCTCCAGG (SEQ ID NO: 530) | 5 | -1 | ATR |
| chr19:53545748 | GGGACATGAGAGCCAGGACCCTG (SEQ ID NO: 531) | 6 | -1 | ATR |
| chr14:69952249 | GGTCTCGGCACTTGGCTCGCTGG (SEQ ID NO: 532) | 0 | -1 | SMOC1 |
| chr19:55654263 | GTTCTCGGCACCTGGCTCTCCGG (SEQ ID NO: 533) | 3 | -1 | SMOC1 |
| chr12:9404796 | GCTCTCAGAACCTGGCTCGCGGG (SEQ ID NO: 534) | 4 | -1 | SMOC1 |
| chr1:110633803 | GGCCTTGGCACCTGGCTCCCAGG (SEQ ID NO: 535) | 4 | -1 | SMOC1 |
| chr15:83164057 | GGAGGCTTCACAGCGCCCTCTGG (SEQ ID NO: 536) | 0 | -1 | RP11-382A20.3 |
| chr10:124613980 | GGAGCCTTCACAGTGCCCTCGGG (SEQ ID NO: 537) | 2 | -1 | RP11-382A20.3 |
| chr10:70537842 | CCAGGCTCCACAGCGCCCTCTGC (SEQ ID NO: 538) | 3 | -1 | RP11-382A20.3 |
| chr16:84309340 | AGAGGCTTCCCAGCACCCTCGGG (SEQ ID NO: 539) | 3 | -1 | RP11-382A20.3 |
| chr14:102524654 | TCAGGCTTCACAGCGCCCCCTGG (SEQ ID NO: 540) | 3 | -1 | RP11-382A20.3 |
| chr2:191245225 | GCCGGCTTCACAGCGCCCCCCGG (SEQ ID NO: 541) | 3 | -1 | RP11-382A20.3 |
| chr2:192251123 | AGAGACTTCACAGCACCCTCTGC (SEQ ID NO: 542) | 3 | -1 | RP11-382A20.3 |
| chr20:41008317 | CATGGCTTCACAGTGCCCTCAGG (SEQ ID NO: 543) | 4 | 0 | RP11-382A20.3 |
| chr4:26229442 | GGTGGCCCCACAGCACCCTCTGG (SEQ ID NO: 544) | 4 | -1 | RP11-382A20.3 |
| chrX:139949884 | ATTGGCTTCACAGTGCCCTCTGG (SEQ ID NO: 545) | 4 | -1 | RP11-382A20.3 |
| chr1:1490177 | GGGGGCTCCTCAGCCCCCTCGGG (SEQ ID NO: 546) | 4 | -1 | RP11-382A20.3 |
| chr2:176135153 | GGAAGCAGCACAGCACCCTCTGG (SEQ ID NO: 547) | 4 | -1 | RP11-382A20.3 |
| chr9:80539236 | AGAGGATGCACAGCACCCTCAGG (SEQ ID NO: 548) | 4 | -1 | RP11-382A20.3 |
| chr20:63160454 | AGAAGCTGCACAGTGCCCTCTGG (SEQ ID NO: 549) | 4 | -1 | RP11-382A20.3 |
| chr5:141668551 | ACAGTCTTCACAGCACCCTCCGG (SEQ ID NO: 550) | 4 | -1 | RP11-382A20.3 |
| chr5:66209533 | AGTGGCTTCCCAGTGCCCTCAGG (SEQ ID NO: 551) | 4 | -1 | RP11-382A20.3 |
| chr2:169799386 | ATAGGCTCCACAGAACCCTCCGG (SEQ ID NO: 552) | 5 | -1 | RP11-382A20.3 |
| chr20:40846370 | AAAGGCTCCCCAGTGCCCTCAGG (SEQ ID NO: 553) | 5 | -1 | RP11-382A20.3 |
| chr16:2828998 | GAGGCCCTCACAGCACCCTCAGG (SEQ ID NO: 554) | 5 | 0 | RP11-382A20.3 |
| chr18:10571777 | AGACACTCCACAGCCCCCTCTGG (SEQ ID NO: 555) | 5 | -1 | RP11-382A20.3 |
| chr19:47259308 | CCTGGCTCCCCAGTGCCCTCAGG (SEQ ID NO: 556) | 6 | -1 | RP11-382A20.3 |
| chr19:925801 | CCCGGCTCCCCAGCGCCCCCGGG (SEQ ID NO: 557) | 6 | -1 | RP11-382A20.3 |
| chr11:72678167 | CAGGGCTCCCCAGTGCCCTCAGG (SEQ ID NO: 558) | 6 | -1 | RP11-382A20.3 |
| chr3:49706381 | CCTGGCTCCACTGCACCCTCCGG (SEQ ID NO: 559) | 6 | -1 | RP11-382A20.3 |
| chr9:127868711 | CATGGCTCCCCAGTGCCCTCAGG (SEQ ID NO: 560) | 6 | -1 | RP11-382A20.3 |
| chr3:184365170 | GCTAGTACCTTGTATGAAGATGG (SEQ ID NO: 561) | 0 | -1 | POLR2H |
| chr13:50338526 | TCTAGTGCCTTGTATGAAGTTGG (SEQ ID NO: 562) | 3 | -1 | POLR2H |
| chr3:58513943 | ACTAGTACCCTGCAAGAAGATGG (SEQ ID NO: 563) | 4 | -1 | POLR2H |
| chr10:73237068 | ACTGGTATCTTATAAGAAGAGGG (SEQ ID NO: 564) | 5 | -1 | POLR2H |
| chr4:41650411 | GACGGGAAAGTCAGTGTGAATGG (SEQ ID NO: 565) | 0 | -1 | LIMCH1 |
| chr1:38941382 | GGAGGGAAAGCCAGTGTGAAGGG (SEQ ID NO: 566) | 3 | 0 | LIMCH1 |
| chr5:127657762 | GTTCGACCATGCCCTTGCTTAGG (SEQ ID NO: 567) | 0 | -1 | CTXN3 |
| chr1:199352406 | TGTAGACCATGCCATTGCTTTGG (SEQ ID NO: 568) | 4 | -1 | CTXN3 |
| chr16:713763 | GCTCGGCCAGCCCCTTGCTCTGG (SEQ ID NO: 569) | 5 | -1 | CTXN3 |
| chr1:31619705 | GGCAGAGCTCACCTGTAGATAGG (SEQ ID NO: 570) | 0 | -1 | HCRTR1 |
| chr1:4408639 | CAAAGAGCTCACCTGTAGATCAG (SEQ ID NO: 571) | 3 | -1 | HCRTR1 |
| chr8:97032246 | AGCAGAGCCCTACTGTAGATTGG (SEQ ID NO: 572) | 4 | -1 | HCRTR1 |
| chr17:76226063 | CACAGAGAACACCTGGAGATGGG (SEQ ID NO: 573) | 5 | -1 | HCRTR1 |
| chr22:39522289 | CACAGAGAACACCTGGAGATGGG (SEQ ID NO: 574) | 5 | -1 | HCRTR1 |
| chr7:107593998 | GCTGGTGGAGCTCTTCTCAATGG (SEQ ID NO: 575) | 0 | -1 | BCAP29 |
| chr10:123687944 | GCTAGTGGAGCTCTTCTCCACGG (SEQ ID NO: 576) | 2 | 0 | BCAP29 |
| chr7:128098718 | GCTGGTGGGGCTCTTCTCAGAAG (SEQ ID NO: 577) | 2 | -1 | BCAP29 |
| chr20:38006300 | TGTGGTGGTGCTCTTCTCAAGAG (SEQ ID NO: 578) | 3 | 0 | BCAP29 |
| chr6:92171764 | CCTGGTGGTTCTCTTCTCAATGG (SEQ ID NO: 579) | 3 | -1 | BCAP29 |
| chr12:120978195 | GCTGGGCTAGCTCTTCTCAAGGG (SEQ ID NO: 580) | 3 | -1 | BCAP29 |
| chr4:141367193 | CTTGGGGGAGCTCTTCTCAAGGA (SEQ ID NO: 581) | 3 | -1 | BCAP29 |
| chr19:37313286 | GCTGGAGAGGCTCTTCTCAAGGA (SEQ ID NO: 582) | 3 | -1 | BCAP29 |
| chr20:21362935 | ACTGGAGCAGCCCTTCTCAATGG (SEQ ID NO: 583) | 4 | -1 | BCAP29 |
| chr2:102186472 | ACTGGTCAAGCTCTTCCCAACGG (SEQ ID NO: 584) | 4 | -1 | BCAP29 |
| chr9:136671847 | GCTTGTGGAGCCCTTCCCAGGGG (SEQ ID NO: 585) | 4 | 0 | BCAP29 |
| chr6:33927138 | ACTGGTGAAGCTCTAGTCAAAGG (SEQ ID NO: 586) | 4 | -1 | BCAP29 |
| chr1:201391878 | GCTGGGGGAGCCCTTCTCTGTGG (SEQ ID NO: 587) | 4 | 0 | BCAP29 |
| chr7:157754655 | TCTGGGGGGGCCCTTCTCAAGGG (SEQ ID NO: 588) | 4 | 0 | BCAP29 |
| chr4:189344074 | ACCAGAGGAGCTCTTCTCAAAGG (SEQ ID NO: 589) | 4 | 0 | BCAP29 |
| chr16:4682690 | GCTGGTGATGCCCTTCTCCAGGG (SEQ ID NO: 590) | 4 | 0 | BCAP29 |
| chr3:11726423 | GCTGCCAGAGCCCTTCTCAAAAG (SEQ ID NO: 591) | 4 | -1 | BCAP29 |
| chr2:86572609 | GCTGATGGTGCCCTTCTAAAAGG (SEQ ID NO: 592) | 4 | -1 | BCAP29 |
| chr16:69586 | GCTGGTGACCCCCTTCTCAAGGG (SEQ ID NO: 593) | 4 | -1 | BCAP29 |
| chr15:75652896 | AGGGGTGGAGCCCTTCTCAAAGA (SEQ ID NO: 594) | 4 | 0 | BCAP29 |
| chr4:180505414 | TATGGTGGAGGACTTCTCAAAGG (SEQ ID NO: 595) | 4 | -1 | BCAP29 |
| chr2:227889449 | AATGGTGGAGCCCTTCTGAATGG (SEQ ID NO: 596) | 4 | -1 | BCAP29 |
| chr8:144441012 | GCTAGGGGACCTCTTCTCCAAGG (SEQ ID NO: 597) | 4 | -1 | BCAP29 |
| chr3:55406561 | GAGGGTGGAGCCCTTATCAATGG (SEQ ID NO: 598) | 4 | -1 | BCAP29 |
| chr17:6549115 | CCTGGAGAAGCTCTTCTCCAGGG (SEQ ID NO: 599) | 4 | -1 | BCAP29 |
| chr22:38235223 | ACTGGAGGAGCTCCTCTCAGAGG (SEQ ID NO: 600) | 4 | 0 | BCAP29 |
| chr9:61939297 | GCTGGGGAGGCCCTTCTCAAGGA (SEQ ID NO: 601) | 4 | -1 | BCAP29 |
| chr20:20165131 | GCTGTTGGACCCCTTCTCAGAGG (SEQ ID NO: 602) | 4 | -1 | BCAP29 |
| chr9:88954076 | GCTGGGAGGGCTCTTCCCAATGG (SEQ ID NO: 603) | 4 | -1 | BCAP29 |
| chr16:15208059 | AAGGGTGGAGCCCTTATCAATGG (SEQ ID NO: 604) | 5 | -1 | BCAP29 |
| chr17:51426052 | TTTGGGGAAGCCCTTCTCAAGGG (SEQ ID NO: 605) | 5 | -1 | BCAP29 |
| chr5:168839089 | TTCTGAGGAGCTCTTCTCAAGGG (SEQ ID NO: 606) | 5 | -1 | BCAP29 |
| chr17:2064999 | GTCAGTGGAGCCCTTCTCAGGGG (SEQ ID NO: 607) | 5 | -1 | BCAP29 |
| chr14:91315897 | ACTGATGGGTCTTTTCTCAAGGG (SEQ ID NO: 608) | 5 | -1 | BCAP29 |
| chr3:51942833 | GCTGTAGAAGCCCTTCCCAATGG (SEQ ID NO: 609) | 5 | -1 | BCAP29 |
| chr12:132746996 | GCGGGCACAGCTCTTCTAAAGGG (SEQ ID NO: 610) | 5 | -2 | BCAP29 |
| chr16:18119679 | AAGGGTGGAGCCCTCATCAATGG (SEQ ID NO: 611) | 6 | -1 | BCAP29 |
| chr12:124940141 | GCTGGCGCAGCCCCTTCCAAGGG (SEQ ID NO: 612) | 6 | -1 | BCAP29 |
| chr7:137928331 | GGAGCTGACCCAAGACGTTCTGG (SEQ ID NO: 613) | 0 | -1 | CREB3L2 |
| chr5:122390428 | AGAGCTGACTGAAGACGTTCCGG (SEQ ID NO: 614) | 3 | -1 | CREB3L2 |
| chr9:36143630 | ACAACTGACCCAAGACGTGCAGG (SEQ ID NO: 615) | 4 | -1 | CREB3L2 |
| chr4:71357031 | GTTGACCATCAGATTGAGACAGG (SEQ ID NO: 616) | 0 | 0 | SLC4A4 |
| chr4:108167564 | GCTCACCTCGTGTCCGTTGCTGG (SEQ ID NO: 617) | 0 | -1 | LEF1 |
| chr4:184659355 | GGACGTTCATGTATTTGCTTTGG (SEQ ID NO: 618) | 0 | -1 | CCDC111 |
| chr12:54500702 | AGATGTTCATGTATTTGCTTAAA (SEQ ID NO: 619) | 2 | -1 | CCDC111 |
| chr12:70307436 | ACACACTCATGTATTTGCTTAGG (SEQ ID NO: 620) | 4 | -1 | CCDC111 |
| chr5:41862667 | GCTGTAAAAGACATCCCTGATGG (SEQ ID NO: 621) | 0 | -1 | OXCT1 |
| chr11:133063288 | GCTGGAAAAGGCATCCCTGAGGG (SEQ ID NO: 622) | 2 | -1 | OXCT1 |
| chr17:65894010 | TCTGTAAGAGACATCCCTGATGT (SEQ ID NO: 623) | 2 | -1 | OXCT1 |
| chr3:52624560 | TCTGTAAAAGGCATCCCTGAAAG (SEQ ID NO: 624) | 2 | -1 | OXCT1 |
| chr8:8563818 | GCAGTGAAAGACATCCCTGTGGG (SEQ ID NO: 625) | 3 | -1 | OXCT1 |
| chr11:14182335 | GCTGTAGAAGACATCCCAGTAAG (SEQ ID NO: 626) | 3 | -1 | OXCT1 |
| chr19:1592539 | ATAGTAAAAGACATCCCTGTGGC (SEQ ID NO: 627) | 4 | -1 | OXCT1 |
| chr5:43277173 | GGGTCTCCACCACTTCGTAAAGG (SEQ ID NO: 628) | 0 | -1 | AC114947.1 |
| chr16:29713006 | GAGTCTCCACCATTTCATAATGG (SEQ ID NO: 629) | 3 | -1 | AC114947.1 |
| chr11:78139568 | GGCGGCGCTCACAATTGCCACGG (SEQ ID NO: 630) | 0 | -1 | ALG8 |
| chr1:112341503 | GGTAGAGCTCACAATTGCCAAGG (SEQ ID NO: 631) | 3 | -1 | ALG8 |
| chr4:68194512 | AGGGGCGCCCACAATTGCCAAGG (SEQ ID NO: 632) | 3 | -1 | ALG8 |
| chr2:169399634 | AGGGGCGCTCAGAATTGCCAAGG (SEQ ID NO: 633) | 3 | -1 | ALG8 |
| chr10:99449728 | GGAGCCACTCACAATTGCCAAGG (SEQ ID NO: 634) | 3 | -1 | ALG8 |
| chrX:73185300 | AGGGGCACCCACAATTGCCAAGG (SEQ ID NO: 635) | 4 | -1 | ALG8 |
| chr3:99294178 | AGGGGCGCCCACAATTGCCCAGG (SEQ ID NO: 636) | 4 | -1 | ALG8 |
| chr9:90192643 | AGGGGCACCCACAATTGCCAAGG (SEQ ID NO: 637) | 4 | -1 | ALG8 |
| chr6:86731841 | AGGGGCGCCCACAATTGCCTAGG (SEQ ID NO: 638) | 4 | -1 | ALG8 |
| chr6:86283827 | AGGGGTGCCCACAATTGCCAAGG (SEQ ID NO: 639) | 4 | -1 | ALG8 |
| chrX:64484062 | AGGGGCCCCCACAATTGCCAAGG (SEQ ID NO: 640) | 4 | -1 | ALG8 |
| chr6:52861283 | AGGGGCGCCCACCATTGCCAAGG (SEQ ID NO: 641) | 4 | -1 | ALG8 |
| chrX:55811741 | AGGGGCGCCCACAATTGCCTAGA (SEQ ID NO: 642) | 4 | -1 | ALG8 |
| chr6:72164084 | AGGGGCGCCCACCATTGCCAAGG (SEQ ID NO: 643) | 4 | -1 | ALG8 |
| chr5:88313697 | AGGGGCGCCCACCATTGCCAAGG (SEQ ID NO: 644) | 4 | -1 | ALG8 |
| chr2:85964247 | AGGGGCGCCCACCATTGCCAAGG (SEQ ID NO: 645) | 4 | -1 | ALG8 |
| chr4:92944267 | AGGGGCACCCACAATTGCCCAGG (SEQ ID NO: 646) | 5 | -1 | ALG8 |
| chr6:86057508 | AGGGGCACCCACAATTGCCCAGT (SEQ ID NO: 647) | 5 | -1 | ALG8 |
| chr12:89521784 | AGCACCATTCACAATTGCCAAGG (SEQ ID NO: 648) | 5 | -1 | ALG8 |
| chr5:131087608 | AGGGGCGCCCGCCATTGCCAAGG (SEQ ID NO: 649) | 5 | -1 | ALG8 |
| chr4:78118512 | AGGGGTGCCCACCATTGCCAAGT (SEQ ID NO: 650) | 5 | -1 | ALG8 |
| chr11:50199456 | TGGGGCACCCACAATTTCCAAGG (SEQ ID NO: 651) | 5 | -2 | ALG8 |
| chr6:52096649 | AGGGGCGCCCGCCATTGCCAAGG (SEQ ID NO: 652) | 5 | -1 | ALG8 |
| chrX:91627551 | AGGGGGGCCCACAATTGCCCAGG (SEQ ID NO: 653) | 5 | -1 | ALG8 |
| chr8:43350131 | AGGGGCACCCACAATTGCTCAGG (SEQ ID NO: 654) | 6 | -1 | ALG8 |
| chr14:59409903 | AGGGGCACCCACAATTGCTGAGG (SEQ ID NO: 655) | 6 | -1 | ALG8 |
| chr4:69664461 | AGGGGCGCCCACCATTGACCAGG (SEQ ID NO: 656) | 6 | -1 | ALG8 |
| chr14:105961812 | AGGGGTGCCCACAATTGCTGAGG (SEQ ID NO: 657) | 6 | -1 | ALG8 |
| chr18:33787333 | AGGGGTGCCCGCCATTGCCAAGG (SEQ ID NO: 658) | 6 | -1 | ALG8 |
| chr20:45693526 | AGGGGCGCCCACCATTGCACAGG (SEQ ID NO: 659) | 6 | -1 | ALG8 |
| chr5:46193866 | AGGGGCACCCACTATTGCCCAGG (SEQ ID NO: 660) | 6 | -1 | ALG8 |
| chr11:111515537 | GGTACTTACTGTTACTCGCAAGG (SEQ ID NO: 661) | 0 | -1 | C11orf88 |
| chr5:115721586 | GGTACTTACTGCTACTCTCCAGG (SEQ ID NO: 662) | 3 | -1 | C11orf88 |
| chr12:57608619 | GACGCTGGTCAAACGCCTTGCGG (SEQ ID NO: 663) | 0 | -1 | DTX3 |
| chr1:236739590 | GACCCAGGTCAAACGCCTTTAGG (SEQ ID NO: 664) | 3 | -1 | DTX3 |
| chr16:67179435 | GGCATGCTGCGGCATGAGATAGG (SEQ ID NO: 665) | 0 | -1 | KIAA0895 L |
| chr18:10725455 | GGCATGCTGTGGCATGAAATAGG (SEQ ID NO: 666) | 2 | -1 | KIAA0895 L |
| chr2:229369146 | GGCTTGCTGCAGCATGAGTTAGG (SEQ ID NO: 667) | 3 | 0 | KIAA0895 L |
| chr22:37524224 | GGAATGCTGCGGCATGATCTTGG (SEQ ID NO: 668) | 3 | -1 | KIAA0895 L |
| chrX:135174521 | CGGATGCTGCAGCAAGAGATTGG (SEQ ID NO: 669) | 4 | -1 | KIAA0895 L |
| chr10:78907705 | CACATGATGCAGCATGAGATGGG (SEQ ID NO: 670) | 4 | -1 | KIAA0895 L |
| chrX:135221008 | CGGATGCTGCAGCAAGAGATTGG (SEQ ID NO: 671) | 4 | -1 | KIAA0895 L |
| chr19:48628075 | GACGGGCTGCTCCATGAGGTAGA (SEQ ID NO: 672) | 6 | -1 | KIAA0895 L |
| chr18:26227083 | GGCTCCACGCAGACGCTGACAGG (SEQ ID NO: 673) | 0 | -1 | TAF4B |
| chr2:231711896 | GTCGAGGAGAATGAGGAAAATGG (SEQ ID NO: 674) | 0 | -1 | PTMA |
| chr12:45223775 | TTAGAGGAGAATGAGGAAAAGAG (SEQ ID NO: 675) | 2 | -1 | PTMA |
| chr8:39584236 | GTGGAGGAGAAAGAGGAAAAGGG (SEQ ID NO: 676) | 2 | -1 | PTMA |
| chr4:169422685 | GTAGAGGAGTATGAGGAAAAGAG (SEQ ID NO: 677) | 2 | -1 | PTMA |
| chr5:157259662 | GTTGAGGAGAAGGAGGAAAAGGA (SEQ ID NO: 678) | 2 | 0 | PTMA |
| chrX:69115918 | GTCCAGGAGAATGAGGAAAGGAG (SEQ ID NO: 679) | 2 | 1 | PTMA |
| chr13:32593798 | GTTGAGGAGAAGGAGGAAAAGGA (SEQ ID NO: 680) | 2 | 0 | PTMA |
| chr7:145356277 | GTTGAGTAGAATGAGGAAAAGGA (SEQ ID NO: 681) | 2 | -1 | PTMA |
| chr11:123108690 | AGGGAGGAGAATGAGGAAAAGGG (SEQ ID NO: 682) | 3 | -1 | PTMA |
| chr11:25976719 | GAGGAGGAGAAAGAGGAAAAGGG (SEQ ID NO: 683) | 3 | 0 | PTMA |
| chr5:107677158 | GAAGGGGAGAATGAGGAAAAGGG (SEQ ID NO: 684) | 3 | -1 | PTMA |
| chr20:49290142 | GCCAAGGAGAATGAGAAAAAGAG (SEQ ID NO: 685) | 3 | -1 | PTMA |
| chr12:106656688 | GGAGAGGAGAATGAGGAGAAGGG (SEQ ID NO: 686) | 3 | -1 | PTMA |
| chr20:10429657 | GATGAGGAGCATGAGGAAAAGGG (SEQ ID NO: 687) | 3 | -1 | PTMA |
| chr5:95007120 | GAAGAGGAGAATGAGAAAAAGGG (SEQ ID NO: 688) | 3 | 0 | PTMA |
| chr8:73415385 | CTGGAGAAGAATGAGGAAAAAGG (SEQ ID NO: 689) | 3 | -1 | PTMA |
| chr4:30802717 | GTTGAGGGGAATGAGGATAAGGG (SEQ ID NO: 690) | 3 | -1 | PTMA |
| chr17:79296708 | GAGGAGGAGAAAGAGGAAAAAAG (SEQ ID NO: 691) | 3 | -1 | PTMA |
| chr3:103906656 | GACGAAGAGAAAGAGGAAAAGAG (SEQ ID NO: 692) | 3 | -1 | PTMA |
| chr9:78720991 | CTCGAGGGGAATGAGGAGAAGGG (SEQ ID NO: 693) | 3 | -1 | PTMA |
| chr4:163769948 | GTTGAGGAGAAAAAGGAAAAGGG (SEQ ID NO: 694) | 3 | -1 | PTMA |
| chr11:130687297 | ACAGAGGAGAATGAGGAAAAAGA (SEQ ID NO: 695) | 3 | -1 | PTMA |
| chr6:90438937 | GATGAGGGGAATGAGGAAAACAG (SEQ ID NO: 696) | 3 | -1 | PTMA |
| chr8:101411662 | GAGGAAGAGAATGAGGAAAAGGA (SEQ ID NO: 697) | 3 | -1 | PTMA |
| chrX:108119774 | GGTGAGGAGAAGGAGGAAAAGGA (SEQ ID NO: 698) | 3 | -1 | PTMA |
| chr2:62564410 | GAAGAGGAGAAGGAGGAAAAGGA (SEQ ID NO: 699) | 3 | 0 | PTMA |
| chr17:59193640 | GTGGAGGAGGAGGAGGAAAATGG (SEQ ID NO: 700) | 3 | -1 | PTMA |
| chr10:61198920 | GCTGAGGAGAAGGAGGAAAAGGA (SEQ ID NO: 701) | 3 | 0 | PTMA |
| chr14:33399434 | AACAAGGAGAATGAGGAAAAAGC (SEQ ID NO: 702) | 3 | 0 | PTMA |
| chr4:90840258 | GTGGAGAAGAATGAGGAGAAAGG (SEQ ID NO: 703) | 3 | 0 | PTMA |
| chr10:7505297 | GTGGAGGAGGAGGAGGAAAAGGG (SEQ ID NO: 704) | 3 | -1 | PTMA |
| chr5:147928310 | GAAGAGGAGAATGAGGACAAGAG (SEQ ID NO: 705) | 3 | -1 | PTMA |
| chr3:34408131 | GAAGAGGAGAATGAGAAAAAGGA (SEQ ID NO: 706) | 3 | 0 | PTMA |
| chr8:74460850 | GTGGAGGAGAAAGAGGAGAAGAG (SEQ ID NO: 707) | 3 | 0 | PTMA |
| chr10:122543164 | GTGGAAGAGAATGAAGAAAAGAG (SEQ ID NO: 708) | 3 | 0 | PTMA |
| chr18:29500361 | GCTGAGGAGAAGGAGGAAAAGGA (SEQ ID NO: 709) | 3 | 0 | PTMA |
| chr5:149683682 | GTTGCAGAGAATGAGGAAAAGGG (SEQ ID NO: 710) | 3 | -1 | PTMA |
| chr15:40876038 | GCTGAGGAGAAGGAGGAAAAGGA (SEQ ID NO: 711) | 3 | 0 | PTMA |
| chr14:65350141 | GCTGAGGAGAATGAGGAGAACAG (SEQ ID NO: 712) | 3 | 0 | PTMA |
| chr13:40385569 | GAAGAGGAGAAGGAGGAAAAAGA (SEQ ID NO: 713) | 3 | 0 | PTMA |
| chr1:78293196 | GCTGAGGAGAAGGAGGAAAAGGA (SEQ ID NO: 714) | 3 | -1 | PTMA |
| chr15:24067371 | GCAGAGGAGAAAGAGGAAAAAGA (SEQ ID NO: 715) | 3 | -1 | PTMA |
| chr7:130835025 | ATGGAGGAGAATGAAGAAAAAAG (SEQ ID NO: 716) | 3 | -1 | PTMA |
| chr7:51094241 | GTAGAGGAGAGAGAGGAAAAGAG (SEQ ID NO: 717) | 3 | -1 | PTMA |
| chr4:36663573 | GTAGAGGAGAAAGAGAAAAAGAG (SEQ ID NO: 718) | 3 | -1 | PTMA |
| chr4:180190828 | ACTGAGGAGAAAGAGGAAAATGG (SEQ ID NO: 719) | 4 | -1 | PTMA |
| chr2:182860557 | AGTGAGGGGAATGAGGAAAAAGG (SEQ ID NO: 720) | 4 | 0 | PTMA |
| chr7:100883368 | AATGAGGAGTATGAGGAAAAGGG (SEQ ID NO: 721) | 4 | -1 | PTMA |
| chr11:33473717 | AGAGGGGAGAATGAGGAAAATGG (SEQ ID NO: 722) | 4 | -1 | PTMA |
| chr21:44966689 | ACAGAGGGGAATGAGGAAAAGGG (SEQ ID NO: 723) | 4 | -1 | PTMA |
| chr15:58590555 | AAGGAGGAGAAAGAGGAAAATGG (SEQ ID NO: 723) | 4 | -1 | PTMA |
| chr1:54321788 | TAAGAGCAGAATGAGGAAAAGGG (SEQ ID NO: 725) | 4 | 0 | PTMA |
| chr1:154159113 | GAGGAGGAGAAAGAGAAAAAGGG (SEQ ID NO: 726) | 4 | 0 | PTMA |
| chr6:154255624 | AAAGAAGAGAATGAGGAAAATGG (SEQ ID NO: 727) | 4 | -1 | PTMA |
| chr5:154682833 | GGGGAGGAGAAAGAGGAAAGGGG (SEQ ID NO: 728) | 4 | -1 | PTMA |
| chr4:155280123 | AGAGAGGAGAAGGAGGAAAAAGG (SEQ ID NO: 729) | 4 | 0 | PTMA |
| chr19:35694227 | GAGGAGGAGAAAGAGAAAAAAGG (SEQ ID NO: 730) | 4 | -1 | PTMA |
| chr2:178388909 | TGGGAGGAGAATGAGGGAAAAGG (SEQ ID NO: 731) | 4 | -1 | PTMA |
| chrX:125204528 | GAGGAGGAGAAAGAGGAGAAGGG (SEQ ID NO: 732) | 4 | 0 | PTMA |
| chr3:28055643 | AAGGAGCAGAATGAGGAAAAAGG (SEQ ID NO: 733) | 4 | -1 | PTMA |
| chr11:133825402 | GAGGAGGAGAAAGAGGAATAGGG (SEQ ID NO: 734) | 4 | -1 | PTMA |
| chr1:60539324 | CTGGAGGAGAAAGAGGAATAGGG (SEQ ID NO: 735) | 4 | 0 | PTMA |
| chr8:120581188 | GCAAAGGAGAATGAGAAAAAAGG (SEQ ID NO: 736) | 4 | 0 | PTMA |
| chr5:74251417 | CCAGAGGAGACTGAGGAAAATGG (SEQ ID NO: 737) | 4 | -1 | PTMA |
| chr15:43928320 | GGTGAGGGGAATGAGGAAAGAGG (SEQ ID NO: 738) | 4 | 0 | PTMA |
| chr7:84196472 | GAGGGGGAGAATGGGGAAAAGGG (SEQ ID NO: 739) | 4 | -1 | PTMA |
| chr20:4185198 | ATTGAGGAGAAAGAGGAGAATGG (SEQ ID NO: 740) | 4 | 0 | PTMA |
| chr3:93984475 | GCTGAGGAGAAAGAGGAAGAGGG (SEQ ID NO: 741) | 4 | -1 | PTMA |
| chr17:79476918 | AAAGAGGAGAAAGAGGAAAAGGA (SEQ ID NO: 742) | 4 | 0 | PTMA |
| chr2:198709174 | GAGGAAGAGAAAGAGGAAAATGG (SEQ ID NO: 743) | 4 | -1 | PTMA |
| chr7:117282486 | GAGGAGGAGAAAGAAGAAAAAGG (SEQ ID NO: 744) | 4 | 0 | PTMA |
| chr18:59032314 | ACCGAAGAGAATGAGGAAACAAG (SEQ ID NO: 745) | 4 | -1 | PTMA |
| chr1:84083389 | GAGGAGGAGAATAAGAAAAATGG (SEQ ID NO: 746) | 4 | -1 | PTMA |
| chr7:101837984 | ATAGAGTAGAATGAGGAAAGGGG (SEQ ID NO: 747) | 4 | -1 | PTMA |
| chr22:28401159 | AAGGAGGAGAAAGAGGAAAAGGA (SEQ ID NO: 748) | 4 | 0 | PTMA |
| chr7:93571911 | AAAGAGGAGAAAGAGGAAAATAG (SEQ ID NO: 749) | 4 | -1 | PTMA |
| chr9:26301977 | GCCAAGGAGAAAGAGGAAGAGGG (SEQ ID NO: 750) | 4 | -1 | PTMA |
| chr12:111257272 | GAGGAGGAGGAAGAGGAAAAGGG (SEQ ID NO: 751) | 4 | -2 | PTMA |
| chr2:127309056 | GAGGAGGAGAAAGGGGAAAAGGG (SEQ ID NO: 752) | 4 | 0 | PTMA |
| chr20:63226610 | GCTGAGGAGAAGGAGGAAAGGGG (SEQ ID NO: 753) | 4 | -1 | PTMA |
| chr14:80385345 | GGTGAAGAGAATGAGGAAAGAGG (SEQ ID NO: 754) | 4 | -1 | PTMA |
| chr14:92235140 | TATGAGGAGAATGAGGAGAAGAG (SEQ ID NO: 755) | 4 | -1 | PTMA |
| chr6:60556386 | GGGGAGGAGAAAGAAGAAAAGGG (SEQ ID NO: 756) | 4 | 0 | PTMA |
| chr11:87142779 | AAGGAGGAGAAAGAGGAAAAAGA (SEQ ID NO: 757) | 4 | -1 | PTMA |
| chrX:102738253 | GAGGAGGAAAAAGAGGAAAAGGG (SEQ ID NO: 758) | 4 | 0 | PTMA |
| chr13:76411635 | GAGGAGGAGAAGGAGGAGAACGG (SEQ ID NO: 759) | 4 | 0 | PTMA |
| chr1:239662869 | GAAGAGGAGAAAGAGGAGAAAGG (SEQ ID NO: 760) | 4 | -1 | PTMA |
| chr17:13458972 | CTAGAGGAGAATGAGAAGAATGG (SEQ ID NO: 761) | 4 | -1 | PTMA |
| chr18:4247129 | GAGGAAGAGAAAGAGGAAAATGG (SEQ ID NO: 762) | 4 | -1 | PTMA |
| chr10:129464785 | GCAGAGGGGAAAGAGGAAAAAGG (SEQ ID NO: 763) | 4 | -1 | PTMA |
| chr7:68255184 | GAGGAGGAGAAAGAGGAGAAAGG (SEQ ID NO: 764) | 4 | -1 | PTMA |
| chr4:6935550 | GGAGAGGAGGAAGAGGAAAAGGG (SEQ ID NO: 765) | 4 | -1 | PTMA |
| chr21:35688790 | TTAGAGGAGAAAGAGGAAGAAGG (SEQ ID NO: 766) | 4 | -1 | PTMA |
| chr6:31973228 | GGAGAGGAGAGTGAGGAAGAGGG (SEQ ID NO: 767) | 4 | 0 | PTMA |
| chr20:23814421 | AGTAAGGAGAATGAGGAAAAAGC (SEQ ID NO: 768) | 4 | -1 | PTMA |
| chr6:57657607 | GGGGAGGAGAAAGAAGAAAAGGG (SEQ ID NO: 769) | 4 | -1 | PTMA |
| chr16:66873925 | GAGGAGGAGAAGGAGGAGAAGGG (SEQ ID NO: 770) | 4 | -2 | PTMA |
| chr12:115143574 | GAGGAGGAGAAAGAAGAAAACGG (SEQ ID NO: 771) | 4 | -1 | PTMA |
| chr19:29843380 | GCAGAGGAGGAGGAGGAAAAGGG (SEQ ID NO: 772) | 4 | -1 | PTMA |
| chr17:33004459 | GAGGAGGAGAAGGAGGAGAAGGG (SEQ ID NO: 773) | 4 | 0 | PTMA |
| chr3:160171017 | GCTGAGAAGAATGAGGAAAGGGG (SEQ ID NO: 774) | 4 | 0 | PTMA |
| chr3:53149304 | GCAGAGGAGAACAAGGAAAAGAG (SEQ ID NO: 775) | 4 | -1 | PTMA |
| chr8:105133771 | GAGGAGGAGAAAGAGGAACAGGG (SEQ ID NO: 776) | 4 | -1 | PTMA |
| chr6:18263848 | GAGGAGGAGGAGGAGGAAAAAGG (SEQ ID NO: 777) | 4 | -2 | PTMA |
| chr1:34748046 | GCCAAGGGGAATGAGGCAAAGGG (SEQ ID NO: 778) | 4 | -1 | PTMA |
| chr12:71135523 | GAGGAGGAGAAGGAGGAGAAGGG (SEQ ID NO: 779) | 4 | 0 | PTMA |
| chr3:50154013 | AGAGAGGAGAAGGAGGAAAAGGA (SEQ ID NO: 780) | 4 | -1 | PTMA |
| chr6:87746360 | AAGGAGGAGAATGAGGAGAAGGA (SEQ ID NO: 781) | 4 | -1 | PTMA |
| chr18:29751454 | GAAGAGGAGAAGGAGGAGAAGGG (SEQ ID NO: 782) | 4 | 0 | PTMA |
| chr20:57928833 | GAGGAGGAGGATGAGGAGAAGGG (SEQ ID NO: 783) | 4 | -2 | PTMA |
| chr3:146015656 | GAGGAGGAGGAAGAGGAAAAGGA (SEQ ID NO: 784) | 4 | -2 | PTMA |
| chr1:247337438 | GAGGAGGAGAAGGAGGAAGAGGG (SEQ ID NO: 785) | 4 | -1 | PTMA |
| chr5:167629931 | GAGGAGGAGAAAGAGGAAGAGGG (SEQ ID NO: 786) | 4 | -1 | PTMA |
| chr5:77818701 | GGAGAGGAGAATGAGGAGGAGGG (SEQ ID NO: 787) | 4 | -1 | PTMA |
| chrX:103832428 | GGGGAGGAGAAGGAGGACAAGGG (SEQ ID NO: 788) | 4 | -1 | PTMA |
| chr16:34642948 | GGTGAGGAGAAGGAAGAAAAAGG (SEQ ID NO: 789) | 4 | 0 | PTMA |
| chr2:51087233 | GGAGAAGAGAATGAGAAAAATGG (SEQ ID NO: 790) | 4 | 0 | PTMA |
| chr20:49483476 | GGGGAGGAGAAGGAGGAGAAGGG (SEQ ID NO: 791) | 4 | -2 | PTMA |
| chr16:46552887 | GCTGAGGAGAAGGAGGAAGAAGG (SEQ ID NO: 792) | 4 | -1 | PTMA |
| chr17:75840490 | GGTGAGGAGGATGAGGAAAGGGG (SEQ ID NO: 793) | 4 | -1 | PTMA |
| chr3:91362742 | GGGGAGGAGAAAGAAGAAAAGGG (SEQ ID NO: 794) | 4 | -1 | PTMA |
| chr10:64614803 | AAAGAGGAGAAAGAGGAAAAGGA (SEQ ID NO: 795) | 4 | 0 | PTMA |
| chr15:68387067 | AGGGAGGAGAATGAGGAGAAAAG (SEQ ID NO: 796) | 4 | 0 | PTMA |
| chr1:227077487 | GTAGAGGAGAACCAGGAGAAGGG (SEQ ID NO: 797) | 4 | -1 | PTMA |
| chr5:135503303 | GCCCAGGAGAAAGAGAAAAATGG (SEQ ID NO: 798) | 4 | -1 | PTMA |
| chr2:224576711 | GGGGAGGAGAAGGAGGAGAAAGG (SEQ ID NO: 799) | 4 | 0 | PTMA |
| chr1:21183420 | AAGGAGGAGAAGGAGGAAAAGGA (SEQ ID NO: 800) | 4 | -1 | PTMA |
| chr10:32581441 | AAAGAGGAGAATGAGGAGAAGGA (SEQ ID NO: 801) | 4 | -1 | PTMA |
| chr16:70048190 | AGTGAGGAGAATGAGGAATATGA (SEQ ID NO: 802) | 4 | -1 | PTMA |
| chr2:10278758 | GCCGAGGAGGAAGAGGAGAAGGG (SEQ ID NO: 803) | 4 | -1 | PTMA |
| chr2:2279418 | GAAGAGGAGAAGGAGGAAGAGGG (SEQ ID NO: 804) | 4 | -1 | PTMA |
| chr2:99546605 | GGGGAGGAGGATAAGGAAAAGGG (SEQ ID NO: 805) | 4 | -1 | PTMA |
| chr4:129690902 | CTAGAAGAGAGTGAGGAAAAAGG (SEQ ID NO: 806) | 4 | -1 | PTMA |
| chr8:65830066 | GCAGAGGGGAATGAGGTAAAGGG (SEQ ID NO: 807) | 4 | -1 | PTMA |
| chrX:153109805 | GTCAAAGAGAAAGAGAAAAAAGG (SEQ ID NO: 808) | 4 | -1 | PTMA |
| chrX:93490959 | CTAGAGGAGGAAGAGGAAAAAGG (SEQ ID NO: 809) | 4 | -1 | PTMA |
| chr17:32022971 | TTAAAGGAGAATGAGGAGAAGGG (SEQ ID NO: 810) | 4 | 0 | PTMA |
| chr20:19412536 | CAGGAGGAGAAGGAGGAAAAGAG (SEQ ID NO: 811) | 4 | 0 | PTMA |
| chr10:119291821 | AAAGAGGAGAATGAGGATAAGGA (SEQ ID NO: 812) | 4 | -3 | PTMA |
| chr19:6429332 | GAGGAGGAGAAAGAGGTAAAGGG (SEQ ID NO: 813) | 4 | -1 | PTMA |
| chr20:50700530 | GTGGAGGAGGATGAGAAAACAGG (SEQ ID NO: 814) | 4 | -1 | PTMA |
| chr3:165439835 | GATGAGAAGAATGAGGAAGAAGG (SEQ ID NO: 815) | 4 | -1 | PTMA |
| chr1:41096799 | CATGAGAAGAATGAGAAAAAAGG (SEQ ID NO: 816) | 5 | -1 | PTMA |
| chr12:31424114 | TGAGAGGAGAAAGAGAAAAAGGG (SEQ ID NO: 817) | 5 | 0 | PTMA |
| chr1:111166467 | AGGGAAGAGAAAGAGGAAAAAGG (SEQ ID NO: 818) | 5 | 0 | PTMA |
| chr4:20115462 | AAGGAGGAGAAAGAGGAAAGAGG (SEQ ID NO: 819) | 5 | -1 | PTMA |
| chr1:27985454 | CAGGAGGAGAATGAGAAGAATGG (SEQ ID NO: 820) | 5 | -2 | PTMA |
| chr3:102223652 | CCTGAGGAGAATGAGAAGAAGGG (SEQ ID NO: 821) | 5 | 0 | PTMA |
| chr2:208236440 | CAGGAGGAGAAAGAGAAAAATGG (SEQ ID NO: 822) | 5 | 0 | PTMA |
| chr5:21934753 | AAGGGGGAGAAAGAGGAAAAGGG (SEQ ID NO: 823) | 5 | -1 | PTMA |
| chr6:13410817 | AGTGAGGAGAAAGAGGAAGAAGG (SEQ ID NO: 824) | 5 | 0 | PTMA |
| chr2:238694236 | AGAGAGGAGAAAGAGGAAGAGGG (SEQ ID NO: 825) | 5 | -1 | PTMA |
| chr18:74078648 | TGTGAGGAGAAAGAGGAAAGGGG (SEQ ID NO: 826) | 5 | -1 | PTMA |
| chr8:89071706 | AGGGAGGAGAAGAAGGAAAAGGG (SEQ ID NO: 827) | 5 | -1 | PTMA |
| chr7:103054825 | AAGGAGGAGAAAGAGGAAAGGGG (SEQ ID NO: 828) | 5 | 0 | PTMA |
| chr22:22991275 | AAGGAGGAGAAAGAGAAAAAAGG (SEQ ID NO: 829) | 5 | 0 | PTMA |
| chr6:28729397 | AGAAAGGAGAATGAAGAAAATGG (SEQ ID NO: 830) | 5 | -1 | PTMA |
| chr11:110578633 | TGTGAGGAGAAAGAAGAAAATGG (SEQ ID NO: 831) | 5 | -1 | PTMA |
| chr4:158406504 | TATTAGGAGAAAGAGGAAAAGGG (SEQ ID NO: 832) | 5 | -1 | PTMA |
| chr12:107530079 | TGTTAGGAGAATGAAGAAAAGGG (SEQ ID NO: 833) | 5 | 0 | PTMA |
| chr11:121117573 | CAGGAAGAGAATGAGGAAAGGGG (SEQ ID NO: 834) | 5 | -1 | PTMA |
| chr7:138453331 | AGAGAGGAAAAAGAGGAAAAAGG (SEQ ID NO: 835) | 5 | -1 | PTMA |
| chr21:38795221 | AAAGAGGAGAATGAGGAAGGGGG (SEQ ID NO: 836) | 5 | -1 | PTMA |
| chr4:159221593 | TCTAAGGAGAAAGAGGAAAATGG (SEQ ID NO: 837) | 5 | -1 | PTMA |
| chr6:88322711 | AGTGAGGAGAAAGAGGGAAAGGG (SEQ ID NO: 838) | 5 | -1 | PTMA |
| chr20:10789674 | TGTTAGGAGAAAGAGGAAAATGG (SEQ ID NO: 839) | 5 | -1 | PTMA |
| chr1:41888462 | AGAGAGGAGAAGGAGGAGAAAGG (SEQ ID NO: 840) | 5 | 0 | PTMA |
| chr19:12366479 | CAGGAGGGGAAAGAGGAAAAGGG (SEQ ID NO: 841) | 5 | -1 | PTMA |
| chr20:55957570 | AGAGAGGAGAAAGAGGAGAAGGG (SEQ ID NO: 842) | 5 | -1 | PTMA |
| chr3:35326792 | TGTGAGGAGTATAAGGAAAATGG (SEQ ID NO: 843) | 5 | -1 | PTMA |
| chr18:62898018 | AAAGAGGAGAAAGAGGAGAAGGG (SEQ ID NO: 844) | 5 | -1 | PTMA |
| chr4:88719518 | AAGGAGGAGAAGGAGGAGAAGGG (SEQ ID NO: 845) | 5 | -1 | PTMA |
| chrX:25806484 | TGAGAGGAGAAAAAGGAAAAAGG (SEQ ID NO: 846) | 5 | -1 | PTMA |
| chr10:121694208 | ACAGAGGAGAAGAAGGAAAAAGG (SEQ ID NO: 847) | 5 | -1 | PTMA |
| chr7:143933116 | AAGGAGGAGAAGGAGAAAAAGGG (SEQ ID NO: 848) | 5 | -1 | PTMA |
| chr7:155087773 | CAGGAGGAGAAAGAGGAAGATGG (SEQ ID NO: 849) | 5 | -1 | PTMA |
| chr20:34893184 | TGAAAGGAGAAAGAGGAAAAAGG (SEQ ID NO: 850) | 5 | -1 | PTMA |
| chr1:85309585 | AGGGAGGAGAGGGAGGAAAAGGG (SEQ ID NO: 851) | 5 | -1 | PTMA |
| chr7:24251938 | AAGGAGAAGAAAGAGGAAAAGGG (SEQ ID NO: 852) | 5 | -1 | PTMA |
| chr21:46414384 | CCAGAGGAGAAGGAGGAGAAGGG (SEQ ID NO: 853) | 5 | -1 | PTMA |
| chr18:24596717 | TGGGAAGAGAATGGGGAAAAGGG (SEQ ID NO: 854) | 5 | 0 | PTMA |
| chr1:33441531 | AAGGAGGAGAAAGAGGAAGAAGG (SEQ ID NO: 855) | 5 | -1 | PTMA |
| chr7:132563387 | GAGGAGGAGAAAGAGGAGGAGGA (SEQ ID NO: 856) | 5 | -1 | PTMA |
| chr7:48476925 | TCGGAGGGGAAAGAGGAAAAGGG (SEQ ID NO: 857) | 5 | -1 | PTMA |
| chr7:15492786 | GGTGGGGAGAAAGAGAAAAAGGG (SEQ ID NO: 858) | 5 | 0 | PTMA |
| chr1:69596851 | AAAGAGGAGAAAGAGGAACATGG (SEQ ID NO: 859) | 5 | -1 | PTMA |
| chr16:84618740 | GGTGGGGAGAATGAGGAAGGGGG (SEQ ID NO: 860) | 5 | -1 | PTMA |
| chr22:21003367 | AAGGAGGAGAAGGAGGAAGAAGG (SEQ ID NO: 861) | 5 | -1 | PTMA |
| chr17:64461015 | GGTGAGGAGAAAGAGAAAAGGGG (SEQ ID NO: 862) | 5 | 0 | PTMA |
| chr6:25815519 | AATGAGGAGCAAGAGGAAAAGGG (SEQ ID NO: 863) | 5 | -1 | PTMA |
| chr7:70387134 | AGTGAAGAGAATGAGAAAAAGAG (SEQ ID NO: 864) | 5 | -1 | PTMA |
| chr4:158408520 | TATTAGGAGAAGGAGGAAAAGGG (SEQ ID NO: 865) | 5 | 0 | PTMA |
| chr7:108432973 | AAGGAGGAGAAAGAGAAAAAGAG (SEQ ID NO: 866) | 5 | -1 | PTMA |
| chr10:132381769 | ACTGAGGAGAAAGAGGAGAAAGG (SEQ ID NO: 867) | 5 | 0 | PTMA |
| chr13:34217068 | ACAGAGGAGAGAGAGGAAAAGGG (SEQ ID NO: 868) | 5 | 0 | PTMA |
| chr1:33150117 | CCAGAGGAGAAGGAGGAAACTGG (SEQ ID NO: 869) | 5 | -1 | PTMA |
| chr11:84095245 | GGTAAGGAGAAAGGGGAAAACGG (SEQ ID NO: 870) | 5 | -1 | PTMA |
| chr2:20379139 | AAAGAGGAGAAAGAGGAGAAAGA (SEQ ID NO: 871) | 5 | -1 | PTMA |
| chr6:89951248 | AGTGAAGAGAATGAGGAAGAGAG (SEQ ID NO: 872) | 5 | -1 | PTMA |
| chr7:142900112 | AAGGAGGAGGAAGAGGAAAAAGG (SEQ ID NO: 873) | 5 | -1 | PTMA |
| chrX:24601192 | TGTTAGGAGAATGAGGAAACAAG (SEQ ID NO: 874) | 5 | -1 | PTMA |
| chr1:66643080 | AGAGAGGAGAAAGAGAAAAACGT (SEQ ID NO: 875) | 5 | 0 | PTMA |
| chr2:115321627 | CAAGAGGAGAGAGAGGAAAAGGG (SEQ ID NO: 876) | 5 | 0 | PTMA |
| chr10:2939550 | ATGAAGGAGAAAGAGGAAATGGG (SEQ ID NO: 877) | 5 | -1 | PTMA |
| chr10:58607493 | AGAGAGGAGAAGGAGGATAAAGG (SEQ ID NO: 878) | 5 | -1 | PTMA |
| chr11:36376309 | TGGGAGGAGAAGGAGGAAGAGGG (SEQ ID NO: 879) | 5 | -1 | PTMA |
| chr17:49225505 | CAAAAGGAGAATGAGGAAACTGG (SEQ ID NO: 880) | 5 | -1 | PTMA |
| chr18:10889760 | AGGGAGGAGAATGAGGATGAGGG (SEQ ID NO: 881) | 5 | -1 | PTMA |
| chr3:128557772 | AGCAAGGAGAAAGAGGAAAGGGG (SEQ ID NO: 882) | 5 | -1 | PTMA |
| chr3:179798170 | AAAGAGAAGAATGAGGAAAGTGG (SEQ ID NO: 883) | 5 | -1 | PTMA |
| chr3:24258124 | AGGGAGGAGAATGAGGTGAAAGG (SEQ ID NO: 884) | 5 | -1 | PTMA |
| chr5:68385100 | CAGGAAGAGAATGAGGTAAATGG (SEQ ID NO: 885) | 5 | -1 | PTMA |
| chr7:1526478 | AAAGAGGAGGAAGAGGAAAAAGG (SEQ ID NO: 886) | 5 | -1 | PTMA |
| chr22:31192641 | ATCAAGGAGAAGGAGAAAAGGGG (SEQ ID NO: 887) | 5 | -3 | PTMA |
| chr1:66155277 | AAAGAGGAGCAAGAGGAAAATGG (SEQ ID NO: 888) | 5 | -1 | PTMA |
| chr11:130318956 | CATGTAGAGAATGAGGAAAAGGG (SEQ ID NO: 889) | 5 | -1 | PTMA |
| chr18:30811124 | CAAGAGAAGAATGAGGAAAGAGG (SEQ ID NO: 890) | 5 | -1 | PTMA |
| chr4:48796514 | TGAGAGGAGAATGAGAATAAAGG (SEQ ID NO: 891) | 5 | -1 | PTMA |
| chr6:12673713 | CACGAGGAGAAAGAGAAAAGTGG (SEQ ID NO: 892) | 5 | -1 | PTMA |
| chr7:94503877 | AGGGAGGGGGATGAGGAAAAAGG (SEQ ID NO: 893) | 5 | -1 | PTMA |
| chrX:143499018 | AGAGAGAAGAATGAGGAAAGAGG (SEQ ID NO: 894) | 5 | -1 | PTMA |
| chr9:96910199 | GGGAATGCTAATGAGGAAAATGG (SEQ ID NO: 895) | 6 | 0 | PTMA |
| chr9:108272602 | AAAGAGGAGAAAGAGAAAAGGGG (SEQ ID NO: 896) | 6 | 0 | PTMA |
| chr4:77548211 | CAGGAGGAGAAAGAGACAAATGG (SEQ ID NO: 897) | 6 | 0 | PTMA |
| chr2:26512079 | AATAAGGAGAATGAGAAAAGTGG (SEQ ID NO: 898) | 6 | -1 | PTMA |
| chr1: 155209712 | AGTGAGGAGGAAGAGGAGAAGGG (SEQ ID NO: 899) | 6 | -1 | PTMA |
| chr1:237282826 | CATAAGGAGAATGAGAACAAAGG (SEQ ID NO: 900) | 6 | -1 | PTMA |
| chr16:18341220 | AGGGAGGGGAAGGAGGATAAGGG (SEQ ID NO: 901) | 6 | -1 | PTMA |
| chr1:30692932 | AGTGGGGAGAAAGAGAAAAAAGG (SEQ ID NO: 902) | 6 | 0 | PTMA |
| chr22:36231417 | GCAGATTCTCTCTGCTCACTTGG (SEQ ID NO: 903) | 0 | -1 | APOL2 |
| chr5:135449913 | GATGGTACAGGCTCACTCGCAGG (SEQ ID NO: 904) | 0 | -1 | TIFAB |
| chr10:32650622 | AGTGGTACAGGCTCACAAGCTGG (SEQ ID NO: 905) | 4 | -1 | TIFAB |
| chrX:142119565 | CATGGCACAGGCTCACCTGCAGG (SEQ ID NO: 906) | 4 | -1 | TIFAB |
| chr16:86207516 | GGTGGCACAGGTTCACTCGTTGG (SEQ ID NO: 907) | 4 | -1 | TIFAB |
| chr1:17929687 | GATGGCACAGTCTCACTCAGGGG (SEQ ID NO: 908) | 4 | -1 | TIFAB |
| chr4:1337650 | GAAGGGACAGACTCAGTCGCAGG (SEQ ID NO: 909) | 4 | -1 | TIFAB |
| chr7:95545100 | CGTGGTACAGACTCACTCTCTGA (SEQ ID NO: 910) | 4 | -1 | TIFAB |
| chr9:133064727 | GCACCCAAATGTTGAGGTACAGG (SEQ ID NO: 911) | 0 | -1 | CEL |
| chr12:13402927 | TATCCCAAATGTTGAGGTACTGG (SEQ ID NO: 912) | 3 | -1 | CEL |
| chr11:33544912 | GTCATCGAACTGCTCTTAGCTGG (SEQ ID NO: 913) | 0 | -1 | C11orf41 |
| chr4:41319008 | GTCATTGAACTGCTCTTAGCCTG (SEQ ID NO: 914) | 1 | -1 | C11orf41 |
| chr12:6315139 | GCCTGACCATCGAGAAGTCCTGG (SEQ ID NO: 915) | 0 | -1 | PLEKHG6 |
| chr17:17977652 | GGACGATGACATGCTCAAGCTGG (SEQ ID NO: 916) | 0 | -1 | LRRC48 |
| chr8:144258090 | GGTCGATGCCAGGCTCAAGCTGG (SEQ ID NO: 917) | 3 | -1 | LRRC48 |
| chr7:26178897 | GGAAGGGGACATGCTAAAGCAGG (SEQ ID NO: 918) | 4 | -1 | LRRC48 |
| chr19:19147702 | GAGTCACTTACATACAGCCGGGG (SEQ ID NO: 919) | 0 | -1 | MEF2B |
| chr20:47984798 | GTGTCACTAACATACAGCCAGGG (SEQ ID NO: 920) | 3 | -1 | MEF2B |
| chr15:90561461 | AAGGCACTAACATACAGCCTGGT (SEQ ID NO: 921) | 4 | -1 | MEF2B |
| chr1:154342469 | ACATCACCTACATACAGCCAGGG (SEQ ID NO: 922) | 5 | -1 | MEF2B |
| chr18:62325422 | GCGCTCCTTACCTGCAGCCGGGC (SEQ ID NO: 923) | 6 | -2 | MEF2B |
| chr19:35715992 | GAGATGGAAGAGTCTGATCAGGG (SEQ ID NO: 924) | 0 | -1 | ZBTB32 |
| chr4:56088102 | GAGATGGAGGAGCCTGATCATAG (SEQ ID NO: 925) | 2 | -1 | ZBTB32 |
| chr17:28733256 | GAGATGGAAGAGACTGAGCAAGG (SEQ ID NO: 926) | 2 | 0 | ZBTB32 |
| chr2:112196653 | ATCATGGAAGAGTCTGATCAGGG (SEQ ID NO: 927) | 3 | 0 | ZBTB32 |
| chr10:61659261 | AAGGTGGAAGAGTGAGATCAGGG (SEQ ID NO: 928) | 4 | -1 | ZBTB32 |
| chr17:10490996 | AAGATGGAAGGATCTGATTATGG (SEQ ID NO: 929) | 4 | -1 | ZBTB32 |
| chr19:39934568 | GTCTGACTTACCCCACAGGAGGG (SEQ ID NO: 930) | 0 | 0 | FCGBP |
| chr3:139302401 | GTCTGACTCACCCCACAGGAGTG (SEQ ID NO: 931) | 1 | 0 | FCGBP |
| chr9:85011928 | GCCTGACCTACCCCACAGGACTA (SEQ ID NO: 932) | 2 | -1 | FCGBP |
| chr15:80889701 | GGCTGACCTACCTCACAGGAGGG (SEQ ID NO: 933) | 3 | -1 | FCGBP |
| chr3:52765742 | GTCTGACCTTCCCCACAGAAGGG (SEQ ID NO: 934) | 3 | 0 | FCGBP |
| chr7:124206614 | GCCTGACTTACTCCACAGAAAGG (SEQ ID NO: 935) | 3 | 0 | FCGBP |
| chr5:77308531 | GTCTGACCTACCCAGCAGGAAGG (SEQ ID NO: 936) | 3 | -1 | FCGBP |
| chr22:48587654 | GCCTGGCCTACCCCACAGGGCGG (SEQ ID NO: 937) | 4 | -1 | FCGBP |
| chr7:151079605 | GTGTGACCTGCTCCACAGGAGGG (SEQ ID NO: 938) | 4 | -1 | FCGBP |
| chr3:128904444 | GTATGACCTACCTCACAGCAGGG (SEQ ID NO: 939) | 4 | 0 | FCGBP |
| chr21:38853553 | CGCTGACTCACCCCACAGGCGGG (SEQ ID NO: 940) | 4 | -1 | FCGBP |
| chr1:37433580 | CCCAGACCTACCCCACAGGAGGG (SEQ ID NO: 941) | 4 | -1 | FCGBP |
| chr1:54334643 | ATATGACCTACCTCAAAGGATGG (SEQ ID NO: 942) | 5 | -1 | FCGBP |
| chr8:143042333 | GCCTGGCCCACACCACAGGATGG (SEQ ID NO: 943) | 5 | -1 | FCGBP |
| chr19:48628043 | GATGGCATCGTCACGGTCTCGGG (SEQ ID NO: 944) | 0 | -1 | SPHK2 |
| chr1:40251589 | GTCCATCACATTTCAAATGGGGG (SEQ ID NO: 945) | 0 | -1 | TMCO2 |
| chr6:70667602 | GACCATCACATCTCAAAAGGGGG (SEQ ID NO: 946) | 3 | -1 | TMCO2 |
| chr13:63934298 | ACACATCACATTCCAAATGGTGG (SEQ ID NO: 947) | 4 | -1 | TMCO2 |
| chr4:163585753 | GGATACTGTACCTTCCGGAGGGG (SEQ ID NO: 948) | 0 | -1 | MARCH1 |
| chr6:60930559 | AGGTACTGTACCCTCCAGAGGGG (SEQ ID NO: 949) | 4 | -1 | MARCH1 |
| chr6:58176025 | AGGTACTGTACCCTCCAGAGGGG (SEQ ID NO: 950) | 4 | 0 | MARCH1 |
| chr11:65109980 | GGGTACTGTCCCTTCAAGAGGGG (SEQ ID NO: 951) | 4 | 0 | MARCH1 |
| chr9:12453142 | CCATATTGTACCTTCCAGAGAGG (SEQ ID NO: 952) | 4 | -1 | MARCH1 |
| chr7:123147469 | AGATACTGTACCTTCCTTTGAGG (SEQ ID NO: 953) | 4 | 0 | MARCH1 |
| chr14:20990072 | GTAGGCACTCACCCGGGCCTGGG (SEQ ID NO: 954) | 0 | -1 | METTL17 |
| chr11:25515687 | CTAAGCACTCACCCGGGCCTCTG (SEQ ID NO: 955) | 2 | -1 | METTL17 |
| chr2:176106521 | CTAGGCACTCACCCAGGCCGGGG (SEQ ID NO: 956) | 3 | -1 | METTL17 |
| chr11:49783972 | GTAGGCCACCACCCGGGCCTTGG (SEQ ID NO: 957) | 3 | -1 | METTL17 |
| chr1:161726988 | GCAGGCACTCACCCGGCCCCGGG (SEQ ID NO: 958) | 3 | -1 | METTL17 |
| chr11:77150032 | GTGGCCACTCACCCAGGCCTGGG (SEQ ID NO: 959) | 3 | -1 | METTL17 |
| chr3:126433305 | CAGGGCACTCACCCGGGCCTTGT (SEQ ID NO: 960) | 3 | -1 | METTL17 |
| chr10:77614058 | CTAGACACCCACCCAGGCCTGGG (SEQ ID NO: 961) | 4 | -1 | METTL17 |
| chr11:88850005 | GCAGGCCACCACCCGGGCCTTGG (SEQ ID NO: 962) | 4 | -1 | METTL17 |
| chr1:44113979 | GTAGACACACACCTAGGCCTGGG (SEQ ID NO: 963) | 4 | -1 | METTL17 |
| chr14:105143241 | CTAGCCACACACCCAGGCCTGGG (SEQ ID NO: 964) | 4 | -1 | METTL17 |
| chr14:85631482 | CTGGGCACCCACCAGGGCCTGGG (SEQ ID NO: 965) | 4 | -1 | METTL17 |
| chr16:53510147 | GTAACCACCCACCCGGGCCGGGG (SEQ ID NO: 966) | 4 | -1 | METTL17 |
| chr19:17112844 | CCAGGCACTCACCCAGCCCTTGG (SEQ ID NO: 967) | 4 | -1 | METTL17 |
| chr12:132258616 | TTAGGCACACGCCCGGGCTTCGG (SEQ ID NO: 968) | 4 | -1 | METTL17 |
| chr9:135493198 | GCGGGCACACGCCCGGGCCTGGG (SEQ ID NO: 969) | 4 | -1 | METTL17 |
| chr9:114330013 | CCAGGCACTCACCCGGTCCAGGG (SEQ ID NO: 970) | 4 | -1 | METTL17 |
| chr2:156519800 | AAAGGCACTCACCCTGGCCCAGG (SEQ ID NO: 971) | 4 | -1 | METTL17 |
| chr10:77804600 | GTAGACACACACCAGGGCCCTGG (SEQ ID NO: 972) | 4 | -1 | METTL17 |
| chr10:52609924 | TCAGGCAGCCACTCGGGCCTTGG (SEQ ID NO: 973) | 5 | -1 | METTL17 |
| chr2:238346362 | CCTGGCACCCACCAGGGCCTAGG (SEQ ID NO: 974) | 5 | -1 | METTL17 |
| chr17:41786110 | ATAGGGCCCCACCCAGGCCTGGG (SEQ ID NO: 975) | 5 | -1 | METTL17 |
| chr19:40407911 | GGGCACTCACCTCGGCACTCCGG (SEQ ID NO: 976) | 0 | -1 | PRX |
| chr16:75205532 | AGGGCCTCACCCCGGCACTCTGG (SEQ ID NO: 977) | 4 | -1 | PRX |
| chr17:50270542 | TGGCACTCACCTCGGGCCTGGGG (SEQ ID NO: 978) | 4 | -2 | PRX |
| chr7:148290756 | CATCACTCACCCTGGCACTCAGG (SEQ ID NO: 979) | 5 | -1 | PRX |
| chr1:206110310 | GCTGACCCGCTCCAGCTGCCCGG (SEQ ID NO: 980) | 0 | -1 | AVPR1B |
| chr9:82746451 | ACTGACCAGATCCAGCTGCCTGG (SEQ ID NO: 981) | 3 | 0 | AVPR1B |
| chr8:130122054 | TATGACCTGTTCCAGCTGCCTGG (SEQ ID NO: 982) | 4 | 0 | AVPR1B |
| chr17:15422592 | ACTCACCCGCCCCAGCTCCCCGG (SEQ ID NO: 983) | 4 | -1 | AVPR1B |
| chr1:16693073 | ACGGACGCCCCCCGGCTGCCGGT (SEQ ID NO: 984) | 6 | 0 | AVPR1B |
| chr20:44960284 | GTTGCGGAAACTCTCATTGCCGG (SEQ ID NO: 985) | 0 | -1 | TOMM34 |
| chr19:54938954 | CTTGCAGAAACTCTCACTGCAGG (SEQ ID NO: 986) | 3 | -1 | TOMM34 |
| chr8:87877263 | GTAACGCAAACTCTCATTGCTGG (SEQ ID NO: 987) | 3 | -1 | TOMM34 |
| chr18:28291123 | CTTGAGGAAACTCTCATTGAGGG (SEQ ID NO: 988) | 3 | 0 | TOMM34 |
| chr7:159246905 | GAAATGGAAACTCTCATTGCTGG (SEQ ID NO: 989) | 4 | -1 | TOMM34 |
| chr9:37848113 | ATTGCTGAAACCCACATTGCTGG (SEQ ID NO: 990) | 4 | -1 | TOMM34 |
| chr11:63817990 | GATGTGCGAGCGAGCTGTGTCGG (SEQ ID NO: 991) | 0 | -1 | C11orf84 |
| chr11:113221500 | GATGAGCAAGCAAGCTGTGTTGG (SEQ ID NO: 992) | 3 | -1 | C11orf84 |
| chr12:11001461 | GATGTGCCAGCAACCTGTGTGGG (SEQ ID NO: 993) | 3 | -1 | C11orf84 |
| chr4:114345044 | AATGTGCAGGTGAGCTGTGTGGG (SEQ ID NO: 994) | 4 | -1 | C11orf84 |
| chr2:47391782 | AATGTGTGAGCAAGCAGTGTGGG (SEQ ID NO: 995) | 4 | -1 | C11orf84 |
| chr19:4017126 | GAAGTGCCAGCGGGCTGAGTGGG (SEQ ID NO: 996) | 4 | -1 | C11orf84 |
| chr3:177383169 | TGTGTGCGAGTGAGCTGTCTTGG (SEQ ID NO: 997) | 4 | -1 | C11orf84 |
| chr3:185154321 | AGAGTGCGAGCCAACTGTGTGGG (SEQ ID NO: 998) | 5 | -1 | C11orf84 |

Table 6. Sequences of guide RNAs and pegRNAs used in this study (related to STAR Methods).

TABLE 6A

gRNAs used in TTISS to test 8 specificity variants and WT SpCas9
These were also used when measuring indel frequencies for activity scores

| | | |
|---|---|---|
| Gene | Spacer Sequence | Target Site with PAM |
| ALDH1A3 | GGAGAGGGACCGCGCCACCT (SEQ ID NO: 999) | GGAGAGGGACCGCGCCACCTtgg (SEQ ID NO: 1000) |
| CACNG3 | GAACTTACGCAGGAGATATT (SEQ ID NO: 1001) | GAACTTACGCAGGAGATATTcgg (SEQ ID NO: 1002) |
| ADORA2B | GTTCCGGTAAGCATAGACAA (SEQ ID NO: 1003) | GTTCCGGTAAGCATAGACAAtgg (SEQ ID NO: 1004) |
| PEX12 | GAGACCCGCTCTTCAGCATG (SEQ ID NO: 1005) | GAGACCCGCTCTTCAGCATGtgg (SEQ ID NO: 1006) |
| CRABP2 | GAGAGGGCCCCAAGACCTCG (SEQ ID NO: 1007) | GAGAGGGCCCCAAGACCTCGtgg (SEQ ID NO: 1008) |
| TWSG1 | GCGCCTTATTCCAGTGACAA (SEQ ID NO: 1009) | GCGCCTTATTCCAGTGACAAagg (SEQ ID NO: 1010) |
| HCN2 | GCAGATCCTCATCACCGCGC (SEQ ID NO: 1011) | GCAGATCCTCATCACCGCGCtgg (SEQ ID NO: 1012) |
| EEF2 | GCATGTCGACTTCTCCTCGG (SEQ ID NO: 1013) | GCATGTCGACTTCTCCTCGGagg (SEQ ID NO: 1014) |
| IL29 | GCTGGTCTAGGACGTCCTCC (SEQ ID NO: 1015) | GCTGGTCTAGGACGTCCTCCagg (SEQ ID NO: 1016) |
| FGF21 | GGAAACTCACCGATCCATAC (SEQ ID NO: 1017) | GGAAACTCACCGATCCATACagg (SEQ ID NO: 1018) |
| METTL18 | GCCAGCAAAGCACATTATTT (SEQ ID NO: 1019) | GCCAGCAAAGCACATTATTTtgg (SEQ ID NO: 1020) |
| RIMS4 | GGCCCGTCTCCGTGCTCCTC (SEQ ID NO: 1021) | GGCCCGTCTCCGTGCTCCTCtgg (SEQ ID NO: 1022) |
| EEF1A2 | GCGCTACGACGAGATCGTCA (SEQ ID NO: 1023) | GCGCTACGACGAGATCGTCAagg (SEQ ID NO: 1024) |
| FAM5C | GAGAATAAGATTCAGTTGCA (SEQ ID NO: 1025) | GAGAATAAGATTCAGTTGCAagg (SEQ ID NO: 1026) |
| EHD3 | GTTTCTTGGGATCCACCACC (SEQ ID NO: 1027) | GTTTCTTGGGATCCACCACCagg (SEQ ID NO: 1028) |
| PRKCE | GTAGGTGGGCTGCCGAAGAT (SEQ ID NO: 1029) | GTAGGTGGGCTGCCGAAGATagg (SEQ ID NO: 1030) |
| DIRC1 | GTAATTAGGTAAGGCTTAGT (SEQ ID NO: 1031) | GTAATTAGGTAAGGCTTAGTtgg (SEQ ID NO: 1032) |
| SDPR | GCTCTTTGACCGCGCGCGTG (SEQ ID NO: 1033) | GCTCTTTGACCGCGCGCGTGtgg (SEQ ID NO: 1034) |
| CTNNB1 | GAAACAGCTCGTTGTACCGC (SEQ ID NO: 1035) | GAAACAGCTCGTTGTACCGCtgg (SEQ ID NO: 1036) |
| CCDC80 | GCAACAACGTGATGAATATC (SEQ ID NO: 1037) | GCAACAACGTGATGAATATCtgg (SEQ ID NO: 1038) |
| PRDM2 | GTCGCTGTGACTTTCTAATT (SEQ ID NO: 1039) | GTCGCTGTGACTTTCTAATTtgg (SEQ ID NO: 1040) |
| CSF1 | GGTGTTATCTCTGAAGCGCA (SEQ ID NO: 1041) | GGTGTTATCTCTGAAGCGCAtgg (SEQ ID NO: 1042) |
| ATR | GGATCATGGAAGCCAGCTCC (SEQ ID NO: 1043) | GGATCATGGAAGCCAGCTCCagg (SEQ ID NO: 1044) |
| SMOC1 | GGTCTCGGCACTTGGCTCGC (SEQ ID NO: 1045) | GGTCTCGGCACTTGGCTCGCtgg (SEQ ID NO: 1046) |
| RP11-382A20.3 | GGAGGCTTCACAGCGCCCTC (SEQ ID NO: 1047) | GGAGGCTTCACAGCGCCCTCtgg (SEQ ID NO: 1048) |
| POLR2H | GCTAGTACCTTGTATGAAGA (SEQ ID NO: 1049) | GCTAGTACCTTGTATGAAGAtgg (SEQ ID NO: 1050) |
| LIMCH1 | GACGGGAAAGTCAGTGTGAA (SEQ ID NO: 1051) | GACGGGAAAGTCAGTGTGAAtgg (SEQ ID NO: 1052) |
| CTXN3 | GTTCGACCATGCCCTTGCTT (SEQ ID NO: 1053) | GTTCGACCATGCCCTTGCTTagg (SEQ ID NO: 1054) |
| HCRTR1 | GGCAGAGCTCACCTGTAGAT (SEQ ID NO: 1055) | GGCAGAGCTCACCTGTAGATagg (SEQ ID NO: 1056) |
| BCAP29 | GCTGGTGGAGCTCTTCTCAA (SEQ ID NO: 1057) | GCTGGTGGAGCTCTTCTCAAtgg (SEQ ID NO: 1058) |
| CREB3L2 | GGAGCTGACCCAAGACGTTC (SEQ ID NO: 1059) | GGAGCTGACCCAAGACGTTCtgg (SEQ ID NO: 1060) |
| SLC4A4 | GTTGACCATCAGATTGAGAC (SEQ ID NO: 1061) | GTTGACCATCAGATTGAGACagg (SEQ ID NO: 1062) |
| LEF1 | GCTCACCTCGTGTCCGTTGC (SEQ ID NO: 1063) | GCTCACCTCGTGTCCGTTGCtgg (SEQ ID NO: 1064) |
| CCDC111 | GGACGTTCATGTATTTGCTT (SEQ ID NO: 1065) | GGACGTTCATGTATTTGCTTtgg (SEQ ID NO: 1066) |
| OXCT1 | GCTGTAAAAGACATCCCTGA (SEQ ID NO: 1067) | GCTGTAAAAGACATCCCTGAtgg (SEQ ID NO: 1068) |
| AC114947.1 | GGGTCTCCACCACTTCGTAA (SEQ ID NO: 1069) | GGGTCTCCACCACTTCGTAAagg (SEQ ID NO: 1070) |
| ALG8 | GGCGGCGCTCACAATTGCCA (SEQ ID NO: 1071) | GGCGGCGCTCACAATTGCCAcgg (SEQ ID NO: 1072) |
| C11orf88 | GGTACTTACTGTTACTCGCA (SEQ ID NO: 1073) | GGTACTTACTGTTACTCGCAagg (SEQ ID NO: 1074) |
| DTX3 | GACGCTGGTCAAACGCCTTG (SEQ ID NO: 1075) | GACGCTGGTCAAACGCCTTGcgg (SEQ ID NO: 1076) |
| KIAA0895L | GGCATGCTGCGGCATGAGAT (SEQ ID NO: 1077) | GGCATGCTGCGGCATGAGATagg (SEQ ID NO: 1078) |
| TAF4B | GGCTCCACGCAGACGCTGAC (SEQ ID NO: 1079) | GGCTCCACGCAGACGCTGACagg (SEQ ID NO: 1080) |
| PTMA | GTCGAGGAGAATGAGGAAAA (SEQ ID NO: 1081) | GTCGAGGAGAATGAGGAAAAtgg (SEQ ID NO: 1082) |
| APOL2 | GCAGATTCTCTCTGCTCACT (SEQ ID NO: 1083) | GCAGATTCTCTCTGCTCACTtgg (SEQ ID NO: 1084) |
| TIFAB | GATGGTACAGGCTCACTCGC (SEQ ID NO: 1085) | GATGGTACAGGCTCACTCGCagg (SEQ ID NO: 1086) |
| CEL | GCACCCAAATGTTGAGGTAC (SEQ ID NO: 1087) | GCACCCAAATGTTGAGGTACagg (SEQ ID NO: 1088) |
| C11orf41 | GTCATCGAACTGCTCTTAGC (SEQ ID NO: 1089) | GTCATCGAACTGCTCTTAGCtgg (SEQ ID NO: 1090) |
| PLEKHG6 | GCCTGACCATCGAGAAGTCC (SEQ ID NO: 1091) | GCCTGACCATCGAGAAGTCCtgg (SEQ ID NO: 1092) |
| LRRC48 | GGACGATGACATGCTCAAGC (SEQ ID NO: 1093) | GGACGATGACATGCTCAAGCtgg (SEQ ID NO: 1094) |
| MEF2B | GAGTCACTTACATACAGCCG (SEQ ID NO: 1095) | GAGTCACTTACATACAGCCGggg (SEQ ID NO: 1096) |
| ZBTB32 | GAGATGGAAGAGTCTGATCA (SEQ ID NO: 1097) | GAGATGGAAGAGTCTGATCAggg (SEQ ID NO: 1098) |
| FCGBP | GTCTGACTTACCCCACAGGA (SEQ ID NO: 1099) | GTCTGACTTACCCCACAGGAggg (SEQ ID NO: 1100) |
| SPHK2 | GATGGCATCGTCACGGTCTC (SEQ ID NO: 1101) | GATGGCATCGTCACGGTCTCggg (SEQ ID NO: 1102) |
| TMCO2 | GTCCATCACATTTCAAATGG (SEQ ID NO: 1103) | GTCCATCACATTTCAAATGGggg (SEQ ID NO: 1104) |
| MARCH1 | GGATACTGTACCTTCCGGAG (SEQ ID NO: 1105) | GGATACTGTACCTTCCGGAGggg (SEQ ID NO: 1106) |
| METTL17 | GTAGGCACTCACCCGGGCCT (SEQ ID NO: 1107) | GTAGGCACTCACCCGGGCCTggg (SEQ ID NO: 1108) |
| PRX | GGGCACTCACCTCGGCACTC (SEQ ID NO: 1109) | GGGCACTCACCTCGGCACTCcgg (SEQ ID NO: 1110) |
| AVPR1B | GCTGACCCGCTCCAGCTGCC (SEQ ID NO: 1111) | GCTGACCCGCTCCAGCTGCCcgg (SEQ ID NO: 1112) |
| TOMM34 | GTTGCGGAAACTCTCATTGC (SEQ ID NO: 1112) | GTTGCGGAAACTCTCATTGCcgg (SEQ ID NO: 1114) |
| C11orf84 | GATGTGCGAGCGAGCTGTGT (SEQ ID NO: 1115) | GATGTGCGAGCGAGCTGTGTcgg (SEQ ID NO: 1116) |

TABLE 6B

gRNAs used in lentiviral screen for SpCas9 mutants

| Guide Name | Gene | Spacer Sequence | (Off-)Target Site with PAM |
|---|---|---|---|
| g1 | (lentivirus) | GACCACTGACAATACCTC CC (SEQ ID NO: 1117) | GACCACTGACAATACCTCCC tgg (SEQ ID NO: 1118) |
| g2 | (lentivirus) | GCGAGTCTTCACTGAGTG TA (SEQ ID NO: 1119) | GCGAGTCTTCACTGAGTGTA agg (SEQ ID NO: 1120) |
| g3 | (lentivirus) | GAGTCCGAGCAGAAGAA GAA (SEQ ID NO: 1121) | GAGTtaGAGCAGAAGAAGAA agg (SEQ ID NO: 1122) |
| g4 | (lentivirus) | GGTGAGTGAGTGTGTGCG TG (SEQ ID NO: 1123) | aGTGAGTGAGTGTGTGtGTGg gg (SEQ ID NO: 1124) |
| g5 | RNF103-CHMP3 | GTGCATTTCACCACTGAA AT (SEQ ID NO: 1125) | GTGCATTTCACCACTGAAATt gg (SEQ ID NO: 1126) |
| g6 | RGS8 | GACCCTCAGGCCATGAGG AC (SEQ ID NO: 1127) | GACCCTCAGGCCATGAGGA Ctgg (SEQ ID NO: 1128) |
| g7 | GTPBP2 | GTTTCTTTTCAGGCTGAA GA (SEQ ID NO: 1129) | GTTTCTTTTCAGGCTGAAGAt gg (SEQ ID NO: 1130) |
| g8 | SYNPO | GGGCGTCCCAGCACGAC GAC (SEQ ID NO: 1131) | GGGCGTCCCAGCACGACGA Cagg (SEQ ID NO: 1132) |
| g9 | TTLL 11 | GCTTGCCTTGTGACATCT AC (SEQ ID NO: 1133) | GCTTGCCTTGTGACATCTACt gg (SEQ ID NO: 1134) |
| g10 | CLIC3 | GACAGACACGCTGCAGA TCG (SEQ ID NO: 1135) | GACAGACACGCTGCAGATC Gagg (SEQ ID NO: 1136) |
| g11 | DYNC1H1 | GCGAGTCTTCACTGAGTG TA (SEQ ID NO: 1137) | GCGAGTCTTCACTGAGTGTA agg (SEQ ID NO: 1138) |
| VEGFA | VEGFA | GGTGAGTGAGTGTGTGCG TG (SEQ ID NO: 1139) | GGTGAGTGAGTGTGTGCGTG tgg (SEQ ID NO: 1110) |
| VEGFA OT1 | -- | GGTGAGTGAGTGTGTGCG TG (SEQ ID NO: 1141) | GGTGAGTGAGTGTGTGtGTGa gg (SEQ ID NO: 1142) |
| VEGFA OT2 | -- | GGTGAGTGAGTGTGTGCG TG (SEQ ID NO: 1143) | aGTGAGTGAGTGTGTGtGTGg gg (SEQ ID NO: 1144) |
| VEGFA OT3 | -- | GGTGAGTGAGTGTGTGCG TG (SEQ ID NO: 1145) | tGTGgGTGAGTGTGTGCGTGa gg (SEQ ID NO: 1146) |
| VEGFA OT4 | -- | GGTGAGTGAGTGTGTGCG TG (SEQ ID NO: 1147) | GGTGAGTGAGTGcGTGCGgGt gg (SEQ ID NO: 1148) |
| VEGFA OT5 | -- | GGTGAGTGAGTGTGTGCG TG (SEQ ID NO: 1149) | GcTGAGTGAGTGTaTGCGTGt gg (SEQ ID NO: 1150) |
| EMX1 | EMX1 | GAGTCCGAGCAGAAGAA GAA (SEQ ID NO: 1151) | GAGTCCGAGCAGAAGAAGA Aggg (SEQ ID NO: 1152) |
| EMX1 OT1 | -- | GAGTCCGAGCAGAAGAA GAA (SEQ ID NO: 1153) | GAGTtaGAGCAGAAGAAGAA agg (SEQ ID NO: 1154) |
| EMX1 OT2 | -- | GAGTCCGAGCAGAAGAA GAA (SEQ ID NO: 1155) | GAGTCtaAGCAGAAGAAGAA gag (SEQ ID NO: 1156) |
| OT | MIA3 | GTGTAGGTTGGACGCACT TT (SEQ ID NO: 1157) | GTaTAGGTTGGACGCACTTTt gg (SEQ ID NO: 1158) |

TABLE 6C

gRNAs used in HEK293T multiplexing experiment

| Gene | Spacer Sequence | Target Site with PAM | 1 gRNA sample | 3 gRNA sample | 10 gRNA sample | 30 gRNA sample | 60 gRNA sample |
|---|---|---|---|---|---|---|---|
| EMX1 | GAGTCCGAGCA GAAGAAGAA (SEQ ID NO: 1159) | GAGTCCGAGCAGA AGAAGAAggg (SEQ ID NO: 1160) | Yes | Yes | Yes | Yes | Yes |
| TTLL 11 | GCTTGCCTTGTG ACATCTAC (SEQ ID NO: 1161) | GCTTGCCTTGTGAC ATCTACtgg (SEQ ID NO: 1162) | | Yes | Yes | Yes | Yes |
| CLIC3 | GACAGACACGCT GCAGATCG (SEQ ID NO: 1163) | GACAGACACGCTG CAGATCGagg (SEQ ID NO: 1164) | | Yes | Yes | Yes | Yes |
| RNF1 03-CHM P3 | GTGCATTTCACC ACTGAAAT (SEQ ID NO: 1165) | GTGCATTTCACCAC TGAAATtgg (SEQ ID NO: 1166) | | | Yes | Yes | Yes |
| RGS8 | GACCCTCAGGCC ATGAGGAC (SEQ ID NO: 1167) | GACCCTCAGGCCA TGAGGACtgg (SEQ ID NO: 1168) | | | Yes | Yes | Yes |
| GTPB P2 | GTTTCTTTTCAG GCTGAAGA (SEQ ID NO: 1169) | GTTTCTTTTCAGGC TGAAGAtgg (SEQ ID NO: 1170) | | | Yes | Yes | Yes |
| SYNP O | GGGCGTCCCAGC ACGACGAC (SEQ ID NO: 1171) | GGGCGTCCCAGCA CGACGACagg (SEQ ID NO: 1172) | | | Yes | Yes | Yes |
| VEGF A | GGTGAGTGAGTG TGTGCGTG (SEQ ID NO: 1173) | GGTGAGTGAGTGT GTGCGTGtgg (SEQ ID NO: 1174) | | | Yes | Yes | Yes |
| ALDH 1A3 | GGAGAGGGACC GCGCCACCT (SEQ ID NO: 1175) | GGAGAGGGACCGC GCCACCTtgg (SEQ ID NO: 1176) | | | Yes | Yes | Yes |
| CACN G3 | GAACTTACGCAG GAGATATT (SEQ ID NO: 1177) | GAACTTACGCAGG AGATATTcgg (SEQ ID NO: 1178) | | | Yes | Yes | Yes |
| ADO RA2B | GTTCCGGTAAGC ATAGACAA (SEQ ID NO: 1179) | GTTCCGGTAAGCA TAGACAAtgg (SEQ ID NO: 1180) | | | | Yes | Yes |
| PEX1 2 | GAGACCCGCTCT TCAGCATG (SEQ ID NO: 1181) | GAGACCCGCTCTTC AGCATGtgg (SEQ ID NO: 1182) | | | | Yes | Yes |
| CRAB P2 | GAGAGGGCCCC AAGACCTCG (SEQ ID NO: 1183) | GAGAGGGCCCCAA GACCTCGtgg (SEQ ID NO: 1184) | | | | Yes | Yes |
| TWS G1 | GCGCCTTATTCC AGTGACAA (SEQ ID NO: 1185) | GCGCCTTATTCCAG TGACAAagg (SEQ ID NO: 1186) | | | | Yes | Yes |
| HCN2 | GCAGATCCTCAT CACCGCGC (SEQ ID NO: 1187) | GCAGATCCTCATC ACCGCGCtgg (SEQ ID NO: 1188) | | | | Yes | Yes |
| EEF2 | GCATGTCGACTT CTCCTCGG (SEQ ID NO: 1189) | GCATGTCGACTTCT CCTCGGagg (SEQ ID NO: 1190) | | | | Yes | Yes |
| IL29 | GCTGGTCTAGGA CGTCCTCC (SEQ ID NO: 1191) | GCTGGTCTAGGAC GTCCTCCagg (SEQ ID NO: 1192) | | | | Yes | Yes |
| FGF2 1 | GGAAACTCACCG ATCCATAC (SEQ ID NO: 1193) | GGAAACTCACCGA TCCATACagg (SEQ ID NO: 1194) | | | | Yes | Yes |
| METT L18 | GCCAGCAAAGC ACATTATTT (SEQ ID NO: 1195) | GCCAGCAAAGCAC ATTATTTtgg (SEQ ID NO: 1196) | | | | Yes | Yes |
| RIMS 4 | GGCCCGTCTCCG TGCTCCTC (SEQ ID NO: 1197) | GGCCCGTCTCCGTG CTCCTCtgg (SEQ ID NO: 1198) | | | | Yes | Yes |
| EEF1 A2 | GCGCTACGACGA GATCGTCA (SEQ ID NO: 1199) | GCGCTACGACGAG ATCGTCAagg (SEQ ID NO: 1200) | | | | Yes | Yes |
| FAM5 C | GAGAATAAGATT CAGTTGCA (SEQ ID NO: 1201) | GAGAATAAGATTC AGTTGCAagg (SEQ ID NO: 1202) | | | | Yes | Yes |
| EHD3 | GTTTCTTGGGAT CCACCACC (SEQ ID NO: 1203) | GTTTCTTGGGATCC ACCACCagg (SEQ ID NO: 1204) | | | | Yes | Yes |
| PRKC E | GTAGGTGGGCTG CCGAAGAT (SEQ ID NO: 1205) | GTAGGTGGGCTGC CGAAGATagg (SEQ ID NO: 1206) | | | | Yes | Yes |
| DIRC 1 | GTAATTAGGTAA GGCTTAGT (SEQ ID NO: 1207) | GTAATTAGGTAAG GCTTAGTtgg (SEQ ID NO: 1208) | | | | Yes | Yes |
| SDPR | GCTCTTTGACCG CGCGCGTG (SEQ ID NO: 1209) | GCTCTTTGACCGCG CGCGTGtgg (SEQ ID NO: 1210) | | | | Yes | Yes |
| CTNN B1 | GAAACAGCTCGT TGTACCGC (SEQ ID NO: 1211) | GAAACAGCTCGTT GTACCGCtgg (SEQ ID NO: 1212) | | | | Yes | Yes |
| CCDC 80 | GCAACAACGTG ATGAATATC (SEQ ID NO: 1213) | GCAACAACGTGAT GAATATCtgg (SEQ ID NO: 1214) | | | | Yes | Yes |
| PRD M2 | GTCGCTGTGACT TTCTAATT (SEQ ID NO: 1215) | GTCGCTGTGACTTT CTAATTtgg (SEQ ID NO: 1216) | | | | Yes | Yes |
| CSF1 | GGTGTTATCTCT GAAGCGCA (SEQ ID NO: 1217) | GGTGTTATCTCTGA AGCGCAtgg (SEQ ID NO: 1218) | | | | Yes | Yes |
| ATR | GGATCATGGAA GCCAGCTCC (SEQ ID NO: 1219) | GGATCATGGAAGC CAGCTCCagg (SEQ ID NO: 1220) | | | | | Yes |
| SMOC1 | GGTCTCGGCACTTGGCTCGC (SEQ ID NO: 1221) | GGTCTCGGCACTTGGCTCGCtgg (SEQ ID NO: 1222) | | | | | Yes |
| RP11-382A2 0.3 | GGAGGCTTCACA GCGCCCTC (SEQ ID NO: 1223) | GGAGGCTTCACAG CGCCCTCtgg (SEQ ID NO: 1224) | | | | | Yes |
| POLR 2H | GCTAGTACCTTG TATGAAGA (SEQ ID NO: 1225) | GCTAGTACCTTGTA TGAAGAtgg (SEQ ID NO: 1226) | | | | | Yes |
| LIMC H1 | GACGGGAAAGT CAGTGTGAA (SEQ ID NO: 1227) | GACGGGAAAGTCA GTGTGAAtgg (SEQ ID NO: 1228) | | | | | Yes |
| CTXN 3 | GTTCGACCATGC CCTTGCTT (SEQ ID NO: 1229) | GTTCGACCATGCCC TTGCTTagg (SEQ ID NO: 1230) | | | | | Yes |
| HCRT R1 | GGCAGAGCTCAC CTGTAGAT (SEQ ID NO: 1231) | GGCAGAGCTCACC TGTAGATagg (SEQ ID NO: 1232) | | | | | Yes |
| BCAP 29 | GCTGGTGGAGCT CTTCTCAA (SEQ ID NO: 1233) | GCTGGTGGAGCTC TTCTCAAtgg (SEQ ID NO: 1234) | | | | | Yes |
| CREB 3L2 | GGAGCTGACCCA AGACGTTC (SEQ ID NO: 1235) | GGAGCTGACCCAA GACGTTCtgg (SEQ ID NO: 1236) | | | | | Yes |
| SLC4 A4 | GTTGACCATCAG ATTGAGAC (SEQ ID NO: 1237) | GTTGACCATCAGA TTGAGACagg (SEQ ID NO: 1238) | | | | | Yes |
| LEF1 | GCTCACCTCGTG TCCGTTGC (SEQ ID NO: 1239) | GCTCACCTCGTGTC CGTTGCtgg (SEQ ID NO: 1240) | | | | | Yes |
| CCDC 111 | GGACGTTCATGT ATTTGCTT (SEQ ID NO: 1241) | GGACGTTCATGTAT TTGCTTtgg (SEQ ID NO: 1242) | | | | | Yes |
| OXCT 1 | GCTGTAAAAGAC ATCCCTGA (SEQ ID NO: 1243) | GCTGTAAAAGACA TCCCTGAtgg (SEQ ID NO: 1244) | | | | | Yes |
| AC11 4947.1 | GGGTCTCCACCA CTTCGTAA (SEQ ID NO: 1245) | GGGTCTCCACCACT TCGTAAagg (SEQ ID NO: 1246) | | | | | Yes |
| ALG8 | GGCGGCGCTCAC AATTGCCA (SEQ ID NO: 1247) | GGCGGCGCTCACA ATTGCCAcgg (SEQ ID NO: 1248) | | | | | Yes |
| C11or f88 | GGTACTTACTGT TACTCGCA (SEQ ID NO: 1249) | GGTACTTACTGTTA CTCGCAagg (SEQ ID NO: 1250) | | | | | Yes |
| DTX3 | GACGCTGGTCAA ACGCCTTG (SEQ ID NO: 1251) | GACGCTGGTCAAA CGCCTTGcgg (SEQ ID NO: 1252) | | | | | Yes |
| KIAA 0895L | GGCATGCTGCGG CATGAGAT (SEQ ID NO: 1253) | GGCATGCTGCGGC ATGAGATagg (SEQ ID NO: 1254) | | | | | Yes |
| TAF4 B | GGCTCCACGCAG ACGCTGAC (SEQ ID NO: 1255) | GGCTCCACGCAGA CGCTGACagg (SEQ ID NO: 1256) | | | | | Yes |
| PTMA | GTCGAGGAGAA TGAGGAAAA (SEQ ID NO: 1257) | GTCGAGGAGAATG AGGAAAAtgg (SEQ ID NO: 1258) | | | | | Yes |
| APOL 2 | GCAGATTCTCTC TGCTCACT (SEQ ID NO: 1259) | GCAGATTCTCTCTG CTCACTtgg (SEQ ID NO: 1260) | | | | | Yes |
| TIFA B | GATGGTACAGGC TCACTCGC (SEQ ID NO: 1261) | GATGGTACAGGCT CACTCGCagg (SEQ ID NO: 1262) | | | | | Yes |
| CEL | GCACCCAAATGT TGAGGTAC (SEQ ID NO: 1263) | GCACCCAAATGTT GAGGTACagg (SEQ ID NO: 1264) | | | | | Yes |
| C11or f41 | GTCATCGAACTG CTCTTAGC (SEQ ID NO: 1265) | GTCATCGAACTGCT CTTAGCtgg (SEQ ID NO: 1266) | | | | | Yes |
| PLEK HG6 | GCCTGACCATCG AGAAGTCC (SEQ ID NO: 1267) | GCCTGACCATCGA GAAGTCCtgg (SEQ ID NO: 1268) | | | | | Yes |
| LRRC 48 | GGACGATGACAT GCTCAAGC (SEQ ID NO: 1269) | GGACGATGACATG CTCAAGCtgg (SEQ ID NO: 1270) | | | | | Yes |
| GDF1 5 | GCGCGTGCATGT TTGCCGCC (SEQ ID NO: 1271) | GCGCGTGCATGTTT GCCGCCcgg (SEQ ID NO: 1272) | | | | | Yes |
| HEK2 93 site | GGCACTGCGGCT GGAGGTGG (SEQ ID NO: 1273) | GGCACTGCGGCTG GAGGTGGggg (SEQ ID NO: 1274) | | | | | Yes |
| FANC F | GCTGCAGAAGG GATTCCATG (SEQ ID NO: 1275) | GCTGCAGAAGGGA TTCCATGagg (SEQ ID NO: 1276) | | | | | Yes |
| DYN C1H1 | GCGAGTCTTCAC TGAGTGTA (SEQ ID NO: 1277) | GCGAGTCTTCACTG AGTGTAagg (SEQ ID NO: 1278) | | | | | Yes |

TABLE 6D

gRNAs used for comparison with other off-target detection techniques

| | | | |
|---|---|---|---|
| Name | Spacer | Target Site with PAM | Method |
| EMX1 | GAGTCCGAGCAGAAGAAGA A (SEQ ID NO: 1279) | GAGTCCGAGCAGAAGAAGAAg gg (SEQ ID NO: 1280) | GUIDE-seq |
| VEGFA 3 | GGTGAGTGAGTGTGTGCGTG (SEQ ID NO: 1281) | GGTGAGTGAGTGTGTGCGTGtgg (SEQ ID NO: 1282) | GUIDE-seq |
| RNF2 | GTCATCTTAGTCATTACCTG (SEQ ID NO: 1283) | GTCATCTTAGTCATTACCTGagg (SEQ ID NO: 1284) | DISCOV ER-seq |
| VEGFA | GACCCCCTCCACCCCGCCTC (SEQ ID NO: 1285) | GACCCCCTCCACCCCGCCTCcgg (SEQ ID NO: 1286) | DISCOV ER-seq |

TABLE 6E

gRNAs used for prime editing specificity test

| | | |
|---|---|---|
| Target | pegRNA spacer sequence | pegRNA 3′ extension |
| HEK3 | GGCCCAGACTGAG CACGTGA (SEQ ID NO: 1287) | TGGAGGAAGCAGGGCTTCCTTTCCTCTGCCATC ACTTATCGTCGTCATCCTTGTAATCCGTGCTCAG TCTG (SEQ ID NO: 1288) |
| DNMT1 | GGTGCCAGAAACA GGGGTGA (SEQ ID NO: 1289) | GTGCCTGCTAAGGACTAGTTCTGCCCTCCAGTC AGGCTTGTCGACGACGGCGGTCTCCGTCGTCAG GATCATCCCCTGTTTCTGGCA (SEQ ID NO: 1290) |
| EMX1 | gTGCTCCAGAGGCC CCCCTTG (SEQ ID NO: 1291) | GTGCTGTAGCCTGCCCTCTGCACCTCCTCACCA AGGCTTGTCGACGACGGCGGTCTCCGTCGTCAG GATCATGGGGGGCCTCTGGAG (SEQ ID NO: 1292) |

### REFERENCES

Allen, F., Crepaldi, L., Alsinet, C., Strong, A.J., Kleshchevnikov, V., De Angeli,= P., Páleníková, P., Khodak, A., Kiselev, V., Kosicki, M., et al. (2018). Predicting the mutations generated by repair of Cas9-induced double-strand breaks. Nature Biotechnology 37, 64-72.

Anzalone, A.V., Randolph, P.B., Davis, J.R., Sousa, A.A., Koblan, L.W., Levy, J.M., Chen, P.J., Wilson, C., Newby, G.A., Raguram, A., et al. (2019). Search-and-replace genome editing without double-strand breaks or donor DNA. Nature 576, 149-157.

Cameron, P., Fuller, C.K., Donohoue, P.D., Jones, B.N., Thompson, M.S., Carter, M.M., Gradia, S., Vidal, B., Garner, E., Slorach, E.M., et al. (2017). Mapping the genomic landscape of CRISPR-Cas9 cleavage. Nat Meth 14, 600-606.

Casini, A., Olivieri, M., Petris, G., Montagna, C., Reginato, G., Maule, G., Lorenzin, F., Prandi, D., Romanel, A., Demichelis, F., et al. (2018). A highly specific SpCas9 variant is identified by in vivo screening in yeast. Nature Biotechnology 36, 265-271.

Chen, J.S., Dagdas, Y.S., Kleinstiver, B.P., Welch, M.M., Sousa, A.A., Harrington, L.B., Sternberg, S.H., Joung, J.K., Yildiz, A., and Doudna, J.A. (2017). Enhanced proofreading governs CRISPR-Cas9 targeting accuracy. Nature 550, 407-410.

Chen, W., McKenna, A., Schreiber, J., Haeussler, M., Yin, Y., Agarwal, V., Noble, W.S., and Shendure, J. (2019). Massively parallel profiling and predictive modeling of the outcomes of CRISPR/Cas9-mediated double-strand break repair. Nucl. Acids Res. 47, 7989-8003.

Gao, L., Cox, D.B.T., Yan, W.X., Manteiga, J.C., Schneider, M.W., Yamano, T., Nishimasu, H., Nureki, O., Crosetto, N., and Zhang, F. (2017). Engineered Cpf1 variants with altered PAM specificities. Nature Biotechnology 163, 759.

Hu, J.H., Miller, S.M., Geurts, M.H., Tang, W., Chen, L., Sun, N., Zeina, C.M., Gao, X., Rees, H.A., Lin, Z., et al. (2018). Evolved Cas9 variants with broad PAM compatibility and high DNA specificity. Nature 556, 57-63.

Kim, D., Bae, S., Park, J., Kim, E., Kim, S., Yu, H.R., Hwang, J., Kim, J.-I., and Kim, J.-S. (2015). Digenome-seq: genome-wide profiling of CRISPR-Cas9 off-target effects in human cells. Nat Meth 12, 237-243.

Kleinstiver, B.P., Pattanayak, V., Prew, M.S., Tsai, S.Q., Nguyen, N.T., Zheng, Z., and Joung, J.K. (2016). High-fidelity CRISPR-Cas9 nucleases with no detectable genome-wide off-target effects. Nature 529, 490-495.

Lee, J.K., Jeong, E., Lee, J., Jung, M., Shin, E., Kim, Y.-H., Lee, K., Jung, I., Kim, D., Kim, S., et al. (2018). Directed evolution of CRISPR-Cas9 to increase its specificity. Nature Communications 9, 3048.

Listgarten, J., Weinstein, M., Kleinstiver, B.P., Sousa, A.A., Joung, J.K., Crawford, J., Gao, K., Hoang, L., Elibol, M., Doench, J.G., et al. (2018). Prediction of off-target activities for the end-to-end design of CRISPR guide RNAs. Nature Biomedical Engineering 2018 2:7 2, 38-47.

Palermo, G., Miao, Y., Walker, R.C., Jinek, M., and McCammon, J.A. (2016). Striking Plasticity of CRISPR-Cas9 and Key Role of Non-target DNA, as Revealed by Molecular Simulations. ACS Cent Sci 2, 756-763.

Perez, A.R., Pritykin, Y., Vidigal, J.A., Chhangawala, S., Zamparo, L., Leslie, C.S., and Ventura, A. (2017). GuideScan software for improved single and paired CRISPR guide RNA design. Nature Biotechnology 35, 347-349.

Picelli, S., Björklund, A.K., Reinius, B., Sagasser, S., Winberg, G., and Sandberg, R. (2014). Tn5 transposase and tagmentation procedures for massively scaled sequencing projects. Genome Res. 24, 2033-2040.

Ran, F.A., Hsu, P.D., Wright, J., Agarwala, V., Scott, D.A., and Zhang, F. (2013). Genome engineering using the CRISPR-Cas9 system. Nature Protocols 8, 2281-2308.

Ribeiro, L.F., Ribeiro, L. F. C., Barreto, M. Q. and Ward, R. J. (2018). Protein engineering strategies to expand CRISPR-Cas9 applications. Intl J. Genomics Vol. 2018, Article ID 1652567 (12 pages); doi.org/10.1155/2018/1652567.

Schmid-Burgk, J.L., and Hornung, V. (2015). BrowserGenome.org: web-based RNA-seq data analysis and visualization. Nat Meth 12, 1001-1001.

Schmid-Burgk, J.L., Schmidt, T., Gaidt, M.M., Pelka, K., Latz, E., Ebert, T.S., and Hornung, V. (2014). OutKnocker: a web tool for rapid and simple genotyping of designer nuclease edited cell lines. Genome Res. 24, 1719-1723.

Shalem, O., Sanjana, N.E., Hartenian, E., Shi, X., Scott, D.A., Mikkelsen, T.S., Heckl, D., Ebert, B.L., Root, D.E., Doench, J.G., et al. (2014). Genome-scale CRISPR-Cas9 knockout screening in human cells. Science 343, 84-87.

Shen, M.W., Arbab, M., Hsu, J.Y., Worstell, D., Culbertson, S.J., Krabbe, O., Cassa, C.A., Liu, D.R., Gifford, D.K., and Sherwood, R.I. (2018). Predictable and precise template-free CRISPR editing of pathogenic variants. Nature 563, 646-651.

Slaymaker, I.M., Gao, L., Zetsche, B., Scott, D.A., Yan, W.X., and Zhang, F. (2015). Rationally engineered Cas9 nucleases with improved specificity. Science 351, 84-88.

Strecker, J., Jones, S., Koopal, B., Schmid-Burgk, J., Zetsche, B., Gao, L., Makarova, K.S., Koonin, E.V., and Zhang, F. (2019a). Engineering of CRISPR-Cas12b for human genome editing. Nature Communications 10, 866.

Strecker, J., Ladha, A., Gardner, Z., Schmid-Burgk, J.L., Makarova, K.S., Koonin, E.V., and Zhang, F. (2019b). RNA-guided DNA insertion with CRISPR-associated transposases. Science eaax9181.

Tsai, S.Q., and Joung, J.K. (2016). Defining and improving the genome-wide specificities of CRISPR-Cas9 nucleases. Nature Publishing Group 17, 300-312.

Tsai, S.Q., Nguyen, N.T., Malagon-Lopez, J., Topkar, V.V., Aryee, M.J., and Joung, J.K. (2017). CIRCLE-seq: a highly sensitive in vitro screen for genome-wide CRISPR-Cas9 nuclease off-targets. Nat Meth 14, 607-614.

Tsai, S.Q., Zheng, Z., Nguyen, N.T., Liebers, M., Topkar, V.V., Thapar, V., Wyvekens, N., Khayter, C., Iafrate, A.J., Le, L.P., et al. (2015). GUIDE-seq enables genome-wide profiling of off-target cleavage by CRISPR-Cas nucleases. Nature Biotechnology 33, 187-197.

Vakulskas, C.A., Dever, D.P., Rettig, G.R., Turk, R., Jacobi, A.M., Collingwood, M.A., Bode, N.M., McNeill, M.S., Yan, S., Camarena, J., et al. (2018). A high-fidelity Cas9 mutant delivered as a ribonucleoprotein complex enables efficient gene editing in human hematopoietic stem and progenitor cells. Nat Med 24, 1216-1224.

Wienert, B., Wyman, S.K., Richardson, C.D., Yeh, C.D., Akcakaya, P., Porritt, M.J., Morlock, M., Vu, J.T., Kazane, K.R., Watry, H.L., et al. (2019). Unbiased detection of CRISPR off-targets in vivo using DISCOVER-Seq. Science 364, 286-289.

Zuo, Z., and Liu, J. (2016). Cas9-catalyzed DNA Cleavage Generates Staggered Ends: Evidence from Molecular Dynamics Simulations. Scientific Reports 6, 37584.

### Supplementary Methods 1

### Step 1: Tn5 Purification

Grew E. coli cells (NEB C3013) harboring the plasmid pTBX1-Tn5 in terrific broth to an OD of 0.65

Added IPTG to a concentration of 0.25 mM and shake at 23° C. overnight

Harvested cells by centrifugation and stored at -80° C. until purification

Lysed 20 g of A. coli pellet in 200 mL HEGX buffer (20 mM HEPES-KOH pH 7.2, 800 mM NaCl, 1 mM EDTA, 0.2% Triton, 10% glycerol) with cOmplete protease inhibitor (Roche) and 10 µL of Benzonase (Sigma-Aldrich), using an LM20 microfluidizer device (Microfluidics)

Cleared the lysate by centrifugation at max speed for 30 min

Added 5.25 mL of 10% PEI (pH 7) dropwise to a stirring solution to remove E.coli DNA. For 10 min

Added cleared supernatant to 30 mL of equilibrated chitin resin (NEB) and mix end-over-end for 30 min

Added mixture to column, wash with 1 L HEGX buffer

Added 75 mL HEGX buffer with 100 mM DTT to column, drew 30 mL through the resin before sealing the column and storing at 4° C. for 48 h to allow for intein cleavage and elution of free Tn5

Dialyzed eluted Tn5 into 2xTn5 dialysis buffer (100 HEPES, 200 NaCl, 2 EDTA, 0.2 Triton, 20% glycerol), with two exchanges of 1 L of buffer

Concentrated the final solution to 50 mg/mL as determined by A280 absorbance (A280 = 1 = 0.616 mg/mL = 11.56 mM)

### Step 2: Flash-Freeze in Liquid Nitrogen Before Storage at -80°

Annealed oligonucleotides Transposon ME and Transposon read 2 at a concentration of 42 µM each in annealing buffer (1.5 mM Tris-HCl pH 8.0, 150 µM EDTA, 30 mM NaCl) by heating to 95C for 3 minutes, and subsequently ramping the temperature from 70C to 25C at a rate of 1C per minute

Incubated 1 ml of purified Tn5 (50 mg/ml) with 355 µl of annealed oligonucleotides for 1 hour at room temperature. Of note, loaded Tn5 can crash out as white precipitate, but retains activity.

Stored loaded Tn5 at 20C, ready to be thawed on ice for later use. Resuspend before use.

### Step 3: Cell Transfection

Seeded HEK293T cells in poly-D-lysine coated 96-well plates (Corning) at a density of 25,000 cells in 100 µl medium per well

Annealed TTISS donor sense and TTISS donor antisense in 0.1x IDT Nuclease-Free Duplex Buffer by ramping the temperature from 95° C. to 25° C. at a rate of 1° C. per minute

The next day, mixed 250 µl OptiMEM (Thermo) with 1 µg of annealed oligonucleotide donor, 750 ng Cas9 expression plasmid, and a total of 250 ng of 1-60 different gRNA expression plasmids for each condition

In parallel, mixed 250 µl OptiMEM with 5 µl GeneJuice (Millipore) and incubated at room temperature for 5 minutes for each condition

Mixed all components for each condition and incubate them for 20 minutes

Added 50 µl drop-wise per 96-well of cells in a total of ten wells per condition

### Step 4: Cell Lysis and Genome Tagmentation

Two to three days after transfection, washed cells with PBS, trypsinized, and washed again with PBS in a 1.5 ml tube

Lysed pelleted cells by re-suspending one million cells in 100 µl lysis buffer (1 mM CaCl2, 3 mM MgCl2, 1 mM EDTA, 1% Triton X-100, 10 mM Tris pH 7.5, 8 units/ml Proteinase K (NEB))

Heated lysates to 65° C. for 10 minutes, then kept on ice

For tagmentation, mixed 80 µl crude lysate with 25 µl 5x TAPS buffer (50 mM TAPS-NaOH pH 8.5 at room temperature, 25 mM MgCl2) and 20 µl hyperactive loaded Tn5 transposase. Heat to 55° C. for 10 minutes.

Mixed reactions with 625 µl PB buffer (Qiagen) and bound to a mini-prep silica spin column. Washed with 750 µl buffer PE (Qiagen), spun dry, and eluted DNA in 50 µl water (typical concentration: 200-300 ng/µl).

Ran 3 µl of the eluate on a 2% Agarose gel to check size range

If size range was outside the range of 300 to 1,000 bp, repeated with adjusted amounts of Tn5 and noted adjustments for future use of the Tn5 batch. Alternatively, performed a titration of loaded Tn5 at the start using extra cell lysate to determine optimal tagmentation conditions.

### Step 5: PCR Amplification

Denatured total eluates at 95° C. for 5 minutes, then snap-cool on ice

Amplified in 200 µl PCR reactions using KOD Hot Start polymerase (Millipore) according to the manufacturer’s protocol (12 cycles, Ta = 60° C., one minute elongation, primers: TTISS PCR fwd 1, Transposon read 2)

For each sample, performed a secondary 50 µl KOD PCR templated with 3 µl of the first PCR reaction and a unique barcoding primer (20 cycles, Ta = 65° C., one minute elongation, primers: TTISS PCR fwd 2, TTISS PCR rev BC1-24)

### Step 6: Deep Sequencing

Pooled PCRs on ice, column-purified on a mini-prep silica gel column, and purified fragments within a size range of 250-1,000 bp using a 2% agarose gel

Performed two consecutive column purifications (first with buffer QG (Qiagen) and isopropanol added to the gel slice before loading, second with buffer PB and the eluate from the previous column)

Quantified the library using a NanoDrop spectrometer (Thermo)

Sequenced using an Illumina NextSeq 500 sequencer with a 75-cycle high-output v2 kit (cycle numbers: read 1 = 59, index 1 = 8, read 2 = 25, no index 2)

### Step 7: Read Mapping

Opened in a web browser the site www.BrowserGenome.org

Clicked the “Map deep sequencing data” tab

Under point 2 clicked “Browse” to choose the human genome file “hg38.2bit” on hard drive (download from http://hgdownload.cse.ucsc.edu/goldenPath/hg38/bigZips/hg38.2bit)

Under point 3 clicked “Browse” to choose all un-compressed FASTQ files to be analyzed

Under point 4, entered the filter values 0 bp, NNNNNNNNNNNNNNNNNNNNNNNAAC (SEQ ID NO: 1293)

Under point 5 entered forward mapping start = 26 bp

Under point 6 entered forward mapping length = 25 bp

Under point 7 entered reverse mapping length = 15 bp

Under point 8 entered max forward/reverse span = 1000 bp

Clicked “Start mapping”, which took about one hour per ten million reads

When all data was processed, clicked “Save all” on bottom right to save mapping data files

Clicked on the “Process” tab, then “Remove single read noise” and “Enforce antisense-overlap reads” for basic noise reduction and off-target site identification

Clicked “Export peak list” to save a list of detected cleavage sites, which can be opened in a text or spreadsheet editor for further analysis

For more complex analyses (such as gRNA multiplexing or indel distribution prediction), refer to the Read Me on the Github repository available at URL: github. com/schmidburgk/tti ss.

The sequence of the plasmid used for expressing LZ3 Cas9, with annotations of the sequences of LZ3 Cas9 is shown below. The map of the plasmid is shown in FIG. 7.

FEATURES Location/Qualifiers
primer_bind complement(8096..8115)
/note=”pRS vectors, use to sequence yeast selectable
marker”
/locus_tag=”pRS-marker”
/label=”pRS-marker”
/ApEinfo_label=”pRS-marker”
/ApEinfo_fwdcolor=”#14c0bd”
/ApEinfo_revcolor=”#4ec02b”
/ApEinfo_graphicformat=”arrow_data {{0 1 2 0 0 -1} {} 0}
width 5 offset 0”
rep_origin 7624..8079
/direction=LEFT
/note=”f1 bacteriophage origin of replication; arrow
indicates direction of (+) strand synthesis”
/locus_tag=”f1 ori”
/label=”f1 ori”
/ApEinfo_label=”f1 ori”
/ApEinfo_fwdcolor=”#999999”
/ApEinfo_revcolor=”#999999”
/ApEinfo_graphicformat=”arrow_data {{0 1 2 0 0 -1} {} 0}
width 5 offset 0”
primer_bind 7921..7942
/note=”F 1 origin, forward primer”
/locus_tag=”F1ori-F”
/label=”F1ori-F”
/ApEinfo_label=”F1 ori-F”
/ApEinfo_fwdcolor=”#14c0bd”
/ApEinfo_revcolor=”#4ec02b”
/ApEinfo_graphicformat=”arrow_data {{0 1 2 0 0 -1} {} 0}
width 5 offset 0”
primer_bind complement(7711..7730)
/note=”F 1 origin, reverse primer”
/locus_tag=”F1ori-R”
/label=”F1ori-R”
/ApEinfo_label=”F1 ori-R”
/ApEinfo_fwdcolor=”#14c0bd”
/ApEinfo_revcolor=”#4ec02b”
/ApEinfo_graphicformat=”arrow_data {{0 1 2 0 0 -1} {} 0}
width 5 offset 0”
repeat_region complement(7409..7549)
/note=”inverted terminal repeat of adeno-associated virus
serotype 2”
/locus_tag=”AAV2 ITR”
/label=”AAV2 ITR”
/ApEinfo_label=”AAV2 ITR”
/ApEinfo_fwdcolor=”#0dfff7”
/ApEinfo_revcolor=”#0dfff7”
/ApEinfo_graphicformat=”arrow_data {{0 1 2 0 0 -1} {} 0}
width 5 offset 0”
repeat_region complement(7409..7538)
/locus_tag=” AAV2 ITR(1)”
/label=”AAV2 ITR(1)”
/ApEinfo_label=”AAV2 ITR”
/ApEinfo_fwdcolor=”#0dfff7”
/ApEinfo_revcolor=”#0dfff7”
/ApEinfo_graphicformat=”arrow_data {{0 1 2 0 0 -1} { } 0}
width 5 offset 0”
polyA_signal complement(7193..7400)
/note=”bovine growth hormone polyadenylation signal”
/locus_tag=”bGH poly(A) signal”
/label=”bGH poly(A) signal”
/ApEinfo_label=”bGH poly(A) signal”
/ApEinfo _fwdcolor=”#ff3eee”
/ApEinfo _revcolor=”#ff3eee”
/ApEinfo_graphicformat=”arrow_data { {0 1 2 0 0 -1} { } 0}
width 5 offset 0”
primer_bind complement(7187..7204)
/note=”Bovine growth hormone terminator, reverse primer.
Also called BGH reverse”
/locus_tag=”BGH-rev”
/label =”BGH -rev”
/ApEinfo_label=”BGH-rev”
/ApEinfo _fwdcolor=”#14c0bd”
/ApEinfo_revcolor=”#4ec02b”
/ApEinfo_graphicformat=”arrow_data { {0 1 2 0 0 -1} {} 0}
width 5 offset 0”
CDS 7112..7159
/codon_start=1
/product=”bipartite nuclear localization signal from
nucleoplasmin”
/translation=”KRPAATKKAGQAKKKK” (SEQ ID NO: 1294)
/locus _tag=”nucleoplasmin NLS”
/label=”nucleoplasmin NLS”
/ApEinfo_label=”nucleoplasmin NLS”
/ApEinfo_fwdcolor=”#e9d024”
/ApEinfo_revcolor=”#e9d024”
/ApEinfo_graphicformat=”arrow_data { {0 1 2 0 0 -1} {} 0}
width 5 offset 0”
CDS 2966..2986
/codon_start=1
/product=”nuclear localization signal of SV40 (simian
virus 40) large T antigen”
/translation=”PKKKRKV” (SEQ ID NO: 1295)
/locus _tag=”SV40 NLS”
/label=”SV40 NLS”
/ApEinfo_label=”SV40 NLS”
/ApEinfo_fwdcolor=”#e9d024”
/ApEinfo_revcolor=”#e9d024”
/ApEinfo_graphicformat=”arrow_data { {0 1 2 0 0 -1} {} 0}
width 5 offset 0”
CDS 2894..2959
/codon_start=1
/product=”three tandem FLAGI epitope tags, followed by
an enterokinase cleavage s”te″
/translati”n=″DYKDHDGDYKDHDIDYKDD”DK″ (SEQ ID NO: 1296)
/locus_t”g=″3xF”AG″
/lab”1=″3xF”AG″
/ApEinfo_lab”1=″3xF”AG″
/ ApEinfo _fwdcol”r=″#e9d”24″
/ApEinfo_revcol”r=″#e9d”24″
/ApEinfo_graphicform”t=″arrow_data { {0 1 2 0 0 -1} {} 0}
width 5 offse” 0″
regulatory complement(2885..2894)
/regulatory_cl a”s=″ot”er″
/no”e=″vertebrate consensus sequence for strong initiation
of translation (Kozak, 19”7)″
/locus t”g= ″vertebrate consensus sequence for strong
initiation of translation (Kozak, 19”7)″
/lab”1=″vertebrate consensus sequence for strong
initiation of translation (Kozak, 19”7)″
/ApEinfo_lab”1=″vertebrate consensus sequence for strong
initiation of translation (Kozak, 19”7)″
/ApEinfo fwdcol”t=″p”nk″
/ApEinfo_revcol”r=″p”nk″
/ApEinfo_graphicform”t=″arrow_data { {0 1 2 0 0 -1} {} 0}
width 5 offse” 0″
intron complement(2646..2873)
/no”e=″hybrid between chicken beta-actin (CBA) and minute
virus of mice (MMV) introns (Gray et al., 20”1)″
/locus_t”g=″hybrid int”on″
/lab”1=″hybrid int”on″
/ApEinfo_1ab”1=″hybrid int”on″
/ApEinfo_fwdcol”t=″#eb6”6c″
/ApEinfo_revcol″r=”#eb6”6c″
/ApEinfo_graphicform”t=″arrow_data { {0 1 2 0 0 -1} {} 0}
width 5 offse” 0″
promoter 2368..2645
/locust”g=″chicken beta-actin promo”er″
/lab”1=″chicken beta-actin promo”er″
/ApEinfo_lab”1=″chicken beta-actin promo”er″
/ApEinfo _fwdcol”r=″#346”e0″
/ApEinfo_revcol”r=″#346” e0″
/ApEinfo_graphicform”t=″arrow_data { {0 1 2 0 0 -1} {} 0}
width 5 offse” 0″
enhancer complement(2081..2366)
/no”e=″human cytomegalovirus immediate early enhancer;
contains an 18-bp deletion relative to the standard CMV
enhan”er″
/locus_t”g=″CMV enhan”er″
/lab”1=″CMV enhan”er″
/ApEinfo_lab”1=″CMV enhan”er″
/ApEinfo_fwdcol”r=″#5ac”fa″
/ApEinfo_revcol”r=″#5ac”fa″
/ApEinfo_graphicform”t=″arrow_data { {0 1 2 0 0 -1} {} 0}
width 5 offse” 0″
repeat _region complement(1933..2062)
/no”e=″Functional equivalent of wild-type AAV2 ”TR″
/locus _t”g=″AAV2 ITR (alternae)″
/lab”1=″AAV2 ITR (alterna”e)″
/ApEinfo_lab”l=″AAV2 ITR (alterna”e)″
/ApEinfo-fwdcol”r=″#Odf”f7″
/ApEinfo_revcol”r=″#0df”f7″
/ApEinfo_graphicform”t=″arrow_data { {0 1 2 0 0 -1} {} 0}
width 5 offse” 0″
rep_origin 1283..1871
/direction=LEFT
/no”e=″high-copy-number ColE1/pMB1/pBR322/pUC origin of
replicat”on″
/locus _t”g=″”ri″
/lab”1=″”ri″
/ApEinfo_lab”l=″”ri″
/ApEinfo_fwdcol”r=″#999”99″
/ApEinfo_revcol”r=″#999”99″
/ApEinfo_graphicform”t=″arrow_data {{0 1 2 0 0 -1} {} 0}
width 5 offse” 0″
primer_bind 1772..1791
/no”e=″pBR322 origin, forward pri”er″
/locus _t”g=″pBR322or”-F″
/lab”l=″pBR322or”-F″
/ApEinfo_lab”1=″pBR322or”-F″
/ApEinfo _fwdcol”r=″#14c”bd″
/ApEinfo_revcol”r=″#4ec”2b″
/ApEinfo_graphicform”t″”arrow_data {{0 1 2 0 0 -1} {} 0}
width 5 offse” 0″
CDS 252..1112
/codon _start=1
/ge”e=″”la″
/produ”t=″beta -lactam”se″
/no”e=″confers resistance to ampicillin, carbenicillin,
and related antibiot”cs″
/translati”n=″MSIQHFRVALIPFFAAFCLPVFAHPETLVKVKDAEDQLGARVGY
I
ELDLNSGKILESFRPEERFPMMSTFKVLLCGAVLSRIDAGQEQLGRRIHYSQNDLVEY
S
PVTEKHLTDGMTVRELCSAAITMSDNTAANLLLTTIGGPKELTAFLHNMGDHBTRL
DR
W
EPELNEAIPNDERDTTMPVAMATTLRKLLTGELLTLASRQQLIDWMEADKVAGPLL
RS
A
LPAGWFIADKSGAGERGSRGIIAALGPDGKPSRIVVIYTTGSQATMDERNRQIAEIGA
S LI”HW″ (SEQ ID NO: 1297)
/locus _t”g=″A”pR″
/lab”1=″A”pR″
/ApEinfo_lab”l=″A”pR″
/ApEinfo_fwdcol”r=″#e9d”24″
/ApEinfo_evcol”r=″#e9d”24″
/ApEinfo_graphicform”t=″arrow_data { {0 1 2 0 0 -1} {} 0}
width 5 offse” 0″
primer_bind complement(470..489)
/no”e=″Ampicillin resistance gene, reverse pri”er″
/locus _t”g=″Am”-R″
/lab”1=″Am”-R″
/ApEinfo_lab”1=″Am”-R″
/ApEinfo _fwdcol”r=″#14c”bd″
/ApEinfo _revcol”r=″#4ec”2b″
/ApEinfo_graphicform”t=″arrow_data {{0 1 2 0 0 -1} {} 0}
width 5 offse” 0″
promoter 147..251
/ge”e=″”1a″
/locus _t”g=″AmpR promo”er″
/lab”1=″AmpR promo”er″
/ApEinfo_lab”1=″AmpR promo”er″
/ApEinfo _fwdcol”r=″#346”e0″
/ApEinfo_revcol”r=″#346”e0″
/ApEinfo_graphicform”t=″arrow_data { {0 1 2 0 0 -1} {} 0}
width 5 offse” 0″
primer_bind complement(61..79)
/no”e=″pBR322 vectors, upsteam of EcoRI site, forward
pri”er″
/locus _t”g=″pBRfor”co″
/lab”1=″pBRfor”co″
/ApEinfo_lab”1=″pBRfor”co″
/ApEinfo _fwdcol”r=″#14c”bd″
/ApEinfo_revcol”t=″#4ec”2b″
/ApEinfo_graphicform”t=″arrow_data { {0 1 2 0 0 -1} {} 0}
width 5 offse” 0″
primer_bind 1..23
/no”e=″pGEX vectors, reverse pri”er″
/locus _t”g=″pGE’”3‴
/lab”1=″pGE’”3‴
/ApEinfo_lab”1=″pGE’”3‴
/ApEinfo _fwdcol”r=″#14c”bd″
/ApEinfo_revcol”r=″#4ec”2b″
/ApEinfo_graphicform”t=″arrow_data { {0 1 2 0 0 -1} {} 0}
width 5 offse” 0″
misc_feature 2891..2893
/locus _t” g=″ST”RT″
/lab”1=″ST”RT″
/ApEinfo _lab”1=″ST”RT″
/ApEinfo_fwdcol”r=″c”an″
/ApEinfo_revcol”r=″gr”en″
/ApEinfo_graphicform”t=″arrow_data { {0 1 2 0 0 -1} {} 0}
width 5 offse” 0″
misc_feature 7160.. 7162
/locus _t”g=″S”OP″
/lab”1=″S”OP″
/ApEinfo _lab”1=″S”OP″
/ApEinfo_fwdcol”r=″c”an″
/ApEinfo_revcol”r=″gr”en″
/ApEinfo_graphicform”t=″arrow_data { {0 1 2 0 0 -1} {} 0}
width 5 offse” 0″
misc_feature 3011..7111
/locus_t”g=″LZ3 C”s9″
/lab”1=″LZ3 C”s9″
/ApEinfo_lab”1=″LZ3 C”s9″
/ApEinfo_fwdcol”r=″#00f”00″
/ApEinfo_revcol”r=″gr”en″
/ApEinfo_graphicform”t=″arrow_data { {0 1 2 0 0 -1} {} 0}
width 5 offse” 0″

### pX165-LZ3-Cas9 Sequence

ORIGIN

1 ccgggagctg catgtgtcag aggttttcac cgtcatcacc gaaacgcgcg agacgaaagg
61 gcctcgtgat acgcctattt ttataggtta atgtcatgat aataatggtt tcttagacgt
121 caggtggcac ttttcgggga aatgtgcgcg gaacccctat ttgtttattt ttctaaatac
181 attcaaatat gtatccgctc atgagacaat aaccctgata aatgcttcaa taatattgaa
241 aaaggaagag tatgagtatt caacatttcc gtgtcgccct tattcccttt tttgcggcat
301 tttgccttcc tgtttttgct cacccagaaa cgctggtgaa agtaaaagat gctgaagatc
361 agttgggtgc acgagtgggt tacatcgaac tggatctcaa cagcggtaag atccttgaga
421 gttttcgccc cgaagaacgt tttccaatga tgagcacttt taaagttctg ctatgtggcg
481 cggtattatc ccgtattgac gccgggcaag agcaactcgg tcgccgcata cactattctc
541 agaatgactt ggttgagtac tcaccagtca cagaaaagca tcttacggat ggcatgacag
601 taagagaatt atgcagtgct gccataacca tgagtgataa cactgcggcc aacttacttc
661 tgacaacgat cggaggaccg aaggagctaa ccgctttttt gcacaacatg ggggatcatg
721 taactcgcct tgatcgttgg gaaccggagc tgaatgaagc cataccaaac gacgagcgtg
781 acaccacgat gcctgtagca atggcaacaa cgttgcgcaa actattaact ggcgaactac
841 ttactctagc ttcccggcaa caattaatag actggatgga ggcggataaa gttgcaggac
901 cacttctgcg ctcggccctt ccggctggct ggtttattgc tgataaatct ggagccggtg
961 agcgtggaag ccgcggtatc attgcagcac tggggccaga tggtaagccc tcccgtatcg
1021 tagttatcta cacgacgggg agtcaggcaa ctatggatga acgaaataga cagatcgctg
1081 agataggtgc ctcactgatt aagcattggt aactgtcaga ccaagtttac tcatatatac
1141 tttagattga tttaaaactt catttttaat ttaaaaggat ctaggtgaag atcctttttg
1201 ataatctcat gaccaaaatc ccttaacgtg agttttcgtt ccactgagcg tcagaccccg
1261 tagaaaagat caaaggatct tcttgagatc ctttttttct gcgcgtaatc tgctgcttgc
1321 aaacaaaaaa accaccgcta ccagcggtgg tttgtttgcc ggatcaagag ctaccaactc
1381 tttttccgaa ggtaactggc ttcagcagag cgcagatacc aaatactgtt cttctagtgt
1441 agccgtagtt aggccaccac ttcaagaact ctgtagcacc gcctacatac ctcgctctgc
1501 taatcctgtt accagtggct gctgccagtg gcgataagtc gtgtcttacc gggttggact
1561 caagacgata gttaccggat aaggcgcagc ggtcgggctg aacggggggt tcgtgcacac
1621 agcccagctt ggagcgaacg acctacaccg aactgagata cctacagcgt gagctatgag
1681 aaagcgccac gcttcccgaa gggagaaagg cggacaggta tccggtaagc ggcagggtcg
1741 gaacaggaga gcgcacgagg gagcttccag ggggaaacgc ctggtatctt tatagtcctg
1801 tcgggtttcg ccacctctga cttgagcgtc gatttttgtg atgctcgtca ggggggcgga
1861 gcctatggaa aaacgccagc aacgcggcct ttttacggtt cctggccttt tgctggcctt
1921 ttgctcacat gtcctgcagg cagctgcgcg ctcgctcgct cactgaggcc gcccgggcgt
1981 cgggcgacct ttggtcgccc ggcctcagtg agcgagcgag cgcgcagaga gggagtggcc
2041 aactccatca ctaggggttc ctgcggcctc tagaggtacc cgttacataa cttacggtaa
2101 atggcccgcc tggctgaccg cccaacgacc cccgcccatt gacgtcaata gtaacgccaa
2161 tagggacttt ccattgacgt caatgggtgg agtatttacg gtaaactgcc cacttggcag
2221 tacatcaagt gtatcatatg ccaagtacgc cccctattga cgtcaatgac ggtaaatggc
2281 ccgcctggca ttgtgcccag tacatgacct tatgggactt tcctacttgg cagtacatct
2341 acgtattagt catcgctatt accatggtcg aggtgagccc cacgttctgc ttcactctcc
2401 ccatctcccc cccctcccca cccccaattt tgtatttatt tattttttaa ttattttgtg
2461 cagcgatggg ggcggggggg gggggggggc gcgcgccagg cggggcgggg cggggcgagg
2521 ggcggggcgg ggcgaggcgg agaggtgcgg cggcagccaa tcagagcggc gcgctccgaa
2581 agtttccttt tatggcgagg cggcggcggc ggcggcccta taaaaagcga agcgcgcggc
2641 gggcgggagt cgctgcgcgc tgccttcgcc ccgtgccccg ctccgccgcc gcctcgcgcc
2701 gcccgccccg gctctgactg accgcgttac tcccacaggt gagcggcgg gacggccctt
2761 ctcctccggg ctgtaattag ctgagcaaga ggtaagggtt taagggatgg ttggttggtg
2821 gggtattaat gtttaattac ctggagcacc tgcctgaaat cacttttttt caggttggac
2881 cggtgccacc atggactata aggaccacga cggagactac aaggatcatg atattgatta
2941 caaagacgat gacgataaga tggccccaaa gaagaagcgg aaggtcggta tccacggagt
3001 cccagcagcc GACAAGAAGT ACAGCATCGG CCTGGACATC GGCACCAACTCTGTGGGCTG
3061 GGCCGTGATC ACCGACGAGT ACAAGGTGCC CAGCAAGAAATTCAAGGTGC TGGGCAACAC
3121 CGACCGGCAC AGCATCAAGA AGAACCTGAT CGGAGCCCTGCTGTTCGACA GCGGCGAAAC
3181 AGCCGAGGCC ACCCGGCTGA AGAGAACCGC CAGAAGAAGATACACCAGAC GGAAGAACCG
3241 GATCTGCTAT CTGCAAGAGA TCTTCAGCAA CGAGATGGCCAAGGTGGACG ACAGCTTCTT
3301 CCACAGACTG GAAGAGTCCT TCCTGGTGGA AGAGGATAAGAAGCACGAGC GGCACCCCAT
3361 CTTCGGCAAC ATCGTGGACG AGGTGGCCTA CCACGAGAAGTACCCCACCA TCTACCACCT
3421 GAGAAAGAAA CTGGTGGACA GCACCGACAA GGCCGACCTGCGGCTGATCT ATCTGGCCCT
3481 GGCCCACATG ATCAAGTTCC GGGGCCACTT CCTGATCGAGGGCGACCTGA ACCCCGACAA
3541 CAGCGACGTG GACAAGCTGT TCATCCAGCT GGTGCAGACCTACAACCAGC TGTTCGAGGA
3601 AAACCCCATC AACGCCAGCG GCGTGGACGC CAAGGCCATCCTGTCTGCCA GACTGAGCAA
3661 GAGCAGACGG CTGGAAAATC TGATCGCCCA GCTGCCCGGCGAGAAGAAGA ATGGCCTGTT
3721 CGGAAACCTG ATTGCCCTGA GCCTGGGCCT GACCCCCAACTTCAAGAGCA ACTTCGACCT
3781 GGCCGAGGAT GCCAAACTGC AGCTGAGCAA GGACACCTACGACGACGACC TGGACAACCT
3841 GCTGGCCCAG ATCGGCGACC AGTACGCCGA CCTGTTTCTGGCCGCCAAGA ACCTGTCCGA
3901 CGCCATCCTG CTGAGCGACA TCCTGAGAGT GAACACCGAGATCACCAAGG CCCCCCTGAG
3961 CGCCTCTATG ATCAAGAGAT ACGACGAGCA CCACCAGGACCTGACCCTGC TGAAAGCTCT
4021 CGTGCGGCAG CAGCTGCCTG AGAAGTACAA AGAGATTTTCTTCGACCAGA GCAAGAACGG
4081 CTACGCCGGC TACATTGACG GCGGAGCCAG CCAGGAAGAGTTCTACAAGT TCATCAAGCC
4141 CATCCTGGAA AAGATGGACG GCACCGAGGA ACTGCTCGTGAAGCTGAACA GAGAGGACCT
4201 GCTGCGGAAG CAGCGGACCT TCGACAACGG CAGCATCCCCACCAGATCC ACCTGGGAGA
4261 GCTGCACGCC ATTCTGCGGC GGCAGGAAGA TTTTTACCCATTCCTGAAGG ACAACCGGGA
4321 AAAGATCGAG AAGATCCTGA CCTTCCGCAT CCCCTACTACGTGGGCCCTC TGGCCAGGGG
4381 AAACAGCAGA TTCGCCTGGA TGACCAGAAA GAGCGAGGAAACCATCACCC CCTGGAACTT
4441 CGAGGAAGTG GTGGACAAGG GCGCTTCCGC CCAGAGCTTCATCGAGCGGA TGACCAACTT
4501 CGATAAGAAC CTGCCCAACG AGAAGGTGCT GCCCAAGCACAGCCTGCTGT ACGAGTACTT
4561 CACCGTGTAT AACGAGCTGA CCAAAGTGAA ATACGTGACCGAGGGAATGA GAAAGCCCGC
4621 CTTCCTGAGC GGCGAGCAGA AAAAGGCCAT CGTGGACCTGCTGTTCAAGA CCAACCGGAA
4681 AGTGACCGTG AAGCAGCTGA AAGAGGACTA CTTCAAGAAAATCGAGTGCT TCGACTCCGT
4741 GGAAATCTCC GGCGTGGAAG ATCGGTTCAA CGCCTCCCTGGCACATACC ACGATCTGCT
4801 GAAAATTATC AAGGACAAGG ACTTCCTGGA CAATGAGGAAAACGAGGACA TTCTGGAAGA
4861 TATCGTGCTG ACCCTGACAC TGTTTGAGGA CAGAGAGATGATCGAGGAAC GGCTGAAAAC
4921 CTATGCCCAC CTGTTCGACG ACAAAGTGAT GAAGCAGCTGAAGCGGCGGA GATACACCGG
4981 CTGGGGCAGG CTGAGCCGGA AGCTGATCAA CGGCATCCGGGACAAGCAGT CCGGCAAGAC
5041 AATCCTGGAT TTCCTGAAGT CCGACGGCTT CGCCTGCAGAAACTTCATGC AGCTGATCCA
5101 CGACGACAGC CTGACCTTTA AAGAGGACAT CCAGAAAGCCCAGGTGTCCG GCCAGGGCGA
5161 TAGCCTGCAC GAGCACATTG CCAATCTGGC CGGCAGCCCCGCCATTAAGA AGGGCATCCT
5221 GCAGACAGTG AAGGTGGTGG ACGAGCTCGT GAAAGTGATGGGCCGGCACA AGCCCGAGAA
5281 CATCGTGATC GAAATGGCCA GAGAGAACCA GATCACCCAGAAGGGACAGA AGAACAGCCG
5341 CGAGAGAATG AAGCGGATCG AAGAGGGCAT CAAAGAGCTGGGCAGCCAGA TCCTGAAAGA
5401 ACACCCCGTG GAAAACACCC AGCTGCAGAA CGAGAAGCTGTACCTGTACT ACCTGCAGAA
5461 TGGGCGGGAT ATGTACGTGG ACCAGGAACT GGACATCAACCGGCTGTCCG ACTACGATGT
5521 GGACCATATC GTGCCTCAGA GCTTTCTGAA GGACGACTCCATCGACAACA AGGTGCTGAC
5581 CAGAAGCGAC AAGAACCGGG GCAAGAGCGA CAACGTGCCCTCCGAAGAGG TCGTGAAGAA
5641 GATGAAGAAC TACTGGCGGC AGCTGCTGAA CGCCAAGCTGATTACCCAGA GAAAGTTCGA
5701 CAATCTGACC AAGGCCGAGA GAGGCGGCCT GAGCGAACTGGATAAGGCCA TGTTCATCAA
5761 GAGACAGCTG GTGGAAACCC GGCAGATCAC AAAGCACGTGGCACAGATCC TGGACTCCCG
5821 GATGAACACT AAGTACGACG AGAATGACAA GCTGATCCGGGAAGTGAAAG TGATCACCCT
5881 GAAGTCCAAG CTGGTGTCCG ATTTCCGGAA GGATTTCCAGTTTTACAAAG TGCGCGAGAT
5941 CAACAAATAC CACCACGCCC ACGACGCCTA CCTGAACGCGTCGTGGGAA CCGCCCTGAT
6001 CAAAAAGTAC CCTAAGCTGG AAAGCGAGTT CGTGTACGGCGACTACAAGG TGTACGACGT
6061 GCGGAAGATG ATCGCCAAGA GCGAGCAGGA AATCGGCAAGCTACCGCCA AGTACTTCTT
6121 CTACAGCAAC ATCATGAACT TTTTCAAGAC CGAGATTACCCTGGCCAACG GCGAGATCCG
6181 GAAGCGGCCT CTGATCGAGA CAAACGGCGA AACCGGGGAGATCGTGTGGG ATAAGGGCCG
6241 GGATTTTGCC ACCGTGCGGA AAGTGCTGAG CATGCCCCAAGTGAATATCG TGAAAAAGAC
6301 CGAGGTGCAG ACAGGCGGCT TCAGCAAAGA GTCTATCCTGCCCAAGAGGA ACAGCGATAA
6361 GCTGATCGCC AGAAAGAAGG ACTGGGACCC TAAGAAGTACGGCGGCTTCG ACAGCCCCAC
6421 CGTGGCCTAT TCTGTGCTGG TGGTGGCCAA AGTGGAAAAGGGCAAGTCCA AGAAACTGAA
6481 GAGTGTGAAA GAGCTGCTGG GGATCACCAT CATGGAAAGAAGCAGCTTCG AGAAGAATCC
6541 CATCGACTTT CTGGAAGCCA AGGGCTACAA AGAAGTGAAAAAGGACCTGA TCATCAAGCT
6601 GCCTAAGTAC TCCCTGTTCG AGCTGGAAAA CGGCCGGAAGAGAATGCTGG CCTCTGCCGG
6661 CGAACTGCAG AAGGGAAACG AACTGGCCCT GCCCTCCAAATATGTGAACT TCCTGTACCT
6721 GGCCAGCCAC TATGAGAAGC TGAAGGGCTC CCCCGAGGATAATGAGCAGA AACAGCTGTT
6781 TGTGGAACAG CACAAGCACT ACCTGGACGA GATCATCGAGCAGATCAGCG AGTTCTCCAA
6841 GAGAGTGATC CTGGCCGACG CTAATCTGGA CAAAGTGCTGTCCGCCTACA ACAAGCACCG
6901 GGATAAGCCC ATCAGAGAGC AGGCCGAGAATATCATCCACCTGTTTACCC TGACCAATCT
6961 GGGAGCCCCT GCCGCCTTCA AGTACTTTGA CACCACCATCGACCGGAAGA GGTACACCAG
7021 CACCAAAGAG GTGCTGGACG CCACCCTGAT CCACCAGAGCATCACCGGCCTGTACGAGAC
7081 ACGGATCGAC CTGTCTCAGC TGGGAGGCGA Caaaaggccg gcggccacga aaaaggccgg
7141 ccaggcaaaa aagaaaaagt aagaattcct agagctcgct gatcagcctc gactgtgcct
7201 tctagttgcc agccatctgt tgtttgcccc tcccccgtgc cttccttgac cctggaaggt
7261 gccactccca ctgtcctttc ctaataaaat gaggaaattg catcgcattg tctgagtagg
7321 tgtcattcta ttctgggggg tggggtgggg caggacagca agggggagga ttgggaagag
7381 aatagcaggc atgctgggga gcggccgcag gaacccctag tgatggagtt ggccactccc
7441 tctctgcgcg ctcgctcgct cactgaggcc gggcgaccaa aggtcgcccg acgcccgggc
7501 tttgcccggg cggcctcagt gagcgagcga gcgcgcagct gcctgcaggg gcgcctgatg
7561 cggtattttc tccttacgca tctgtgcggt atttcacacc gcatacgtca aagcaaccat
7621 agtacgcgcc ctgtagcggc gcattaagcg cggcgggtgt ggtggttacg cgcagcgtga
7681 ccgctacact tgccagcgcc ttagcgcccg ctcctttcgc tttcttccct tcctttctcg
7741 ccacgttcgc cggctttccc cgtcaagctc taaatcgggg gctcccttta gggttccgat
7801 ttagtgcttt acggcacctc gaccccaaaa aacttgattt gggtgatggt tcacgtagtg
7861 ggccatcgcc ctgatagacg gtttttcgcc ctttgacgtt ggagtccacg ttctttaata
7921 gtggactctt gttccaaact ggaacaacac tcaactctat ctcgggctat tcttttgatt
7981 tataagggat tttgccgatt tcggtctatt ggttaaaaaa tgagctgatt taacaaaaat
8041 ttaacgcgaa ttttaacaaa atattaacgt ttacaatttt atggtgcact ctcagtacaa
8101 tctgctctga tgccgcatag ttaagccagc cccgacaccc gccaacaccc gctgacgcgc
8161 cctgacgggc ttgtctgctc ccggcatccg cttacagaca agctgtgacc gtct** (SEQ ID NO: 1298)

LZ3-Cas9 nucleotide (4,101 nt) and amino acid (1,367 aa) sequences

gacaagaagtacagcatcggcctggacatcggcaccaactctgtgggctg
ggccgtgatcaccgacgagtacaaggtgcccagcaagaaattcaaggtgc
tgggcaacaccgaccggcacagcatcaagaagaacctgatcggagccctg
ctgttcgacagcggcgaaacagccgaggccacccggctgaagagaaccgc
cagaagaagatacaccagacggaagaaccggatctgctatctgcaagaga
tcttcagcaacgagatggccaaggtggacgacagcttcttccacagactg
gaagagtccttcctggtggaagaggataagaagcacgagcggcaccccat
cttcggcaacatcgtggacgaggtggcctaccacgagaagtaccccacca
tctaccacctgagaaagaaactggtggacagcaccgacaaggccgacctg
cggctgatctatctggccctggcccacatgatcaagttccggggccactt
cctgatcgagggcgacctgaaccccgacaacagcgacgtggacaagctgt
tcatccagctggtgcagacctacaaccagctgttcgaggaaaaccccatc
aacgccagcggcgtggacgccaaggccatcctgtctgccagactgagcaa
gagcagacggctggaaaatctgatcgcccagctgcccggcgagaagaaga
atggcctgttcggaaacctgattgccctgagcctgggcctgacccccaac
ttcaagagcaacttcgacctggccgaggatgccaaactgcagctgagcaa
ggacacctacgacgacgacctggacaacctgctggcccagatcggcgacc
agtacgccgacctgtttctggccgccaagaacctgtccgacgccatcctg
ctgagcgacatcctgagagtgaacaccgagatcaccaaggcccccctgag
cgcctctatgatcaagagatacgacgagcaccaccaggacctgaccctgc
tgaaagctctcgtgcggcagcagctgcctgagaagtacaaagagattttc
ttcgaccagagcaagaacggctacgccggctacattgacggcggagccag
ccaggaagagttctacaagttcatcaagcccatcctggaaaagatggacg
gcaccgaggaactgctcgtgaagctgaacagagaggacctgctgcggaag
cagcggaccttcgacaacggcagcatcccccaccagatccacctgggaga
gctgcacgccattctgcggcggcaggaagatttttacccattcctgaagg
acaaccgggaaaagatcgagaagatcctgaccttccgcatcccctactac
gtgggccctctggccaggggaaacagcagattcgcctggatgaccagaaa
gagcgaggaaaccatcaccccctggaacttcgaggaagtggtggacaagg
gcgcttccgcccagagcttcatcgagcggatgaccaacttcgataagaac
ctgcccaacgagaaggtgctgcccaagcacagcctgctgtacgagtactt
caccgtgtataacgagctgaccaaagtgaaatacgtgaccgagggaatga
gaaagcccgccttcctgagcggcgagcagaaaaaggccatcgtggacctg
ctgttcaagaccaaccggaaagtgaccgtgaagcagctgaaagaggacta
cttcaagaaaatcgagtgcttcgactccgtggaaatctccggcgtggaag
atcggttcaacgcctccctgggcacataccacgatctgctgaaaattatc
aaggacaaggacttcctggacaatgaggaaaacgaggacattctggaaga
tatcgtgctgaccctgacactgtttgaggacagagagatgatcgaggaac
ggctgaaaacctatgcccacctgttcgacgacaaagtgatgaagcagctg
aagcggcggagatacaccggctggggcaggctgagccggaagctgatcaa
cggcatccgggacaagcagtccggcaagacaatcctggatttcctgaagt
ccgacggcttcgcctgcagaaacttcatgcagctgatccacgacgacagc
ctgacctttaaagaggacatccagaaagcccaggtgtccggccagggcga
tagcctgcacgagcacattgccaatctggccggcagccccgccattaaga
agggcatcctgcagacagtgaaggtggtggacgagctcgtgaaagtgatg
ggccggcacaagcccgagaacatcgtgatcgaaatggccagagagaacca
gatcacccagaagggacagaagaacagccgcgagagaatgaagcggatcg
aagagggcatcaaagagctgggcagccagatcctgaaagaacaccccgtg
gaaaacacccagctgcagaacgagaagctgtacctgtactacctgcagaa
tgggcgggatatgtacgtggaccaggaactggacatcaaccggctgtccg
actacgatgtggaccatatcgtgcctcagagctttctgaaggacgactcc
atcgacaacaaggtgctgaccagaagcgacaagaaccggggcaagagcga
caacgtgccctccgaagaggtcgtgaagaagatgaagaactactggcggc
agctgctgaacgccaagctgattacccagagaaagttcgacaatctgacc
aaggccgagagaggcggcctgagcgaactggataaggccatgttcatcaa
gagacagctggtggaaacccggcagatcacaaagcacgtggcacagatcc
tggactcccggatgaacactaagtacgacgagaatgacaagctgatccgg
gaagtgaaagtgatcaccctgaagtccaagctggtgtccgatttccggaa
ggatttccagttttacaaagtgcgcgagatcaacaaataccaccacgccc
acgacgcctacctgaacgccgtcgtgggaaccgccctgatcaaaaagtac
cctaagctggaaagcgagttcgtgtacggcgactacaaggtgtacgacgt
gcggaagatgatcgccaagagcgagcaggaaatcggcaaggctaccgcca
agtacttcttctacagcaacatcatgaactttttcaagaccgagattacc
ctggccaacggcgagatccggaagcggcctctgatcgagacaaacggcga
aaccggggagatcgtgtgggataagggccgggattttgccaccgtgcgga
aagtgctgagcatgccccaagtgaatatcgtgaaaaagaccgaggtgcag
acaggcggcttcagcaaagagtctatcctgcccaagaggaacagcgataa
gctgatcgccagaaagaaggactgggaccctaagaagtacggcggcttcg
acagccccaccgtggcctattctgtgctggtggtggccaaagtggaaaag
ggcaagtccaagaaactgaagagtgtgaaagagctgctggggatcaccat
catggaaagaagcagcttcgagaagaatcccatcgactttctggaagcca
agggctacaaagaagtgaaaaaggacctgatcatcaagctgcctaagtac
tccctgttcgagctggaaaacggccggaagagaatgctggcctctgccgg
cgaactgcagaagggaaacgaactggccctgccctccaaatatgtgaact
tcctgtacctggccagccactatgagaagctgaagggctcccccgaggat
aatgagcagaaacagctgtttgtggaacagcacaagcactacctggacga
gatcatcgagcagatcagcgagttctccaagagagtgatcctggccgacg
ctaatctggacaaagtgctgtccgcctacaacaagcaccgggataagccc
atcagagagcaggccgagaatatcatccacctgtttaccctgaccaatct
gggagcccctgccgccttcaagtactttgacaccaccatcgaccggaaga
ggtacaccagcaccaaagaggtgctggacgccaccctgatccaccagagc
atcaccggcctgtacgagacacggatcgacctgtctcagctgggaggcga
c (SEQ ID NO: 1299)

DKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKIL TFRIPY
YVGPLARGNSRF A WMTRKSEETITPWNFEEVVDKGASAQSFIERMTNF
DKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVM
KQLKRRRYTGWGRLSRKLINGIRDKQSGKTILDFLKSDGFACRNFMQLIH
DDSLTFKEDIQKAQVSGQGDSLHEHIANLAGSPAIKKGILQTVKVVDELV
KVMGRHKPENIVIEMARENQITQKGQKNSRERMKRIEEGIKELGSQILKE
HPVENTQLQNEKLYLYYLQNGRDMYVDQELDINRLSDYDVDHIVPQSFLK
DDSIDNKVLTRSDKNRGKSDNVPSEEVVKKMKNYWRQLLNAKLITQRKFD
NLTKAERGGLSELDAKAMFIKRQLVETRQITKHVAQILDSRMNTKYDEND
KLIREVKVITLKSKLVSDFRKDFQFYKVREINKYHHAHDAYLNAVVGTAL
IKKYPKLESEFVYGDYKVVRKMIAKSEQEIGKATAKYFFYSNIMNFFKTE
ITLANGEIRKRPLIETNGETGEIVWDKGRDFATVRKVLSMPQVNIVKKTE
VQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLP
KYSLFELENGRKRMLASAGELQKGNELALPSKYVNFLYLASHYEKLKGSP
EDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIH
QSITGLYETRIDLSQLGGD (SEQ ID NO:1300)

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A composition comprising an engineered Cas protein that comprises a RuvC domain and a HNH domain, wherein the engineered Cas protein has a nuclease activity substantially the same as a wildtype counterpart Cas protein and a specificity of at least between 15% and 30% higher than the wildtype counterpart Cas protein.

2. The composition of claim 1, wherein the engineered Cas protein further comprises a first linker domain and a second linker domain that connects the RuvC domain and the HNH domain, and the engineered Cas protein comprises mutations in the RuvC domain, the first linker domain, and the second linker domain compared to the wildtype counterpart Cas protein.

3. The composition of claim 1, wherein the engineered Cas protein is an engineered class 2, Type II Cas protein.

4. The composition of claim 3, wherein the engineered class 2, Type II Cas protein is an engineered Cas9 protein.

5. The composition of claim 4, wherein the engineered Cas9 protein comprises one or more mutations of amino acids corresponding to the following amino acids of SpCas9: N690, T769, G915, and N980 based on the amino acids at the sequence positions of wildtype SpCas9, optionally wherein the mutations of amino acids correspond to N690C, T769I, G915M, N980K.

6. The composition of claim 4, wherein the engineered Cas9 protein comprises SEQ ID NO: 1300 or is encoded by SEQ ID NO: 1299.

7. The composition of claim 1, wherein the engineered Cas protein is capable of generating a staggered 1 nucleotide overhang on a target polynucleotide.

8. The composition of claim 7, wherein the 1 nucleotide overhang is a 5′ overhang.

9. The composition of claim 7, wherein the engineered Cas protein has a +1 insertion frequency different from the wildtype counterpart Cas protein.

10. The composition of claim 9, wherein the +1 insertion frequency when a guanine is present in the -2 position with respect to a PAM, is higher than the +1 insertion frequency when a thymidine, a cytidine, or an adenine is present in the -2 position with respect to the PAM.

11. The composition of claim 1, further comprising: i) one or more guide sequences capable of complexing with the engineered Cas protein and directing binding of the guide-Cas protein complex to one or more target polynucleotides; and ii) a donor polynucleotide.

12. The composition of claim 11, wherein the donor polynucleotide:
a. introduces one or more mutations to the target polynucleotide;
b. corrects a premature stop codon in the target polynucleotide;
c. disrupts a splicing site;
d. restores a splicing site;
e. corrects a naturally occurring 1-bp deletion;
f. compensates for a naturally occurring frameshift mutation; or
g. a combination thereof.

13. The composition of claim 12, wherein the one or more mutations introduced by the donor polynucleotide comprises substitutions, deletions, insertions, or a combination thereof.

14. The composition of claim 12, wherein the one or more mutations causes a shift in an open reading frame in the target polynucleotide.

15. An engineered cell comprising the composition of any one of claims 1-14.

16. A method of modifying a target polynucleotide sequence in a cell, comprising introducing the composition of any one of claims 1-14 to the cell.

17. The method of any one of claims 1-14, wherein the cell is a prokaryotic cell, a eukaryotic cell, a mammalian cell, a plant cell, a cell of a non-human primate, or a human cell.

18. A method comprising:
a. introducing into one or more cells:
i. a Cas protein or a coding sequence thereof;
ii. a plurality of guide RNAs or coding sequences thereof; and
iii. a donor sequence;
wherein the guide RNAs are capable of directing the Cas protein to cleave target polynucleotides in the one or more cells and the donor sequence is inserted into the cleaved target polynucleotides, thereby generating a plurality of donor-integrated target polynucleotides;
b. tagmenting the donor-integrated target polynucleotides with a transposase or a transposon complex;
c. sequencing the tagmented donor-integrated target polynucleotides; and
d. analyzing specificity and activity of the Cas protein based on the sequences of the tagmented donor-integrated target polynucleotides.

19. The method of claim 18, comprising introducing one or more polynucleotides into one or more cells, the one or more polynucleotides comprising: a coding sequence of a Cas protein; a plurality of guide RNAs or coding sequences thereof; and a donor sequence.

20. The method of claim 18, wherein the donor sequence is a double-stranded DNA sequence.

21. The method of claim 18, wherein the donor sequence comprises one or more modifications.

22. The method of claim 21, wherein the one or more modifications comprises 5′ phosphorylation, phosphorothioate stabilization, or a combination thereof.

23. The method of claim 18, wherein the tagmenting is performed using a Tn5 transposase or transposon complex.

24. The method of claim 23, wherein the Tn5 transposase is a hyperactive variant.

25. The method of claim 18, further comprising, prior to (b), lysing the one or more cells.

26. The method of claim 18, wherein the sequencing comprises performing nested PCR.

27. The method of claim 18, wherein (i), (ii), and (iii) are introduced using a viral vector.


SEQUENCE LISTING (1300 sequences)

SEQ ID NO: 1 - DNA, 54 bp, Artificial Sequence
  Synthetic
  misc_feature (1)..(1): /5phos/ - 5' phosphorylation
  misc_feature (6)..(6): h is a, c, or t
  misc_feature (9)..(9): n is a, c, g, or t
  misc_feature (12)..(12): n is a, c, g, or t
  misc_feature (15)..(15): n is a, c, g, or t
  misc_feature (18)..(18): y is t or c
  misc_feature (21)..(21): n is a, c, g, or t
  misc_feature (24)..(24): n is a, c, g, or t
  misc_feature (27)..(27): n is a, c, g, or t
  misc_feature (30)..(30): n is a, c, g, or t
ggcgghtcnggnggnagyggnggnggntcngtgagcaagggcgaggaggataac

SEQ ID NO: 2 - DNA, 26 bp, Homo sapiens
  misc_feature (1)..(23): n is a, c, g, or t
nnnnnnnnnnnnnnnnnnnnnnnaac

SEQ ID NO: 3 - DNA, 24 bp, Homo sapiens
cttatcgtcgtcatccttgtaatc

SEQ ID NO: 4 - DNA, 24 bp, Homo sapiens
gattacaaggatgacgacgataag

SEQ ID NO: 5 - DNA, 27 bp, Homo sapiens
gacggcggtctccgtcgtcaggatcat

SEQ ID NO: 6 - DNA, 27 bp, Homo sapiens
gacggagaccgccgtcgtcgacaagcc

SEQ ID NO: 7 - DNA, 14 bp, Artificial Sequence
  Synthetic oligonucleotide
  MISC_FEATURE (1)..(1): /5phos/ - 5' phosphorylation
  MISC_FEATURE (14)..(14): /3ddC/ - dideoxycytidine 3' chain terminator
ctgtctcttataca

SEQ ID NO: 8 - DNA, 34 bp, Artificial Sequence
  Synthetic oligonucleotide
gtctcgtgggctcggagatgtgtataagagacag

SEQ ID NO: 9 - DNA, 47 bp, Artificial Sequence
  Synthetic
  MISC_FEATURE (1)..(1): /5phos/ - 5' phosphorylation
gttgtgagcaagggcgaggaggataacgcctctctcccagcgactat

SEQ ID NO: 10 - DNA, 47 bp, Artificial Sequence
  Synthetic
  MISC_FEATURE (1)..(1): /5phos/ - 5' phosphorylation
atagtcgctgggagagaggcgttatcctcctcgcccttgctcacaac

SEQ ID NO: 11 - DNA, 23 bp, Artificial Sequence
  Synthetic oligonucleotide
gtcgctgggagagaggcgttatc

SEQ ID NO: 12 - DNA, 93 bp, Artificial Sequence
  Synthetic
aatgatacggcgaccaccgagatctacactatagcctacactctttccctacacgacgctcttccgatctttatcctcctcgcccttgctcac

SEQ ID NO: 13 - DNA, 55 bp, Artificial Sequence
  Synthetic
caagcagaagacggcatacgagatcgagtaatgtctcgtgggctcggagatgtgt

SEQ ID NO: 14 - DNA, 55 bp, Artificial Sequence
  Synthetic
caagcagaagacggcatacgagattctccggagtctcgtgggctcggagatgtgt

SEQ ID NO: 15 - DNA, 55 bp, Artificial Sequence
  Synthetic
caagcagaagacggcatacgagataatgagcggtctcgtgggctcggagatgtgt

SEQ ID NO: 16 - DNA, 55 bp, Artificial Sequence
  Synthetic
caagcagaagacggcatacgagatggaatctcgtctcgtgggctcggagatgtgt

SEQ ID NO: 17 - DNA, 55 bp, Artificial Sequence
  Synthetic
caagcagaagacggcatacgagatttctgaatgtctcgtgggctcggagatgtgt

SEQ ID NO: 18 - DNA, 55 bp, Artificial Sequence
  Synthetic
caagcagaagacggcatacgagatacgaattcgtctcgtgggctcggagatgtgt

SEQ ID NO: 19 - DNA, 55 bp, Artificial Sequence
  Synthetic
caagcagaagacggcatacgagatagcttcaggtctcgtgggctcggagatgtgt

SEQ ID NO: 20 - DNA, 55 bp, Artificial Sequence
  Synthetic
caagcagaagacggcatacgagatgcgcattagtctcgtgggctcggagatgtgt

SEQ ID NO: 21 - DNA, 55 bp, Artificial Sequence
  Synthetic
caagcagaagacggcatacgagatcatagccggtctcgtgggctcggagatgtgt

SEQ ID NO: 22 - DNA, 55 bp, Artificial Sequence
  Synthetic
caagcagaagacggcatacgagatttcgcggagtctcgtgggctcggagatgtgt

SEQ ID NO: 23 - DNA, 55 bp, Artificial Sequence
  Synthetic
caagcagaagacggcatacgagatgcgcgagagtctcgtgggctcggagatgtgt

SEQ ID NO: 24 - DNA, 55 bp, Artificial Sequence
  Synthetic
caagcagaagacggcatacgagatctatcgctgtctcgtgggctcggagatgtgt

SEQ ID NO: 25 - DNA, 55 bp, Artificial Sequence
  Synthetic
caagcagaagacggcatacgagattgtagtgcgtctcgtgggctcggagatgtgt

SEQ ID NO: 26 - DNA, 55 bp, Artificial Sequence
  Synthetic
caagcagaagacggcatacgagatgcgtcgacgtctcgtgggctcggagatgtgt

SEQ ID NO: 27 - DNA, 55 bp, Artificial Sequence
  Synthetic
caagcagaagacggcatacgagatggtcttctgtctcgtgggctcggagatgtgt

SEQ ID NO: 28 - DNA, 55 bp, Artificial Sequence
  Synthetic
caagcagaagacggcatacgagataaatgtccgtctcgtgggctcggagatgtgt

SEQ ID NO: 29 - DNA, 55 bp, Artificial Sequence
  Synthetic
caagcagaagacggcatacgagatgttgaaacgtctcgtgggctcggagatgtgt

SEQ ID NO: 30 - DNA, 55 bp, Artificial Sequence
  Synthetic
caagcagaagacggcatacgagattctttacggtctcgtgggctcggagatgtgt

SEQ ID NO: 31 - DNA, 55 bp, Artificial Sequence
  Synthetic
caagcagaagacggcatacgagatatgcctgggtctcgtgggctcggagatgtgt

SEQ ID NO: 32 - DNA, 55 bp, Artificial Sequence
  Synthetic
caagcagaagacggcatacgagatcaataagggtctcgtgggctcggagatgtgt

SEQ ID NO: 33 - DNA, 55 bp, Artificial Sequence
  Synthetic
caagcagaagacggcatacgagatcgccgtaagtctcgtgggctcggagatgtgt

SEQ ID NO: 34 - DNA, 55 bp, Artificial Sequence
  Synthetic
caagcagaagacggcatacgagattaaggcttgtctcgtgggctcggagatgtgt

SEQ ID NO: 35 - DNA, 55 bp, Artificial Sequence
  Synthetic
caagcagaagacggcatacgagatttgctgccgtctcgtgggctcggagatgtgt

SEQ ID NO: 36 - DNA, 55 bp, Artificial Sequence
  Synthetic
caagcagaagacggcatacgagatctcaatgtgtctcgtgggctcggagatgtgt

SEQ ID NO: 37 - DNA, 90 bp, Artificial Sequence
  Synthetic
aatgatacggcgaccaccgagatctacactatagcctacactctttccctacacgacgctcttccgatctcttatcgtcgtcatccttgt

SEQ ID NO: 38 - DNA, 90 bp, Artificial Sequence
  Synthetic
aatgatacggcgaccaccgagatctacactatagcctacactctttccctacacgacgctcttccgatctgattacaaggatgacgacga

SEQ ID NO: 39 - DNA, 21 bp, Artificial Sequence
  Synthetic oligonucleotide
ggcttgtcgacgacggcggtc

SEQ ID NO: 40 - DNA, 91 bp, Artificial Sequence
  Synthetic
aatgatacggcgaccaccgagatctacactatagcctacactctttccctacacgacgctcttccgatctgacggcggtctccgtcgtcag

SEQ ID NO: 41 - DNA, 22 bp, Artificial Sequence
  Synthetic oligonucleotide
atgatcctgacgacggagaccg

SEQ ID NO: 42 - DNA, 91 bp, Artificial Sequence
  Synthetic
aatgatacggcgaccaccgagatctacactatagcctacactctttccctacacgacgctcttccgatctgacggagaccgccgtcgtcga

SEQ ID NO: 43 - DNA, 23 bp, Artificial Sequence
  Synthetic oligonucleotide
gagtccgagcagaagaagaaggg

SEQ ID NO: 44 - DNA, 23 bp, Artificial Sequence
  Synthetic oligonucleotide
gagtccgagcagaagaagaaggg

SEQ ID NO: 45 - DNA, 23 bp, Artificial Sequence
  Synthetic oligonucleotide
gagttagagcagaagaagaaagg

SEQ ID NO: 46 - DNA, 23 bp, Artificial Sequence
  Synthetic oligonucleotide
gagtctaagcagaagaagaagag

SEQ ID NO: 47 - DNA, 23 bp, Artificial Sequence
  Synthetic oligonucleotide
gaggccgagcagaagaaagacgg

SEQ ID NO: 48 - DNA, 23 bp, Artificial Sequence
  Synthetic oligonucleotide
gagtcctagcaggagaagaagag

SEQ ID NO: 49 - DNA, 23 bp, Artificial Sequence
  Synthetic oligonucleotide
aagtctgagcacaagaagaatgg

SEQ ID NO: 50 - DNA, 23 bp, Artificial Sequence
  Synthetic oligonucleotide
gagtccgggaaggagaagaaagg

SEQ ID NO: 51 - DNA, 23 bp, Artificial Sequence
  Synthetic oligonucleotide
gagccggagcagaagaaggaggg

SEQ ID NO: 52 - DNA, 23 bp, Artificial Sequence
  Synthetic oligonucleotide
aagtccgaggagaggaagaaagg

SEQ ID NO: 53 - DNA, 23 bp, Artificial Sequence
  Synthetic oligonucleotide
gaatccaagcaggagaagaagga

SEQ ID NO: 54 - DNA, 23 bp, Artificial Sequence
  Synthetic oligonucleotide
acgtctgagcagaagaagaatgg

SEQ ID NO: 55 - DNA, 23 bp, Artificial Sequence
  Synthetic oligonucleotide
gagtagcgagcagagaagaagga

SEQ ID NO: 56 - DNA, 23 bp, Artificial Sequence
  Synthetic oligonucleotide
aagtcccggcagaggaagaaggg

SEQ ID NO: 57 - DNA, 23 bp, Artificial Sequence
  Synthetic oligonucleotide
tcatccaagcagaagaagaagag

SEQ ID NO: 58 - DNA, 23 bp, Artificial Sequence
  Synthetic oligonucleotide
gagcacgagcaagagaagaaggg

SEQ ID NO: 59 - DNA, 23 bp, Artificial Sequence
  Synthetic oligonucleotide
gagtctaagcaggagaataaagg

SEQ ID NO: 60 - DNA, 23 bp, Artificial Sequence
  Synthetic oligonucleotide
gaggccgggcaggagaaggaggg

SEQ ID NO: 61 - DNA, 23 bp, Artificial Sequence
  Synthetic oligonucleotide
aagtcagagcagaaagaaggagg

SEQ ID NO: 62 - DNA, 23 bp, Artificial Sequence
  Synthetic oligonucleotide
aagcccgagcaaaggaagaaagg

SEQ ID NO: 63 - DNA, 23 bp, Artificial Sequence
  Synthetic oligonucleotide
aagcccgagcagaagaagttgag

SEQ ID NO: 64 - DNA, 23 bp, Artificial Sequence
  Synthetic oligonucleotide
ggtgagtgagtgtgtgcgtgtgg

SEQ ID NO: 65 - DNA, 23 bp, Artificial Sequence
  Synthetic oligonucleotide
agtgagtgagtgtgtgtgtgggg

SEQ ID NO: 66 - DNA, 23 bp, Artificial Sequence
  Synthetic oligonucleotide
agagagtgagtgtgtgcatgagg

SEQ ID NO: 67 - DNA, 23 bp, Artificial Sequence
  Synthetic oligonucleotide
ggtgagtgagtgtgtgcgtgtgg

SEQ ID NO: 68 - DNA, 23 bp, Artificial Sequence
  Synthetic oligonucleotide
tgtgggtgagtgtgtgcgtgagg

SEQ ID NO: 69 - DNA, 23 bp, Artificial Sequence
  Synthetic oligonucleotide
gctgagtgagtgtatgcgtgtgg

SEQ ID NO: 70 - DNA, 23 bp, Artificial Sequence
  Synthetic oligonucleotide
ggtgagtgagtgcgtgcgggtgg

SEQ ID NO: 71 - DNA, 23 bp, Artificial Sequence
  Synthetic oligonucleotide
gttgagtgaatgtgtgcgtgagg

SEQ ID NO: 72 - DNA, 23 bp, Artificial Sequence
  Synthetic oligonucleotide
agtgaatgagtgtgtgtgtgtgg

SEQ ID NO: 73 - DNA, 23 bp, Artificial Sequence
  Synthetic oligonucleotide
tgtgagtaagtgtgtgtgtgtgg

SEQ ID NO: 74 - DNA, 23 bp, Artificial Sequence
  Synthetic oligonucleotide
actgtgtgagtgtgtgcgtgagg

SEQ ID NO: 75 - DNA, 23 bp, Artificial Sequence
  Synthetic oligonucleotide
agcgagtgggtgtgtgcgtgggg

SEQ ID NO: 76 - DNA, 23 bp, Artificial Sequence
  Synthetic oligonucleotide
agtgtgtgagtgtgtgcgtgtgg

SEQ ID NO: 77 - DNA, 23 bp, Artificial Sequence
  Synthetic oligonucleotide
tgtgggtgagtgtgtgcgtgaga

SEQ ID NO: 78 - DNA, 23 bp, Artificial Sequence
  Synthetic oligonucleotide
cgcgagtgagtgtgtgcgcgggg

SEQ ID NO: 79 - DNA, 23 bp, Artificial Sequence
  Synthetic oligonucleotide
ggtgagtgagtgtgtgtgtgagg

SEQ ID NO: 80 - DNA, 23 bp, Artificial Sequence
  Synthetic oligonucleotide
ctggagtgagtgtgtgtgtgtgg

SEQ ID NO: 81 - DNA, 23 bp, Artificial Sequence
  Synthetic oligonucleotide
agcgagtgagtgtgtgtgtgggg

SEQ ID NO: 82 - DNA, 23 bp, Artificial Sequence
  Synthetic oligonucleotide
ggtgagtcagtgtgtgagtgagg

SEQ ID NO: 83 - DNA, 23 bp, Artificial Sequence
  Synthetic oligonucleotide
ggtgtgtgagtgtgtgtgtgtgg

SEQ ID NO: 84 - DNA, 23 bp, Artificial Sequence
  Synthetic oligonucleotide
tgtgagtgagtgtgtgtgtgtga

SEQ ID NO: 85 - DNA, 23 bp, Artificial Sequence
  Synthetic oligonucleotide
tgtgagtgagtgtgtgcatgggg

SEQ ID NO: 86 - DNA, 23 bp, Artificial Sequence
  Synthetic oligonucleotide
agtgagtgagtgagtgagtgagg

SEQ ID NO: 87 - DNA, 23 bp, Artificial Sequence
  Synthetic oligonucleotide
tgtgggtgagcatgtgcgtgagg

SEQ ID NO: 88 - DNA, 23 bp, Artificial Sequence
  Synthetic oligonucleotide
gtagagtgagtgtgtgtgtgtgg

SEQ ID NO: 89 - DNA, 23 bp, Artificial Sequence
  Synthetic oligonucleotide
ggtggatgagtgtgtgtgtgggg

SEQ ID NO: 90 - DNA, 23 bp, Artificial Sequence
  Synthetic oligonucleotide
tgtgagtgagtgtgtgcgtgtga

SEQ ID NO: 91 - DNA, 23 bp, Artificial Sequence
  Synthetic oligonucleotide
catgagtgagtgtgtgggtgggg

SEQ ID NO: 92 - DNA, 23 bp, Artificial Sequence
  Synthetic oligonucleotide
agtgagtatgtgtgtgtgtgggg

SEQ ID NO: 93 - DNA, 23 bp, Artificial Sequence
  Synthetic oligonucleotide
gatgagtgagtgagtgagtgggg

SEQ ID NO: 94 - DNA, 23 bp, Artificial Sequence
  Synthetic oligonucleotide
tgtgagtgagtgtgtgtgtgtga

SEQ ID NO: 95 - DNA, 23 bp, Artificial Sequence
  Synthetic oligonucleotide
ggagagtgagtgtgtgtgtgaga

SEQ ID NO: 96 - DNA, 23 bp, Artificial Sequence
  Synthetic oligonucleotide
ggtgagtaagtgtgagcgtaagg

SEQ ID NO: 97 - DNA, 23 bp, Artificial Sequence
  Synthetic oligonucleotide
ggtgagtgtgtgtgtgcatgtgg

SEQ ID NO: 98 - DNA, 23 bp, Artificial Sequence
  Synthetic oligonucleotide
agtgagtgagtgagtgagtgggg

SEQ ID NO: 99 - DNA, 23 bp, Artificial Sequence
  Synthetic oligonucleotide
gatgagtgtgtgtgtgtgtgagg

SEQ ID NO: 100 - DNA, 23 bp, Artificial Sequence
  Synthetic oligonucleotide
ggtgaatgagtgtgtgctctggg

SEQ ID NO: 101 - DNA, 23 bp, Artificial Sequence
  Synthetic oligonucleotide
agggagtgactgtgtgcgtgtgg

SEQ ID NO: 102 - DNA, 23 bp, Artificial Sequence
  Synthetic oligonucleotide
agtgagtgagtgtgtgtgtgaga

SEQ ID NO: 103 - DNA, 23 bp, Artificial Sequence
  Synthetic oligonucleotide
ggtgagcaagtgtgtgtgtgtgg

SEQ ID NO: 104 - DNA, 23 bp, Artificial Sequence
  Synthetic oligonucleotide
cgtgagtgagtgtgtacctgggg

SEQ ID NO: 105 - DNA, 23 bp, Artificial Sequence
  Synthetic oligonucleotide
ggtggatgactgtgtgtgtgggg

SEQ ID NO: 106 - DNA, 23 bp, Artificial Sequence
  Synthetic oligonucleotide
tgtgggtgagtgtgtgtgtgtgg

SEQ ID NO: 107 - DNA, 23 bp, Artificial Sequence
  Synthetic oligonucleotide
ggtgtatgagtgtgtgtgtgagg

SEQ ID NO: 108 - DNA, 23 bp, Artificial Sequence
  Synthetic oligonucleotide
tgtgagtgagtatgtacatgtgg

SEQ ID NO: 109 - DNA, 23 bp, Artificial Sequence
  Synthetic oligonucleotide
agtgagtaagtgagtgagtgagg

SEQ ID NO: 110 - DNA, 23 bp, Artificial Sequence
  Synthetic oligonucleotide
tgtgagtgggtgtgtgtgtgggg

SEQ ID NO: 111 - DNA, 23 bp, Artificial Sequence
  Synthetic oligonucleotide
aatgagtgagtgtgtgtgtgtga

SEQ ID NO: 112 - DNA, 23 bp, Artificial Sequence
  Synthetic oligonucleotide
ggtgagagagtgtgtgcgtagga

SEQ ID NO: 113 - DNA, 23 bp, Artificial Sequence
  Synthetic oligonucleotide
tgtgagtgagtgtgtgtgtgtga

SEQ ID NO: 114 - DNA, 23 bp, Artificial Sequence
  Synthetic oligonucleotide
agtgagaaagtgtgtgcatgcgg

SEQ ID NO: 115 - DNA, 23 bp, Artificial Sequence
  Synthetic oligonucleotide
agtgagtgagtgtgtgtgtgtga

SEQ ID NO: 116 - DNA, 23 bp, Artificial Sequence
  Synthetic oligonucleotide
tgtgagtgagtgtgtgtgtgtga

SEQ ID NO: 117 - DNA, 23 bp, Artificial Sequence
  Synthetic oligonucleotide
agtgagtgagtgtgagtgcgggg

SEQ ID NO: 118 - DNA, 23 bp, Artificial Sequence
  Synthetic oligonucleotide
ggagagtgagtgtgtgcatgtgc

SEQ ID NO: 119 - DNA, 23 bp, Artificial Sequence
  Synthetic oligonucleotide
ggtgtgtgggtgggggtgtgtgg

SEQ ID NO: 120 - DNA, 23 bp, Artificial Sequence
  Synthetic oligonucleotide
agggagtgagtgtgagagtgcgg

SEQ ID NO: 121 - DNA, 23 bp, Artificial Sequence
  Synthetic oligonucleotide
ggtgagagagtgtgtgcacgggg

SEQ ID NO: 122 - DNA, 23 bp, Artificial Sequence
  Synthetic oligonucleotide
tgtgagagagtgtgtgtgtggag

SEQ ID NO: 123 - DNA, 23 bp, Artificial Sequence
  Synthetic oligonucleotide
tgtgagagagagtgtgcgtgtgg

SEQ ID NO: 124 - DNA, 23 bp, Artificial Sequence
  Synthetic oligonucleotide
ggggggtgagtgtgtgtgtgggg

SEQ ID NO: 125 - DNA, 23 bp, Artificial Sequence
  Synthetic oligonucleotide
ggggaatgagtgtgtgcatggag

SEQ ID NO: 126 - DNA, 23 bp, Artificial Sequence
  Synthetic oligonucleotide
tgtgagtgagtgtgtgtgtggag

SEQ ID NO: 127 - DNA, 23 bp, Artificial Sequence
  Synthetic oligonucleotide
agtgaatgagtgtgtgcatgtga

SEQ ID NO: 128 - DNA, 23 bp, Artificial Sequence
  Synthetic oligonucleotide
ggtgagtgagtgagtgagtgagt

SEQ ID NO: 129 - DNA, 23 bp, Artificial Sequence
  Synthetic oligonucleotide
gatgagtgagtgtgtgagtgaga

SEQ ID NO: 130 - DNA, 23 bp, Artificial Sequence
  Synthetic oligonucleotide
ggtgtgtgagtgtgtgtgtgtga

SEQ ID NO: 131 - DNA, 23 bp, Artificial Sequence
  Synthetic oligonucleotide
agtgagtgagtgagtgagtgagg

SEQ ID NO: 132 - DNA, 23 bp, Artificial Sequence
  Synthetic oligonucleotide
ggtgtgtgagtgagtgtgtgtat

SEQ ID NO: 133 - DNA, 23 bp, Artificial Sequence
  Synthetic oligonucleotide
agtgagtgagtgagtgagtgagg

SEQ ID NO: 134 - DNA, 23 bp, Artificial Sequence
  Synthetic oligonucleotide
ggtgagtaggtgtgtgtgtgggg

SEQ ID NO: 135 - DNA, 23 bp, Artificial Sequence
  Synthetic oligonucleotide
ggtgagtgcgtgtgtgcgtgcgc

SEQ ID NO: 136 - DNA, 23 bp, Artificial Sequence
  Synthetic oligonucleotide
ggtgggtgaatgggtgcgtgggg

SEQ ID NO: 137 - DNA, 23 bp, Artificial Sequence
  Synthetic oligonucleotide
ggtgagtgagagtgtgtgtgtgg

SEQ ID NO: 138 - DNA, 23 bp, Artificial Sequence
  Synthetic oligonucleotide
ggtgagtgagtgagtgagtgagg

SEQ ID NO: 139 - DNA, 23 bp, Artificial Sequence
  Synthetic oligonucleotide
ggtgagtgtgtgtgtgggggggc

SEQ ID NO: 140 - DNA, 23 bp, Artificial Sequence
  Synthetic oligonucleotide
tgtgtgtgagtgtgtgagtgtga

SEQ ID NO: 141 - DNA, 23 bp, Artificial Sequence
  Synthetic oligonucleotide
ggtgggtgagtgagtgagtgagg

SEQ ID NO: 142 - DNA, 23 bp, Artificial Sequence
  Synthetic oligonucleotide
agagagtgagtgtgtgtgttggg

SEQ ID NO: 143 - DNA, 23 bp, Artificial Sequence
  Synthetic oligonucleotide
ggtgtgtgagtgtgtgcattggg

SEQ ID NO: 144 - DNA, 23 bp, Artificial Sequence
  Synthetic oligonucleotide
actgagtgagtgtgagtgtgagg

SEQ ID NO: 145 - DNA, 23 bp, Artificial Sequence
  Synthetic oligonucleotide
tgtgagtgagtgtgtgcatgtga

SEQ ID NO: 146 - DNA, 23 bp, Artificial Sequence
  Synthetic oligonucleotide
agtgagtgagtgtgagcgtgaag

SEQ ID NO: 147 - DNA, 23 bp, Artificial Sequence
  Synthetic oligonucleotide
tgtgagtgagtgtgggtgtgtgg

SEQ ID NO: 148 - DNA, 23 bp, Artificial Sequence
  Synthetic oligonucleotide
agagagtgagtgtgtgtgtgaga

SEQ ID NO: 149 - DNA, 23 bp, Artificial Sequence
  Synthetic oligonucleotide
agtgagtgagtgtgtgtgtgaag

SEQ ID NO: 150 - DNA, 23 bp, Artificial Sequence
  Synthetic oligonucleotide
tgtgagtgggtgtgtgcgggggg

SEQ ID NO: 151 - DNA, 23 bp, Artificial Sequence
  Synthetic oligonucleotide
tgtgagtgagtgcatgtgtgtgg

SEQ ID NO: 152 - DNA, 23 bp, Artificial Sequence
  Synthetic oligonucleotide
agagagtgagtgtgtgtgtgaga

SEQ ID NO: 153 - DNA, 23 bp, Artificial Sequence
  Synthetic oligonucleotide
ggtgagtgagtgagtgagtgagg

SEQ ID NO: 154 - DNA, 23 bp, Artificial Sequence
  Synthetic oligonucleotide
ggagagtgagtatgtgtgtgtgt

SEQ ID NO: 155 - DNA, 23 bp, Artificial Sequence
  Synthetic oligonucleotide
ggagagcgagtgtgtgcgtgtga

SEQ ID NO: 156 - DNA, 23 bp, Artificial Sequence
  Synthetic oligonucleotide
aatgagtgagtgtgtgagtggag

SEQ ID NO: 157 - DNA, 23 bp, Artificial Sequence
  Synthetic oligonucleotide
ggtggatgagtgaatgcgtgagg

SEQ ID NO: 158 - DNA, 23 bp, Artificial Sequence
  Synthetic oligonucleotide
attgagtgagtatgtgtgtgagg

SEQ ID NO: 159 - DNA, 23 bp, Artificial Sequence
  Synthetic oligonucleotide
tttgagtgagtgtgtgtgtgtgt

SEQ ID NO: 160 - DNA, 23 bp, Artificial Sequence
  Synthetic oligonucleotide
tgtgagtgagtgtgtgtgtgtga

SEQ ID NO: 161 - DNA, 23 bp, Artificial Sequence
  Synthetic oligonucleotide
tgtgagtgagagtgtgcattggg

SEQ ID NO: 162 - DNA, 23 bp, Artificial Sequence
  Synthetic oligonucleotide
agtgagggagtgagtgcgagggg

SEQ ID NO: 163 - DNA, 23 bp, Artificial Sequence
  Synthetic oligonucleotide
cttgagtgagagtgagcgtgagg

SEQ ID NO: 164 - DNA, 23 bp, Artificial Sequence
  Synthetic oligonucleotide
agtgtgtgagtgtgtgtgtgagg

SEQ ID NO: 165 - DNA, 23 bp, Artificial Sequence
  Synthetic oligonucleotide
ggtgtgtgagtgtgagtgtgggg

SEQ ID NO: 166 - DNA, 23 bp, Artificial Sequence
  Synthetic oligonucleotide
ggagagtcagtgtgtgcgtatgg

SEQ ID NO: 167 - DNA, 23 bp, Artificial Sequence
  Synthetic oligonucleotide
aatgagtgagtgtgtgagtgaag

SEQ ID NO: 168 - DNA, 23 bp, Artificial Sequence
  Synthetic oligonucleotide
agtgagtgagtatgtgtgtatgg

SEQ ID NO: 169 - DNA, 23 bp, Artificial Sequence
  Synthetic oligonucleotide
agagagtgagtgtgtgtgtagga

SEQ ID NO: 170 - DNA, 23 bp, Artificial Sequence
  Synthetic oligonucleotide
tgtgagtgggtgtgtgcatgtgg

SEQ ID NO: 171 - DNA, 23 bp, Artificial Sequence
  Synthetic oligonucleotide
ggtgagtcagtgagtgagtgggg

SEQ ID NO: 172 - DNA, 23 bp, Artificial Sequence
  Synthetic oligonucleotide
ggggaatgagtgtgtgtgtgggg

SEQ ID NO: 173 - DNA, 23 bp, Artificial Sequence
  Synthetic oligonucleotide
ggtgagtgggtgtgtgtgggggg

SEQ ID NO: 174 - DNA, 23 bp, Artificial Sequence
  Synthetic oligonucleotide
ggggaatgagcatgtgcctgagg

SEQ ID NO: 175 - DNA, 23 bp, Artificial Sequence
  Synthetic oligonucleotide
ggtgagtcagtgagtgagtgggg

SEQ ID NO: 176 - DNA, 23 bp, Artificial Sequence
  Synthetic oligonucleotide
ggtgagtgtgtgtgtgtgttggg

SEQ ID NO: 177 - DNA, 23 bp, Artificial Sequence
  Synthetic oligonucleotide
agtgagtgagtgagtgaatgagg

SEQ ID NO: 178 - DNA, 23 bp, Artificial Sequence
  Synthetic oligonucleotide
tgtgagtgagtgtgtgtgtgagc

SEQ ID NO: 179 - DNA, 23 bp, Artificial Sequence
  Synthetic oligonucleotide
gatgagtgtgtgtgtgtgcgggt

SEQ ID NO: 180 - DNA, 23 bp, Artificial Sequence
  Synthetic oligonucleotide
agtgagtgagtgtgtgtgtgtgt

SEQ ID NO: 181 - DNA, 23 bp, Artificial Sequence
  Synthetic oligonucleotide
agagagtgagtgtgtgtgtgtga

SEQ ID NO: 182 - DNA, 23 bp, Artificial Sequence
  Synthetic oligonucleotide
ggtgagtgtgtgtgtgagtgtgg

SEQ ID NO: 183 - DNA, 23 bp, Artificial Sequence
  Synthetic oligonucleotide
agtgactgagtctgtgcctgggg

SEQ ID NO: 184 - DNA, 23 bp, Artificial Sequence
  Synthetic oligonucleotide
ggggagagagagtgagcgtgggg

SEQ ID NO: 185 - DNA, 23 bp, Artificial Sequence
  Synthetic oligonucleotide
ggtgagtgaatgtgtgtgtgtga

SEQ ID NO: 186 - DNA, 23 bp, Artificial Sequence
  Synthetic oligonucleotide
ggtgagtgagtgtgtgtggggtg

SEQ ID NO: 187 - DNA, 23 bp, Artificial Sequence
  Synthetic oligonucleotide
tgtgagtgggtgtgtgagtgagg

SEQ ID NO: 188 - DNA, 23 bp, Artificial Sequence
  Synthetic oligonucleotide
ggtgagtgtgtgtgtctgggccg

SEQ ID NO: 189 - DNA, 23 bp, Artificial Sequence
  Synthetic oligonucleotide
gataagtgagtatgtgtgtgtgg

SEQ ID NO: 190 - DNA, 23 bp, Artificial Sequence
  Synthetic oligonucleotide
ggtgagtgggtgtgtgtgttggg

SEQ ID NO: 191 - DNA, 23 bp, Artificial Sequence
  Synthetic oligonucleotide
ggtgagtgtgagtgtgtgtgggg

SEQ ID NO: 192 - DNA, 23 bp, Artificial Sequence
  Synthetic oligonucleotide
tgtgagtgagtatgtgcttgcga

SEQ ID NO: 193 - DNA, 23 bp, Artificial Sequence
  Synthetic oligonucleotide
tatgagtgagtgtgagcgtgggt

SEQ ID NO: 194 - DNA, 23 bp, Artificial Sequence
  Synthetic oligonucleotide
tgcgagtgcgtgtgtgtgtttgt

SEQ ID NO: 195 - DNA, 23 bp, Artificial Sequence
  Synthetic oligonucleotide
agcgagtgagtgagtgagtgggg

SEQ ID NO: 196 - DNA, 23 bp, Artificial Sequence
  Synthetic oligonucleotide
agtgagtgagtgagtgagtgagg

SEQ ID NO: 197 - DNA, 23 bp, Artificial Sequence
  Synthetic oligonucleotide
agagagagagtgtgtgtgtgagg

SEQ ID NO: 198 - DNA, 23 bp, Artificial Sequence
  Synthetic oligonucleotide
ggagagagagagtgtgtgtgtga

SEQ ID NO: 199 - DNA, 23 bp, Artificial Sequence
  Synthetic oligonucleotide
agggagtgagtgtgtaagtgtgg

SEQ ID NO: 200 - DNA, 23 bp, Artificial Sequence
  Synthetic oligonucleotide
tgtgagagagtgtgtgcgtgtga

SEQ ID NO: 201 - DNA, 23 bp, Artificial Sequence
  Synthetic oligonucleotide
agtgagtgggtgtgagcttgtgg

SEQ ID NO: 202 - DNA, 23 bp, Artificial Sequence
  Synthetic oligonucleotide
ggtgagtgagtgactgagtgagt

SEQ ID NO: 203 - DNA, 23 bp, Artificial Sequence
  Synthetic oligonucleotide
gtcatcttagtcattacctgagg

SEQ ID NO: 204 - DNA, 23 bp, Artificial Sequence
  Synthetic oligonucleotide
gtcatcttagtcattacctgagg

SEQ ID NO: 205 - DNA, 23 bp, Artificial Sequence
  Synthetic oligonucleotide
gaccccctccaccccgcctccgg

SEQ ID NO: 206 - DNA, 23 bp, Artificial Sequence
  Synthetic oligonucleotide
gaccccctccaccccgcctccgg

SEQ ID NO: 207 - DNA, 23 bp, Artificial Sequence
  Synthetic oligonucleotide
ctacccctccaccccgcctccgg

SEQ ID NO: 208 - DNA, 23 bp, Artificial Sequence
  Synthetic oligonucleotide
attcccccccaccccgcctcagg

SEQ ID NO: 209 - DNA, 23 bp, Artificial Sequence
  Synthetic oligonucleotide
gggcccctccaccccgcctctgg

SEQ ID NO: 210 - DNA, 23 bp, Artificial Sequence
  Synthetic oligonucleotide
ctccccacccaccccgcctcagg

SEQ ID NO: 211 - DNA, 23 bp, Artificial Sequence
  Synthetic oligonucleotide
cctcccccacaccccgcatccgg

SEQ ID NO: 212 - DNA, 23 bp, Artificial Sequence
  Synthetic oligonucleotide
ctcccccccctccccgcctcggg

SEQ ID NO: 213 - DNA, 23 bp, Artificial Sequence
  Synthetic oligonucleotide
cgccctccccaccccgcctccgg

SEQ ID NO: 214 - DNA, 23 bp, Artificial Sequence
  Synthetic oligonucleotide
gcccccacccaccccgcctctgg

SEQ ID NO: 215 - DNA, 23 bp, Artificial Sequence
  Synthetic oligonucleotide
tgcccctcccaccccgcctctgg

SEQ ID NO: 216 - DNA, 23 bp, Artificial Sequence
  Synthetic oligonucleotide
acacccccccaccccgcctcagg

SEQ ID NO: 217 - DNA, 23 bp, Artificial Sequence
  Synthetic oligonucleotide
gacacaccccaccccacctcagg

SEQ ID NO: 218 - DNA, 23 bp, Artificial Sequence
  Synthetic oligonucleotide
aggcccccccgccccgcctcagg

SEQ ID NO: 219 - DNA, 23 bp, Artificial Sequence
  Synthetic oligonucleotide
cccccccccccccccgcctccgg

SEQ ID NO: 220 - DNA, 23 bp, Artificial Sequence
  Synthetic oligonucleotide
tgccccccccaccccacctctgg

SEQ ID NO: 221 - DNA, 23 bp, Artificial Sequence
  Synthetic oligonucleotide
gcttccctccaccccgcatccgg

SEQ ID NO: 222 - DNA, 23 bp, Artificial Sequence
  Synthetic oligonucleotide
cgccctccccattccgccccggg

SEQ ID NO: 223 - DNA, 23 bp, Artificial Sequence
  Synthetic oligonucleotide
cacccccccccccccacctccgg

SEQ ID NO: 224 - DNA, 23 bp, Artificial Sequence
  Synthetic oligonucleotide
taccccccacaccccgcctctgg

SEQ ID NO: 225 - DNA, 23 bp, Artificial Sequence
  Synthetic oligonucleotide
cagtccccccaccccacctctgg

SEQ ID NO: 226 - DNA, 23 bp, Artificial Sequence
  Synthetic oligonucleotide
cactccccccaccccgccccagg

SEQ ID NO: 227 - DNA, 23 bp, Artificial Sequence
  Synthetic oligonucleotide
cccccccccccccccgcctcagg

SEQ ID NO: 228 - DNA, 23 bp, Artificial Sequence
  Synthetic oligonucleotide
cattccccccaccccacctcagg

SEQ ID NO: 229 - DNA, 23 bp, Artificial Sequence
  Synthetic oligonucleotide
agtagcccccaccccgcctcggg

SEQ ID NO: 230 - DNA, 23 bp, Artificial Sequence
  Synthetic oligonucleotide
ttctccctcctccccgcctcggg

SEQ ID NO: 231 - DNA, 23 bp, Artificial Sequence
  Synthetic oligonucleotide
gaccctgtccaccccacctcagg

SEQ ID NO: 232 - DNA, 23 bp, Artificial Sequence
  Synthetic oligonucleotide
tgccccccccaccccgcccccgg

SEQ ID NO: 233 - DNA, 23 bp, Artificial Sequence
  Synthetic oligonucleotide
gacccctcccaccccgactccgg

SEQ ID NO: 234 - DNA, 23 bp, Artificial Sequence
  Synthetic oligonucleotide
caacccccccaccccgcttcagg

SEQ ID NO: 235 - DNA, 23 bp, Artificial Sequence
  Synthetic oligonucleotide
gaccccccccaccccgcccccgg

SEQ ID NO: 236 - DNA, 23 bp, Artificial Sequence
  Synthetic oligonucleotide
gagctcccccaccccgccccggg

SEQ ID NO: 237 - DNA, 23 bp, Artificial Sequence
  Synthetic oligonucleotide
ccgcccctccaccccgccactgg

SEQ ID NO: 238 - DNA, 23 bp, Artificial Sequence
  Synthetic oligonucleotide
agtcccatccaccccgcctaagg

SEQ ID NO: 239 - DNA, 23 bp, Artificial Sequence
  Synthetic oligonucleotide
aagcccccccaccccgccccggg

SEQ ID NO: 240 - DNA, 23 bp, Artificial Sequence
  Synthetic oligonucleotide
tcccccccacccccgacctcagg

SEQ ID NO: 241 - DNA, 23 bp, Artificial Sequence
  Synthetic oligonucleotide
gacccctccctccccacctcagg

SEQ ID NO: 242 - DNA, 23 bp, Artificial Sequence
  Synthetic oligonucleotide
ctcaccccccaccccacctctgg

SEQ ID NO: 243 - DNA, 23 bp, Artificial Sequence
  Synthetic oligonucleotide
aggcccccacaccccgcctcagg

SEQ ID NO: 244 - DNA, 23 bp, Artificial Sequence
  Synthetic oligonucleotide
agccaaccccaccccgcctctgg

SEQ ID NO: 245 - DNA, 23 bp, Artificial Sequence
  Synthetic oligonucleotide
cggccccacctccccgcctctgg

SEQ ID NO: 246 - DNA, 23 bp, Artificial Sequence
  Synthetic oligonucleotide
tccaccccccaccccgccccggg

SEQ ID NO: 247 - DNA, 23 bp, Artificial Sequence
  Synthetic oligonucleotide
tgcaccccccaccccgcccctgg

SEQ ID NO: 248 - DNA, 23 bp, Artificial Sequence
  Synthetic oligonucleotide
ctgcccccccaccccgccactgg

SEQ ID NO: 249 - DNA, 23 bp, Artificial Sequence
  Synthetic oligonucleotide
agccccccccaccccgactcagg

SEQ ID NO: 250 - DNA, 23 bp, Artificial Sequence
  Synthetic oligonucleotide
ccccacccccaccccgcctcagg

SEQ ID NO: 251 - DNA, 23 bp, Artificial Sequence
  Synthetic oligonucleotide
cataccccccaccccgccccggg

SEQ ID NO: 252 - DNA, 23 bp, Artificial Sequence
  Synthetic oligonucleotide
gacaccttccaccccgtctctgg

SEQ ID NO: 253 - DNA, 23 bp, Artificial Sequence
  Synthetic oligonucleotide
gacccgccccgccccgcctctgg

SEQ ID NO: 254 - DNA, 23 bp, Artificial Sequence
  Synthetic oligonucleotide
cccttcattcacccagcctctgg

SEQ ID NO: 255 - DNA, 23 bp, Artificial Sequence
  Synthetic oligonucleotide
aacccctgcctcccgggctcaag

SEQ ID NO: 256 - DNA, 23 bp, Artificial Sequence
  Synthetic oligonucleotide
gctccacaccacccccactctgg

SEQ ID NO: 257 - DNA, 23 bp, Artificial Sequence
  Synthetic oligonucleotide
aacctccacctcccggattcaag

SEQ ID NO: 258 - DNA, 23 bp, Artificial Sequence
  Synthetic oligonucleotide
gccccccaccaccccacctcggg

SEQ ID NO: 259 - DNA, 23 bp, Artificial Sequence
  Synthetic oligonucleotide
gtaccccaccaccccgccccagg

SEQ ID NO: 260 - DNA, 23 bp, Artificial Sequence
  Synthetic oligonucleotide
ccacccccccaccccgccccagg

SEQ ID NO: 261 - DNA, 23 bp, Artificial Sequence
  Synthetic oligonucleotide
gtcactccccaccccgcctctgg

SEQ ID NO: 262 - DNA, 23 bp, Artificial Sequence
  Synthetic oligonucleotide
tcagacctccaccccgcctcagg

SEQ ID NO: 263 - DNA, 23 bp, Artificial Sequence
  Synthetic oligonucleotide
ctcccccgccaccccgccccagg

SEQ ID NO: 264 - DNA, 23 bp, Artificial Sequence
  Synthetic oligonucleotide
gccaccccgcaccccgccccccg

SEQ ID NO: 265 - DNA, 23 bp, Artificial Sequence
  Synthetic oligonucleotide
accccaccccaccccgtctccgg

SEQ ID NO: 266 - DNA, 23 bp, Artificial Sequence
  Synthetic oligonucleotide
gacccccccaccccaccctccgg

SEQ ID NO: 267 - DNA, 23 bp, Artificial Sequence
  Synthetic oligonucleotide
atccccctccaccccacccctgg

SEQ ID NO: 268 - DNA, 23 bp, Artificial Sequence
  Synthetic oligonucleotide
gacccctcacaccccgcccctgg

SEQ ID NO: 269 - DNA, 23 bp, Artificial Sequence
  Synthetic oligonucleotide
taccccccccaccccgccacagg

SEQ ID NO: 270 - DNA, 23 bp, Artificial Sequence
  Synthetic oligonucleotide
ccccccccccaccccgcccccgg

SEQ ID NO: 271 - DNA, 23 bp, Artificial Sequence
  Synthetic oligonucleotide
ctccctccccaccccacctctgg

SEQ ID NO: 272 - DNA, 23 bp, Artificial Sequence
  Synthetic oligonucleotide
accccccccccccccgcccccgg

SEQ ID NO: 273 - DNA, 23 bp, Artificial Sequence
  Synthetic oligonucleotide
cccccccaccaccccgccccggg

SEQ ID NO: 274 - DNA, 23 bp, Artificial Sequence
  Synthetic oligonucleotide
gaccccccccaccccaccccagg

SEQ ID NO: 275 - DNA, 23 bp, Artificial Sequence
  Synthetic oligonucleotide
tccccaccccgccccgcctctgg

SEQ ID NO: 276 - DNA, 23 bp, Artificial Sequence
  Synthetic oligonucleotide
actcccctccaccccggctcggg

SEQ ID NO: 277 - DNA, 23 bp, Artificial Sequence
  Synthetic oligonucleotide
agcccccacctccccgcctcggg

SEQ ID NO: 278 - DNA, 23 bp, Artificial Sequence
  Synthetic oligonucleotide
actcccctccaccccacctctga

SEQ ID NO: 279 - DNA, 23 bp, Artificial Sequence
  Synthetic oligonucleotide
catcccccccaccccaccccggg

SEQ ID NO: 280 - DNA, 23 bp, Artificial Sequence
  Synthetic oligonucleotide
aaccacccccaccccaccccagg

SEQ ID NO: 281 - DNA, 23 bp, Artificial Sequence
  Synthetic oligonucleotide
cacacccaccaccccgcctcaga

SEQ ID NO: 282 - DNA, 23 bp, Artificial Sequence
  Synthetic oligonucleotide
cccccctcccaccccgcctctag

SEQ ID NO: 283 - DNA, 23 bp, Artificial Sequence
  Synthetic oligonucleotide
cccccccccccccccgccccggg

SEQ ID NO: 284 - DNA, 23 bp, Artificial Sequence
  Synthetic oligonucleotide
aacctcccccaccccaccccagg

SEQ ID NO: 285 - DNA, 23 bp, Artificial Sequence
  Synthetic oligonucleotide
caccacccccaccccgcccctgg

SEQ ID NO: 286 - DNA, 23 bp, Artificial Sequence
  Synthetic oligonucleotide
cttcccccacaccccgccccagg

SEQ ID NO: 287 - DNA, 23 bp, Artificial Sequence
  Synthetic oligonucleotide
ccgcccccccaccccgccgccgg

SEQ ID NO: 288 - DNA, 23 bp, Artificial Sequence
  Synthetic oligonucleotide
ggccccacccaccccgccttctg

SEQ ID NO: 289 - DNA, 23 bp, Artificial Sequence
  Synthetic oligonucleotide
cccaccccccaccccgccccagg

SEQ ID NO: 290 - DNA, 23 bp, Artificial Sequence
  Synthetic oligonucleotide
ggccctctccactccacctcagg

SEQ ID NO: 291 - DNA, 23 bp, Artificial Sequence
  Synthetic oligonucleotide
cccccccccccccccgcctcagg

SEQ ID NO: 292 - DNA, 23 bp, Artificial Sequence
  Synthetic oligonucleotide
taccccctccaccccgctccagg

SEQ ID NO: 293 - DNA, 23 bp, Artificial Sequence
  Synthetic oligonucleotide
cgcccaccccaccccacctcagg

SEQ ID NO: 294 - DNA, 23 bp, Artificial Sequence
  Synthetic oligonucleotide
aagaccccccaccccgccccagg

SEQ ID NO: 295 - DNA, 23 bp, Artificial Sequence
  Synthetic oligonucleotide
ggaccccccccccccgcccccgg

SEQ ID NO: 296 - DNA, 23 bp, Artificial Sequence
  Synthetic oligonucleotide
ggctccctccgccccgccccggg

SEQ ID NO: 297 - DNA, 23 bp, Artificial Sequence
  Synthetic oligonucleotide
ccacccccccaccccgcccctgg

SEQ ID NO: 298 - DNA, 23 bp, Artificial Sequence
  Synthetic oligonucleotide
ctccccctccaccccacctccag

SEQ ID NO: 299 - DNA, 23 bp, Artificial Sequence
  Synthetic oligonucleotide
ccccccccccgccccgcctccag

SEQ ID NO: 300 - DNA, 23 bp, Artificial Sequence
  Synthetic oligonucleotide
ctacccccactccccgcctccgg

SEQ ID NO: 301 - DNA, 23 bp, Artificial Sequence
  Synthetic oligonucleotide
caccccctcccctccgcctcagg

SEQ ID NO: 302 - DNA, 23 bp, Artificial Sequence
  Synthetic oligonucleotide
atgccccacccccccgcccccgg

SEQ ID NO: 303 - DNA, 23 bp, Artificial Sequence
  Synthetic oligonucleotide
aacaccccccaccccaccccggg

SEQ ID NO: 304 - DNA, 23 bp, Artificial Sequence
  Synthetic oligonucleotide
agacccccccaccccaccccagg

SEQ ID NO: 305 - DNA, 23 bp, Artificial Sequence
  Synthetic oligonucleotide
gactccctccgccccgcttccag

SEQ ID NO: 306 - DNA, 23 bp, Artificial Sequence
  Synthetic oligonucleotide
cccccccctcaccccgcccctgg

SEQ ID NO: 307 - DNA, 23 bp, Artificial Sequence
  Synthetic oligonucleotide
gaccccacctaccccacctcagg

SEQ ID NO: 308 - DNA, 23 bp, Artificial Sequence
  Synthetic oligonucleotide
gtccccctcctccccacctccgg

SEQ ID NO: 309 - DNA, 23 bp, Artificial Sequence
  Synthetic oligonucleotide
gtcctccaccaccccgcctctgg

SEQ ID NO: 310 - DNA, 23 bp, Artificial Sequence
  Synthetic oligonucleotide
ggagagggaccgcgccaccttgg

SEQ ID NO: 311 - DNA, 23 bp, Artificial Sequence
  Synthetic oligonucleotide
ggtgaggcaccgtgccacctggg

SEQ ID NO: 312 - DNA, 23 bp, Artificial Sequence
  Synthetic oligonucleotide
ggagaggcaccgccccacatggg

SEQ ID NO: 313 - DNA, 23 bp, Artificial Sequence
  Synthetic oligonucleotide
ggggaggcaccgggccaccttgg

SEQ ID NO: 314 - DNA, 23 bp, Artificial Sequence
  Synthetic oligonucleotide
ggtgagggaccgagtcacctagg

SEQ ID NO: 315 - DNA, 23 bp, Artificial Sequence
  Synthetic oligonucleotide
caagaggaacggcgccacctggg

SEQ ID NO: 316 - DNA, 23 bp, Artificial Sequence
  Synthetic oligonucleotide
agaaagtgacagcgccacctagg

SEQ ID NO: 317 - DNA, 23 bp, Artificial Sequence
  Synthetic oligonucleotide
ggggaggggctgtgccacctggg

SEQ ID NO: 318 - DNA, 23 bp, Artificial Sequence
  Synthetic oligonucleotide
ggaggaggactgcgccacttcgg

SEQ ID NO: 319 - DNA, 23 bp, Artificial Sequence
  Synthetic oligonucleotide
ggaaagggaccccaccacccagg

SEQ ID NO: 320 - DNA, 23 bp, Artificial Sequence
  Synthetic oligonucleotide
agggaggggccgcgccgccttgg

SEQ ID NO: 321 - DNA, 23 bp, Artificial Sequence
  Synthetic oligonucleotide
ggagctggaccacgccaccctgg

SEQ ID NO: 322 - DNA, 23 bp, Artificial Sequence
  Synthetic oligonucleotide
caagaggggcagcgccaccttgg

SEQ ID NO: 323 - DNA, 23 bp, Artificial Sequence
  Synthetic oligonucleotide
ggaaagggcccccaccacctggg

SEQ ID NO: 324 - DNA, 23 bp, Artificial Sequence
  Synthetic oligonucleotide
ggagagcaatggcgccacctcgg

SEQ ID NO: 325 - DNA, 23 bp, Artificial Sequence
  Synthetic oligonucleotide
ggggagggactgcaccaccacgg

SEQ ID NO: 326 - DNA, 23 bp, Artificial Sequence
  Synthetic oligonucleotide
tgggagtgaccgccccacctggg

SEQ ID NO: 327 - DNA, 23 bp, Artificial Sequence
  Synthetic oligonucleotide
gcagaggggccaccccacctggg

SEQ ID NO: 328 - DNA, 23 bp, Artificial Sequence
  Synthetic oligonucleotide
ggtgagggatccagccacctggg

SEQ ID NO: 329 - DNA, 23 bp, Artificial Sequence
  Synthetic oligonucleotide
cttgagggactgcgccacctgga

SEQ ID NO: 330 - DNA, 23 bp, Artificial Sequence
  Synthetic oligonucleotide
ggagagaggccgccctacctggg

SEQ ID NO: 331 - DNA, 23 bp, Artificial Sequence
  Synthetic oligonucleotide
ggacagggcccccgccacccagg

SEQ ID NO: 332 - DNA, 23 bp, Artificial Sequence
  Synthetic oligonucleotide
ggtgaggcatcgccccacctggg

SEQ ID NO: 333 - DNA, 23 bp, Artificial Sequence
  Synthetic oligonucleotide
ggacaggaaccactccacctgag

SEQ ID NO: 334 - DNA, 23 bp, Artificial Sequence
  Synthetic oligonucleotide
ggagagcaaaggcgccacctcgg

SEQ ID NO: 335 - DNA, 23 bp, Artificial Sequence
  Synthetic oligonucleotide
gcagagggacagcactaccttgg

SEQ ID NO: 336 - DNA, 23 bp, Artificial Sequence
  Synthetic oligonucleotide
ggagagggtgagcaccaccttgg

SEQ ID NO: 337 - DNA, 23 bp, Artificial Sequence
  Synthetic oligonucleotide
gcagagggacggcaccacccagg

SEQ ID NO: 338 - DNA, 23 bp, Artificial Sequence
  Synthetic oligonucleotide
ggtgatggacagccccacctagg

SEQ ID NO: 339 - DNA, 23 bp, Artificial Sequence
  Synthetic oligonucleotide
ggggaagagccccgccacctggg

SEQ ID NO: 340 - DNA, 23 bp, Artificial Sequence
  Synthetic oligonucleotide
ggaggaagaccacgccaccctgg

SEQ ID NO: 341 - DNA, 23 bp, Artificial Sequence
  Synthetic oligonucleotide
actgagggactgctccacctggg

SEQ ID NO: 342 - DNA, 23 bp, Artificial Sequence
  Synthetic oligonucleotide
cctgagggacctcgccaccctgg

SEQ ID NO: 343 - DNA, 23 bp, Artificial Sequence
  Synthetic oligonucleotide
aaagagggacagccccacccggg

SEQ ID NO: 344 - DNA, 23 bp, Artificial Sequence
  Synthetic oligonucleotide
gattaaggacagcgccacctggg

SEQ ID NO: 345 - DNA, 23 bp, Artificial Sequence
  Synthetic oligonucleotide
tgaaggggaccacgccaccctgg

SEQ ID NO: 346 - DNA, 23 bp, Artificial Sequence
  Synthetic oligonucleotide
agagaaggaccccgcctccccgg

SEQ ID NO: 347 - DNA, 23 bp, Artificial Sequence
  Synthetic oligonucleotide
gcagaaggaccatgccacccggg

SEQ ID NO: 348 - DNA, 23 bp, Artificial Sequence
  Synthetic oligonucleotide
aaggagggaccccgccaccccag

SEQ ID NO: 349 - DNA, 23 bp, Artificial Sequence
  Synthetic oligonucleotide
agagagaggctgccccacctggg

SEQ ID NO: 350 - DNA, 23 bp, Artificial Sequence
  Synthetic oligonucleotide
agaggggtacagtgccaccttgg

SEQ ID NO: 351 - DNA, 23 bp, Artificial Sequence
  Synthetic oligonucleotide
agagacgggctgcgccacccggg

SEQ ID NO: 352 - DNA, 23 bp, Artificial Sequence
  Synthetic oligonucleotide
ggggagagaccccaccccctagg

SEQ ID NO: 353 - DNA, 23 bp, Artificial Sequence
  Synthetic oligonucleotide
cgggaggggccgtcccacctcgg

SEQ ID NO: 354 - DNA, 23 bp, Artificial Sequence
  Synthetic oligonucleotide
ggagaaaggccaagccacctagg

SEQ ID NO: 355 - DNA, 23 bp, Artificial Sequence
  Synthetic oligonucleotide
ggagaggagtccccccacctagg

SEQ ID NO: 356 - DNA, 23 bp, Artificial Sequence
  Synthetic oligonucleotide
aaggaggggccccaccacctggg

SEQ ID NO: 357 - DNA, 23 bp, Artificial Sequence
  Synthetic oligonucleotide
ccagggggacaaggccacctagg

SEQ ID NO: 358 - DNA, 23 bp, Artificial Sequence
  Synthetic oligonucleotide
ggagaggttcctgggcaccccag

SEQ ID NO: 359 - DNA, 23 bp, Artificial Sequence
  Synthetic oligonucleotide
ccagagcagccgctccacctcgg

SEQ ID NO: 360 - DNA, 23 bp, Artificial Sequence
  Synthetic oligonucleotide
ggagtgggcaggtgccaccgtgg

SEQ ID NO: 361 - DNA, 23 bp, Artificial Sequence
  Synthetic oligonucleotide
gaacttacgcaggagatattcgg

SEQ ID NO: 362 - DNA, 23 bp, Artificial Sequence
  Synthetic oligonucleotide
gcatttaggcaggagatatttgg

SEQ ID NO: 363 - DNA, 23 bp, Artificial Sequence
  Synthetic oligonucleotide
ccccttacgcaggggatatttgg

SEQ ID NO: 364 - DNA, 23 bp, Artificial Sequence
  Synthetic oligonucleotide
gttccggtaagcatagacaatgg

SEQ ID NO: 365 - DNA, 23 bp, Artificial Sequence
  Synthetic oligonucleotide
attacagcaagcatagacaatgg

SEQ ID NO: 366 - DNA, 23 bp, Artificial Sequence
  Synthetic oligonucleotide
gagacccgctcttcagcatgtgg

SEQ ID NO: 367 - DNA, 23 bp, Artificial Sequence
  Synthetic oligonucleotide
gagccccgctcctcagcatctgg

SEQ ID NO: 368 - DNA, 23 bp, Artificial Sequence
  Synthetic oligonucleotide
gggacccgatcttcagcttgtgg

SEQ ID NO: 369 - DNA, 23 bp, Artificial Sequence
  Synthetic oligonucleotide
gagacccattgttcagcatgcgg

SEQ ID NO: 370 - DNA, 23 bp, Artificial Sequence
  Synthetic oligonucleotide
gagactcgcccctcagcatcggg

SEQ ID NO: 371 - DNA, 23 bp, Artificial Sequence
  Synthetic oligonucleotide
aaaacccgctcctaagcatgtgg

SEQ ID NO: 372 - DNA, 23 bp, Artificial Sequence
  Synthetic oligonucleotide
ggctcccgctctccagcatgcgg

SEQ ID NO: 373 - DNA, 23 bp, Artificial Sequence
  Synthetic oligonucleotide
gagagggccccaagacctcgtgg

SEQ ID NO: 374 - DNA, 23 bp, Artificial Sequence
  Synthetic oligonucleotide
gggagggtcccaagaccccgggg

SEQ ID NO: 375 - DNA, 23 bp, Artificial Sequence
  Synthetic oligonucleotide
aatagggccccaaggcctcgggg

SEQ ID NO: 376 - DNA, 23 bp, Artificial Sequence
  Synthetic oligonucleotide
gagagggacccaaggcctccggg

SEQ ID NO: 377 - DNA, 23 bp, Artificial Sequence
  Synthetic oligonucleotide
aagagggccccaagaccgcagag

SEQ ID NO: 378 - DNA, 23 bp, Artificial Sequence
  Synthetic oligonucleotide
gagggggccccaagaccccaagc

SEQ ID NO: 379 - DNA, 23 bp, Artificial Sequence
  Synthetic oligonucleotide
cagagggccccaagacccccaag

SEQ ID NO: 380 - DNA, 23 bp, Artificial Sequence
  Synthetic oligonucleotide
gagaggggctcaggacctcgtgg

SEQ ID NO: 381 - DNA, 23 bp, Artificial Sequence
  Synthetic oligonucleotide
gagagggcccccaggcctccggg

SEQ ID NO: 382 - DNA, 23 bp, Artificial Sequence
  Synthetic oligonucleotide
gagggggccccaagacctgcagg

SEQ ID NO: 383 - DNA, 23 bp, Artificial Sequence
  Synthetic oligonucleotide
aagagggcccctagagctcgagg

SEQ ID NO: 384 - DNA, 23 bp, Artificial Sequence
  Synthetic oligonucleotide
gagggggccccaagtcctcaagg

SEQ ID NO: 385 - DNA, 23 bp, Artificial Sequence
  Synthetic oligonucleotide
aagaggtgcccaagacctcaggg

SEQ ID NO: 386 - DNA, 23 bp, Artificial Sequence
  Synthetic oligonucleotide
gagaggacaccaagaccccaggg

SEQ ID NO: 387 - DNA, 23 bp, Artificial Sequence
  Synthetic oligonucleotide
gagggagccccaggacctctggg

SEQ ID NO: 388 - DNA, 23 bp, Artificial Sequence
  Synthetic oligonucleotide
gggaaggccccaggaccccgtgg

SEQ ID NO: 389 - DNA, 23 bp, Artificial Sequence
  Synthetic oligonucleotide
cccagggccccaaggcctcgggg

SEQ ID NO: 390 - DNA, 23 bp, Artificial Sequence
  Synthetic oligonucleotide
cagaggaccccaagacccccagg

SEQ ID NO: 391 - DNA, 23 bp, Artificial Sequence
  Synthetic oligonucleotide
gatagagctccaagacctctgag

SEQ ID NO: 392 - DNA, 23 bp, Artificial Sequence
  Synthetic oligonucleotide
tagagggtcccaggaccttgtgg

SEQ ID NO: 393 - DNA, 23 bp, Artificial Sequence
  Synthetic oligonucleotide
gatggggccccaggacctcaagg

SEQ ID NO: 394 - DNA, 23 bp, Artificial Sequence
  Synthetic oligonucleotide
aagagggctccaagatctcatgg

SEQ ID NO: 395 - DNA, 23 bp, Artificial Sequence
  Synthetic oligonucleotide
atgagaggcccaagacctcgggg

SEQ ID NO: 396 - DNA, 23 bp, Artificial Sequence
  Synthetic oligonucleotide
gaggggacaccaaggcctcgagg

SEQ ID NO: 397 - DNA, 23 bp, Artificial Sequence
  Synthetic oligonucleotide
gaggtggccccagggcctcgagg

SEQ ID NO: 398 - DNA, 23 bp, Artificial Sequence
  Synthetic oligonucleotide
ttgagggtcccaagaccccaggg

SEQ ID NO: 399 - DNA, 23 bp, Artificial Sequence
  Synthetic oligonucleotide
aagaagactccaagacctcacgg

SEQ ID NO: 400 - DNA, 23 bp, Artificial Sequence
  Synthetic oligonucleotide
ggaggaggcccaagacctcgggg

SEQ ID NO: 401 - DNA, 23 bp, Artificial Sequence
  Synthetic oligonucleotide
gagagggactcaccacctgggtg

SEQ ID NO: 402 - DNA, 23 bp, Artificial Sequence
  Synthetic oligonucleotide
aggtgggccccaggacctctggg

SEQ ID NO: 403 - DNA, 23 bp, Artificial Sequence
  Synthetic oligonucleotide
aagaaggccctaagacccctagg

SEQ ID NO: 404 - DNA, 23 bp, Artificial Sequence
  Synthetic oligonucleotide
gagagggccctgccacctgggcc

SEQ ID NO: 405 - DNA, 23 bp, Artificial Sequence
  Synthetic oligonucleotide
aagaaagccccaagaccttatgg

SEQ ID NO: 406 - DNA, 23 bp, Artificial Sequence
  Synthetic oligonucleotide
cccagggcccccagaccccgggg

SEQ ID NO: 407 - DNA, 23 bp, Artificial Sequence
  Synthetic oligonucleotide
ggccgggccccgggccctcgacc

SEQ ID NO: 408 - DNA, 23 bp, Artificial Sequence
  Synthetic oligonucleotide
gcgccttattccagtgacaaagg

SEQ ID NO: 409 - DNA, 23 bp, Artificial Sequence
  Synthetic oligonucleotide
gcagatcctcatcaccgcgctgg

SEQ ID NO: 410 - DNA, 23 bp, Artificial Sequence
  Synthetic oligonucleotide
gcagaaccgcatcaccgcgctgg

SEQ ID NO: 411 - DNA, 23 bp, Artificial Sequence
  Synthetic oligonucleotide
gcagaaccgcatcaccgcgctgg

SEQ ID NO: 412 - DNA, 23 bp, Artificial Sequence
  Synthetic oligonucleotide
gcagactctcatcaccgctcagg

SEQ ID NO: 413 - DNA, 23 bp, Artificial Sequence
  Synthetic oligonucleotide
gcagactctcatcaccgctcagg

SEQ ID NO: 414 - DNA, 23 bp, Artificial Sequence
  Synthetic oligonucleotide
gcagactctcatcaccgctcagg

SEQ ID NO: 415 - DNA, 23 bp, Artificial Sequence
  Synthetic oligonucleotide
gcagactctcatcaccgctcagg

SEQ ID NO: 416 - DNA, 23 bp, Artificial Sequence
  Synthetic oligonucleotide
gcagactctcatcaccgctcagg

SEQ ID NO: 417 - DNA, 23 bp, Artificial Sequence
  Synthetic oligonucleotide
aaagatcctcatcaccgccctag

SEQ ID NO: 418 - DNA, 23 bp, Artificial Sequence
  Synthetic oligonucleotide
gcagactatcatcaccgctcagg

SEQ ID NO: 419 - DNA, 23 bp, Artificial Sequence
  Synthetic oligonucleotide
gcagatgcccaccaccacgctgg

SEQ ID NO: 420 - DNA, 23 bp, Artificial Sequence
  Synthetic oligonucleotide
ccagatccacatcaccaagctgg

SEQ ID NO: 421 - DNA, 23 bp, Artificial Sequence
  Synthetic oligonucleotide
gcagaacatcaccaccacgcggg

SEQ ID NO: 422 - DNA, 23 bp, Artificial Sequence
  Synthetic oligonucleotide
acagatgctcaccaccacgccgg

SEQ ID NO: 423 - DNA, 23 bp, Artificial Sequence
  Synthetic oligonucleotide
acagacccccaccaccgcgcctg

SEQ ID NO: 424 - DNA, 23 bp, Artificial Sequence
  Synthetic oligonucleotide
gcagagccccaccacagcgctgg

SEQ ID NO: 425 - DNA, 23 bp, Artificial Sequence
  Synthetic oligonucleotide
acagatactcaccaccacgcagg

SEQ ID NO: 426 - DNA, 23 bp, Artificial Sequence
  Synthetic oligonucleotide
acagacgcccaccaccgcgccgg

SEQ ID NO: 427 - DNA, 23 bp, Artificial Sequence
  Synthetic oligonucleotide
acagacccgcaccaccacgctgg

SEQ ID NO: 428 - DNA, 23 bp, Artificial Sequence
  Synthetic oligonucleotide
acaggtactcaccaccacgcagg

SEQ ID NO: 429 - DNA, 23 bp, Artificial Sequence
  Synthetic oligonucleotide
gcagatgcccaccaccaagcccg

SEQ ID NO: 430 - DNA, 23 bp, Artificial Sequence
  Synthetic oligonucleotide
acagacacccaccaccgcgcctg

SEQ ID NO: 431 - DNA, 23 bp, Artificial Sequence
  Synthetic oligonucleotide
acaggtacacatcaccacgccgg

SEQ ID NO: 432 - DNA, 23 bp, Artificial Sequence
  Synthetic oligonucleotide
gcagactctcatcgccactcagg

SEQ ID NO: 433 - DNA, 23 bp, Artificial Sequence
  Synthetic oligonucleotide
acagatgctcaccaccacggaca

SEQ ID NO: 434 - DNA, 23 bp, Artificial Sequence
  Synthetic oligonucleotide
acaggtcctcaccaccatgccgg

SEQ ID NO: 435 - DNA, 23 bp, Artificial Sequence
  Synthetic oligonucleotide
acagatgttcaccaccacgccgg

SEQ ID NO: 436 - DNA, 23 bp, Artificial Sequence
  Synthetic oligonucleotide
gtagattcccatcaccaagctgg

SEQ ID NO: 437 - DNA, 23 bp, Artificial Sequence
  Synthetic oligonucleotide
acaggtccgcaccaccacgccgg

SEQ ID NO: 438 - DNA, 23 bp, Artificial Sequence
  Synthetic oligonucleotide
acagacacccaccaccgcgccag

SEQ ID NO: 439 - DNA, 23 bp, Artificial Sequence
  Synthetic oligonucleotide
acagacctgaaccaccgcgccgg

SEQ ID NO: 440 - DNA, 23 bp, Artificial Sequence
  Synthetic oligonucleotide
acagacgcccaccaccgtgccca

SEQ ID NO: 441 - DNA, 23 bp, Artificial Sequence
  Synthetic oligonucleotide
acagacgcccaccaccgtgcccg

SEQ ID NO: 442 - DNA, 23 bp, Artificial Sequence
  Synthetic oligonucleotide
acagacacccaccaccacgcggg

SEQ ID NO: 443 - DNA, 23 bp, Artificial Sequence
  Synthetic oligonucleotide
acagacccacaccaccacactgg

SEQ ID NO: 444 - DNA, 23 bp, Artificial Sequence
  Synthetic oligonucleotide
acagacacacaccaccacgccgg

SEQ ID NO: 445 - DNA, 23 bp, Artificial Sequence
  Synthetic oligonucleotide
acaaatccacaccaccatgcagg

SEQ ID NO: 446 - DNA, 23 bp, Artificial Sequence
  Synthetic oligonucleotide
acagacacgcaccaccacgctgg

SEQ ID NO: 447 - DNA, 23 bp, Artificial Sequence
  Synthetic oligonucleotide
acagatacccaccaccacaccgg

SEQ ID NO: 448 - DNA, 23 bp, Artificial Sequence
  Synthetic oligonucleotide
gcatgtcgacttctcctcggagg

SEQ ID NO: 449 - DNA, 23 bp, Artificial Sequence
  Synthetic oligonucleotide
ttatgtctacttctcctaggagg

SEQ ID NO: 450 - DNA, 23 bp, Artificial Sequence
  Synthetic oligonucleotide
agatgccgacctctcctcgaagg

SEQ ID NO: 451 - DNA, 23 bp, Artificial Sequence
  Synthetic oligonucleotide
acatgtgaactactcctcagggg

SEQ ID NO: 452 - DNA, 23 bp, Artificial Sequence
  Synthetic oligonucleotide
ctctgcggacttctcctcggggg

SEQ ID NO: 453 - DNA, 23 bp, Artificial Sequence
  Synthetic oligonucleotide
gcaccccgacgcctcctcggaag

SEQ ID NO: 454 - DNA, 23 bp, Artificial Sequence
  Synthetic oligonucleotide
acgtgccgacccctcctctgggg

SEQ ID NO: 455 - DNA, 23 bp, Artificial Sequence
  Synthetic oligonucleotide
gcaggacggcccctccccggggg

SEQ ID NO: 456 - DNA, 23 bp, Artificial Sequence
  Synthetic oligonucleotide
gcacgccggcgcctccccggagg

SEQ ID NO: 457 - DNA, 23 bp, Artificial Sequence
  Synthetic oligonucleotide
gcacgccggcacctccccggagg

SEQ ID NO: 458 - DNA, 23 bp, Artificial Sequence
  Synthetic oligonucleotide
acaggcccatttctccccggggg

SEQ ID NO: 459 - DNA, 23 bp, Artificial Sequence
  Synthetic oligonucleotide
gctggtctaggacgtcctccagg

SEQ ID NO: 460 - DNA, 23 bp, Artificial Sequence
  Synthetic oligonucleotide
cctggtctatgacgtcctcctgc

SEQ ID NO: 461 - DNA, 23 bp, Artificial Sequence
  Synthetic oligonucleotide
gctggtccaggacatcccccagg

SEQ ID NO: 462 - DNA, 23 bp, Artificial Sequence
  Synthetic oligonucleotide
gctggtccaagacgtccaccagg

SEQ ID NO: 463 - DNA, 23 bp, Artificial Sequence
  Synthetic oligonucleotide
gctgggctagggcctcctccagg

SEQ ID NO: 464 - DNA, 23 bp, Artificial Sequence
  Synthetic oligonucleotide
gctggtctccggcgtcctcccgg

SEQ ID NO: 465 - DNA, 23 bp, Artificial Sequence
  Synthetic oligonucleotide
actggccgaggaagtcctccagg

SEQ ID NO: 466 - DNA, 23 bp, Artificial Sequence
  Synthetic oligonucleotide
gctggggaaggacgtcccccggg

SEQ ID NO: 467 - DNA, 23 bp, Artificial Sequence
  Synthetic oligonucleotide
gctggtccaagacatcccccagg

SEQ ID NO: 468 - DNA, 23 bp, Artificial Sequence
  Synthetic oligonucleotide
gctgggttagaatgtcctccagg

SEQ ID NO: 469 - DNA, 23 bp, Artificial Sequence
  Synthetic oligonucleotide
actggtttataacgtcctcctgg

SEQ ID NO: 470 - DNA, 23 bp, Artificial Sequence
  Synthetic oligonucleotide
gctagtccagaacggcctccagg

SEQ ID NO: 471 - DNA, 23 bp, Artificial Sequence
  Synthetic oligonucleotide
actggtctaggacattcccccgg

SEQ ID NO: 472 - DNA, 23 bp, Artificial Sequence
  Synthetic oligonucleotide
gcaggcccagagcgtcctcctgg

SEQ ID NO: 473 - DNA, 23 bp, Artificial Sequence
  Synthetic oligonucleotide
ggaaactcaccgatccatacagg

SEQ ID NO: 474 - DNA, 23 bp, Artificial Sequence
  Synthetic oligonucleotide
gccagcaaagcacattattttgg

SEQ ID NO: 475 - DNA, 23 bp, Artificial Sequence
  Synthetic oligonucleotide
ggcccgtctccgtgctcctctgg

SEQ ID NO: 476 - DNA, 23 bp, Artificial Sequence
  Synthetic oligonucleotide
ggcccgcctccctcctcctctgg

SEQ ID NO: 477 - DNA, 23 bp, Artificial Sequence
  Synthetic oligonucleotide
ggggtgcctccgggctcctcggg

SEQ ID NO: 478 - DNA, 23 bp, Artificial Sequence
  Synthetic oligonucleotide
gcgctacgacgagatcgtcaagg

SEQ ID NO: 479 - DNA, 23 bp, Artificial Sequence
  Synthetic oligonucleotide
gagaataagattcagttgcaagg

SEQ ID NO: 480 - DNA, 23 bp, Artificial Sequence
  Synthetic oligonucleotide
gagaaagagtttcagttgcaggg

SEQ ID NO: 481 - DNA, 23 bp, Artificial Sequence
  Synthetic oligonucleotide
aagaataagagtcagttgtaggg

SEQ ID NO: 482 - DNA, 23 bp, Artificial Sequence
  Synthetic oligonucleotide
gtttcttgggatccaccaccagg

SEQ ID NO: 483 - DNA, 23 bp, Artificial Sequence
  Synthetic oligonucleotide
gtttattaggatccaccacctga

SEQ ID NO: 484 - DNA, 23 bp, Artificial Sequence
  Synthetic oligonucleotide
gctgctcgggatccaccaccagg

SEQ ID NO: 485 - DNA, 23 bp, Artificial Sequence
  Synthetic oligonucleotide
gcttcttgggagtcaccaccagg

SEQ ID NO: 486 - DNA, 23 bp, Artificial Sequence
  Synthetic oligonucleotide
gctccttgggatccaccgcctgg

SEQ ID NO: 487 - DNA, 23 bp, Artificial Sequence
  Synthetic oligonucleotide
gtttctaggaatccaccatccgg

SEQ ID NO: 488 - DNA, 23 bp, Artificial Sequence
  Synthetic oligonucleotide
tgttctagggacccaccaccagg

SEQ ID NO: 489 - DNA, 23 bp, Artificial Sequence
  Synthetic oligonucleotide
cttcctggggacccaccacctgg

SEQ ID NO: 490 - DNA, 23 bp, Artificial Sequence
  Synthetic oligonucleotide
gcctcaagggatccaccacctgg

SEQ ID NO: 491 - DNA, 23 bp, Artificial Sequence
  Synthetic oligonucleotide
tgtgctggggatccaccaccggg

SEQ ID NO: 492 - DNA, 23 bp, Artificial Sequence
  Synthetic oligonucleotide
gcttcctggcatccacccccagg

SEQ ID NO: 493 - DNA, 23 bp, Artificial Sequence
  Synthetic oligonucleotide
actacctgggatccaccaccaga

SEQ ID NO: 494 - DNA, 23 bp, Artificial Sequence
  Synthetic oligonucleotide
agaccttgggatccaccacctgt

SEQ ID NO: 495 - DNA, 23 bp, Artificial Sequence
  Synthetic oligonucleotide
ccagcttgggacccaccacccgc

SEQ ID NO: 496 - DNA, 23 bp, Artificial Sequence
  Synthetic oligonucleotide
gattccaggcacccaccacctgg

SEQ ID NO: 497 - DNA, 23 bp, Artificial Sequence
  Synthetic oligonucleotide
ccatcatggcatccaccaccagg

SEQ ID NO: 498 - DNA, 23 bp, Artificial Sequence
  Synthetic oligonucleotide
gtaggtgggctgccgaagatagg

SEQ ID NO: 499 - DNA, 23 bp, Artificial Sequence
  Synthetic oligonucleotide
gtaattaggtaaggcttagttgg

SEQ ID NO: 500 - DNA, 23 bp, Artificial Sequence
  Synthetic oligonucleotide
ccatttaggtaaagcttagtggg

SEQ ID NO: 501 - DNA, 23 bp, Artificial Sequence
  Synthetic oligonucleotide
gtgatagggttagggttagggtt

SEQ ID NO: 502 - DNA, 23 bp, Artificial Sequence
  Synthetic oligonucleotide
gctctttgaccgcgcgcgtgtgg

SEQ ID NO: 503 - DNA, 23 bp, Artificial Sequence
  Synthetic oligonucleotide
gatcttggactgctcccctggca

SEQ ID NO: 504 - DNA, 23 bp, Artificial Sequence
  Synthetic oligonucleotide
gaaacagctcgttgtaccgctgg

SEQ ID NO: 505 - DNA, 23 bp, Artificial Sequence
  Synthetic oligonucleotide
gaagcagcttgttgtacctctgg

SEQ ID NO: 506 - DNA, 23 bp, Artificial Sequence
  Synthetic oligonucleotide
gaagcagcccattgtactgcagg

SEQ ID NO: 507 - DNA, 23 bp, Artificial Sequence
  Synthetic oligonucleotide
gaaacacctccttgtggggaact

SEQ ID NO: 508 - DNA, 23 bp, Artificial Sequence
  Synthetic oligonucleotide
gcaacaacgtgatgaatatctgg

SEQ ID NO: 509 - DNA, 23 bp, Artificial Sequence
  Synthetic oligonucleotide
gtcgctgtgactttctaatttgg

SEQ ID NO: 510 - DNA, 23 bp, Artificial Sequence
  Synthetic oligonucleotide
ggtgttatctctgaagcgcatgg

SEQ ID NO: 511 - DNA, 23 bp, Artificial Sequence
  Synthetic oligonucleotide
gtggttatctctgaagcacatgg

SEQ ID NO: 512 - DNA, 23 bp, Artificial Sequence
  Synthetic oligonucleotide
agtgttgtctctgaagagcatgg

SEQ ID NO: 513 - DNA, 23 bp, Artificial Sequence
  Synthetic oligonucleotide
agtcctatctctgaagcccaggg

SEQ ID NO: 514 - DNA, 23 bp, Artificial Sequence
  Synthetic oligonucleotide
agtcctatctctgaagcccaggg

SEQ ID NO: 515 - DNA, 23 bp, Artificial Sequence
  Synthetic oligonucleotide
ggatcatggaagccagctccagg

SEQ ID NO: 516 - DNA, 23 bp, Artificial Sequence
  Synthetic oligonucleotide
ggatcagggaagccagcccctgg

SEQ ID NO: 517 - DNA, 23 bp, Artificial Sequence
  Synthetic oligonucleotide
tgatcaaggaagccagctccagg

SEQ ID NO: 518 - DNA, 23 bp, Artificial Sequence
  Synthetic oligonucleotide
ggagcatggaggccagctctggg

SEQ ID NO: 519 - DNA, 23 bp, Artificial Sequence
  Synthetic oligonucleotide
ggaacagggaggccagctccagg

SEQ ID NO: 520 - DNA, 23 bp, Artificial Sequence
  Synthetic oligonucleotide
agaacaaggaagccagctccagg

SEQ ID NO: 521 - DNA, 23 bp, Artificial Sequence
  Synthetic oligonucleotide
ggataatagaagccagctgctgg

SEQ ID NO: 522 - DNA, 23 bp, Artificial Sequence
  Synthetic oligonucleotide
ggattatggaagtaagctcctgg

SEQ ID NO: 523 - DNA, 23 bp, Artificial Sequence
  Synthetic oligonucleotide
gtagcatggaagtcagccccagg

SEQ ID NO: 524 - DNA, 23 bp, Artificial Sequence
  Synthetic oligonucleotide
ggatcatgaagaccagcccctgg

SEQ ID NO: 525 - DNA, 23 bp, Artificial Sequence
  Synthetic oligonucleotide
agatcacagcagccagctcctgg

SEQ ID NO: 526 - DNA, 23 bp, Artificial Sequence
  Synthetic oligonucleotide
gaatcagggaagccaccaccagg

SEQ ID NO: 527 - DNA, 23 bp, Artificial Sequence
  Synthetic oligonucleotide
ggaagacggaagccagatccagg

SEQ ID NO: 528 - DNA, 23 bp, Artificial Sequence
  Synthetic oligonucleotide
ggatcaagtaagtcagcaccagg

SEQ ID NO: 529 - DNA, 23 bp, Artificial Sequence
  Synthetic oligonucleotide
agatcataaaagtcagcacctgg

SEQ ID NO: 530 - DNA, 23 bp, Artificial Sequence
  Synthetic oligonucleotide
cagcaatggaagccagctccagg

SEQ ID NO: 531 - DNA, 23 bp, Artificial Sequence
  Synthetic oligonucleotide
gggacatgagagccaggaccctg

SEQ ID NO: 532 - DNA, 23 bp, Artificial Sequence
  Synthetic oligonucleotide
ggtctcggcacttggctcgctgg

SEQ ID NO: 533 - DNA, 23 bp, Artificial Sequence
  Synthetic oligonucleotide
gttctcggcacctggctctccgg

SEQ ID NO: 534 - DNA, 23 bp, Artificial Sequence
  Synthetic oligonucleotide
gctctcagaacctggctcgcggg

SEQ ID NO: 535 - DNA, 23 bp, Artificial Sequence
  Synthetic oligonucleotide
ggccttggcacctggctcccagg

SEQ ID NO: 536 - DNA, 23 bp, Artificial Sequence
  Synthetic oligonucleotide
ggaggcttcacagcgccctctgg

SEQ ID NO: 537 - DNA, 23 bp, Artificial Sequence
  Synthetic oligonucleotide
ggagccttcacagtgccctcggg

SEQ ID NO: 538 - DNA, 23 bp, Artificial Sequence
  Synthetic oligonucleotide
ccaggctccacagcgccctctgc

SEQ ID NO: 539 - DNA, 23 bp, Artificial Sequence
  Synthetic oligonucleotide
agaggcttcccagcaccctcggg

SEQ ID NO: 540 - DNA, 23 bp, Artificial Sequence
  Synthetic oligonucleotide
tcaggcttcacagcgccccctgg

SEQ ID NO: 541 - DNA, 23 bp, Artificial Sequence
  Synthetic oligonucleotide
gccggcttcacagcgccccccgg

SEQ ID NO: 542 - DNA, 23 bp, Artificial Sequence
  Synthetic oligonucleotide
agagacttcacagcaccctctgc

SEQ ID NO: 543 - DNA, 23 bp, Artificial Sequence
  Synthetic oligonucleotide
catggcttcacagtgccctcagg

SEQ ID NO: 544 - DNA, 23 bp, Artificial Sequence
  Synthetic oligonucleotide
ggtggccccacagcaccctctgg

SEQ ID NO: 545 - DNA, 23 bp, Artificial Sequence
  Synthetic oligonucleotide
attggcttcacagtgccctctgg

SEQ ID NO: 546 - DNA, 23 bp, Artificial Sequence
  Synthetic oligonucleotide
gggggctcctcagccccctcggg

SEQ ID NO: 547 - DNA, 23 bp, Artificial Sequence
  Synthetic oligonucleotide
ggaagcagcacagcaccctctgg

SEQ ID NO: 548 - DNA, 23 bp, Artificial Sequence
  Synthetic oligonucleotide
agaggatgcacagcaccctcagg

SEQ ID NO: 549 - DNA, 23 bp, Artificial Sequence
  Synthetic oligonucleotide
agaagctgcacagtgccctctgg

SEQ ID NO: 550 - DNA, 23 bp, Artificial Sequence
  Synthetic oligonucleotide
acagtcttcacagcaccctccgg

SEQ ID NO: 551 - DNA, 23 bp, Artificial Sequence
  Synthetic oligonucleotide
agtggcttcccagtgccctcagg

SEQ ID NO: 552 - DNA, 23 bp, Artificial Sequence
  Synthetic oligonucleotide
ataggctccacagaaccctccgg

SEQ ID NO: 553 - DNA, 23 bp, Artificial Sequence
  Synthetic oligonucleotide
aaaggctccccagtgccctcagg

SEQ ID NO: 554 - DNA, 23 bp, Artificial Sequence
  Synthetic oligonucleotide
gaggccctcacagcaccctcagg

SEQ ID NO: 555 - DNA, 23 bp, Artificial Sequence
  Synthetic oligonucleotide
agacactccacagccccctctgg

SEQ ID NO: 556 - DNA, 23 bp, Artificial Sequence
  Synthetic oligonucleotide
cctggctccccagtgccctcagg

SEQ ID NO: 557 - DNA, 23 bp, Artificial Sequence
  Synthetic oligonucleotide
cccggctccccagcgcccccggg

SEQ ID NO: 558 - DNA, 23 bp, Artificial Sequence
  Synthetic oligonucleotide
cagggctccccagtgccctcagg

SEQ ID NO: 559 - DNA, 23 bp, Artificial Sequence
  Synthetic oligonucleotide
cctggctccactgcaccctccgg

SEQ ID NO: 560 - DNA, 23 bp, Artificial Sequence
  Synthetic oligonucleotide
catggctccccagtgccctcagg

SEQ ID NO: 561 - DNA, 23 bp, Artificial Sequence
  Synthetic oligonucleotide
gctagtaccttgtatgaagatgg

SEQ ID NO: 562 - DNA, 23 bp, Artificial Sequence
  Synthetic oligonucleotide
tctagtgccttgtatgaagttgg

SEQ ID NO: 563 - DNA, 23 bp, Artificial Sequence
  Synthetic oligonucleotide
actagtaccctgcaagaagatgg

SEQ ID NO: 564 - DNA, 23 bp, Artificial Sequence
  Synthetic oligonucleotide
actggtatcttataagaagaggg

SEQ ID NO: 565 - DNA, 23 bp, Artificial Sequence
  Synthetic oligonucleotide
gacgggaaagtcagtgtgaatgg

SEQ ID NO: 566 - DNA, 23 bp, Artificial Sequence
  Synthetic oligonucleotide
ggagggaaagccagtgtgaaggg

SEQ ID NO: 567 - DNA, 23 bp, Artificial Sequence
  Synthetic oligonucleotide
gttcgaccatgcccttgcttagg

SEQ ID NO: 568 - DNA, 23 bp, Artificial Sequence
  Synthetic oligonucleotide
tgtagaccatgccattgctttgg

SEQ ID NO: 569 - DNA, 23 bp, Artificial Sequence
  Synthetic oligonucleotide
gctcggccagccccttgctctgg

SEQ ID NO: 570 - DNA, 23 bp, Artificial Sequence
  Synthetic oligonucleotide
ggcagagctcacctgtagatagg

SEQ ID NO: 571 - DNA, 23 bp, Artificial Sequence
  Synthetic oligonucleotide
caaagagctcacctgtagatcag

SEQ ID NO: 572 - DNA, 23 bp, Artificial Sequence
  Synthetic oligonucleotide
agcagagccctactgtagattgg

SEQ ID NO: 573 - DNA, 23 bp, Artificial Sequence
  Synthetic oligonucleotide
cacagagaacacctggagatggg

SEQ ID NO: 574 - DNA, 23 bp, Artificial Sequence
  Synthetic oligonucleotide
cacagagaacacctggagatggg

SEQ ID NO: 575 - DNA, 23 bp, Artificial Sequence
  Synthetic oligonucleotide
gctggtggagctcttctcaatgg

SEQ ID NO: 576 - DNA, 23 bp, Artificial Sequence
  Synthetic oligonucleotide
gctagtggagctcttctccacgg

SEQ ID NO: 577 - DNA, 23 bp, Artificial Sequence
  Synthetic oligonucleotide
gctggtggggctcttctcagaag

SEQ ID NO: 578 - DNA, 23 bp, Artificial Sequence
  Synthetic oligonucleotide
tgtggtggtgctcttctcaagag

SEQ ID NO: 579 - DNA, 23 bp, Artificial Sequence
  Synthetic oligonucleotide
cctggtggttctcttctcaatgg

SEQ ID NO: 580 - DNA, 23 bp, Artificial Sequence
  Synthetic oligonucleotide
gctgggctagctcttctcaaggg

SEQ ID NO: 581 - DNA, 23 bp, Artificial Sequence
  Synthetic oligonucleotide
cttgggggagctcttctcaagga

SEQ ID NO: 582 - DNA, 23 bp, Artificial Sequence
  Synthetic oligonucleotide
gctggagaggctcttctcaagga

SEQ ID NO: 583 - DNA, 23 bp, Artificial Sequence
  Synthetic oligonucleotide
actggagcagcccttctcaatgg

SEQ ID NO: 584 - DNA, 23 bp, Artificial Sequence
  Synthetic oligonucleotide
actggtcaagctcttcccaacgg

SEQ ID NO: 585 - DNA, 23 bp, Artificial Sequence
  Synthetic oligonucleotide
gcttgtggagcccttcccagggg

SEQ ID NO: 586 - DNA, 23 bp, Artificial Sequence
  Synthetic oligonucleotide
actggtgaagctctagtcaaagg

SEQ ID NO: 587 - DNA, 23 bp, Artificial Sequence
  Synthetic oligonucleotide
gctgggggagcccttctctgtgg

SEQ ID NO: 588 - DNA, 23 bp, Artificial Sequence
  Synthetic oligonucleotide
tctgggggggcccttctcaaggg

SEQ ID NO: 589 - DNA, 23 bp, Artificial Sequence
  Synthetic oligonucleotide
accagaggagctcttctcaaagg

SEQ ID NO: 590 - DNA, 23 bp, Artificial Sequence
  Synthetic oligonucleotide
gctggtgatgcccttctccaggg

SEQ ID NO: 591 - DNA, 23 bp, Artificial Sequence
  Synthetic oligonucleotide
gctgccagagcccttctcaaaag

SEQ ID NO: 592 - DNA, 23 bp, Artificial Sequence
  Synthetic oligonucleotide
gctgatggtgcccttctaaaagg

SEQ ID NO: 593 - DNA, 23 bp, Artificial Sequence
  Synthetic oligonucleotide
gctggtgacccccttctcaaggg

SEQ ID NO: 594 - DNA, 23 bp, Artificial Sequence
  Synthetic oligonucleotide
aggggtggagcccttctcaaaga

SEQ ID NO: 595 - DNA, 23 bp, Artificial Sequence
  Synthetic oligonucleotide
tatggtggaggacttctcaaagg

SEQ ID NO: 596 - DNA, 23 bp, Artificial Sequence
  Synthetic oligonucleotide
aatggtggagcccttctgaatgg

SEQ ID NO: 597 - DNA, 23 bp, Artificial Sequence
  Synthetic oligonucleotide
gctaggggacctcttctccaagg

SEQ ID NO: 598 - DNA, 23 bp, Artificial Sequence
  Synthetic oligonucleotide
gagggtggagcccttatcaatgg

SEQ ID NO: 599 - DNA, 23 bp, Artificial Sequence
  Synthetic oligonucleotide
cctggagaagctcttctccaggg

SEQ ID NO: 600 - DNA, 23 bp, Artificial Sequence
  Synthetic oligonucleotide
actggaggagctcctctcagagg

SEQ ID NO: 601 - DNA, 23 bp, Artificial Sequence
  Synthetic oligonucleotide
gctggggaggcccttctcaagga

SEQ ID NO: 602 - DNA, 23 bp, Artificial Sequence
  Synthetic oligonucleotide
gctgttggaccccttctcagagg

SEQ ID NO: 603 - DNA, 23 bp, Artificial Sequence
  Synthetic oligonucleotide
gctgggagggctcttcccaatgg

SEQ ID NO: 604 - DNA, 23 bp, Artificial Sequence
  Synthetic oligonucleotide
aagggtggagcccttatcaatgg

SEQ ID NO: 605 - DNA, 23 bp, Artificial Sequence
  Synthetic oligonucleotide
tttggggaagcccttctcaaggg

SEQ ID NO: 606 - DNA, 23 bp, Artificial Sequence
  Synthetic oligonucleotide
ttctgaggagctcttctcaaggg

SEQ ID NO: 607 - DNA, 23 bp, Artificial Sequence
  Synthetic oligonucleotide
gtcagtggagcccttctcagggg

SEQ ID NO: 608 - DNA, 23 bp, Artificial Sequence
  Synthetic oligonucleotide
actgatgggtcttttctcaaggg

SEQ ID NO: 609 - DNA, 23 bp, Artificial Sequence
  Synthetic oligonucleotide
gctgtagaagcccttcccaatgg

SEQ ID NO: 610 - DNA, 23 bp, Artificial Sequence
  Synthetic oligonucleotide
gcgggcacagctcttctaaaggg

SEQ ID NO: 611 - DNA, 23 bp, Artificial Sequence
  Synthetic oligonucleotide
aagggtggagccctcatcaatgg

SEQ ID NO: 612 - DNA, 23 bp, Artificial Sequence
  Synthetic oligonucleotide
gctggcgcagccccttccaaggg

SEQ ID NO: 613 - DNA, 23 bp, Artificial Sequence
  Synthetic oligonucleotide
ggagctgacccaagacgttctgg

SEQ ID NO: 614 - DNA, 23 bp, Artificial Sequence
  Synthetic oligonucleotide
agagctgactgaagacgttccgg

SEQ ID NO: 615 - DNA, 23 bp, Artificial Sequence
  Synthetic oligonucleotide
acaactgacccaagacgtgcagg

SEQ ID NO: 616 - DNA, 23 bp, Artificial Sequence
  Synthetic oligonucleotide
gttgaccatcagattgagacagg

SEQ ID NO: 617 - DNA, 23 bp, Artificial Sequence
  Synthetic oligonucleotide
gctcacctcgtgtccgttgctgg

SEQ ID NO: 618 - DNA, 23 bp, Artificial Sequence
  Synthetic oligonucleotide
ggacgttcatgtatttgctttgg

SEQ ID NO: 619 - DNA, 23 bp, Artificial Sequence
  Synthetic oligonucleotide
agatgttcatgtatttgcttaaa

SEQ ID NO: 620 - DNA, 23 bp, Artificial Sequence
  Synthetic oligonucleotide
acacactcatgtatttgcttagg

SEQ ID NO: 621 - DNA, 23 bp, Artificial Sequence
  Synthetic oligonucleotide
gctgtaaaagacatccctgatgg

SEQ ID NO: 622 - DNA, 23 bp, Artificial Sequence
  Synthetic oligonucleotide
gctggaaaaggcatccctgaggg

SEQ ID NO: 623 - DNA, 23 bp, Artificial Sequence
  Synthetic oligonucleotide
tctgtaagagacatccctgatgt

SEQ ID NO: 624 - DNA, 23 bp, Artificial Sequence
  Synthetic oligonucleotide
tctgtaaaaggcatccctgaaag

SEQ ID NO: 625 - DNA, 23 bp, Artificial Sequence
  Synthetic oligonucleotide
gcagtgaaagacatccctgtggg

SEQ ID NO: 626 - DNA, 23 bp, Artificial Sequence
  Synthetic oligonucleotide
gctgtagaagacatcccagtaag

SEQ ID NO: 627 - DNA, 23 bp, Artificial Sequence
  Synthetic oligonucleotide
atagtaaaagacatccctgtggc

SEQ ID NO: 628 - DNA, 23 bp, Artificial Sequence
  Synthetic oligonucleotide
gggtctccaccacttcgtaaagg

SEQ ID NO: 629 - DNA, 23 bp, Artificial Sequence
  Synthetic oligonucleotide
gagtctccaccatttcataatgg

SEQ ID NO: 630 - DNA, 23 bp, Artificial Sequence
  Synthetic oligonucleotide
ggcggcgctcacaattgccacgg

SEQ ID NO: 631 - DNA, 23 bp, Artificial Sequence
  Synthetic oligonucleotide
ggtagagctcacaattgccaagg

SEQ ID NO: 632 - DNA, 23 bp, Artificial Sequence
  Synthetic oligonucleotide
aggggcgcccacaattgccaagg

SEQ ID NO: 633 - DNA, 23 bp, Artificial Sequence
  Synthetic oligonucleotide
aggggcgctcagaattgccaagg

SEQ ID NO: 634 - DNA, 23 bp, Artificial Sequence
  Synthetic oligonucleotide
ggagccactcacaattgccaagg

SEQ ID NO: 635 - DNA, 23 bp, Artificial Sequence
  Synthetic oligonucleotide
aggggcacccacaattgccaagg

SEQ ID NO: 636 - DNA, 23 bp, Artificial Sequence
  Synthetic oligonucleotide
aggggcgcccacaattgcccagg

SEQ ID NO: 637 - DNA, 23 bp, Artificial Sequence
  Synthetic oligonucleotide
aggggcacccacaattgccaagg

SEQ ID NO: 638 - DNA, 23 bp, Artificial Sequence
  Synthetic oligonucleotide
aggggcgcccacaattgcctagg

SEQ ID NO: 639 - DNA, 23 bp, Artificial Sequence
  Synthetic oligonucleotide
aggggtgcccacaattgccaagg

SEQ ID NO: 640 - DNA, 23 bp, Artificial Sequence
  Synthetic oligonucleotide
aggggcccccacaattgccaagg

SEQ ID NO: 641 - DNA, 23 bp, Artificial Sequence
  Synthetic oligonucleotide
aggggcgcccaccattgccaagg

SEQ ID NO: 642 - DNA, 23 bp, Artificial Sequence
  Synthetic oligonucleotide
aggggcgcccacaattgcctaga

SEQ ID NO: 643 - DNA, 23 bp, Artificial Sequence
  Synthetic oligonucleotide
aggggcgcccaccattgccaagg

SEQ ID NO: 644 - DNA, 23 bp, Artificial Sequence
  Synthetic oligonucleotide
aggggcgcccaccattgccaagg

SEQ ID NO: 645 - DNA, 23 bp, Artificial Sequence
  Synthetic oligonucleotide
aggggcgcccaccattgccaagg

SEQ ID NO: 646 - DNA, 23 bp, Artificial Sequence
  Synthetic oligonucleotide
aggggcacccacaattgcccagg

SEQ ID NO: 647 - DNA, 23 bp, Artificial Sequence
  Synthetic oligonucleotide
aggggcacccacaattgcccagt

SEQ ID NO: 648 - DNA, 23 bp, Artificial Sequence
  Synthetic oligonucleotide
agcaccattcacaattgccaagg

SEQ ID NO: 649 - DNA, 23 bp, Artificial Sequence
  Synthetic oligonucleotide
aggggcgcccgccattgccaagg

SEQ ID NO: 650 - DNA, 23 bp, Artificial Sequence
  Synthetic oligonucleotide
aggggtgcccaccattgccaagt

SEQ ID NO: 651 - DNA, 23 bp, Artificial Sequence
  Synthetic oligonucleotide
tggggcacccacaatttccaagg

SEQ ID NO: 652 - DNA, 23 bp, Artificial Sequence
  Synthetic oligonucleotide
aggggcgcccgccattgccaagg

SEQ ID NO: 653 - DNA, 23 bp, Artificial Sequence
  Synthetic oligonucleotide
aggggggcccacaattgcccagg

SEQ ID NO: 654 - DNA, 23 bp, Artificial Sequence
  Synthetic oligonucleotide
aggggcacccacaattgctcagg

SEQ ID NO: 655 - DNA, 23 bp, Artificial Sequence
  Synthetic oligonucleotide
aggggcacccacaattgctgagg

SEQ ID NO: 656 - DNA, 23 bp, Artificial Sequence
  Synthetic oligonucleotide
aggggcgcccaccattgaccagg

SEQ ID NO: 657 - DNA, 23 bp, Artificial Sequence
  Synthetic oligonucleotide
aggggtgcccacaattgctgagg

SEQ ID NO: 658 - DNA, 23 bp, Artificial Sequence
  Synthetic oligonucleotide
aggggtgcccgccattgccaagg

SEQ ID NO: 659 - DNA, 23 bp, Artificial Sequence
  Synthetic oligonucleotide
aggggcgcccaccattgcacagg

SEQ ID NO: 660 - DNA, 23 bp, Artificial Sequence
  Synthetic oligonucleotide
aggggcacccactattgcccagg

SEQ ID NO: 661 - DNA, 23 bp, Artificial Sequence
  Synthetic oligonucleotide
ggtacttactgttactcgcaagg

SEQ ID NO: 662 - DNA, 23 bp, Artificial Sequence
  Synthetic oligonucleotide
ggtacttactgctactctccagg

SEQ ID NO: 663 - DNA, 23 bp, Artificial Sequence
  Synthetic oligonucleotide
gacgctggtcaaacgccttgcgg

SEQ ID NO: 664 - DNA, 23 bp, Artificial Sequence
  Synthetic oligonucleotide
gacccaggtcaaacgcctttagg

SEQ ID NO: 665 - DNA, 23 bp, Artificial Sequence
  Synthetic oligonucleotide
ggcatgctgcggcatgagatagg

SEQ ID NO: 666 - DNA, 23 bp, Artificial Sequence
  Synthetic oligonucleotide
ggcatgctgtggcatgaaatagg

SEQ ID NO: 667 - DNA, 23 bp, Artificial Sequence
  Synthetic oligonucleotide
ggcttgctgcagcatgagttagg

SEQ ID NO: 668 - DNA, 23 bp, Artificial Sequence
  Synthetic oligonucleotide
ggaatgctgcggcatgatcttgg

SEQ ID NO: 669 - DNA, 23 bp, Artificial Sequence
  Synthetic oligonucleotide
cggatgctgcagcaagagattgg

SEQ ID NO: 670 - DNA, 23 bp, Artificial Sequence
  Synthetic oligonucleotide
cacatgatgcagcatgagatggg

SEQ ID NO: 671 - DNA, 23 bp, Artificial Sequence
  Synthetic oligonucleotide
cggatgctgcagcaagagattgg

SEQ ID NO: 672 - DNA, 23 bp, Artificial Sequence
  Synthetic oligonucleotide
gacgggctgctccatgaggtaga

SEQ ID NO: 673 - DNA, 23 bp, Artificial Sequence
  Synthetic oligonucleotide
ggctccacgcagacgctgacagg

SEQ ID NO: 674 - DNA, 23 bp, Artificial Sequence
  Synthetic oligonucleotide
gtcgaggagaatgaggaaaatgg

SEQ ID NO: 675 - DNA, 23 bp, Artificial Sequence
  Synthetic oligonucleotide
ttagaggagaatgaggaaaagag

SEQ ID NO: 676 - DNA, 23 bp, Artificial Sequence
  Synthetic oligonucleotide
gtggaggagaaagaggaaaaggg

SEQ ID NO: 677 - DNA, 23 bp, Artificial Sequence
  Synthetic oligonucleotide
gtagaggagtatgaggaaaagag

SEQ ID NO: 678 - DNA, 23 bp, Artificial Sequence
  Synthetic oligonucleotide
gttgaggagaaggaggaaaagga

SEQ ID NO: 679 - DNA, 23 bp, Artificial Sequence
  Synthetic oligonucleotide
gtccaggagaatgaggaaaggag

SEQ ID NO: 680 - DNA, 23 bp, Artificial Sequence
  Synthetic oligonucleotide
gttgaggagaaggaggaaaagga

SEQ ID NO: 681 - DNA, 23 bp, Artificial Sequence
  Synthetic oligonucleotide
gttgagtagaatgaggaaaagga

SEQ ID NO: 682 - DNA, 23 bp, Artificial Sequence
  Synthetic oligonucleotide
agggaggagaatgaggaaaaggg

SEQ ID NO: 683 - DNA, 23 bp, Artificial Sequence
  Synthetic oligonucleotide
gaggaggagaaagaggaaaaggg

SEQ ID NO: 684 - DNA, 23 bp, Artificial Sequence
  Synthetic oligonucleotide
gaaggggagaatgaggaaaaggg

SEQ ID NO: 685 - DNA, 23 bp, Artificial Sequence
  Synthetic oligonucleotide
gccaaggagaatgagaaaaagag

SEQ ID NO: 686 - DNA, 23 bp, Artificial Sequence
  Synthetic oligonucleotide
ggagaggagaatgaggagaaggg

SEQ ID NO: 687 - DNA, 23 bp, Artificial Sequence
  Synthetic oligonucleotide
gatgaggagcatgaggaaaaggg

SEQ ID NO: 688 - DNA, 23 bp, Artificial Sequence
  Synthetic oligonucleotide
gaagaggagaatgagaaaaaggg

SEQ ID NO: 689 - DNA, 23 bp, Artificial Sequence
  Synthetic oligonucleotide
ctggagaagaatgaggaaaaagg

SEQ ID NO: 690 - DNA, 23 bp, Artificial Sequence
  Synthetic oligonucleotide
gttgaggggaatgaggataaggg

SEQ ID NO: 691 - DNA, 23 bp, Artificial Sequence
  Synthetic oligonucleotide
gaggaggagaaagaggaaaaaag

SEQ ID NO: 692 - DNA, 23 bp, Artificial Sequence
  Synthetic oligonucleotide
gacgaagagaaagaggaaaagag

SEQ ID NO: 693 - DNA, 23 bp, Artificial Sequence
  Synthetic oligonucleotide
ctcgaggggaatgaggagaaggg

SEQ ID NO: 694 - DNA, 23 bp, Artificial Sequence
  Synthetic oligonucleotide
gttgaggagaaaaaggaaaaggg

SEQ ID NO: 695 - DNA, 23 bp, Artificial Sequence
  Synthetic oligonucleotide
acagaggagaatgaggaaaaaga

SEQ ID NO: 696 - DNA, 23 bp, Artificial Sequence
  Synthetic oligonucleotide
gatgaggggaatgaggaaaacag

SEQ ID NO: 697 - DNA, 23 bp, Artificial Sequence
  Synthetic oligonucleotide
gaggaagagaatgaggaaaagga

SEQ ID NO: 698 - DNA, 23 bp, Artificial Sequence
  Synthetic oligonucleotide
ggtgaggagaaggaggaaaagga

SEQ ID NO: 699 - DNA, 23 bp, Artificial Sequence
  Synthetic oligonucleotide
gaagaggagaaggaggaaaagga

SEQ ID NO: 700 - DNA, 23 bp, Artificial Sequence
  Synthetic oligonucleotide
gtggaggaggaggaggaaaatgg

SEQ ID NO: 701 - DNA, 23 bp, Artificial Sequence
  Synthetic oligonucleotide
gctgaggagaaggaggaaaagga

SEQ ID NO: 702 - DNA, 23 bp, Artificial Sequence
  Synthetic oligonucleotide
aacaaggagaatgaggaaaaagc

SEQ ID NO: 703 - DNA, 23 bp, Artificial Sequence
  Synthetic oligonucleotide
gtggagaagaatgaggagaaagg

SEQ ID NO: 704 - DNA, 23 bp, Artificial Sequence
  Synthetic oligonucleotide
gtggaggaggaggaggaaaaggg

SEQ ID NO: 705 - DNA, 23 bp, Artificial Sequence
  Synthetic oligonucleotide
gaagaggagaatgaggacaagag

SEQ ID NO: 706 - DNA, 23 bp, Artificial Sequence
  Synthetic oligonucleotide
gaagaggagaatgagaaaaagga

SEQ ID NO: 707 - DNA, 23 bp, Artificial Sequence
  Synthetic oligonucleotide
gtggaggagaaagaggagaagag

SEQ ID NO: 708 - DNA, 23 bp, Artificial Sequence
  Synthetic oligonucleotide
gtggaagagaatgaagaaaagag

SEQ ID NO: 709 - DNA, 23 bp, Artificial Sequence
  Synthetic oligonucleotide
gctgaggagaaggaggaaaagga

SEQ ID NO: 710 - DNA, 23 bp, Artificial Sequence
  Synthetic oligonucleotide
gttgcagagaatgaggaaaaggg

SEQ ID NO: 711 - DNA, 23 bp, Artificial Sequence
  Synthetic oligonucleotide
gctgaggagaaggaggaaaagga

SEQ ID NO: 712 - DNA, 23 bp, Artificial Sequence
  Synthetic oligonucleotide
gctgaggagaatgaggagaacag

SEQ ID NO: 713 - DNA, 23 bp, Artificial Sequence
  Synthetic oligonucleotide
gaagaggagaaggaggaaaaaga

SEQ ID NO: 714 - DNA, 23 bp, Artificial Sequence
  Synthetic oligonucleotide
gctgaggagaaggaggaaaagga

SEQ ID NO: 715 - DNA, 23 bp, Artificial Sequence
  Synthetic oligonucleotide
gcagaggagaaagaggaaaaaga

SEQ ID NO: 716 - DNA, 23 bp, Artificial Sequence
  Synthetic oligonucleotide
atggaggagaatgaagaaaaaag

SEQ ID NO: 717 - DNA, 23 bp, Artificial Sequence
  Synthetic oligonucleotide
gtagaggagagagaggaaaagag

SEQ ID NO: 718 - DNA, 23 bp, Artificial Sequence
  Synthetic oligonucleotide
gtagaggagaaagagaaaaagag

SEQ ID NO: 719 - DNA, 23 bp, Artificial Sequence
  Synthetic oligonucleotide
actgaggagaaagaggaaaatgg

SEQ ID NO: 720 - DNA, 23 bp, Artificial Sequence
  Synthetic oligonucleotide
agtgaggggaatgaggaaaaagg

SEQ ID NO: 721 - DNA, 23 bp, Artificial Sequence
  Synthetic oligonucleotide
aatgaggagtatgaggaaaaggg

SEQ ID NO: 722 - DNA, 23 bp, Artificial Sequence
  Synthetic oligonucleotide
agaggggagaatgaggaaaatgg

SEQ ID NO: 723 - DNA, 23 bp, Artificial Sequence
  Synthetic oligonucleotide
acagaggggaatgaggaaaaggg

SEQ ID NO: 724 - DNA, 23 bp, Artificial Sequence
  Synthetic oligonucleotide
aaggaggagaaagaggaaaatgg

SEQ ID NO: 725 - DNA, 23 bp, Artificial Sequence
  Synthetic oligonucleotide
taagagcagaatgaggaaaaggg

SEQ ID NO: 726 - DNA, 23 bp, Artificial Sequence
  Synthetic oligonucleotide
gaggaggagaaagagaaaaaggg

SEQ ID NO: 727 - DNA, 23 bp, Artificial Sequence
  Synthetic oligonucleotide
aaagaagagaatgaggaaaatgg

SEQ ID NO: 728 - DNA, 23 bp, Artificial Sequence
  Synthetic oligonucleotide
ggggaggagaaagaggaaagggg

SEQ ID NO: 729 - DNA, 23 bp, Artificial Sequence
  Synthetic oligonucleotide
agagaggagaaggaggaaaaagg

SEQ ID NO: 730 - DNA, 23 bp, Artificial Sequence
  Synthetic oligonucleotide
gaggaggagaaagagaaaaaagg

SEQ ID NO: 731 - DNA, 23 bp, Artificial Sequence
  Synthetic oligonucleotide
tgggaggagaatgagggaaaagg

SEQ ID NO: 732 - DNA, 23 bp, Artificial Sequence
  Synthetic oligonucleotide
gaggaggagaaagaggagaaggg

SEQ ID NO: 733 - DNA, 23 bp, Artificial Sequence
  Synthetic oligonucleotide
aaggagcagaatgaggaaaaagg

SEQ ID NO: 734 - DNA, 23 bp, Artificial Sequence
  Synthetic oligonucleotide
gaggaggagaaagaggaataggg

SEQ ID NO: 735 - DNA, 23 bp, Artificial Sequence
  Synthetic oligonucleotide
ctggaggagaaagaggaataggg

SEQ ID NO: 736 - DNA, 23 bp, Artificial Sequence
  Synthetic oligonucleotide
gcaaaggagaatgagaaaaaagg

SEQ ID NO: 737 - DNA, 23 bp, Artificial Sequence
  Synthetic oligonucleotide
ccagaggagactgaggaaaatgg

SEQ ID NO: 738 - DNA, 23 bp, Artificial Sequence
  Synthetic oligonucleotide
ggtgaggggaatgaggaaagagg

SEQ ID NO: 739 - DNA, 23 bp, Artificial Sequence
  Synthetic oligonucleotide
gagggggagaatggggaaaaggg

SEQ ID NO: 740 - DNA, 23 bp, Artificial Sequence
  Synthetic oligonucleotide
attgaggagaaagaggagaatgg

SEQ ID NO: 741 - DNA, 23 bp, Artificial Sequence
  Synthetic oligonucleotide
gctgaggagaaagaggaagaggg

SEQ ID NO: 742 - DNA, 23 bp, Artificial Sequence
  Synthetic oligonucleotide
aaagaggagaaagaggaaaagga

SEQ ID NO: 743 - DNA, 23 bp, Artificial Sequence
  Synthetic oligonucleotide
gaggaagagaaagaggaaaatgg

SEQ ID NO: 744 - DNA, 23 bp, Artificial Sequence
  Synthetic oligonucleotide
gaggaggagaaagaagaaaaagg

SEQ ID NO: 745 - DNA, 23 bp, Artificial Sequence
  Synthetic oligonucleotide
accgaagagaatgaggaaacaag

SEQ ID NO: 746 - DNA, 23 bp, Artificial Sequence
  Synthetic oligonucleotide
gaggaggagaataagaaaaatgg

SEQ ID NO: 747 - DNA, 23 bp, Artificial Sequence
  Synthetic oligonucleotide
atagagtagaatgaggaaagggg

SEQ ID NO: 748 - DNA, 23 bp, Artificial Sequence
  Synthetic oligonucleotide
aaggaggagaaagaggaaaagga

SEQ ID NO: 749 - DNA, 23 bp, Artificial Sequence
  Synthetic oligonucleotide
aaagaggagaaagaggaaaatag

SEQ ID NO: 750 - DNA, 23 bp, Artificial Sequence
  Synthetic oligonucleotide
gccaaggagaaagaggaagaggg

SEQ ID NO: 751 - DNA, 23 bp, Artificial Sequence
  Synthetic oligonucleotide
gaggaggaggaagaggaaaaggg

SEQ ID NO: 752 - DNA, 23 bp, Artificial Sequence
  Synthetic oligonucleotide
gaggaggagaaaggggaaaaggg

SEQ ID NO: 753 - DNA, 23 bp, Artificial Sequence
  Synthetic oligonucleotide
gctgaggagaaggaggaaagggg

SEQ ID NO: 754 - DNA, 23 bp, Artificial Sequence
  Synthetic oligonucleotide
ggtgaagagaatgaggaaagagg

SEQ ID NO: 755 - DNA, 23 bp, Artificial Sequence
  Synthetic oligonucleotide
tatgaggagaatgaggagaagag

SEQ ID NO: 756 - DNA, 23 bp, Artificial Sequence
  Synthetic oligonucleotide
ggggaggagaaagaagaaaaggg

SEQ ID NO: 757 - DNA, 23 bp, Artificial Sequence
  Synthetic oligonucleotide
aaggaggagaaagaggaaaaaga

SEQ ID NO: 758 - DNA, 23 bp, Artificial Sequence
  Synthetic oligonucleotide
gaggaggaaaaagaggaaaaggg

SEQ ID NO: 759 - DNA, 23 bp, Artificial Sequence
  Synthetic oligonucleotide
gaggaggagaaggaggagaacgg

SEQ ID NO: 760 - DNA, 23 bp, Artificial Sequence
  Synthetic oligonucleotide
gaagaggagaaagaggagaaagg

SEQ ID NO: 761 - DNA, 23 bp, Artificial Sequence
  Synthetic oligonucleotide
ctagaggagaatgagaagaatgg

SEQ ID NO: 762 - DNA, 23 bp, Artificial Sequence
  Synthetic oligonucleotide
gaggaagagaaagaggaaaatgg

SEQ ID NO: 763 - DNA, 23 bp, Artificial Sequence
  Synthetic oligonucleotide
gcagaggggaaagaggaaaaagg

SEQ ID NO: 764 - DNA, 23 bp, Artificial Sequence
  Synthetic oligonucleotide
gaggaggagaaagaggagaaagg

SEQ ID NO: 765 - DNA, 23 bp, Artificial Sequence
  Synthetic oligonucleotide
ggagaggaggaagaggaaaaggg

SEQ ID NO: 766 - DNA, 23 bp, Artificial Sequence
  Synthetic oligonucleotide
ttagaggagaaagaggaagaagg

SEQ ID NO: 767 - DNA, 23 bp, Artificial Sequence
  Synthetic oligonucleotide
ggagaggagagtgaggaagaggg

SEQ ID NO: 768 - DNA, 23 bp, Artificial Sequence
  Synthetic oligonucleotide
agtaaggagaatgaggaaaaagc

SEQ ID NO: 769 - DNA, 23 bp, Artificial Sequence
  Synthetic oligonucleotide
ggggaggagaaagaagaaaaggg

SEQ ID NO: 770 - DNA, 23 bp, Artificial Sequence
  Synthetic oligonucleotide
gaggaggagaaggaggagaaggg

SEQ ID NO: 771 - DNA, 23 bp, Artificial Sequence
  Synthetic oligonucleotide
gaggaggagaaagaagaaaacgg

SEQ ID NO: 772 - DNA, 23 bp, Artificial Sequence
  Synthetic oligonucleotide
gcagaggaggaggaggaaaaggg

SEQ ID NO: 773 - DNA, 23 bp, Artificial Sequence
  Synthetic oligonucleotide
gaggaggagaaggaggagaaggg

SEQ ID NO: 774 - DNA, 23 bp, Artificial Sequence
  Synthetic oligonucleotide
gctgagaagaatgaggaaagggg

SEQ ID NO: 775 - DNA, 23 bp, Artificial Sequence
  Synthetic oligonucleotide
gcagaggagaacaaggaaaagag

SEQ ID NO: 776 - DNA, 23 bp, Artificial Sequence
  Synthetic oligonucleotide
gaggaggagaaagaggaacaggg

SEQ ID NO: 777 - DNA, 23 bp, Artificial Sequence
  Synthetic oligonucleotide
gaggaggaggaggaggaaaaagg

SEQ ID NO: 778 - DNA, 23 bp, Artificial Sequence
  Synthetic oligonucleotide
gccaaggggaatgaggcaaaggg

SEQ ID NO: 779 - DNA, 23 bp, Artificial Sequence
  Synthetic oligonucleotide
gaggaggagaaggaggagaaggg

SEQ ID NO: 780 - DNA, 23 bp, Artificial Sequence
  Synthetic oligonucleotide
agagaggagaaggaggaaaagga

SEQ ID NO: 781 - DNA, 23 bp, Artificial Sequence
  Synthetic oligonucleotide
aaggaggagaatgaggagaagga

SEQ ID NO: 782 - DNA, 23 bp, Artificial Sequence
  Synthetic oligonucleotide
gaagaggagaaggaggagaaggg

SEQ ID NO: 783 - DNA, 23 bp, Artificial Sequence
  Synthetic oligonucleotide
gaggaggaggatgaggagaaggg

SEQ ID NO: 784 - DNA, 23 bp, Artificial Sequence
  Synthetic oligonucleotide
gaggaggaggaagaggaaaagga

SEQ ID NO: 785 - DNA, 23 bp, Artificial Sequence
  Synthetic oligonucleotide
gaggaggagaaggaggaagaggg

SEQ ID NO: 786 - DNA, 23 bp, Artificial Sequence
  Synthetic oligonucleotide
gaggaggagaaagaggaagaggg

SEQ ID NO: 787 - DNA, 23 bp, Artificial Sequence
  Synthetic oligonucleotide
ggagaggagaatgaggaggaggg

SEQ ID NO: 788 - DNA, 23 bp, Artificial Sequence
  Synthetic oligonucleotide
ggggaggagaaggaggacaaggg

SEQ ID NO: 789 - DNA, 23 bp, Artificial Sequence
  Synthetic oligonucleotide
ggtgaggagaaggaagaaaaagg

SEQ ID NO: 790 - DNA, 23 bp, Artificial Sequence
  Synthetic oligonucleotide
ggagaagagaatgagaaaaatgg

SEQ ID NO: 791 - DNA, 23 bp, Artificial Sequence
  Synthetic oligonucleotide
ggggaggagaaggaggagaaggg

SEQ ID NO: 792 - DNA, 23 bp, Artificial Sequence
  Synthetic oligonucleotide
gctgaggagaaggaggaagaagg

SEQ ID NO: 793 - DNA, 23 bp, Artificial Sequence
  Synthetic oligonucleotide
ggtgaggaggatgaggaaagggg

SEQ ID NO: 794 - DNA, 23 bp, Artificial Sequence
  Synthetic oligonucleotide
ggggaggagaaagaagaaaaggg

SEQ ID NO: 795 - DNA, 23 bp, Artificial Sequence
  Synthetic oligonucleotide
aaagaggagaaagaggaaaagga

SEQ ID NO: 796 - DNA, 23 bp, Artificial Sequence
  Synthetic oligonucleotide
agggaggagaatgaggagaaaag

SEQ ID NO: 797 - DNA, 23 bp, Artificial Sequence
  Synthetic oligonucleotide
gtagaggagaaccaggagaaggg

SEQ ID NO: 798 - DNA, 23 bp, Artificial Sequence
  Synthetic oligonucleotide
gcccaggagaaagagaaaaatgg

SEQ ID NO: 799 - DNA, 23 bp, Artificial Sequence
  Synthetic oligonucleotide
ggggaggagaaggaggagaaagg

SEQ ID NO: 800 - DNA, 23 bp, Artificial Sequence
  Synthetic oligonucleotide
aaggaggagaaggaggaaaagga

SEQ ID NO: 801 - DNA, 23 bp, Artificial Sequence
  Synthetic oligonucleotide
aaagaggagaatgaggagaagga

SEQ ID NO: 802 - DNA, 23 bp, Artificial Sequence
  Synthetic oligonucleotide
agtgaggagaatgaggaatatga

SEQ ID NO: 803 - DNA, 23 bp, Artificial Sequence
  Synthetic oligonucleotide
gccgaggaggaagaggagaaggg

SEQ ID NO: 804 - DNA, 23 bp, Artificial Sequence
  Synthetic oligonucleotide
gaagaggagaaggaggaagaggg

SEQ ID NO: 805 - DNA, 23 bp, Artificial Sequence
  Synthetic oligonucleotide
ggggaggaggataaggaaaaggg

SEQ ID NO: 806 - DNA, 23 bp, Artificial Sequence
  Synthetic oligonucleotide
ctagaagagagtgaggaaaaagg

SEQ ID NO: 807 - DNA, 23 bp, Artificial Sequence
  Synthetic oligonucleotide
gcagaggggaatgaggtaaaggg

SEQ ID NO: 808 - DNA, 23 bp, Artificial Sequence
  Synthetic oligonucleotide
gtcaaagagaaagagaaaaaagg

SEQ ID NO: 809 - DNA, 23 bp, Artificial Sequence
  Synthetic oligonucleotide
ctagaggaggaagaggaaaaagg

SEQ ID NO: 810 - DNA, 23 bp, Artificial Sequence
  Synthetic oligonucleotide
ttaaaggagaatgaggagaaggg

SEQ ID NO: 811 - DNA, 23 bp, Artificial Sequence
  Synthetic oligonucleotide
caggaggagaaggaggaaaagag

SEQ ID NO: 812 - DNA, 23 bp, Artificial Sequence
  Synthetic oligonucleotide
aaagaggagaatgaggataagga

SEQ ID NO: 813 - DNA, 23 bp, Artificial Sequence
  Synthetic oligonucleotide
gaggaggagaaagaggtaaaggg

SEQ ID NO: 814 - DNA, 23 bp, Artificial Sequence
  Synthetic oligonucleotide
gtggaggaggatgagaaaacagg

SEQ ID NO: 815 - DNA, 23 bp, Artificial Sequence
  Synthetic oligonucleotide
gatgagaagaatgaggaagaagg

SEQ ID NO: 816 - DNA, 23 bp, Artificial Sequence
  Synthetic oligonucleotide
catgagaagaatgagaaaaaagg

SEQ ID NO: 817 - DNA, 23 bp, Artificial Sequence
  Synthetic oligonucleotide
tgagaggagaaagagaaaaaggg

SEQ ID NO: 818 - DNA, 23 bp, Artificial Sequence
  Synthetic oligonucleotide
agggaagagaaagaggaaaaagg

SEQ ID NO: 819 - DNA, 23 bp, Artificial Sequence
  Synthetic oligonucleotide
aaggaggagaaagaggaaagagg

SEQ ID NO: 820 - DNA, 23 bp, Artificial Sequence
  Synthetic oligonucleotide
caggaggagaatgagaagaatgg

SEQ ID NO: 821 - DNA, 23 bp, Artificial Sequence
  Synthetic oligonucleotide
cctgaggagaatgagaagaaggg

SEQ ID NO: 822 - DNA, 23 bp, Artificial Sequence
  Synthetic oligonucleotide
caggaggagaaagagaaaaatgg

SEQ ID NO: 823 - DNA, 23 bp, Artificial Sequence
  Synthetic oligonucleotide
aagggggagaaagaggaaaaggg

SEQ ID NO: 824 - DNA, 23 bp, Artificial Sequence
  Synthetic oligonucleotide
agtgaggagaaagaggaagaagg

SEQ ID NO: 825 - DNA, 23 bp, Artificial Sequence
  Synthetic oligonucleotide
agagaggagaaagaggaagaggg

SEQ ID NO: 826 - DNA, 23 bp, Artificial Sequence
  Synthetic oligonucleotide
tgtgaggagaaagaggaaagggg

SEQ ID NO: 827 - DNA, 23 bp, Artificial Sequence
  Synthetic oligonucleotide
agggaggagaagaaggaaaaggg

SEQ ID NO: 828 - DNA, 23 bp, Artificial Sequence
  Synthetic oligonucleotide
aaggaggagaaagaggaaagggg

SEQ ID NO: 829 - DNA, 23 bp, Artificial Sequence
  Synthetic oligonucleotide
aaggaggagaaagagaaaaaagg

SEQ ID NO: 830 - DNA, 23 bp, Artificial Sequence
  Synthetic oligonucleotide
agaaaggagaatgaagaaaatgg

SEQ ID NO: 831 - DNA, 23 bp, Artificial Sequence
  Synthetic oligonucleotide
tgtgaggagaaagaagaaaatgg

SEQ ID NO: 832 - DNA, 23 bp, Artificial Sequence
  Synthetic oligonucleotide
tattaggagaaagaggaaaaggg

SEQ ID NO: 833 - DNA, 23 bp, Artificial Sequence
  Synthetic oligonucleotide
tgttaggagaatgaagaaaaggg

SEQ ID NO: 834 - DNA, 23 bp, Artificial Sequence
  Synthetic oligonucleotide
caggaagagaatgaggaaagggg

SEQ ID NO: 835 - DNA, 23 bp, Artificial Sequence
  Synthetic oligonucleotide
agagaggaaaaagaggaaaaagg

SEQ ID NO: 836 - DNA, 23 bp, Artificial Sequence
  Synthetic oligonucleotide
aaagaggagaatgaggaaggggg

SEQ ID NO: 837 - DNA, 23 bp, Artificial Sequence
  Synthetic oligonucleotide
tctaaggagaaagaggaaaatgg

SEQ ID NO: 838 - DNA, 23 bp, Artificial Sequence
  Synthetic oligonucleotide
agtgaggagaaagagggaaaggg

SEQ ID NO: 839 - DNA, 23 bp, Artificial Sequence
  Synthetic oligonucleotide
tgttaggagaaagaggaaaatgg

SEQ ID NO: 840 - DNA, 23 bp, Artificial Sequence
  Synthetic oligonucleotide
agagaggagaaggaggagaaagg

SEQ ID NO: 841 - DNA, 23 bp, Artificial Sequence
  Synthetic oligonucleotide
caggaggggaaagaggaaaaggg

SEQ ID NO: 842 - DNA, 23 bp, Artificial Sequence
  Synthetic oligonucleotide
agagaggagaaagaggagaaggg

SEQ ID NO: 843 - DNA, 23 bp, Artificial Sequence
  Synthetic oligonucleotide
tgtgaggagtataaggaaaatgg

SEQ ID NO: 844 - DNA, 23 bp, Artificial Sequence
  Synthetic oligonucleotide
aaagaggagaaagaggagaaggg

SEQ ID NO: 845 - DNA, 23 bp, Artificial Sequence
  Synthetic oligonucleotide
aaggaggagaaggaggagaaggg

SEQ ID NO: 846 - DNA, 23 bp, Artificial Sequence
  Synthetic oligonucleotide
tgagaggagaaaaaggaaaaagg

SEQ ID NO: 847 - DNA, 23 bp, Artificial Sequence
  Synthetic oligonucleotide
acagaggagaagaaggaaaaagg

SEQ ID NO: 848 - DNA, 23 bp, Artificial Sequence
  Synthetic oligonucleotide
aaggaggagaaggagaaaaaggg

SEQ ID NO: 849 - DNA, 23 bp, Artificial Sequence
  Synthetic oligonucleotide
caggaggagaaagaggaagatgg

SEQ ID NO: 850 - DNA, 23 bp, Artificial Sequence
  Synthetic oligonucleotide
tgaaaggagaaagaggaaaaagg

SEQ ID NO: 851 - DNA, 23 bp, Artificial Sequence
  Synthetic oligonucleotide
agggaggagagggaggaaaaggg

SEQ ID NO: 852 - DNA, 23 bp, Artificial Sequence
  Synthetic oligonucleotide
aaggagaagaaagaggaaaaggg

SEQ ID NO: 853 - DNA, 23 bp, Artificial Sequence
  Synthetic oligonucleotide
ccagaggagaaggaggagaaggg

SEQ ID NO: 854 - DNA, 23 bp, Artificial Sequence
  Synthetic oligonucleotide
tgggaagagaatggggaaaaggg

SEQ ID NO: 855 - DNA, 23 bp, Artificial Sequence
  Synthetic oligonucleotide
aaggaggagaaagaggaagaagg

SEQ ID NO: 856 - DNA, 23 bp, Artificial Sequence
  Synthetic oligonucleotide
gaggaggagaaagaggaggagga

SEQ ID NO: 857 - DNA, 23 bp, Artificial Sequence
  Synthetic oligonucleotide
tcggaggggaaagaggaaaaggg

SEQ ID NO: 858 - DNA, 23 bp, Artificial Sequence
  Synthetic oligonucleotide
ggtggggagaaagagaaaaaggg

SEQ ID NO: 859 - DNA, 23 bp, Artificial Sequence
  Synthetic oligonucleotide
aaagaggagaaagaggaacatgg

SEQ ID NO: 860 - DNA, 23 bp, Artificial Sequence
  Synthetic oligonucleotide
ggtggggagaatgaggaaggggg

SEQ ID NO: 861 - DNA, 23 bp, Artificial Sequence
  Synthetic oligonucleotide
aaggaggagaaggaggaagaagg

SEQ ID NO: 862 - DNA, 23 bp, Artificial Sequence
  Synthetic oligonucleotide
ggtgaggagaaagagaaaagggg

SEQ ID NO: 863 - DNA, 23 bp, Artificial Sequence
  Synthetic oligonucleotide
aatgaggagcaagaggaaaaggg

SEQ ID NO: 864 - DNA, 23 bp, Artificial Sequence
  Synthetic oligonucleotide
agtgaagagaatgagaaaaagag

SEQ ID NO: 865 - DNA, 23 bp, Artificial Sequence
  Synthetic oligonucleotide
tattaggagaaggaggaaaaggg

SEQ ID NO: 866 - DNA, 23 bp, Artificial Sequence
  Synthetic oligonucleotide
aaggaggagaaagagaaaaagag

SEQ ID NO: 867 - DNA, 23 bp, Artificial Sequence
  Synthetic oligonucleotide
actgaggagaaagaggagaaagg

SEQ ID NO: 868 - DNA, 23 bp, Artificial Sequence
  Synthetic oligonucleotide
acagaggagagagaggaaaaggg

SEQ ID NO: 869 - DNA, 23 bp, Artificial Sequence
  Synthetic oligonucleotide
ccagaggagaaggaggaaactgg

SEQ ID NO: 870 - DNA, 23 bp, Artificial Sequence
  Synthetic oligonucleotide
ggtaaggagaaaggggaaaacgg

SEQ ID NO: 871 - DNA, 23 bp, Artificial Sequence
  Synthetic oligonucleotide
aaagaggagaaagaggagaaaga

SEQ ID NO: 872 - DNA, 23 bp, Artificial Sequence
  Synthetic oligonucleotide
agtgaagagaatgaggaagagag

SEQ ID NO: 873 - DNA, 23 bp, Artificial Sequence
  Synthetic oligonucleotide
aaggaggaggaagaggaaaaagg

SEQ ID NO: 874 - DNA, 23 bp, Artificial Sequence
  Synthetic oligonucleotide
tgttaggagaatgaggaaacaag

SEQ ID NO: 875 - DNA, 23 bp, Artificial Sequence
  Synthetic oligonucleotide
agagaggagaaagagaaaaacgt

SEQ ID NO: 876 - DNA, 23 bp, Artificial Sequence
  Synthetic oligonucleotide
caagaggagagagaggaaaaggg

SEQ ID NO: 877 - DNA, 23 bp, Artificial Sequence
  Synthetic oligonucleotide
atgaaggagaaagaggaaatggg

SEQ ID NO: 878 - DNA, 23 bp, Artificial Sequence
  Synthetic oligonucleotide
agagaggagaaggaggataaagg

SEQ ID NO: 879 - DNA, 23 bp, Artificial Sequence
  Synthetic oligonucleotide
tgggaggagaaggaggaagaggg

SEQ ID NO: 880 - DNA, 23 bp, Artificial Sequence
  Synthetic oligonucleotide
caaaaggagaatgaggaaactgg

SEQ ID NO: 881 - DNA, 23 bp, Artificial Sequence
  Synthetic oligonucleotide
agggaggagaatgaggatgaggg

SEQ ID NO: 882 - DNA, 23 bp, Artificial Sequence
  Synthetic oligonucleotide
agcaaggagaaagaggaaagggg

SEQ ID NO: 883 - DNA, 23 bp, Artificial Sequence
  Synthetic oligonucleotide
aaagagaagaatgaggaaagtgg

SEQ ID NO: 884 - DNA, 23 bp, Artificial Sequence
  Synthetic oligonucleotide
agggaggagaatgaggtgaaagg

SEQ ID NO: 885 - DNA, 23 bp, Artificial Sequence
  Synthetic oligonucleotide
caggaagagaatgaggtaaatgg

SEQ ID NO: 886 - DNA, 23 bp, Artificial Sequence
  Synthetic oligonucleotide
aaagaggaggaagaggaaaaagg

SEQ ID NO: 887 - DNA, 23 bp, Artificial Sequence
  Synthetic oligonucleotide
atcaaggagaaggagaaaagggg

SEQ ID NO: 888 - DNA, 23 bp, Artificial Sequence
  Synthetic oligonucleotide
aaagaggagcaagaggaaaatgg

SEQ ID NO: 889 - DNA, 23 bp, Artificial Sequence
  Synthetic oligonucleotide
catgtagagaatgaggaaaaggg

SEQ ID NO: 890 - DNA, 23 bp, Artificial Sequence
  Synthetic oligonucleotide
caagagaagaatgaggaaagagg

SEQ ID NO: 891 - DNA, 23 bp, Artificial Sequence
  Synthetic oligonucleotide
tgagaggagaatgagaataaagg

SEQ ID NO: 892 - DNA, 23 bp, Artificial Sequence
  Synthetic oligonucleotide
cacgaggagaaagagaaaagtgg

SEQ ID NO: 893 - DNA, 23 bp, Artificial Sequence
  Synthetic oligonucleotide
agggagggggatgaggaaaaagg

SEQ ID NO: 894 - DNA, 23 bp, Artificial Sequence
  Synthetic oligonucleotide
agagagaagaatgaggaaagagg

SEQ ID NO: 895 - DNA, 23 bp, Artificial Sequence
  Synthetic oligonucleotide
gggaatgctaatgaggaaaatgg

SEQ ID NO: 896 - DNA, 23 bp, Artificial Sequence
  Synthetic oligonucleotide
aaagaggagaaagagaaaagggg

SEQ ID NO: 897 - DNA, 23 bp, Artificial Sequence
  Synthetic oligonucleotide
caggaggagaaagagacaaatgg

SEQ ID NO: 898 - DNA, 23 bp, Artificial Sequence
  Synthetic oligonucleotide
aataaggagaatgagaaaagtgg

SEQ ID NO: 899 - DNA, 23 bp, Artificial Sequence
  Synthetic oligonucleotide
agtgaggaggaagaggagaaggg

SEQ ID NO: 900 - DNA, 23 bp, Artificial Sequence
  Synthetic oligonucleotide
cataaggagaatgagaacaaagg

SEQ ID NO: 901 - DNA, 23 bp, Artificial Sequence
  Synthetic oligonucleotide
agggaggggaaggaggataaggg

SEQ ID NO: 902 - DNA, 23 bp, Artificial Sequence
  Synthetic oligonucleotide
agtggggagaaagagaaaaaagg

SEQ ID NO: 903 - DNA, 23 bp, Artificial Sequence
  Synthetic oligonucleotide
gcagattctctctgctcacttgg

SEQ ID NO: 904 - DNA, 23 bp, Artificial Sequence
  Synthetic oligonucleotide
gatggtacaggctcactcgcagg

SEQ ID NO: 905 - DNA, 23 bp, Artificial Sequence
  Synthetic oligonucleotide
agtggtacaggctcacaagctgg

SEQ ID NO: 906 - DNA, 23 bp, Artificial Sequence
  Synthetic oligonucleotide
catggcacaggctcacctgcagg

SEQ ID NO: 907 - DNA, 23 bp, Artificial Sequence
  Synthetic oligonucleotide
ggtggcacaggttcactcgttgg

SEQ ID NO: 908 - DNA, 23 bp, Artificial Sequence
  Synthetic oligonucleotide
gatggcacagtctcactcagggg

SEQ ID NO: 909 - DNA, 23 bp, Artificial Sequence
  Synthetic oligonucleotide
gaagggacagactcagtcgcagg

SEQ ID NO: 910 - DNA, 23 bp, Artificial Sequence
  Synthetic oligonucleotide
cgtggtacagactcactctctga

SEQ ID NO: 911 - DNA, 23 bp, Artificial Sequence
  Synthetic oligonucleotide
gcacccaaatgttgaggtacagg

SEQ ID NO: 912 - DNA, 23 bp, Artificial Sequence
  Synthetic oligonucleotide
tatcccaaatgttgaggtactgg

SEQ ID NO: 913 - DNA, 23 bp, Artificial Sequence
  Synthetic oligonucleotide
gtcatcgaactgctcttagctgg

SEQ ID NO: 914 - DNA, 23 bp, Artificial Sequence
  Synthetic oligonucleotide
gtcattgaactgctcttagcctg

SEQ ID NO: 915 - DNA, 23 bp, Artificial Sequence
  Synthetic oligonucleotide
gcctgaccatcgagaagtcctgg

SEQ ID NO: 916 - DNA, 23 bp, Artificial Sequence
  Synthetic oligonucleotide
ggacgatgacatgctcaagctgg

SEQ ID NO: 917 - DNA, 23 bp, Artificial Sequence
  Synthetic oligonucleotide
ggtcgatgccaggctcaagctgg

SEQ ID NO: 918 - DNA, 23 bp, Artificial Sequence
  Synthetic oligonucleotide
ggaaggggacatgctaaagcagg

SEQ ID NO: 919 - DNA, 23 bp, Artificial Sequence
  Synthetic oligonucleotide
gagtcacttacatacagccgggg

SEQ ID NO: 920 - DNA, 23 bp, Artificial Sequence
  Synthetic oligonucleotide
gtgtcactaacatacagccaggg

SEQ ID NO: 921 - DNA, 23 bp, Artificial Sequence
  Synthetic oligonucleotide
aaggcactaacatacagcctggt

SEQ ID NO: 922 - DNA, 23 bp, Artificial Sequence
  Synthetic oligonucleotide
acatcacctacatacagccaggg

SEQ ID NO: 923 - DNA, 23 bp, Artificial Sequence
  Synthetic oligonucleotide
gcgctccttacctgcagccgggc

SEQ ID NO: 924 - DNA, 23 bp, Artificial Sequence
  Synthetic oligonucleotide
gagatggaagagtctgatcaggg

SEQ ID NO: 925 - DNA, 23 bp, Artificial Sequence
  Synthetic oligonucleotide
gagatggaggagcctgatcatag

SEQ ID NO: 926 - DNA, 23 bp, Artificial Sequence
  Synthetic oligonucleotide
gagatggaagagactgagcaagg

SEQ ID NO: 927 - DNA, 23 bp, Artificial Sequence
  Synthetic oligonucleotide
atcatggaagagtctgatcaggg

SEQ ID NO: 928 - DNA, 23 bp, Artificial Sequence
  Synthetic oligonucleotide
aaggtggaagagtgagatcaggg

SEQ ID NO: 929 - DNA, 23 bp, Artificial Sequence
  Synthetic oligonucleotide
aagatggaaggatctgattatgg

SEQ ID NO: 930 - DNA, 23 bp, Artificial Sequence
  Synthetic oligonucleotide
gtctgacttaccccacaggaggg

SEQ ID NO: 931 - DNA, 23 bp, Artificial Sequence
  Synthetic oligonucleotide
gtctgactcaccccacaggagtg

SEQ ID NO: 932 - DNA, 23 bp, Artificial Sequence
  Synthetic oligonucleotide
gcctgacctaccccacaggacta

SEQ ID NO: 933 - DNA, 23 bp, Artificial Sequence
  Synthetic oligonucleotide
ggctgacctacctcacaggaggg

SEQ ID NO: 934 - DNA, 23 bp, Artificial Sequence
  Synthetic oligonucleotide
gtctgaccttccccacagaaggg

SEQ ID NO: 935 - DNA, 23 bp, Artificial Sequence
  Synthetic oligonucleotide
gcctgacttactccacagaaagg

SEQ ID NO: 936 - DNA, 23 bp, Artificial Sequence
  Synthetic oligonucleotide
gtctgacctacccagcaggaagg

SEQ ID NO: 937 - DNA, 23 bp, Artificial Sequence
  Synthetic oligonucleotide
gcctggcctaccccacagggcgg

SEQ ID NO: 938 - DNA, 23 bp, Artificial Sequence
  Synthetic oligonucleotide
gtgtgacctgctccacaggaggg

SEQ ID NO: 939 - DNA, 23 bp, Artificial Sequence
  Synthetic oligonucleotide
gtatgacctacctcacagcaggg

SEQ ID NO: 940 - DNA, 23 bp, Artificial Sequence
  Synthetic oligonucleotide
cgctgactcaccccacaggcggg

SEQ ID NO: 941 - DNA, 23 bp, Artificial Sequence
  Synthetic oligonucleotide
cccagacctaccccacaggaggg

SEQ ID NO: 942 - DNA, 23 bp, Artificial Sequence
  Synthetic oligonucleotide
atatgacctacctcaaaggatgg

SEQ ID NO: 943 - DNA, 23 bp, Artificial Sequence
  Synthetic oligonucleotide
gcctggcccacaccacaggatgg

SEQ ID NO: 944 - DNA, 23 bp, Artificial Sequence
  Synthetic oligonucleotide
gatggcatcgtcacggtctcggg

SEQ ID NO: 945 - DNA, 23 bp, Artificial Sequence
  Synthetic oligonucleotide
gtccatcacatttcaaatggggg

SEQ ID NO: 946 - DNA, 23 bp, Artificial Sequence
  Synthetic oligonucleotide
gaccatcacatctcaaaaggggg

SEQ ID NO: 947 - DNA, 23 bp, Artificial Sequence
  Synthetic oligonucleotide
acacatcacattccaaatggtgg

SEQ ID NO: 948 - DNA, 23 bp, Artificial Sequence
  Synthetic oligonucleotide
ggatactgtaccttccggagggg

SEQ ID NO: 949 - DNA, 23 bp, Artificial Sequence
  Synthetic oligonucleotide
aggtactgtaccctccagagggg

SEQ ID NO: 950 - DNA, 23 bp, Artificial Sequence
  Synthetic oligonucleotide
aggtactgtaccctccagagggg

SEQ ID NO: 951 - DNA, 23 bp, Artificial Sequence
  Synthetic oligonucleotide
gggtactgtcccttcaagagggg

SEQ ID NO: 952 - DNA, 23 bp, Artificial Sequence
  Synthetic oligonucleotide
ccatattgtaccttccagagagg

SEQ ID NO: 953 - DNA, 23 bp, Artificial Sequence
  Synthetic oligonucleotide
agatactgtaccttcctttgagg

SEQ ID NO: 954 - DNA, 23 bp, Artificial Sequence
  Synthetic oligonucleotide
gtaggcactcacccgggcctggg

SEQ ID NO: 955 - DNA, 23 bp, Artificial Sequence
  Synthetic oligonucleotide
ctaagcactcacccgggcctctg

SEQ ID NO: 956 - DNA, 23 bp, Artificial Sequence
  Synthetic oligonucleotide
ctaggcactcacccaggccgggg

SEQ ID NO: 957 - DNA, 23 bp, Artificial Sequence
  Synthetic oligonucleotide
gtaggccaccacccgggccttgg

SEQ ID NO: 958 - DNA, 23 bp, Artificial Sequence
  Synthetic oligonucleotide
gcaggcactcacccggccccggg

SEQ ID NO: 959 - DNA, 23 bp, Artificial Sequence
  Synthetic oligonucleotide
gtggccactcacccaggcctggg

SEQ ID NO: 960 - DNA, 23 bp, Artificial Sequence
  Synthetic oligonucleotide
cagggcactcacccgggccttgt

SEQ ID NO: 961 - DNA, 23 bp, Artificial Sequence
  Synthetic oligonucleotide
ctagacacccacccaggcctggg

SEQ ID NO: 962 - DNA, 23 bp, Artificial Sequence
  Synthetic oligonucleotide
gcaggccaccacccgggccttgg

SEQ ID NO: 963 - DNA, 23 bp, Artificial Sequence
  Synthetic oligonucleotide
gtagacacacacctaggcctggg

SEQ ID NO: 964 - DNA, 23 bp, Artificial Sequence
  Synthetic oligonucleotide
ctagccacacacccaggcctggg

SEQ ID NO: 965 - DNA, 23 bp, Artificial Sequence
  Synthetic oligonucleotide
ctgggcacccaccagggcctggg

SEQ ID NO: 966 - DNA, 23 bp, Artificial Sequence
  Synthetic oligonucleotide
gtaaccacccacccgggccgggg

SEQ ID NO: 967 - DNA, 23 bp, Artificial Sequence
  Synthetic oligonucleotide
ccaggcactcacccagcccttgg

SEQ ID NO: 968 - DNA, 23 bp, Artificial Sequence
  Synthetic oligonucleotide
ttaggcacacgcccgggcttcgg

SEQ ID NO: 969 - DNA, 23 bp, Artificial Sequence
  Synthetic oligonucleotide
gcgggcacacgcccgggcctggg

SEQ ID NO: 970 - DNA, 23 bp, Artificial Sequence
  Synthetic oligonucleotide
ccaggcactcacccggtccaggg

SEQ ID NO: 971 - DNA, 23 bp, Artificial Sequence
  Synthetic oligonucleotide
aaaggcactcaccctggcccagg

SEQ ID NO: 972 - DNA, 23 bp, Artificial Sequence
  Synthetic oligonucleotide
gtagacacacaccagggccctgg

SEQ ID NO: 973 - DNA, 23 bp, Artificial Sequence
  Synthetic oligonucleotide
tcaggcagccactcgggccttgg

SEQ ID NO: 974 - DNA, 23 bp, Artificial Sequence
  Synthetic oligonucleotide
cctggcacccaccagggcctagg

SEQ ID NO: 975 - DNA, 23 bp, Artificial Sequence
  Synthetic oligonucleotide
atagggccccacccaggcctggg

SEQ ID NO: 976 - DNA, 23 bp, Artificial Sequence
  Synthetic oligonucleotide
gggcactcacctcggcactccgg

SEQ ID NO: 977 - DNA, 23 bp, Artificial Sequence
  Synthetic oligonucleotide
agggcctcaccccggcactctgg

SEQ ID NO: 978 - DNA, 23 bp, Artificial Sequence
  Synthetic oligonucleotide
tggcactcacctcgggcctgggg

SEQ ID NO: 979 - DNA, 23 bp, Artificial Sequence
  Synthetic oligonucleotide
catcactcaccctggcactcagg

SEQ ID NO: 980 - DNA, 23 bp, Artificial Sequence
  Synthetic oligonucleotide
gctgacccgctccagctgcccgg

SEQ ID NO: 981 - DNA, 23 bp, Artificial Sequence
  Synthetic oligonucleotide
actgaccagatccagctgcctgg

SEQ ID NO: 982 - DNA, 23 bp, Artificial Sequence
  Synthetic oligonucleotide
tatgacctgttccagctgcctgg

SEQ ID NO: 983 - DNA, 23 bp, Artificial Sequence
  Synthetic oligonucleotide
actcacccgccccagctccccgg

SEQ ID NO: 984 - DNA, 23 bp, Artificial Sequence
  Synthetic oligonucleotide
acggacgccccccggctgccggt

SEQ ID NO: 985 - DNA, 23 bp, Artificial Sequence
  Synthetic oligonucleotide
gttgcggaaactctcattgccgg

SEQ ID NO: 986 - DNA, 23 bp, Artificial Sequence
  Synthetic oligonucleotide
cttgcagaaactctcactgcagg

SEQ ID NO: 987 - DNA, 23 bp, Artificial Sequence
  Synthetic oligonucleotide
gtaacgcaaactctcattgctgg

SEQ ID NO: 988 - DNA, 23 bp, Artificial Sequence
  Synthetic oligonucleotide
cttgaggaaactctcattgaggg

SEQ ID NO: 989 - DNA, 23 bp, Artificial Sequence
  Synthetic oligonucleotide
gaaatggaaactctcattgctgg

SEQ ID NO: 990 - DNA, 23 bp, Artificial Sequence
  Synthetic oligonucleotide
attgctgaaacccacattgctgg

SEQ ID NO: 991 - DNA, 23 bp, Artificial Sequence
  Synthetic oligonucleotide
gatgtgcgagcgagctgtgtcgg

SEQ ID NO: 992 - DNA, 23 bp, Artificial Sequence
  Synthetic oligonucleotide
gatgagcaagcaagctgtgttgg

SEQ ID NO: 993 - DNA, 23 bp, Artificial Sequence
  Synthetic oligonucleotide
gatgtgccagcaacctgtgtggg

SEQ ID NO: 994 - DNA, 23 bp, Artificial Sequence
  Synthetic oligonucleotide
aatgtgcaggtgagctgtgtggg

SEQ ID NO: 995 - DNA, 23 bp, Artificial Sequence
  Synthetic oligonucleotide
aatgtgtgagcaagcagtgtggg

SEQ ID NO: 996 - DNA, 23 bp, Artificial Sequence
  Synthetic oligonucleotide
gaagtgccagcgggctgagtggg

SEQ ID NO: 997 - DNA, 23 bp, Artificial Sequence
  Synthetic oligonucleotide
tgtgtgcgagtgagctgtcttgg

SEQ ID NO: 998 - DNA, 23 bp, Artificial Sequence
  Synthetic oligonucleotide
agagtgcgagccaactgtgtggg

SEQ ID NO: 999 - DNA, 20 bp, Artificial Sequence
  Synthetic oligonucleotide
ggagagggaccgcgccacct

SEQ ID NO: 1000 - DNA, 23 bp, Artificial Sequence
  Synthetic oligonucleotide
ggagagggaccgcgccaccttgg

SEQ ID NO: 1001 - DNA, 20 bp, Artificial Sequence
  Synthetic oligonucleotide
gaacttacgcaggagatatt

SEQ ID NO: 1002 - DNA, 23 bp, Artificial Sequence
  Synthetic oligonucleotide
gaacttacgcaggagatattcgg

SEQ ID NO: 1003 - DNA, 20 bp, Artificial Sequence
  Synthetic oligonucleotide
gttccggtaagcatagacaa

SEQ ID NO: 1004 - DNA, 23 bp, Artificial Sequence
  Synthetic oligonucleotide
gttccggtaagcatagacaatgg

SEQ ID NO: 1005 - DNA, 20 bp, Artificial Sequence
  Synthetic oligonucleotide
gagacccgctcttcagcatg

SEQ ID NO: 1006 - DNA, 23 bp, Artificial Sequence
  Synthetic oligonucleotide
gagacccgctcttcagcatgtgg

SEQ ID NO: 1007 - DNA, 20 bp, Artificial Sequence
  Synthetic oligonucleotide
gagagggccccaagacctcg

SEQ ID NO: 1008 - DNA, 23 bp, Artificial Sequence
  Synthetic oligonucleotide
gagagggccccaagacctcgtgg

SEQ ID NO: 1009 - DNA, 20 bp, Artificial Sequence
  Synthetic oligonucleotide
gcgccttattccagtgacaa

SEQ ID NO: 1010 - DNA, 23 bp, Artificial Sequence
  Synthetic oligonucleotide
gcgccttattccagtgacaaagg

SEQ ID NO: 1011 - DNA, 20 bp, Artificial Sequence
  Synthetic oligonucleotide
gcagatcctcatcaccgcgc

SEQ ID NO: 1012 - DNA, 23 bp, Artificial Sequence
  Synthetic oligonucleotide
gcagatcctcatcaccgcgctgg

SEQ ID NO: 1013 - DNA, 20 bp, Artificial Sequence
  Synthetic oligonucleotide
gcatgtcgacttctcctcgg

SEQ ID NO: 1014 - DNA, 23 bp, Artificial Sequence
  Synthetic oligonucleotide
gcatgtcgacttctcctcggagg

SEQ ID NO: 1015 - DNA, 20 bp, Artificial Sequence
  Synthetic oligonucleotide
gctggtctaggacgtcctcc

SEQ ID NO: 1016 - DNA, 23 bp, Artificial Sequence
  Synthetic oligonucleotide
gctggtctaggacgtcctccagg

SEQ ID NO: 1017 - DNA, 20 bp, Artificial Sequence
  Synthetic oligonucleotide
ggaaactcaccgatccatac

SEQ ID NO: 1018 - DNA, 23 bp, Artificial Sequence
  Synthetic oligonucleotide
ggaaactcaccgatccatacagg

SEQ ID NO: 1019 - DNA, 20 bp, Artificial Sequence
  Synthetic oligonucleotide
gccagcaaagcacattattt

SEQ ID NO: 1020 - DNA, 23 bp, Artificial Sequence
  Synthetic oligonucleotide
gccagcaaagcacattattttgg

SEQ ID NO: 1021 - DNA, 20 bp, Artificial Sequence
  Synthetic oligonucleotide
ggcccgtctccgtgctcctc

SEQ ID NO: 1022 - DNA, 23 bp, Artificial Sequence
  Synthetic oligonucleotide
ggcccgtctccgtgctcctctgg

SEQ ID NO: 1023 - DNA, 20 bp, Artificial Sequence
  Synthetic oligonucleotide
gcgctacgacgagatcgtca

SEQ ID NO: 1024 - DNA, 23 bp, Artificial Sequence
  Synthetic oligonucleotide
gcgctacgacgagatcgtcaagg

SEQ ID NO: 1025 - DNA, 20 bp, Artificial Sequence
  Synthetic oligonucleotide
gagaataagattcagttgca

SEQ ID NO: 1026 - DNA, 23 bp, Artificial Sequence
  Synthetic oligonucleotide
gagaataagattcagttgcaagg

SEQ ID NO: 1027 - DNA, 20 bp, Artificial Sequence
  Synthetic oligonucleotide
gtttcttgggatccaccacc

SEQ ID NO: 1028 - DNA, 23 bp, Artificial Sequence
  Synthetic oligonucleotide
gtttcttgggatccaccaccagg

SEQ ID NO: 1029 - DNA, 20 bp, Artificial Sequence
  Synthetic oligonucleotide
gtaggtgggctgccgaagat

SEQ ID NO: 1030 - DNA, 23 bp, Artificial Sequence
  Synthetic oligonucleotide
gtaggtgggctgccgaagatagg

SEQ ID NO: 1031 - DNA, 20 bp, Artificial Sequence
  Synthetic oligonucleotide
gtaattaggtaaggcttagt

SEQ ID NO: 1032 - DNA, 23 bp, Artificial Sequence
  Synthetic oligonucleotide
gtaattaggtaaggcttagttgg

SEQ ID NO: 1033 - DNA, 20 bp, Artificial Sequence
  Synthetic oligonucleotide
gctctttgaccgcgcgcgtg

SEQ ID NO: 1034 - DNA, 23 bp, Artificial Sequence
  Synthetic oligonucleotide
gctctttgaccgcgcgcgtgtgg

SEQ ID NO: 1035 - DNA, 20 bp, Artificial Sequence
  Synthetic oligonucleotide
gaaacagctcgttgtaccgc

SEQ ID NO: 1036 - DNA, 23 bp, Artificial Sequence
  Synthetic oligonucleotide
gaaacagctcgttgtaccgctgg

SEQ ID NO: 1037 - DNA, 20 bp, Artificial Sequence
  Synthetic oligonucleotide
gcaacaacgtgatgaatatc

SEQ ID NO: 1038 - DNA, 23 bp, Artificial Sequence
  Synthetic oligonucleotide
gcaacaacgtgatgaatatctgg

SEQ ID NO: 1039 - DNA, 20 bp, Artificial Sequence
  Synthetic oligonucleotide
gtcgctgtgactttctaatt

SEQ ID NO: 1040 - DNA, 23 bp, Artificial Sequence
  Synthetic oligonucleotide
gtcgctgtgactttctaatttgg

SEQ ID NO: 1041 - DNA, 20 bp, Artificial Sequence
  Synthetic oligonucleotide
ggtgttatctctgaagcgca

SEQ ID NO: 1042 - DNA, 23 bp, Artificial Sequence
  Synthetic oligonucleotide
ggtgttatctctgaagcgcatgg

SEQ ID NO: 1043 - DNA, 20 bp, Artificial Sequence
  Synthetic oligonucleotide
ggatcatggaagccagctcc

SEQ ID NO: 1044 - DNA, 23 bp, Artificial Sequence
  Synthetic oligonucleotide
ggatcatggaagccagctccagg

SEQ ID NO: 1045 - DNA, 20 bp, Artificial Sequence
  Synthetic oligonucleotide
ggtctcggcacttggctcgc

SEQ ID NO: 1046 - DNA, 23 bp, Artificial Sequence
  Synthetic oligonucleotide
ggtctcggcacttggctcgctgg

SEQ ID NO: 1047 - DNA, 20 bp, Artificial Sequence
  Synthetic oligonucleotide
ggaggcttcacagcgccctc

SEQ ID NO: 1048 - DNA, 23 bp, Artificial Sequence
  Synthetic oligonucleotide
ggaggcttcacagcgccctctgg

SEQ ID NO: 1049 - DNA, 20 bp, Artificial Sequence
  Synthetic oligonucleotide
gctagtaccttgtatgaaga

SEQ ID NO: 1050 - DNA, 23 bp, Artificial Sequence
  Synthetic oligonucleotide
gctagtaccttgtatgaagatgg

SEQ ID NO: 1051 - DNA, 20 bp, Artificial Sequence
  Synthetic oligonucleotide
gacgggaaagtcagtgtgaa

SEQ ID NO: 1052 - DNA, 23 bp, Artificial Sequence
  Synthetic oligonucleotide
gacgggaaagtcagtgtgaatgg

SEQ ID NO: 1053 - DNA, 20 bp, Artificial Sequence
  Synthetic oligonucleotide
gttcgaccatgcccttgctt

SEQ ID NO: 1054 - DNA, 23 bp, Artificial Sequence
  Synthetic oligonucleotide
gttcgaccatgcccttgcttagg

SEQ ID NO: 1055 - DNA, 20 bp, Artificial Sequence
  Synthetic oligonucleotide
ggcagagctcacctgtagat

SEQ ID NO: 1056 - DNA, 23 bp, Artificial Sequence
  Synthetic oligonucleotide
ggcagagctcacctgtagatagg

SEQ ID NO: 1057 - DNA, 20 bp, Artificial Sequence
  Synthetic oligonucleotide
gctggtggagctcttctcaa

SEQ ID NO: 1058 - DNA, 23 bp, Artificial Sequence
  Synthetic oligonucleotide
gctggtggagctcttctcaatgg

SEQ ID NO: 1059 - DNA, 20 bp, Artificial Sequence
  Synthetic oligonucleotide
ggagctgacccaagacgttc

SEQ ID NO: 1060 - DNA, 23 bp, Artificial Sequence
  Synthetic oligonucleotide
ggagctgacccaagacgttctgg

SEQ ID NO: 1061 - DNA, 20 bp, Artificial Sequence
  Synthetic oligonucleotide
gttgaccatcagattgagac

SEQ ID NO: 1062 - DNA, 23 bp, Artificial Sequence
  Synthetic oligonucleotide
gttgaccatcagattgagacagg

SEQ ID NO: 1063 - DNA, 20 bp, Artificial Sequence
  Synthetic oligonucleotide
gctcacctcgtgtccgttgc

SEQ ID NO: 1064 - DNA, 23 bp, Artificial Sequence
  Synthetic oligonucleotide
gctcacctcgtgtccgttgctgg

SEQ ID NO: 1065 - DNA, 20 bp, Artificial Sequence
  Synthetic oligonucleotide
ggacgttcatgtatttgctt

SEQ ID NO: 1066 - DNA, 23 bp, Artificial Sequence
  Synthetic oligonucleotide
ggacgttcatgtatttgctttgg

SEQ ID NO: 1067 - DNA, 20 bp, Artificial Sequence
  Synthetic oligonucleotide
gctgtaaaagacatccctga

SEQ ID NO: 1068 - DNA, 23 bp, Artificial Sequence
  Synthetic oligonucleotide
gctgtaaaagacatccctgatgg

SEQ ID NO: 1069 - DNA, 20 bp, Artificial Sequence
  Synthetic oligonucleotide
gggtctccaccacttcgtaa

SEQ ID NO: 1070 - DNA, 23 bp, Artificial Sequence
  Synthetic oligonucleotide
gggtctccaccacttcgtaaagg

SEQ ID NO: 1071 - DNA, 20 bp, Artificial Sequence
  Synthetic oligonucleotide
ggcggcgctcacaattgcca

SEQ ID NO: 1072 - DNA, 23 bp, Artificial Sequence
  Synthetic oligonucleotide
ggcggcgctcacaattgccacgg

SEQ ID NO: 1073 - DNA, 20 bp, Artificial Sequence
  Synthetic oligonucleotide
ggtacttactgttactcgca

SEQ ID NO: 1074 - DNA, 23 bp, Artificial Sequence
  Synthetic oligonucleotide
ggtacttactgttactcgcaagg

SEQ ID NO: 1075 - DNA, 20 bp, Artificial Sequence
  Synthetic oligonucleotide
gacgctggtcaaacgccttg

SEQ ID NO: 1076 - DNA, 23 bp, Artificial Sequence
  Synthetic oligonucleotide
gacgctggtcaaacgccttgcgg

SEQ ID NO: 1077 - DNA, 20 bp, Artificial Sequence
  Synthetic oligonucleotide
ggcatgctgcggcatgagat

SEQ ID NO: 1078 - DNA, 23 bp, Artificial Sequence
  Synthetic oligonucleotide
ggcatgctgcggcatgagatagg

SEQ ID NO: 1079 - DNA, 20 bp, Artificial Sequence
  Synthetic oligonucleotide
ggctccacgcagacgctgac

SEQ ID NO: 1080 - DNA, 23 bp, Artificial Sequence
  Synthetic oligonucleotide
ggctccacgcagacgctgacagg

SEQ ID NO: 1081 - DNA, 20 bp, Artificial Sequence
  Synthetic oligonucleotide
gtcgaggagaatgaggaaaa

SEQ ID NO: 1082 - DNA, 23 bp, Artificial Sequence
  Synthetic oligonucleotide
gtcgaggagaatgaggaaaatgg

SEQ ID NO: 1083 - DNA, 20 bp, Artificial Sequence
  Synthetic oligonucleotide
gcagattctctctgctcact

SEQ ID NO: 1084 - DNA, 23 bp, Artificial Sequence
  Synthetic oligonucleotide
gcagattctctctgctcacttgg

SEQ ID NO: 1085 - DNA, 20 bp, Artificial Sequence
  Synthetic oligonucleotide
gatggtacaggctcactcgc

SEQ ID NO: 1086 - DNA, 23 bp, Artificial Sequence
  Synthetic oligonucleotide
gatggtacaggctcactcgcagg

SEQ ID NO: 1087 - DNA, 20 bp, Artificial Sequence
  Synthetic oligonucleotide
gcacccaaatgttgaggtac

SEQ ID NO: 1088 - DNA, 23 bp, Artificial Sequence
  Synthetic oligonucleotide
gcacccaaatgttgaggtacagg

SEQ ID NO: 1089 - DNA, 20 bp, Artificial Sequence
  Synthetic oligonucleotide
gtcatcgaactgctcttagc

SEQ ID NO: 1090 - DNA, 23 bp, Artificial Sequence
  Synthetic oligonucleotide
gtcatcgaactgctcttagctgg

SEQ ID NO: 1091 - DNA, 20 bp, Artificial Sequence
  Synthetic oligonucleotide
gcctgaccatcgagaagtcc

SEQ ID NO: 1092 - DNA, 23 bp, Artificial Sequence
  Synthetic oligonucleotide
gcctgaccatcgagaagtcctgg

SEQ ID NO: 1093 - DNA, 20 bp, Artificial Sequence
  Synthetic oligonucleotide
ggacgatgacatgctcaagc

SEQ ID NO: 1094 - DNA, 23 bp, Artificial Sequence
  Synthetic oligonucleotide
ggacgatgacatgctcaagctgg

SEQ ID NO: 1095 - DNA, 20 bp, Artificial Sequence
  Synthetic oligonucleotide
gagtcacttacatacagccg

SEQ ID NO: 1096 - DNA, 23 bp, Artificial Sequence
  Synthetic oligonucleotide
gagtcacttacatacagccgggg

SEQ ID NO: 1097 - DNA, 20 bp, Artificial Sequence
  Synthetic oligonucleotide
gagatggaagagtctgatca

SEQ ID NO: 1098 - DNA, 23 bp, Artificial Sequence
  Synthetic oligonucleotide
gagatggaagagtctgatcaggg

SEQ ID NO: 1099 - DNA, 20 bp, Artificial Sequence
  Synthetic oligonucleotide
gtctgacttaccccacagga

SEQ ID NO: 1100 - DNA, 23 bp, Artificial Sequence
  Synthetic oligonucleotide
gtctgacttaccccacaggaggg

SEQ ID NO: 1101 - DNA, 20 bp, Artificial Sequence
  Synthetic oligonucleotide
gatggcatcgtcacggtctc

SEQ ID NO: 1102 - DNA, 23 bp, Artificial Sequence
  Synthetic oligonucleotide
gatggcatcgtcacggtctcggg

SEQ ID NO: 1103 - DNA, 20 bp, Artificial Sequence
  Synthetic oligonucleotide
gtccatcacatttcaaatgg

SEQ ID NO: 1104 - DNA, 23 bp, Artificial Sequence
  Synthetic oligonucleotide
gtccatcacatttcaaatggggg

SEQ ID NO: 1105 - DNA, 20 bp, Artificial Sequence
  Synthetic oligonucleotide
ggatactgtaccttccggag

SEQ ID NO: 1106 - DNA, 23 bp, Artificial Sequence
  Synthetic oligonucleotide
ggatactgtaccttccggagggg

SEQ ID NO: 1107 - DNA, 20 bp, Artificial Sequence
  Synthetic oligonucleotide
gtaggcactcacccgggcct

SEQ ID NO: 1108 - DNA, 23 bp, Artificial Sequence
  Synthetic oligonucleotide
gtaggcactcacccgggcctggg

SEQ ID NO: 1109 - DNA, 20 bp, Artificial Sequence
  Synthetic oligonucleotide
gggcactcacctcggcactc

SEQ ID NO: 1110 - DNA, 23 bp, Artificial Sequence
  Synthetic oligonucleotide
gggcactcacctcggcactccgg

SEQ ID NO: 1111 - DNA, 20 bp, Artificial Sequence
  Synthetic oligonucleotide
gctgacccgctccagctgcc

SEQ ID NO: 1112 - DNA, 23 bp, Artificial Sequence
  Synthetic oligonucleotide
gctgacccgctccagctgcccgg

SEQ ID NO: 1113 - DNA, 20 bp, Artificial Sequence
  Synthetic oligonucleotide
gttgcggaaactctcattgc

SEQ ID NO: 1114 - DNA, 23 bp, Artificial Sequence
  Synthetic oligonucleotide
gttgcggaaactctcattgccgg

SEQ ID NO: 1115 - DNA, 20 bp, Artificial Sequence
  Synthetic oligonucleotide
gatgtgcgagcgagctgtgt

SEQ ID NO: 1116 - DNA, 23 bp, Artificial Sequence
  Synthetic oligonucleotide
gatgtgcgagcgagctgtgtcgg

SEQ ID NO: 1117 - DNA, 20 bp, Artificial Sequence
  Synthetic oligonucleotide
gaccactgacaatacctccc

SEQ ID NO: 1118 - DNA, 23 bp, Artificial Sequence
  Synthetic oligonucleotide
gaccactgacaatacctccctgg

SEQ ID NO: 1119 - DNA, 20 bp, Artificial Sequence
  Synthetic oligonucleotide
gcgagtcttcactgagtgta

SEQ ID NO: 1120 - DNA, 23 bp, Artificial Sequence
  Synthetic oligonucleotide
gcgagtcttcactgagtgtaagg

SEQ ID NO: 1121 - DNA, 20 bp, Artificial Sequence
  Synthetic oligonucleotide
gagtccgagcagaagaagaa

SEQ ID NO: 1122 - DNA, 23 bp, Artificial Sequence
  Synthetic oligonucleotide
gagttagagcagaagaagaaagg

SEQ ID NO: 1123 - DNA, 20 bp, Artificial Sequence
  Synthetic oligonucleotide
ggtgagtgagtgtgtgcgtg

SEQ ID NO: 1124 - DNA, 23 bp, Artificial Sequence
  Synthetic oligonucleotide
agtgagtgagtgtgtgtgtgggg

SEQ ID NO: 1125 - DNA, 20 bp, Artificial Sequence
  Synthetic oligonucleotide
gtgcatttcaccactgaaat

SEQ ID NO: 1126 - DNA, 23 bp, Artificial Sequence
  Synthetic oligonucleotide
gtgcatttcaccactgaaattgg

SEQ ID NO: 1127 - DNA, 20 bp, Artificial Sequence
  Synthetic oligonucleotide
gaccctcaggccatgaggac

SEQ ID NO: 1128 - DNA, 23 bp, Artificial Sequence
  Synthetic oligonucleotide
gaccctcaggccatgaggactgg

SEQ ID NO: 1129 - DNA, 20 bp, Artificial Sequence
  Synthetic oligonucleotide
gtttcttttcaggctgaaga

SEQ ID NO: 1130 - DNA, 23 bp, Artificial Sequence
  Synthetic oligonucleotide
gtttcttttcaggctgaagatgg

SEQ ID NO: 1131 - DNA, 20 bp, Artificial Sequence
  Synthetic oligonucleotide
gggcgtcccagcacgacgac

SEQ ID NO: 1132 - DNA, 23 bp, Artificial Sequence
  Synthetic oligonucleotide
gggcgtcccagcacgacgacagg

SEQ ID NO: 1133 - DNA, 20 bp, Artificial Sequence
  Synthetic oligonucleotide
gcttgccttgtgacatctac

SEQ ID NO: 1134 - DNA, 23 bp, Artificial Sequence
  Synthetic oligonucleotide
gcttgccttgtgacatctactgg

SEQ ID NO: 1135 - DNA, 20 bp, Artificial Sequence
  Synthetic oligonucleotide
gacagacacgctgcagatcg

SEQ ID NO: 1136 - DNA, 23 bp, Artificial Sequence
  Synthetic oligonucleotide
gacagacacgctgcagatcgagg

SEQ ID NO: 1137 - DNA, 20 bp, Artificial Sequence
  Synthetic oligonucleotide
gcgagtcttcactgagtgta

SEQ ID NO: 1138 - DNA, 23 bp, Artificial Sequence
  Synthetic oligonucleotide
gcgagtcttcactgagtgtaagg

SEQ ID NO: 1139 - DNA, 20 bp, Artificial Sequence
  Synthetic oligonucleotide
ggtgagtgagtgtgtgcgtg

SEQ ID NO: 1140 - DNA, 23 bp, Artificial Sequence
  Synthetic oligonucleotide
ggtgagtgagtgtgtgcgtgtgg

SEQ ID NO: 1141 - DNA, 20 bp, Artificial Sequence
  Synthetic oligonucleotide
ggtgagtgagtgtgtgcgtg

SEQ ID NO: 1142 - DNA, 23 bp, Artificial Sequence
  Synthetic oligonucleotide
ggtgagtgagtgtgtgtgtgagg

SEQ ID NO: 1143 - DNA, 20 bp, Artificial Sequence
  Synthetic oligonucleotide
ggtgagtgagtgtgtgcgtg

SEQ ID NO: 1144 - DNA, 23 bp, Artificial Sequence
  Synthetic oligonucleotide
agtgagtgagtgtgtgtgtgggg

SEQ ID NO: 1145 - DNA, 20 bp, Artificial Sequence
  Synthetic oligonucleotide
ggtgagtgagtgtgtgcgtg

SEQ ID NO: 1146 - DNA, 23 bp, Artificial Sequence
  Synthetic oligonucleotide
tgtgggtgagtgtgtgcgtgagg

SEQ ID NO: 1147 - DNA, 20 bp, Artificial Sequence
  Synthetic oligonucleotide
ggtgagtgagtgtgtgcgtg

SEQ ID NO: 1148 - DNA, 23 bp, Artificial Sequence
  Synthetic oligonucleotide
ggtgagtgagtgcgtgcgggtgg

SEQ ID NO: 1149 - DNA, 20 bp, Artificial Sequence
  Synthetic oligonucleotide
ggtgagtgagtgtgtgcgtg

SEQ ID NO: 1150 - DNA, 23 bp, Artificial Sequence
  Synthetic oligonucleotide
gctgagtgagtgtatgcgtgtgg

SEQ ID NO: 1151 - DNA, 20 bp, Artificial Sequence
  Synthetic oligonucleotide
gagtccgagcagaagaagaa

SEQ ID NO: 1152 - DNA, 23 bp, Artificial Sequence
  Synthetic oligonucleotide
gagtccgagcagaagaagaaggg

SEQ ID NO: 1153 - DNA, 20 bp, Artificial Sequence
  Synthetic oligonucleotide
gagtccgagcagaagaagaa

SEQ ID NO: 1154 - DNA, 23 bp, Artificial Sequence
  Synthetic oligonucleotide
gagttagagcagaagaagaaagg

SEQ ID NO: 1155 - DNA, 20 bp, Artificial Sequence
  Synthetic oligonucleotide
gagtccgagcagaagaagaa

SEQ ID NO: 1156 - DNA, 23 bp, Artificial Sequence
  Synthetic oligonucleotide
gagtctaagcagaagaagaagag

SEQ ID NO: 1157 - DNA, 20 bp, Artificial Sequence
  Synthetic oligonucleotide
gtgtaggttggacgcacttt

SEQ ID NO: 1158 - DNA, 23 bp, Artificial Sequence
  Synthetic oligonucleotide
gtataggttggacgcacttttgg

SEQ ID NO: 1159 - DNA, 20 bp, Artificial Sequence
  Synthetic oligonucleotide
gagtccgagcagaagaagaa

SEQ ID NO: 1160 - DNA, 23 bp, Artificial Sequence
  Synthetic oligonucleotide
gagtccgagcagaagaagaaggg

SEQ ID NO: 1161 - DNA, 20 bp, Artificial Sequence
  Synthetic oligonucleotide
gcttgccttgtgacatctac

SEQ ID NO: 1162 - DNA, 23 bp, Artificial Sequence
  Synthetic oligonucleotide
gcttgccttgtgacatctactgg

SEQ ID NO: 1163 - DNA, 20 bp, Artificial Sequence
  Synthetic oligonucleotide
gacagacacgctgcagatcg

SEQ ID NO: 1164 - DNA, 23 bp, Artificial Sequence
  Synthetic oligonucleotide
gacagacacgctgcagatcgagg

SEQ ID NO: 1165 - DNA, 20 bp, Artificial Sequence
  Synthetic oligonucleotide
gtgcatttcaccactgaaat

SEQ ID NO: 1166 - DNA, 23 bp, Artificial Sequence
  Synthetic oligonucleotide
gtgcatttcaccactgaaattgg

SEQ ID NO: 1167 - DNA, 20 bp, Artificial Sequence
  Synthetic oligonucleotide
gaccctcaggccatgaggac

SEQ ID NO: 1168 - DNA, 23 bp, Artificial Sequence
  Synthetic oligonucleotide
gaccctcaggccatgaggactgg

SEQ ID NO: 1169 - DNA, 20 bp, Artificial Sequence
  Synthetic oligonucleotide
gtttcttttcaggctgaaga

SEQ ID NO: 1170 - DNA, 23 bp, Artificial Sequence
  Synthetic oligonucleotide
gtttcttttcaggctgaagatgg

SEQ ID NO: 1171 - DNA, 20 bp, Artificial Sequence
  Synthetic oligonucleotide
gggcgtcccagcacgacgac

SEQ ID NO: 1172 - DNA, 23 bp, Artificial Sequence
  Synthetic oligonucleotide
gggcgtcccagcacgacgacagg

SEQ ID NO: 1173 - DNA, 20 bp, Artificial Sequence
  Synthetic oligonucleotide
ggtgagtgagtgtgtgcgtg

SEQ ID NO: 1174 - DNA, 23 bp, Artificial Sequence
  Synthetic oligonucleotide
ggtgagtgagtgtgtgcgtgtgg

SEQ ID NO: 1175 - DNA, 20 bp, Artificial Sequence
  Synthetic oligonucleotide
ggagagggaccgcgccacct

SEQ ID NO: 1176 - DNA, 23 bp, Artificial Sequence
  Synthetic oligonucleotide
ggagagggaccgcgccaccttgg

SEQ ID NO: 1177 - DNA, 20 bp, Artificial Sequence
  Synthetic oligonucleotide
gaacttacgcaggagatatt

SEQ ID NO: 1178 - DNA, 23 bp, Artificial Sequence
  Synthetic oligonucleotide
gaacttacgcaggagatattcgg

SEQ ID NO: 1179 - DNA, 20 bp, Artificial Sequence
  Synthetic oligonucleotide
gttccggtaagcatagacaa

SEQ ID NO: 1180 - DNA, 23 bp, Artificial Sequence
  Synthetic oligonucleotide
gttccggtaagcatagacaatgg

SEQ ID NO: 1181 - DNA, 20 bp, Artificial Sequence
  Synthetic oligonucleotide
gagacccgctcttcagcatg

SEQ ID NO: 1182 - DNA, 23 bp, Artificial Sequence
  Synthetic oligonucleotide
gagacccgctcttcagcatgtgg

SEQ ID NO: 1183 - DNA, 20 bp, Artificial Sequence
  Synthetic oligonucleotide
gagagggccccaagacctcg

SEQ ID NO: 1184 - DNA, 23 bp, Artificial Sequence
  Synthetic oligonucleotide
gagagggccccaagacctcgtgg

SEQ ID NO: 1185 - DNA, 20 bp, Artificial Sequence
  Synthetic oligonucleotide
gcgccttattccagtgacaa

SEQ ID NO: 1186 - DNA, 23 bp, Artificial Sequence
  Synthetic oligonucleotide
gcgccttattccagtgacaaagg

SEQ ID NO: 1187 - DNA, 20 bp, Artificial Sequence
  Synthetic oligonucleotide
gcagatcctcatcaccgcgc

SEQ ID NO: 1188 - DNA, 23 bp, Artificial Sequence
  Synthetic oligonucleotide
gcagatcctcatcaccgcgctgg

SEQ ID NO: 1189 - DNA, 20 bp, Artificial Sequence
  Synthetic oligonucleotide
gcatgtcgacttctcctcgg

SEQ ID NO: 1190 - DNA, 23 bp, Artificial Sequence
  Synthetic oligonucleotide
gcatgtcgacttctcctcggagg

SEQ ID NO: 1191 - DNA, 20 bp, Artificial Sequence
  Synthetic oligonucleotide
gctggtctaggacgtcctcc

SEQ ID NO: 1192 - DNA, 23 bp, Artificial Sequence
  Synthetic oligonucleotide
gctggtctaggacgtcctccagg

SEQ ID NO: 1193 - DNA, 20 bp, Artificial Sequence
  Synthetic oligonucleotide
ggaaactcaccgatccatac

SEQ ID NO: 1194 - DNA, 23 bp, Artificial Sequence
  Synthetic oligonucleotide
ggaaactcaccgatccatacagg

SEQ ID NO: 1195 - DNA, 20 bp, Artificial Sequence
  Synthetic oligonucleotide
gccagcaaagcacattattt

SEQ ID NO: 1196 - DNA, 23 bp, Artificial Sequence
  Synthetic oligonucleotide
gccagcaaagcacattattttgg

SEQ ID NO: 1197 - DNA, 20 bp, Artificial Sequence
  Synthetic oligonucleotide
ggcccgtctccgtgctcctc

SEQ ID NO: 1198 - DNA, 23 bp, Artificial Sequence
  Synthetic oligonucleotide
ggcccgtctccgtgctcctctgg

SEQ ID NO: 1199 - DNA, 20 bp, Artificial Sequence
  Synthetic oligonucleotide
gcgctacgacgagatcgtca

SEQ ID NO: 1200 - DNA, 23 bp, Artificial Sequence
  Synthetic oligonucleotide
gcgctacgacgagatcgtcaagg

SEQ ID NO: 1201 - DNA, 20 bp, Artificial Sequence
  Synthetic oligonucleotide
gagaataagattcagttgca

SEQ ID NO: 1202 - DNA, 23 bp, Artificial Sequence
  Synthetic oligonucleotide
gagaataagattcagttgcaagg

SEQ ID NO: 1203 - DNA, 20 bp, Artificial Sequence
  Synthetic oligonucleotide
gtttcttgggatccaccacc

SEQ ID NO: 1204 - DNA, 23 bp, Artificial Sequence
  Synthetic oligonucleotide
gtttcttgggatccaccaccagg

SEQ ID NO: 1205 - DNA, 20 bp, Artificial Sequence
  Synthetic oligonucleotide
gtaggtgggctgccgaagat

SEQ ID NO: 1206 - DNA, 23 bp, Artificial Sequence
  Synthetic oligonucleotide
gtaggtgggctgccgaagatagg

SEQ ID NO: 1207 - DNA, 20 bp, Artificial Sequence
  Synthetic oligonucleotide
gtaattaggtaaggcttagt

SEQ ID NO: 1208 - DNA, 23 bp, Artificial Sequence
  Synthetic oligonucleotide
gtaattaggtaaggcttagttgg

SEQ ID NO: 1209 - DNA, 20 bp, Artificial Sequence
  Synthetic oligonucleotide
gctctttgaccgcgcgcgtg

SEQ ID NO: 1210 - DNA, 23 bp, Artificial Sequence
  Synthetic oligonucleotide
gctctttgaccgcgcgcgtgtgg

SEQ ID NO: 1211 - DNA, 20 bp, Artificial Sequence
  Synthetic oligonucleotide
gaaacagctcgttgtaccgc

SEQ ID NO: 1212 - DNA, 23 bp, Artificial Sequence
  Synthetic oligonucleotide
gaaacagctcgttgtaccgctgg

SEQ ID NO: 1213 - DNA, 20 bp, Artificial Sequence
  Synthetic oligonucleotide
gcaacaacgtgatgaatatc

SEQ ID NO: 1214 - DNA, 23 bp, Artificial Sequence
  Synthetic oligonucleotide
gcaacaacgtgatgaatatctgg

SEQ ID NO: 1215 - DNA, 20 bp, Artificial Sequence
  Synthetic oligonucleotide
gtcgctgtgactttctaatt

SEQ ID NO: 1216 - DNA, 23 bp, Artificial Sequence
  Synthetic oligonucleotide
gtcgctgtgactttctaatttgg

SEQ ID NO: 1217 - DNA, 20 bp, Artificial Sequence
  Synthetic oligonucleotide
ggtgttatctctgaagcgca

SEQ ID NO: 1218 - DNA, 23 bp, Artificial Sequence
  Synthetic oligonucleotide
ggtgttatctctgaagcgcatgg

SEQ ID NO: 1219 - DNA, 20 bp, Artificial Sequence
  Synthetic oligonucleotide
ggatcatggaagccagctcc

SEQ ID NO: 1220 - DNA, 23 bp, Artificial Sequence
  Synthetic oligonucleotide
ggatcatggaagccagctccagg

SEQ ID NO: 1221 - DNA, 20 bp, Artificial Sequence
  Synthetic oligonucleotide
ggtctcggcacttggctcgc

SEQ ID NO: 1222 - DNA, 23 bp, Artificial Sequence
  Synthetic oligonucleotide
ggtctcggcacttggctcgctgg

SEQ ID NO: 1223 - DNA, 20 bp, Artificial Sequence
  Synthetic oligonucleotide
ggaggcttcacagcgccctc

SEQ ID NO: 1224 - DNA, 23 bp, Artificial Sequence
  Synthetic oligonucleotide
ggaggcttcacagcgccctctgg

SEQ ID NO: 1225 - DNA, 20 bp, Artificial Sequence
  Synthetic oligonucleotide
gctagtaccttgtatgaaga

SEQ ID NO: 1226 - DNA, 23 bp, Artificial Sequence
  Synthetic oligonucleotide
gctagtaccttgtatgaagatgg

SEQ ID NO: 1227 - DNA, 20 bp, Artificial Sequence
  Synthetic oligonucleotide
gacgggaaagtcagtgtgaa

SEQ ID NO: 1228 - DNA, 23 bp, Artificial Sequence
  Synthetic oligonucleotide
gacgggaaagtcagtgtgaatgg

SEQ ID NO: 1229 - DNA, 20 bp, Artificial Sequence
  Synthetic oligonucleotide
gttcgaccatgcccttgctt

SEQ ID NO: 1230 - DNA, 23 bp, Artificial Sequence
  Synthetic oligonucleotide
gttcgaccatgcccttgcttagg

SEQ ID NO: 1231 - DNA, 20 bp, Artificial Sequence
  Synthetic oligonucleotide
ggcagagctcacctgtagat

SEQ ID NO: 1232 - DNA, 23 bp, Artificial Sequence
  Synthetic oligonucleotide
ggcagagctcacctgtagatagg

SEQ ID NO: 1233 - DNA, 20 bp, Artificial Sequence
  Synthetic oligonucleotide
gctggtggagctcttctcaa

SEQ ID NO: 1234 - DNA, 23 bp, Artificial Sequence
  Synthetic oligonucleotide
gctggtggagctcttctcaatgg

SEQ ID NO: 1235 - DNA, 20 bp, Artificial Sequence
  Synthetic oligonucleotide
ggagctgacccaagacgttc

SEQ ID NO: 1236 - DNA, 23 bp, Artificial Sequence
  Synthetic oligonucleotide
ggagctgacccaagacgttctgg

SEQ ID NO: 1237 - DNA, 20 bp, Artificial Sequence
  Synthetic oligonucleotide
gttgaccatcagattgagac

SEQ ID NO: 1238 - DNA, 23 bp, Artificial Sequence
  Synthetic oligonucleotide
gttgaccatcagattgagacagg

SEQ ID NO: 1239 - DNA, 20 bp, Artificial Sequence
  Synthetic oligonucleotide
gctcacctcgtgtccgttgc

SEQ ID NO: 1240 - DNA, 23 bp, Artificial Sequence
  Synthetic oligonucleotide
gctcacctcgtgtccgttgctgg

SEQ ID NO: 1241 - DNA, 20 bp, Artificial Sequence
  Synthetic oligonucleotide
ggacgttcatgtatttgctt

SEQ ID NO: 1242 - DNA, 23 bp, Artificial Sequence
  Synthetic oligonucleotide
ggacgttcatgtatttgctttgg

SEQ ID NO: 1243 - DNA, 20 bp, Artificial Sequence
  Synthetic oligonucleotide
gctgtaaaagacatccctga

SEQ ID NO: 1244 - DNA, 23 bp, Artificial Sequence
  Synthetic oligonucleotide
gctgtaaaagacatccctgatgg

SEQ ID NO: 1245 - DNA, 20 bp, Artificial Sequence
  Synthetic oligonucleotide
gggtctccaccacttcgtaa

SEQ ID NO: 1246 - DNA, 23 bp, Artificial Sequence
  Synthetic oligonucleotide
gggtctccaccacttcgtaaagg

SEQ ID NO: 1247 - DNA, 20 bp, Artificial Sequence
  Synthetic oligonucleotide
ggcggcgctcacaattgcca

SEQ ID NO: 1248 - DNA, 23 bp, Artificial Sequence
  Synthetic oligonucleotide
ggcggcgctcacaattgccacgg

SEQ ID NO: 1249 - DNA, 20 bp, Artificial Sequence
  Synthetic oligonucleotide
ggtacttactgttactcgca

SEQ ID NO: 1250 - DNA, 23 bp, Artificial Sequence
  Synthetic oligonucleotide
ggtacttactgttactcgcaagg

SEQ ID NO: 1251 - DNA, 20 bp, Artificial Sequence
  Synthetic oligonucleotide
gacgctggtcaaacgccttg

SEQ ID NO: 1252 - DNA, 23 bp, Artificial Sequence
  Synthetic oligonucleotide
gacgctggtcaaacgccttgcgg

SEQ ID NO: 1253 - DNA, 20 bp, Artificial Sequence
  Synthetic oligonucleotide
ggcatgctgcggcatgagat

SEQ ID NO: 1254 - DNA, 23 bp, Artificial Sequence
  Synthetic oligonucleotide
ggcatgctgcggcatgagatagg

SEQ ID NO: 1255 - DNA, 20 bp, Artificial Sequence
  Synthetic oligonucleotide
ggctccacgcagacgctgac

SEQ ID NO: 1256 - DNA, 23 bp, Artificial Sequence
  Synthetic oligonucleotide
ggctccacgcagacgctgacagg

SEQ ID NO: 1257 - DNA, 20 bp, Artificial Sequence
  Synthetic oligonucleotide
gtcgaggagaatgaggaaaa

SEQ ID NO: 1258 - DNA, 23 bp, Artificial Sequence
  Synthetic oligonucleotide
gtcgaggagaatgaggaaaatgg

SEQ ID NO: 1259 - DNA, 20 bp, Artificial Sequence
  Synthetic oligonucleotide
gcagattctctctgctcact

SEQ ID NO: 1260 - DNA, 23 bp, Artificial Sequence
  Synthetic oligonucleotide
gcagattctctctgctcacttgg

SEQ ID NO: 1261 - DNA, 20 bp, Artificial Sequence
  Synthetic oligonucleotide
gatggtacaggctcactcgc

SEQ ID NO: 1262 - DNA, 23 bp, Artificial Sequence
  Synthetic oligonucleotide
gatggtacaggctcactcgcagg

SEQ ID NO: 1263 - DNA, 20 bp, Artificial Sequence
  Synthetic oligonucleotide
gcacccaaatgttgaggtac

SEQ ID NO: 1264 - DNA, 23 bp, Artificial Sequence
  Synthetic oligonucleotide
gcacccaaatgttgaggtacagg

SEQ ID NO: 1265 - DNA, 20 bp, Artificial Sequence
  Synthetic oligonucleotide
gtcatcgaactgctcttagc

SEQ ID NO: 1266 - DNA, 23 bp, Artificial Sequence
  Synthetic oligonucleotide
gtcatcgaactgctcttagctgg

SEQ ID NO: 1267 - DNA, 20 bp, Artificial Sequence
  Synthetic oligonucleotide
gcctgaccatcgagaagtcc

SEQ ID NO: 1268 - DNA, 23 bp, Artificial Sequence
  Synthetic oligonucleotide
gcctgaccatcgagaagtcctgg

SEQ ID NO: 1269 - DNA, 20 bp, Artificial Sequence
  Synthetic oligonucleotide
ggacgatgacatgctcaagc

SEQ ID NO: 1270 - DNA, 23 bp, Artificial Sequence
  Synthetic oligonucleotide
ggacgatgacatgctcaagctgg

SEQ ID NO: 1271 - DNA, 20 bp, Artificial Sequence
  Synthetic oligonucleotide
gcgcgtgcatgtttgccgcc

SEQ ID NO: 1272 - DNA, 23 bp, Artificial Sequence
  Synthetic oligonucleotide
gcgcgtgcatgtttgccgcccgg

SEQ ID NO: 1273 - DNA, 20 bp, Artificial Sequence
  Synthetic oligonucleotide
ggcactgcggctggaggtgg

SEQ ID NO: 1274 - DNA, 23 bp, Artificial Sequence
  Synthetic oligonucleotide
ggcactgcggctggaggtggggg

SEQ ID NO: 1275 - DNA, 20 bp, Artificial Sequence
  Synthetic oligonucleotide
gctgcagaagggattccatg

SEQ ID NO: 1276 - DNA, 23 bp, Artificial Sequence
  Synthetic oligonucleotide
gctgcagaagggattccatgagg

SEQ ID NO: 1277 - DNA, 20 bp, Artificial Sequence
  Synthetic oligonucleotide
gcgagtcttcactgagtgta

SEQ ID NO: 1278 - DNA, 23 bp, Artificial Sequence
  Synthetic oligonucleotide
gcgagtcttcactgagtgtaagg

SEQ ID NO: 1279 - DNA, 20 bp, Artificial Sequence
  Synthetic oligonucleotide
gagtccgagcagaagaagaa

SEQ ID NO: 1280 - DNA, 23 bp, Artificial Sequence
  Synthetic oligonucleotide
gagtccgagcagaagaagaaggg

SEQ ID NO: 1281 - DNA, 20 bp, Artificial Sequence
  Synthetic oligonucleotide
ggtgagtgagtgtgtgcgtg

SEQ ID NO: 1282 - DNA, 23 bp, Artificial Sequence
  Synthetic oligonucleotide
ggtgagtgagtgtgtgcgtgtgg

SEQ ID NO: 1283 - DNA, 20 bp, Artificial Sequence
  Synthetic oligonucleotide
gtcatcttagtcattacctg

SEQ ID NO: 1284 - DNA, 23 bp, Artificial Sequence
  Synthetic oligonucleotide
gtcatcttagtcattacctgagg

SEQ ID NO: 1285 - DNA, 20 bp, Artificial Sequence
  Synthetic oligonucleotide
gaccccctccaccccgcctc

SEQ ID NO: 1286 - DNA, 23 bp, Artificial Sequence
  Synthetic oligonucleotide
gaccccctccaccccgcctccgg

SEQ ID NO: 1287 - DNA, 20 bp, Artificial Sequence
  Synthetic oligonucleotide
ggcccagactgagcacgtga

SEQ ID NO: 1288 - DNA, 71 bp, Artificial Sequence
  Synthetic
tggaggaagcagggcttcctttcctctgccatcacttatcgtcgtcatccttgtaatccgtgctcagtctg

SEQ ID NO: 1289 - DNA, 20 bp, Artificial Sequence
  Synthetic oligonucleotide
ggtgccagaaacaggggtga

SEQ ID NO: 1290 - DNA, 87 bp, Artificial Sequence
  Synthetic
gtgcctgctaaggactagttctgccctccagtcaggcttgtcgacgacggcggtctccgtcgtcaggatcatcccctgtttctggca

SEQ ID NO: 1291 - DNA, 21 bp, Artificial Sequence
  Synthetic oligonucleotide
gtgctccagaggccccccttg

SEQ ID NO: 1292 - DNA, 87 bp, Artificial Sequence
  Synthetic
gtgctgtagcctgccctctgcacctcctcaccaaggcttgtcgacgacggcggtctccgtcgtcaggatcatggggggcctctggag

SEQ ID NO: 1293 - DNA, 26 bp, Artificial Sequence
  Synthetic oligonucleotide
  misc_feature (1)..(23): n is a, c, g, or t
nnnnnnnnnnnnnnnnnnnnnnnaac

SEQ ID NO: 1294 - protein, 16 aa, Artificial Sequence
  Synthetic oligopeptide
KRPAATKKAGQAKKKK

SEQ ID NO: 1295 - protein, 7 aa, Simian virus 40
PKKKRKV

SEQ ID NO: 1296 - protein, 22 aa, Artificial Sequence
  Synthetic oligopeptide
DYKDHDGDYKDHDIDYKDDDDK

SEQ ID NO: 1297 - protein, 286 aa, Artificial Sequence
  Synthetic
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 1298 - DNA, 8214 bp, Artificial Sequence
  Synthetic
ccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgcctgcagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagatcacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccatgttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaaataccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtct

SEQ ID NO: 1299 - DNA, 4101 bp, Artificial Sequence
  Synthetic
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgcctgcagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagatcacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccatgttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaaataccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 1300 - protein, 1367 aa, Artificial Sequence
  Synthetic
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFACRNFMQLIHDDSLTFKEDIQKAQVSGQGDSLHEHIANLAGSPAIKKGILQTVKVVDELVKVMGRHKPENIVIEMARENQITQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAMFIKRQLVETRQITKHVAQILDSRMNTKYDENDKLIREVKVITLKSKLVSDFRKDFQFYKVREINKYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD